(19) United States
(12) Patent Application Publication
(10) Pub. No.: US 2022/0144812 A1
(43) Pub. Date: May 12, 2022

(54) QUINOLINE DERIVATIVES AND THEIR USE FOR THE TREATMENT OF CANCER

(71) Applicant: Epizyme, Inc., Cambridgre, MA (US)

(72) Inventors: Eamon Comer, Natick, MA (US); Kenneth W. Duncan, Westwood, MA (US); Alexis Cocozaki, Medford, MA (US); John Campbell, Cambridge, MA (US); Darren Harvey, Acton, MA (US); Michael Munchhof, Corvallis, MT (US)

(73) Assignee: Epizyme, Inc., Cambridgre, MA (US)

(21) Appl. No.: 17/598,707

(22) PCT Filed: Mar. 27, 2020
(86) PCT No.: PCT/US2020/025160

§ 371 Date: Sep. 27, 2021

Related U.S. Application Data
(60) Provisional application No. 62/952,599, filed on Dec. 23, 2019.
(60) Provisional application No. 62/825,507, filed on Mar. 28, 2019.

(51) Int. Cl. C07D 403/14; C07D 403/12; C07D 413/12; C07D 471/04; C07D 401/14; C07D 401/12

(52) U.S. Cl. CPC C07D 403/14; C07D 403/12; C07D 401/12; C07D 471/04; C07D 401/14; C07D 413/12

(57) ABSTRACT

The present disclosure provides novel compounds, compositions comprising the compounds and methods of use thereof.

### CROSS-REFERENCES TO RELATED APPLICATIONS

This application claims the benefit of U.S. Provisional Patent Application No. 62/825,507, filed Mar. 28, 2019, and U.S. Provisional Patent Application No. 62/952,599 filed Dec. 23, 2019, each of which is incorporated herein by reference in its entirety.

### BACKGROUND

There is a need to develop improved therapies for the treatment proliferative disorders, such as cancer.

### SUMMARY

In one aspect, the present disclosure provides a compound of Formula (I):

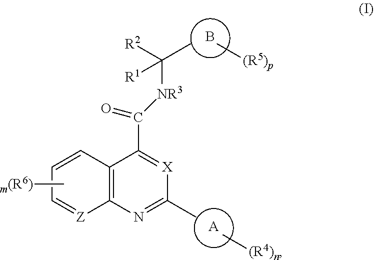

or a pharmaceutically acceptable salt thereof, wherein:

X is CH or N;

Z is N, CH, or CR⁶;

Ring A is a monocyclic or bicyclic aryl or a monocyclic or bicyclic heterocyclyl;

Ring B is a 5-membered N-containing heteroaryl;

R¹ and R² are each independently selected from H, C₁₋₆alkyl, halo, —CN, —C(O)R^{1a}, —C(O)₂R^{1a}, —C(O)N(R^{1a})₂, —N(R^{1a})₂, —N(R^{1a})C(O)R^{1a}, —N(R^{1a})C(O)₂R^{1a}, —N(R^{1a})C(O)N(R^{1a})₂, —N(R^{1a})S(O)₂R^{1a}, —OC(O)R^{1a}, —OC(O)N(R^{1a})₂, —SR^{1a}, —S(O)R^{1a}, —S(O)₂R^{1a}, —S(O)N(R^{1a})₂, and —S(O)₂N(R^{1a})₂;

R^{1a} in each occurrence is independently selected from H, C₁₋₆alkyl, C₂₋₆alkenyl, C₂₋₆alkynyl, carbocyclyl, and heterocyclyl, or two R^{1a} together with the nitrogen atom from which they are attached form a 4 to 7-membered ring, wherein the 4 to 7-membered ring optionally contains 1 or 2 heteroatoms independently selected from N, O, and S;

R³ is H or C₁₋₆alkyl;

R⁴ in each occurrence is independently selected from C₁₋₆alkyl, C₂₋₆alkenyl, C₂₋₆alkynyl, carbocyclyl, heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OC(O)R^{4a}, —OC(O)N(R^{4a})₂, —SR^{4a}, —S(O)R^{4a}, —S(O)₂R^{4a}, —S(O)N(R^{4a})₂, —S(O)₂N(R^{4a})₂, and P(O)(R^{4a})₂;

R^{4a} in each occurrence is independently selected from H, C₁₋₆alkyl, C₂₋₆alkenyl, C₂₋₆alkynyl, carbocyclyl, heterocyclyl, and P(O)(R^{7a})₂ or two R^{4a} together with the nitrogen atom from which they are attached form a 4 to 7-membered ring, wherein the 4 to 7-membered ring optionally contains 1 or 2 heteroatoms independently selected from N, O and S;

R⁵ in each occurrence is independently C₁₋₆alkyl or carbocyclyl, or two R⁵ together with the atoms from which they are attached form a 4 to 7-membered ring, optionally contains 1 or 2 heteroatoms independently selected from N, O and S;

R⁶ in each occurrence is independently selected from C₁₋₆alkyl, C₂₋₆alkenyl, C₂₋₆alkynyl, carbocyclyl, heterocyclyl, halo, —CN, —C(O)R^{6a}, —C(O)₂R^{6a}, —C(O)N(R^{6a})₂, —N(R^{6a})₂, —N(R^{6a})C(O)R^{6a}, —N(R^{6a})C(O)₂R^{6a}, —N(R^{6a})C(O)N(R^{6a})₂, —N(R^{6a})S(O)₂R^{6a}, —OC(O)R^{6a}, —OC(O)N(R^{6a})₂, —SR^{6a}, —S(O)R^{6a}, —S(O)₂R^{6a}, —S(O)N(R^{6a})₂, —S(O)₂N(R^{6a})₂, and —P(O)(R^{6a})₂;

R^{6a} in each occurrence is independently selected from H, C₁₋₆alkyl, C₂₋₆alkenyl, C₂₋₆alkynyl, carbocyclyl, and heterocyclyl; or two R^{6a} together with the nitrogen atom from which they are attached form a 4 to 7-membered ring, wherein the 4 to 7-membered ring optionally contains 1 or 2 heteroatoms independently selected from N, O, and S;

m is 0, 1, 2, or 3;

p is 0, 1, 2 or 3; and

n is 0, 1, 2, 3, 4, 5, or 6;

wherein each C₁₋₆alkyl, C₂₋₆alkenyl, C₂₋₆alkynyl, carbocyclyl, and heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, —SR⁷, —S(O)R⁷, —S(O)₂R⁷, —S(O)N(R⁷)₂, —S(O)₂N(R⁷)₂, and —P(O)(R⁷)₂, and

R⁷ in each occurrence is independently selected from H, C₁₋₆alkyl, C₂₋₆alkenyl, C₂₋₆alkynyl, carbocyclyl, and heterocyclyl, wherein each C₁₋₆alkyl, C₂₋₆alkenyl, C₂₋₆alkynyl, carbocyclyl, and heterocyclyl are optionally substituted with one or more substituents independently selected from R^{7a}, halo, —CN, —C(O)R^{7a}, —C(O)₂R^{7a}, —C(O)N(R^{7a}—)₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)R^{7a}; and

R^{7a} in each occurrence is independently selected from H and C₁₋₄alkyl.

Also provided in the present disclosure is a pharmaceutical composition comprising a compound described herein and a pharmaceutically acceptable carrier or excipient.

The present disclosure also provides a method of treating proliferative disorders (e.g., cancer) in a subject in need thereof comprising administering to the subject a therapeutically effective amount of a compound described herein.

### DETAILED DESCRIPTION

In one aspect, the present disclosure provides compounds or pharmaceutically acceptable salts thereof as described herein. In one embodiment, the compounds or pharmaceutically acceptable salts thereof as described herein, can have activities that are useful for treating proliferative disorders, such as cancer.

In some embodiments, the compounds or pharmaceutically acceptable salts thereof as described herein, are CREBBP and/or EP300 inhibitors (or antagonists).

In one embodiment, the present disclosure provides any one of the compounds disclosed as described herein as a neutral compound or a pharmaceutically acceptable salt thereof.

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

### Definitions

As used herein, the term “alkyl” refers to a fully saturated branched or unbranched hydrocarbon moiety. Preferably the alkyl comprises 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl.

“Alkenyl” refers to an unsaturated hydrocarbon group which may be linear or branched and has at least one carbon-carbon double bond. Alkenyl groups with 2-6 carbon atoms can be preferred. The alkenyl group may contain 1, 2 or 3 carbon-carbon double bonds, or more. Examples of alkenyl groups include ethenyl, n-propenyl, iso-propenyl, n-but-2-enyl, n-hex-3-enyl and the like.

“Alkynyl” refers to an unsaturated hydrocarbon group which may be linear or branched and has at least one carbon-carbon triple bond. Alkynyl groups with 2-6 carbon atoms can be preferred. The alkynyl group may contain 1, 2 or 3 carbon-carbon triple bonds, or more. Examples of alkynyl groups include ethynyl, n-propynyl, n-but-2-ynyl, n-hex-3-ynyl and the like.

The number of carbon atoms in a group is specified herein by the prefix “Cₓ₋ₓₓ”, wherein x and xx are integers. For example, “C₁₋₆alkyl” is an alkyl group which has from 1 to 6 carbon atoms.

“Alkoxy” used herein refers to alkyl-O-, wherein alkyl is defined herein above. Examples of alkoxy include, not are not limited to, methoxy, ethoxy, propoxy, 2-propoxy, butoxy, tert-butoxy, pentyloxy, hexyloxy, and the like.

“Halogen” or “halo” may be fluoro, chloro, bromo or iodo.

As used herein, the term “heterocyclyl” or “heterocycle” refers to a saturated or unsaturated, monocyclic or bicyclic (e.g., fused, bridged or spiro ring systems) ring system which has from 3- to 11-ring members, or in particular 3- to 10-ring members, 3- to 8-ring members, 3- to 7-ring members, 3- to 6-ring members, 4- to 6-ring members, 5- to 7-ring members, 5- to 6-ring members or 4- to 7-ring members, at least one of which is a heteroatom, and up to 4 (e.g., 1, 2, 3, or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. Unsaturated heterocyclic rings include heteroaryl rings.

As used herein, the term “heteroaryl” refers to an aromatic 5- or 6-membered monocyclic ring system or 9- or 10-membered bicyclic ring system, having 1 to 4 heteroatoms independently selected from 0, S and N, and wherein N can be oxidized (e.g., N(O)) or quaternized, and S can be optionally oxidized to sulfoxide and sulfone. Examples of heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, benzotriazole, benzoimidazole, indole, indazole, quinoline, isoquinoline, quinazoline, phthalazine, cinnoline, purine, and pteridine. In one embodiment, the heteroaryl is an aromatic 5- or 6-membered monocyclic ring system. Examples of 5- or 6-membered heteroaryl include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyrazinyl, pyrimidinyl, or triazinyl. As used herein, a “5-membered N-containing heteroaryl” is a 5-membered heteroaryl having at least one nitrogen ring atom. In one embodiment, a 5-membered N-containing heteroaryl may contain one or more heteroatoms other than nitrogen, wherein the heteroatoms other than nitrogen are independently selected from O and S. Non-limiting examples of 5-membered N-containing heteroaryls include pyrrolyl, imidazolyl, pyrazolyl, triazolyl, tetrazolyl, thiazolyl, thiadiazolyl, dithiazolyl, oxadiazolyl, and isoxazole

In one embodiment, a heterocyclyl is a 4-to 7-membered saturated monocyclic or a 4-to 6-membered saturated monocyclic or a 5-to 7-membered saturated monocyclic ring or a 9- to 11-membered or 9- to 10-membered saturated or partially saturated bicyclic ring. In one embodiment, a heterocyclyl is a 4- to 7-membered saturated monocyclic ring. In another embodiment, a heterocyclyl is a 9- to 10-membered bicyclic ring, in which one of ring is aromatic and the other one is non-aromatic. The heterocyclyl group can be attached at a heteroatom or a carbon atom. Examples of heterocyclyls include, but are not limited to, aziridinyl, oxiranyl, thiiranyl, oxaziridinyl, azetidinyl, oxetanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, dihydropyranyl.

The term “fused ring system”, as used herein, is a ring system that has two rings each of which are independently selected from a carbocyclyl or a heterocyclyl, wherein the two ring structures share two adjacent ring atoms. A fused ring system may have from 9 to 12 ring members.

In another embodiment, a heterocyclyl is a saturated 4- to 7-membered monocyclic heterocyclyl. Examples of saturated 4- to 7-membered monocyclic heterocyclic ring systems include, but are not limited to azetidinyl, oxetanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithiinyl, azepanyl, diazepanyl.

In another embodiment, a heterocyclyl is pyridine, benzotriazole, benzoimidazole, thiazole, pyrrole, pyrazole, indole, imidazole, isoxazole, isothiazole, pyrrolidine, piperidine, piperazine, pyrimidine, triazole, 1H-indazole, 2H-indazole, 1,4-diazepane, 4,5,6,7-tetrahydro-1H-imidazo[4,5-c]pyridine, 4,5,6,7-tetrahydro-1H-pyrazolo[3,4-c]pyridine, 4,5,6,7-tetrahydro-2H-pyrazolo[3,4-c]pyridine, 4,5,6,7-tetrahydro-1H-imidazo[4,5-c]pyridine, 5,6,7,8-tetrahydro-[1,2,4]triazolo[1,5-a]pyrazine, 5,6,7,8-tetrahydroimidazo[1,2-a]pyrazine, pyrazole, azetidine, pyrrolidine or morpholine.

As used herein, the term “carbocyclyl” refers to saturated or unsaturated monocyclic or bicyclic hydrocarbon groups of 3-12, 3-7, 3-5, 3-6, 4-6, or 5-7 carbon atoms. The term “carbocyclyl” encompasses cycloalkyl groups and aromatic groups. The term “cycloalkyl” refers to completely saturated monocyclic or bicyclic or spiro hydrocarbon groups of 3-7 carbon atoms, 3-6 carbon atoms, or 5-7 carbon atoms. Exemplary monocyclic carbocyclyl groups include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl, phenyl and cycloheptatrienyl. Exemplary bicyclic carbocyclyl groups include bicyclo[2.1.1]hexyl, bicyclo[2.2.1]heptyl, bicyclo[2.2.1]heptenyl, tricyclo[2.2.1.0^{2,6}]heptanyl, 6,6-[3.1.1]heptyl, or 2,6,6-trimethylbicyclo[3.1.1]heptyl, spiro[2.2]pentanyl, and spiro[3.3]heptanyl. In one embodiment, the carbocyclyl is a 4- to 6-membered monocyclic carbocyclyl. In another embodiment, the carbocyclyl is a C₃₋₅cycloalkyl, such as cyclopropyl, cyclobutyl, or cyclopentyl. In one embodiment, the carbocyclyl is a C₄₋₆ cycloalkyl, such as, cyclobutyl, cyclopentyl or cyclohexyl.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl.

As described herein, compounds of the disclosure may, when specified, contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one sub stituent selected from a specified group, the substituent may be either the same or different at every position. As used herein, “one or more substituents” refers to one, two, three, four or more hydrogens of the designated moiety are replaced with a suitable substituents. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Suitable substituents on a substitutable carbon atom of an “optionally substituted” group are independently halogen; —CN; —C(O)R°, —C(O)₂R°, —C(O)N(R°)₂, —N(R°)₂, —N(R°)C(O)R°, —N(R°)C(O)₂R°, —N(R°)C(O)N(R°)₂, —N(R°)S(O)₂R°, —OR°, —OC(O)R°, —OC(O)N(R°)₂, —S(O)₂R°, —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°)₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR—, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; —OP(O)(OR°)₂; SiR°₃; —(C₁₋₄ straight or branched)alkylene)O—N(R°)₂; or —(C₁₋₄ straight or branched alkylene)C(O)O—N(R°)₂, wherein each R° may be substituted as defined below and is independently hydrogen, C₁₋₆ aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66,1-19, incorporated herein by reference.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl azxervmines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase). When a particular enantiomer of a compound used in the disclosed methods is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “pharmaceutical composition” refers to a composition that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to show a statistically significant probability of achieving a desired therapeutic effect when administered to a subject or population in need thereof. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, a pharmaceutical composition is intended and suitable for administration to a human subject. In some embodiments, a pharmaceutical composition is sterile and substantially pyrogen-free.

As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CIVIL), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In one embodiment, the cancer exhibits a CREBBP loss of function mutation. In another embodiment, the cancer exhibits an EP300 loss of function mutation. In another embodiment, the cancer exhibits a CREBBP loss of function mutation and an EP300 loss of function mutation. In another embodiment, the cancer exhibits a CREBBP loss of function mutation and does not exhibit an EP300 loss of function mutation. In another embodiment, the cancer exhibits an EP300 loss of function mutation and does not exhibit a CREBBP loss of function mutation. In another embodiment, the cancer does not exhibit a CREBBP loss of function mutation or an EP300 loss of function mutation.

As used herein, the term “therapeutically effective amount” refers to an amount that produces a desired effect (e.g., a desired biological, clinical, or pharmacological effect) in a subject or population to which it is administered. In some embodiments, the term refers to an amount statistically likely to achieve the desired effect when administered to a subject in accordance with a particular dosing regimen (e.g., a therapeutic dosing regimen). In some embodiments, the term refers to an amount sufficient to produce the effect in at least a significant percentage (e.g., at least about 25%, about 30%, about 40%, about 50%, about 60%, about 70%, about 80%, about 90%, about 95%, or more) of a population that is suffering from and/or susceptible to a disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be an amount that provides a particular desired response in a significant number of subjects when administered to patients in need of such treatment, e.g., in at least about 25%, about 30%, about 40%, about 50%, about 60%, about 70%, about 80%, about 90%, about 95%, or more patients within a treated patient population. In some embodiments, reference to a therapeutically effective amount may be a reference to an amount sufficient to induce a desired effect as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor. In one embodiment, the tumor exhibits a CREBBP loss of function mutation. In another embodiment, the tumor exhibits an EP300 loss of function mutation. In another embodiment, the tumor exhibits a CREBBP loss of function mutation and an EP300 loss of function mutation. In another embodiment, the tumor exhibits a CREBBP loss of function mutation and does not exhibit an EP300 loss of function mutation. In another embodiment, the tumor exhibits an EP300 loss of function mutation and does not exhibit a CREBBP loss of function mutation. In another embodiment, the tumor does not exhibit a CREBBP loss of function mutation or an EP300 loss of function mutation.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

As used herein, the term “loss of function mutation” means a mutation that results in a protein (gene product) having less function or activity relative to the wild-type protein, or no function or activity at all. In one embodiment, a loss of function mutation results in a truncatedprotein . In one embodiment, a loss of function mutation results in a full length defective protein. In all above embodiments, a loss of function mutation can significantly diminish protein expression. In addition, in some embodiments, a loss of function mutation can resultin complete loss of protein

As used herein, the term “loss of function” means a protein (gene product) having less function or activity relative to the wild-type gene, or no function or activity at all.

### Compounds

In a first embodiment, the present disclosure provides a compound of formula (I):

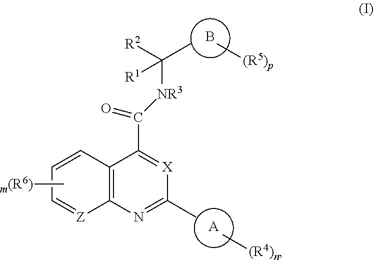

or a pharmaceutically acceptable salt thereof, wherein:

X is CH or N;

Z is N, CH, or CR⁶;

Ring A is a monocyclic or bicyclic aryl or a monocyclic or bicyclic heterocyclyl;

Ring B is a 5-membered N-containing heteroaryl;

R¹ and R² are each independently selected from H, C₁₋₆alkyl, halo, —CN, —C(O)R^{1a}, —C(O)₂R^{1a}, —C(O)N(R^{1a})₂, —N(R^{1a})₂, —N(R^{1a})C(O)R^{1a}, —N(R^{1a})C(O)₂R^{1a}, —N(R^{1a})C(O)N(R^{1a})₂, —N(R^{1a})S(O)₂R^{1a}, —OR^{1a}, —OC(O)R^{1a}, —OC(O)N(R^{1a})₂, —S(O)R^{1a}, —S(O)₂R^{1a}, —S(O)N(R^{1a})₂, and —S(O)₂N(R^{1a})₂;

R^{1a} in each occurrence is independently selected from H, C₁₋₆alkyl, C₂₋₆alkenyl, C₂₋₆alkynyl, carbocyclyl, and heterocyclyl, or two R^{1a} together with the nitrogen atom from which they are attached form a 4 to 7-membered ring, wherein the 4 to 7-membered ring optionally contains 1 or 2 heteroatoms independently selected from N, O, and S;

R³ is H or C₁₋₆alkyl;

R⁴ in each occurrence is independently selected from C₁₋₆alkyl, C₂₋₆alkenyl, C₂₋₆alkynyl, carbocyclyl, heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OC(O)R^{4a}, —OC(O)N(R^{4a})₂, —SR^{4a}, —S(O)R^{4a}, —S(O)₂R^{4a}, —S(O)N(R^{4a})₂, —S(O)₂N(R^{4a})₂, and P(O)(R^{4a})₂;

R^{4a} in each occurrence is independently selected from H, C₁₋₆alkyl, C₂₋₆alkenyl, C₂₋₆alkynyl, carbocyclyl, heterocyclyl, and P(O)(R^{7a})₂, or two R^{4a} together with the nitrogen atom from which they are attached form a 4 to 7-membered ring, wherein the 4 to 7-membered ring optionally contains 1 or 2 heteroatoms independently selected from N, O and S;

R⁵ in each occurrence is independently C₁₋₆alkyl or carbocyclyl, or two R⁵ together with the atoms from which they are attached form a 4 to 7-membered ring, wherein the 4 to 7-membered ring optionally contains 1 or 2 heteroatoms independently selected from N, O and S;

R⁶ in each occurrence is independently selected from C₁₋₆alkyl, C₂₋₆alkenyl, C₂₋₆alkynyl, carbocyclyl, heterocyclyl, halo, —CN, —C(O)R^{6a}, —C(O)₂R^{6a}, —C(O)N(R^{6a})₂, —N(R^{6a})₂, —N(R^{6a})C(O)R^{6a}, —N(R^{6a})C(O)₂R^{6a}, —N(R^{6a})C(O)N(R^{6a})₂, —N(R^{6a})S(O)₂R^{6a}, —OR^{6a}, —OC(O)R^{6a}, —OC(O)N(R^{6a})₂, —SR^{6a}, —S(O)R^{6a}, —S(O)₂R^{6a}, —S(O)N(R^{6a})₂, —S(O)₂N(R^{6a})₂, and —P(O)(R^{6a})₂;

R^{6a} in each occurrence is independently selected from H, C₁₋₆alkyl, C₂₋₆alkenyl, C₂₋₆alkynyl, carbocyclyl, and heterocyclyl; or two R^{6a} together with the nitrogen atom from which they are attached form a 4 to 7-membered ring, wherein the 4 to 7-membered ring optionally contains 1 or 2 heteroatoms independently selected from N, O, and S;

m is 0, 1, 2, or 3;

p is 0, 1, 2 or 3; and

n is 0, 1, 2, 3, 4, 5, or 6;

wherein each C₁₋₆alkyl, C₂₋₆alkenyl, C₂₋₆alkynyl, carbocyclyl, and heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, —SR⁷, —S(O)R⁷, —S(O)₂R⁷, —S(O)N(R⁷)₂, —S(O)₂N(R⁷)₂, and —P(O)(R⁷)₂, and

R⁷ in each occurrence is independently selected from H, C₁₋₆alkyl, C₂₋₆alkenyl, C₂₋₆alkynyl, carbocyclyl, and heterocyclyl, wherein each C₁₋₆alkyl, C₂₋₆alkenyl, C₂₋₆alkynyl, carbocyclyl, and heterocyclyl are optionally substituted with one or more substituents independently selected from R^{7a}, halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)R^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)R^{7a}; and

R^{7a} in each occurrence is independently selected from H and C₁₋₄alkyl. In one embodiment, R⁷ in each occurrence is independently selected from H, C₁₋₆alkyl, C₂₋₆alkenyl, C₂₋₆alkynyl, carbocyclyl, and heterocyclyl, wherein each C₁₋₆alkyl, C₂₋₆alkenyl, C₂₋₆alkynyl, carbocyclyl, and heterocyclyl are optionally substituted with one or more substituents independently selected from halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)R^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)R^{7a}.

In a second embodiment, for compounds of formula (I), or a pharmaceutically acceptable salt thereof, X is N and Z is N; and the remaining variables are as defined in the first embodiment. In yet another embodiment, X is CH and Z is CH or CR⁶.

In a third embodiment, for compounds of formula (I) or a pharmaceutically acceptable salt thereof, only one of X and Z is N, and the remaining variables are as defined in the first embodiment. In yet another embodiment, X is CH and Z is N.

In a fourth embodiment, the compound of the present disclosure is represented by the following formula:

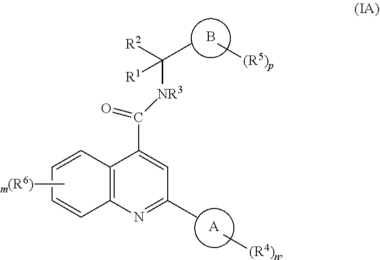

or a pharmaceutically acceptable salt thereof, wherein the variables are as defined in the first embodiment.

In a fifth embodiment, the compound of the present disclosure is represented by the following formula:

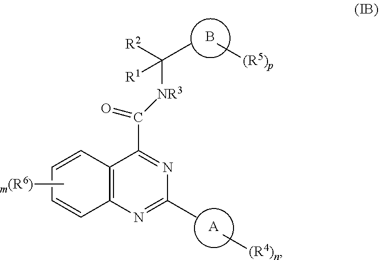

or a pharmaceutically acceptable salt thereof, wherein the variables are as defined in the first embodiment.

In a sixth embodiment, the compound of the present disclosure is represented by the following formula:

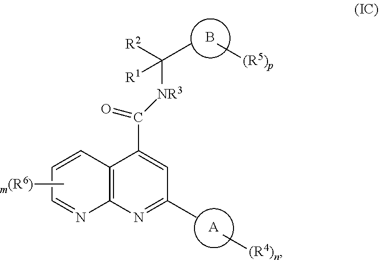

or a pharmaceutically acceptable salt thereof, wherein the variables are as defined in the first embodiment.

In a seventh embodiment, for compounds of formula (I), (IA), (IB) or (IC), or a pharmaceutically acceptable salt thereof, Ring B is a N-containing heteroaryl including one nitrogen atom, and the remaining variables are as defined in the first, second, third, fourth, fifth or sixth embodiment.

In an eighth embodiment, for compounds of formula (I), (IA), (IB) or (IC), or a pharmaceutically acceptable salt thereof, Ring B is a N-containing heteroaryl including two nitrogen atoms, and the remaining variables are as defined in the first, second, third, fourth, fifth or sixth embodiment.

In a ninth embodiment, for compounds of formula (I), (IA), (IB) or (IC), or a pharmaceutically acceptable salt thereof, Ring B is pyrrole, pyrazole, imidazole, oxazole, isoxazole, thiazole or isothiazole, and the remaining variables are as defined in the first, second, third, fourth, fifth or sixth embodiment.

In a tenth embodiment, for compounds of formula (I), (IA), (IB) or (IC), or a pharmaceutically acceptable salt thereof, Ring B is pyrazole or imidazole, and the remaining variables are as defined in the first, second, third, fourth, fifth or sixth embodiment.

In an eleventh embodiment, for compounds of formula (I), (IA), (IB) or (IC), or a pharmaceutically acceptable salt thereof, Ring B is pyrazole, and the remaining variables are as defined in the first, second, third, fourth, fifth or sixth embodiment.

In a twelfth embodiment, for compounds of formula (I), (IA), (IB) or (IC), or a pharmaceutically acceptable salt thereof, Ring B is imidazole, and the remaining variables are as defined in the first, second, third, fourth, fifth or sixth embodiment.

In a thirteenth embodiment, for compounds of formula (I), (IA), (IB) or (IC), or a pharmaceutically acceptable salt thereof, R¹ and R² are each independently selected from H, C₁₋₆alkyl, and halo, and the remaining variables are as defined in the first, second, third, fourth, fifth, sixth, seventh, eighth, ninth, tenth, eleventh or twelfth embodiment.

In a fourteenth embodiment, for compounds of formula (I), (IA), (IB) or (IC), or a pharmaceutically acceptable salt thereof, R¹ is H and R² is C₁₋₆alkyl or halo, and the remaining variables are as defined in the first, second, third, fourth, fifth, sixth, seventh, eighth, ninth, tenth, eleventh, twelfth or thirteenth embodiment.

In a fifteenth embodiment, for compounds of formula (I), (IA), (IB) or (IC), or a pharmaceutically acceptable salt thereof, R¹ and R² are both H, and the remaining variables are as defined in the first, second, third, fourth, fifth, sixth, seventh, eighth, ninth, tenth, eleventh, twelfth or thirteenth embodiment.

In a sixteenth embodiment, for compounds of formula (I), (IA), (IB) or (IC), or a pharmaceutically acceptable salt thereof, R¹ and R² are both H, and R³ is methyl, and the remaining variables are as defined in the first, second, third, fourth, fifth, sixth, seventh, eighth, ninth, tenth, eleventh, twelfth, thirteenth, fourteenth or fifteenth embodiment.

In a seventeenth embodiment, the compound is represented by the following formula:

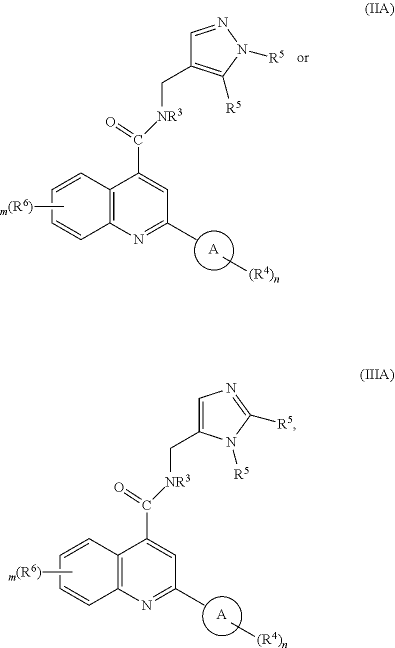

or a pharmaceutically acceptable salt thereof, wherein the variables are as defined in the first, second, third, fourth, fifth, sixth, eighth, ninth, tenth, eleventh, twelfth, thirteenth, fourteenth, fifteenth or sixteenth embodiment.

In an eighteenth embodiment, the compound is represented by the following formula:

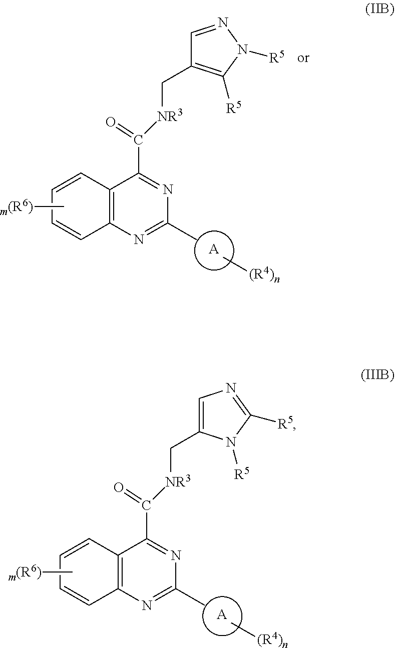

or a pharmaceutically acceptable salt thereof, wherein the variables are as defined in the first, second, third, fourth, fifth, sixth, eighth, ninth, tenth, eleventh, twelfth, thirteenth, fourteenth, fifteenth or sixteenth embodiment.

In a nineteenth embodiment, the compound is represented by the following formula:

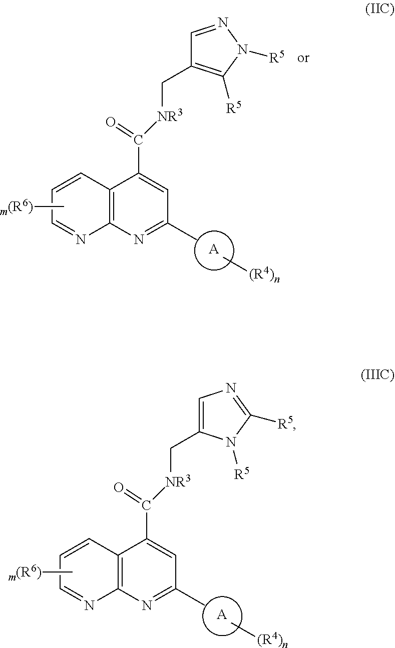

or a pharmaceutically acceptable salt thereof, wherein the variables are as defined in the first, second, third, fourth, fifth, sixth, eighth, ninth, tenth, eleventh, twelfth, thirteenth, fourteenth, fifteenth or sixteenth embodiment.

In a twentieth embodiment, the compound is represented by the following formula:

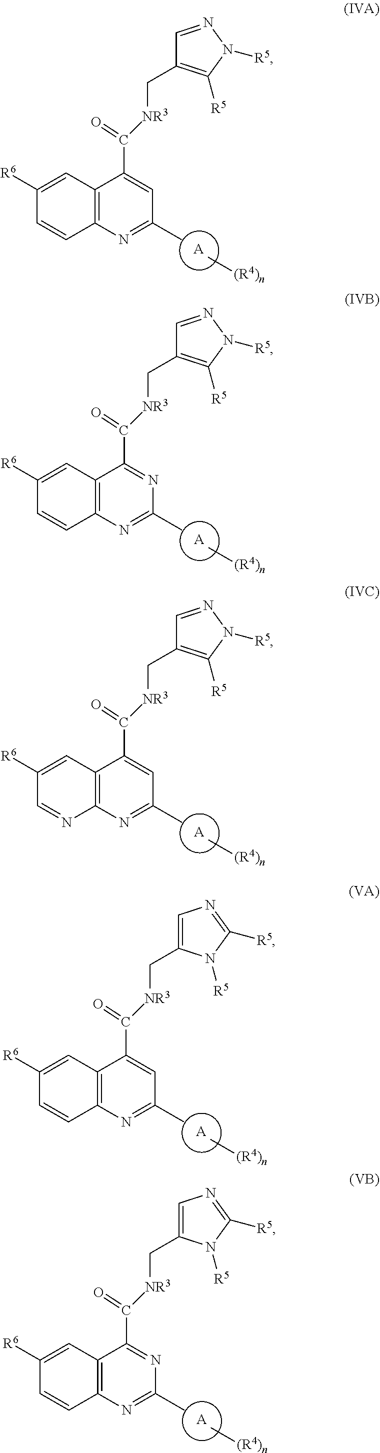

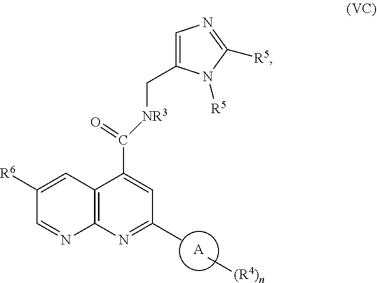

or a pharmaceutically acceptable salt thereof, wherein the variables are as defined in the first, second, third, fourth, fifth, sixth, eighth, ninth, tenth, eleventh, twelfth, thirteenth, fourteenth, fifteenth or sixteenth embodiment.

In a twenty-first embodiment, the compound is represented by the following formula:

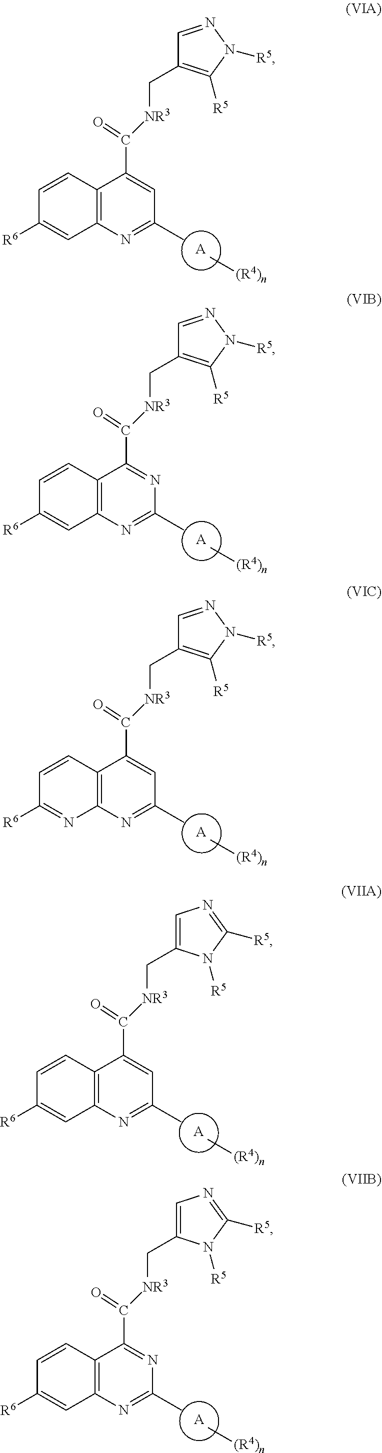

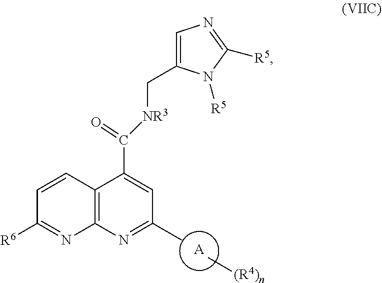

or a pharmaceutically acceptable salt thereof, wherein the variables are as defined in the first, second, third, fourth, fifth, sixth, eighth, ninth, tenth, eleventh, twelfth, thirteenth, fourteenth, fifteenth or sixteenth embodiment.

In a twenty-second embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ in each occurrence is independently selected from C₁₋₆alkyl, phenyl, 4 to 6-membered heterocyclyl, halo, —CN, —OR^{6a}, —N(R^{6a})₂, —S(O)₂R^{6a}, and —P(O)(R^{6a})₂; and

R^{6a} in each occurrence is independently selected from H and C₁₋₆alkyl;

wherein each of the C₁₋₆alkyl, phenyl and 5 to 6-membered heterocyclyl are optionally substituted with one or more substituents independently selected from halo, —N(R⁷)₂, —OR⁷ and phenyl optionally substituted with one or more substituents independently selected from —CN, halo, and —OR^{7a};

R⁷ is H or C₁₋₄alkyl; and

R^{7a} in each occurrence is independently selected from H and C₁₋₄alkyl, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-third embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ is Cl, Br, F, —CN, —OCH₃, —CH₃, —CH₂CH₃, —OCH₂CH₃, —NH₂, —NHCH₃, —N(CH₃)₂, —C₂H₄NHCH₃, —OCH₂CH(OH)CH₂NHCH₃, morpholine, or —CH₂OCH₃, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or twenty-second embodiment.

In a twenty-fourth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ is —OR^{6a}, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-fifth embodiment, or compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VITA), (VIIB) or (VIIC), or a pharmaceutically acceptable salt thereof, R^{6a} is C₁₋₆alkyl, and the remaining variables are as defined in the twenty-fourth embodiment.

In a twenty-sixth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ is C₁₋₆alkyl substituted with —OR⁷, wherein R⁷ is H or C₁₋₆alkyl, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-seventh embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ is halogen, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-eighth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ is fluoro, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a twenty-ninth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁶ is chloro, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment.

In a thirtieth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R³ is H or C₁₋₆alkyl optionally substituted with halo, —OR⁷, or —N(R⁷)₂; and R⁷ is H or C₁₋₃alkyl, and the remaining variables are as defined in any of the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first, twenty-second, twenty-third, twenty-fourth, twenty-fifth, twenty-sixth, twenty-seventh, twenty-eight, or twenty-ninth embodiment.

In a thirty-first embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R³ is C₁₋₃alkyl optionally substituted with halo, —OH or C₁₋₃alkoxy, and the remaining variables are as defined the thirtieth embodiment.

In a thirty-second embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R³ is H, methyl, ethyl, —CH₂CH₂OH, and the remaining variables are as defined in the thirtieth embodiments.

In a thirty-third embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R³ methyl or ethyl, and the remaining variables are as defined in the thirtieth embodiment.

In a thirty-fourth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁵ in each occurrence is independently selected from C₁₋₄alkyl and C₃₋₆cycloalkyl, wherein each of the C₁₋₄alkyl and C₃₋₆cycloalkyl are optionally substituted with one to three halogen, and the remaining variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first, twenty-second, twenty-third, twenty-fourth, twenty-fifth, twenty-sixth, twenty-seventh, twenty-eight, twenty-ninth, thirtieth, thirty-first, thirty-second, or thirty-third embodiment.

In a thirty-fifth embodiment, for compounds of formula (I), (IA), (IB), (IC), (IIA), (IIB), (ITC), (IIIA), (IIIB), (IIIC), (IVA), (IVB), (IVC), (VA), (VB), (VC), (VIA), (VIB), (VIC), (VITA), (VIIB) or (VIIC), or a pharmaceutically acceptable salt thereof, R⁵ in each occurrence is independently selected from methyl, ethyl, propyl, isopropyl, cyclopropyl and —CH₂CF₃, and the remaining variables are as defined in the thirty-fourth embodiment.

In a thirty-sixth embodiment, for compounds of formula (I), (IA), (IB), (IC), (IIA), (IIB), (ITC), (IIIA), (IIIB), (IIIC), (IVA), (IVB), (IVC), (VA), (VB), (VC), (VIA), (VIB), (VIC), (VITA), (VIIB) or (VIIC), or a pharmaceutically acceptable salt thereof, R⁵ in each occurrence is independently C₁₋₄alkyl, and the remaining variables are as defined in thirty-fourth embodiment.

In a thirty-seventh embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof,

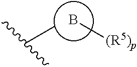

has the structure

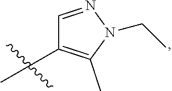 [SMILES: *c1cnn(CC)c1C]

and the remaining variables are as defined in the first, second, third, fourth, fifth, sixth, seventh, eighth, ninth, tenth, eleventh, twelfth, thirteenth, fourteenth, fifteenth, sixteenth, twenty-second, twenty-third, twenty-fourth, twenty-fifth, twenty-sixth, twenty-seventh, twenty-eight, twenty-ninth, thirtieth, thirty-first, thirty-second, or thirty-third, thirty-fourth, thirty-fifth, thirty-sixth embodiment.

In a thirty-eighth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof,

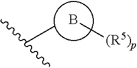

has the structure

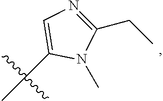 [SMILES: *c1cnc(CC)n1C]

and the remaining variables are as defined in the first, second, third, fourth, fifth, sixth, seventh eighth, ninth, tenth, eleventh, twelfth, thirteenth, fourteenth, fifteenth, sixteenth, twenty-second, twenty-third, twenty-fourth, twenty-fifth, twenty-sixth, twenty-seventh, twenty-eight, twenty-ninth, thirtieth, thirty-first, thirty-second, thirty-third, thirty-fourth, thirty-fifth or thirty-sixth embodiment.

In a thirty-ninth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R¹ and R² are both H; R³ is methyl; and

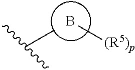

has the structure

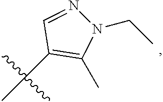 [SMILES: *c1cnn(CC)c1C]

and the remaining variables are as defined in the first, second, third, fourth, fifth, sixth, seventh, eighth, ninth, tenth, eleventh, twelfth, thirteenth, fourteenth, fifteenth, sixteenth, twenty-second, twenty-third, twenty-fourth, twenty-fifth, twenty-sixth, twenty-seventh, twenty-eight, twenty-ninth, thirtieth, thirty-first, thirty-second, thirty-third, thirty-fourth, thirty-fifth or thirty-sixth embodiment.

In a fortieth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R¹ and R² are both H; R³ is methyl;

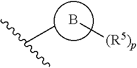

has the structure

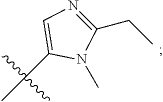 [SMILES: *c1cnc(CC)n1C]

and the remaining variables are as defined in the first, second, third, fourth, fifth, sixth, seventh, eighth, ninth, tenth, eleventh, twelfth, thirteenth, fourteenth, fifteenth, sixteenth, twenty-second, twenty-third, twenty-fourth, twenty-fifth, twenty-sixth, twenty-seventh, twenty-eight, twenty-ninth, thirtieth, thirty-first, thirty-second, thirty-third, thirty-fourth, thirty-fifth or thirty-sixth embodiment.

In a forty-first embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, m is 0, and the remaining variables are as defined in any one of the first to fortieth embodiments. In yet another embodiment, m is 1. In yet another embodiment, m is 2. In yet another embodiment, m is 3. In yet another embodiment, p is 0. In yet another embodiment, p is 1. In yet another embodiment, p is 2. In yet another embodiment, p is 3. The remaining variables are as defined in any one of the above embodiments.

In a forty-second embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, Ring A is phenyl, 5 or 6-membered heteroaryl, 9 or 10-membered bicyclic heteroaryl, 5 to 7-membered saturated monocyclic heterocyclyl, or 9- and 10-membered bicyclic non-aromatic heterocyclyl, and the remaining variables are as defined in any one of the first to forty-first embodiments.

In a forty-third embodiment, for compounds of formula (I), (IA), (IB), (IC), (IIA), (IIB), (ITC), (IIIA), (IIIB), (IIIC), (IVA), (IVB), (IVC), (VA), (VB), (VC), (VIA), (VIB), (VIC), (VITA), (VIIB) or (VIIC), or a pharmaceutically acceptable salt thereof, Ring A is phenyl or 5- or 6-membered heteroaryl, and the remaining variables are as defined in any one of the first to forty-second embodiments.

In a forty-fourth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, Ring A is phenyl, pyridine, benzotriazole, benzoimidazole, thiazole, pyrrole, pyrazole, indole, imidazole, isoxazole, isothiazole, pyrrolidine, piperidine, piperazine, pyrimidine, triazole, 1H-indazole, 2H-indazole, 1,4-diazepane, 4,5,6,7-tetrahydro-1H-imidazo[4,5-c]pyridine, 4,5,6,7-tetrahydro-1H-pyrazolo[3,4-c]pyridine, 4,5,6,7-tetrahydro-2H-pyrazolo[3,4-c]pyridine, 4,5,6,7-tetrahydro-1H-imidazo[4,5-c]pyridine, 5,6,7,8-tetrahydro-[1,2,4]triazolo[1,5-a]pyrazine, or 5,6,7,8-tetrahydroimidazo[1,2-a]pyrazine, and the remaining variables are as defined in any of the first to forty-third embodiments.

In a forty fifth embodiment, for compounds of formula (I), (IA), (IB), (IC), (IIA), (IIB), (ITC), (IIIA), (IIIB), (IIIC), (IVA), (IVB), (IVC), (VA), (VB), (VC), (VIA), (VIB), (VIC), (VIIA), (VIM) or (VIIC), or a pharmaceutically acceptable salt thereof, Ring A is:

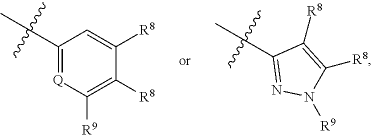

wherein R⁸ in each occurrence is independently selected from C₁₋₆alkyl, C₂₋₆alkenyl, C₂₋₆alkynyl, carbocyclyl, heterocyclyl, halo, —CN, —C(O)R^{8a}, —C(O)₂R^{8a}, —C(O)N(R^{8a})₂, —N(R^{8a})₂, —N(R^{8a})C(O)R^{8a}, —N(R^{8a})C(O)₂R^{8a}, —N(R^{8a})C(O)N(R^{8a})₂, —N(R^{8a})S(O)₂R^{8a}, —OC(O)R^{8a}, —OC(O)N(R^{8a})₂, —SR^{8a}, —S(O)R^{8a}, —S(O)₂R^{8a}, —S(O)N(R^{8a})₂, and —S(O)₂N(R^{8a})₂; or two R⁸ together with the carbon atoms from which they are attached form a 4 to 7-membered ring, wherein the 4 to 7-membered ring optionally contains 1, 2 or 3 heteroatoms independently selected from N, O, and S;

R^{8a} is in each occurrence is independently selected from H, C₁₋₆alkyl, C₂₋₆alkenyl, C₂₋₆alkynyl, carbocyclyl, and heterocyclyl, or two R^{8a} together with the nitrogen atom from which they are attached form a 4 to 7-membered ring, wherein the 4 to 7-membered ring optionally contains 1 or 2 heteroatoms independently selected from N, O and S;

R⁹ is selected from C₁₋₆alkyl, C₂₋₆alkenyl, C₂₋₆alkynyl, carbocyclyl, heterocyclyl, halo, —CN, —C(O)R^{9a}, —C(O)₂R^{9a}, —C(O)N(R^{9a})₂, —N(R^{9a})₂, —N(R^{9a})C(O)R^{9a}, —N(R^{9a})C(O)₂R^{9a}, —N(R^{9a})C(O)N(R^{9a})₂, —N(R^{9a})S(O)₂R^{9a}, —OR^{9a}, —OC(O)R^{9a}, —OC(O)N(R^{9a})₂, —SR^{9a}, —S(O)R^{9a}, —S(O)₂R^{9a}, —S(O)N(R^{9a})₂, —S(O)₂N(R^{9a})₂, and —P(O)(R^{9a})₂;

R^{9a} in each occurrence is independently selected from H, C₁₋₆alkyl, C₂₋₆alkenyl, C₂₋₆alkynyl, carbocyclyl, and heterocyclyl, or two R^{9a} together with the nitrogen atom from which they are attached form a 4 to 7-membered ring, wherein the 4 to 7-membered ring optionally contains 1 or 2 heteroatoms independently selected from N, O and S; and

Q is N, CH or CR⁸;

wherein each C₁₋₆alkyl, C₂₋₆alkenyl, C₂₋₆alkynyl, carbocyclyl, and heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, —SR⁷, —S(O)R⁷, —S(O)₂R⁷, —S(O)N(R⁷)₂, —S(O)₂N(R⁷)₂, and —P(O)(R⁷)₂, and the remaining variables are as defined in any of the first to forty-fourth embodiment.

In one embodiment, two R⁸ together with the carbon atoms from which they are attached form a 5 or 6-membered ring that is aromatic. In another embodiment, two R⁸ together with the carbon atoms from which they are attached form a 5 or 6-membered ring that is non-aromatic.

In a forty-sixth embodiment, for compounds of formula (I), (IA), (IB), (IC), (IIA), (IIB), (ITC), (IIIA), (IIIB), (IIIC), (IVA), (IVB), (IVC), (VA), (VB), (VC), (VIA), (VIB), (VIC), (VITA), (VIIB) or (VIIC), or a pharmaceutically acceptable salt thereof, R⁹ is methyl or halogen, and the remaining variables are as defined in the forty-fifth embodiment.

In a forty -seventh embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁹ is chloro, and the remaining variables are as defined in the forty-fifth embodiment.

In a forty-eighth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁴ in each occurrence is independently selected from C₁₋₆alkyl, C₃₋₆cycloalkyl, 5 to 6-membered heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OR^{4a}, —OC(O)R^{4a}, —OC(O)N(R^{4a})₂, and —S(O)₂R^{4a};

R^{4a} in each occurrence is independently selected from H, C₁₋₆alkyl, C₃₋₆cycloalkyl, and 5 to 6-membered heterocyclyl;

wherein each C₁₋₆alkyl, C₃₋₆cycloalkyl, and 5 to 6-membered heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, and —S(O)₂R⁷, and

R⁷ in each occurrence is independently selected from H, C₁₋₆alkyl, phenyl, C₃₋₆cycloalkyl, and 5 to 6-membered heterocyclyl, wherein each C₁₋₆alkyl, phenyl, C₃₋₆cycloalkyl, and 5 to 6-membered heterocyclyl are optionally substituted with one or more substituents independently selected from R^{7a}, halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)R^{7a}, —OC(O)N(R^{7a})₂ and —S(O)₂R^{7a}; and

R^{7a} in each occurrence is independently selected from H and C₁₋₄alkyl, and the remaining variables are as defined in any of the first to forty-seventh embodiments. In one embodiment, R⁷ in each occurrence is independently selected from H, C₁₋₆alkyl, C₂₋₆alkenyl, C₂₋₆alkynyl, carbocyclyl, and heterocyclyl, wherein each C₁₋₆alkyl, C₂₋₆alkenyl, C₂₋₆alkynyl, carbocyclyl, and heterocyclyl are optionally substituted with one or more substituents independently selected from halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)R^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)R^{7a}.

In a forty-ninth embodiment, for compounds of formula (I), (IA), (IB), (IC), (IIA), (IIB), (IIC), (IIIA), (IIIB), (IIIC), (IVA), (IVB), (IVC), (VA), (VB), (VC), (VIA), (VIB), (VIC), (VIIA), (VIIB) or (VIIC), or a pharmaceutically acceptable salt thereof, R⁴ in each occurrence is independently selected from H, Cl, F, Br, —CN, NH₂, —CH₃, —CH₂CH₃, —CF₃, —CH₂OH, —CH₂OCH₃, —CH₂NHCH₃, —CH₂N(CH₃)₂, —C₂H₄OCH₃, —C₂H₄NHCH₃, —C₃H₆OH, —CH₂—NH-tetrahydopyran, —C₃H₆NHCH₃, -cyclopropyl, pyrazole, azetidine, pyrrolidine, morpholine, —CH₂-pyrrolidine, —C₃H₆-pyrrolidine, —CH₂NH-tetrahydropyran, —CH₂-piperazine, —CH₂-morpholine, —CH₂-phenyl-OCH₃, —CH₂CH₂CN, —OCH₃, —OC₂H₄OH, —OC₃H₆OH, —OC₃H₆-piperidine, —OC₂H₄-pyrrolidine, —OC₃H₆-pyrrolidine, —OC₃H₆-tetrahydropyran, —OCH₂CH(OH)CH₂NHCH₃, —OC₂H₄OCH₃, —OC₂H₄NH₂, —OC₂H₄NHCH₃, —OC₃H₆NHCH₃, —OC₂H₄NHC(O)CH₃, —OC₂H₄N(CH₃)S(O)₂CH₃, —CH₂C(O)NH₂, —CH₂C(O)NHCH₃, —C(O)NHCH₃, —C(O)NHC₃H₆-pyrrolidine, —C(O)NHC₂H₄-pyrrolidine, —C(O)NH₂, —C(O)NHCH₃, —S(O)₂CH₃, —C(O)CH₃, —N(CH₃)₃, —NHC(O)CH₃, —NHCH₃, —NH-piperidine, —NHC₂H₄NHCH₃, —NHC₃H₆NHCH₃, —NHC(O)NHCH₃, —NHC(O)OC₄H₉, —NH(CO)CH₂NHCH₃, —NHC₂H₄N(CH₃)C(O)OC₄H₉, —C₂H₄NHCOOC₄H₉, —CH₂N(CH₃)C(O)OC₄H₉, —C₂H₄N(CH₃)C(O)OC₄H₉, —C₃H₆NHC(O)OC₄H₉, —C₃H₆N(CH₃)C(O)OC₄H₉, —OC₂H₄C(O)NHCH₃, —OC₂H₄NHC(O)OC₄H₉, —OC₂H₄N(CH₃)C(O)OC₄H₉, —OC₃H₆NHC(O)OC₄H₉, —OC₃H₆N(CH₃)C(O)OC₄H₉, —C(O)OC₄H₉, —C₃H₆-pyrrolidine, —CH₂CH₂CH(OH)CH₂-pyrrolidine, —NH-piperidine, —NH—(N-methyl)piperidine, —NH-tetrahydropyran, —OCH₂CH(OH)CH₂NHCH₃—OCH₂CH₂NHCH₃—CH₂CH₂CH(OH)CH₂NHCH₃, —C(O)NH-tetrahydropyridine, —C(O)NH-piperidine, 1-(4-methoxybenzyl), —C(O)NH—C₃H₆-pyrrolidine, —C(O)NH—C₂H₄-pyrrolidine, —O—Ph—CH₂N(CH₃)₂, pyrrolidine-C(O)OC₄H₉, —NH—C₂H₄-pyrrolidine, —OCH₂CH(OH)CH₂-pyrrolidine, —OCH₂CH₂-pyrrolidine, —CO—NH—N-(1-methylpiperidin-4-yl), —OCH₂CH(OH)CH₂-pyrrolidine, and

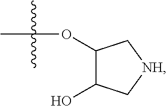 [SMILES: *OC1CNCC1O]

and the remaining variables are as defined in any of the first to forty-eighth embodiments.

In a fiftieth embodiment, the compound is represented by the following formula:

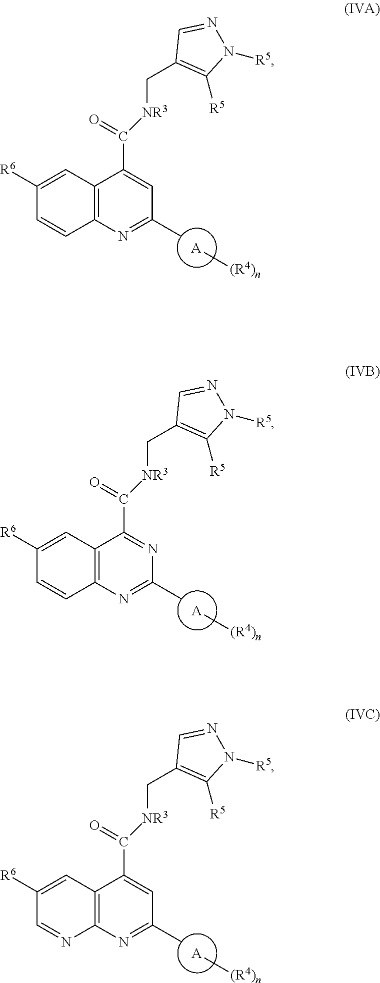

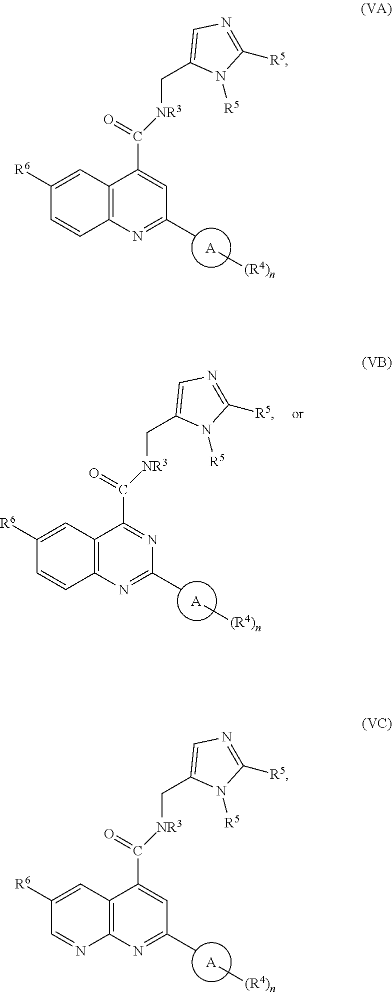

or a pharmaceutically acceptable salt thereof, wherein:

R³ is C₁₋₃alkyl optionally substituted with halo, —OH, or C₁₋₃alkoxy;

R⁵ in each occurrence is independently selected from C₁₋₄alkyl, and C₃₋₆cycloalkyl, wherein the C₁₋₄alkyl and C₃₋₆cycloalkyl are optionally substituted with one to three halogen;

R⁶ is halo, C₁₋₄alkyl, or 4 to 6-membered saturated heterocyclyl, wherein the C₁₋₄alkyl and 4 to 6-membered saturated heterocyclyl are optionally substituted with one or more substituents independently selected from halo, —OR⁷ and —N(R⁷)₂;

R⁷ is H or C₁₋₃alkyl;

Ring A is phenyl or 5 or 6-membered heteroaryl;

R⁴ in each occurrence is independently selected from C₁₋₆alkyl, C₃₋₆cycloalkyl, 5 to 6-membered heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OC(O)R^{4a}, —OC(O)N(R^{4a})₂, and —S(O)₂R^{4a};

R^{4a} in each occurrence is independently selected from H, C₁₋₆alkyl, C₃₋₆cycloalkyl, and 5 to 6-membered heterocyclyl;

wherein each C₁₋₆alkyl, C₃₋₆cycloalkyl, and 5 to 6-membered heterocyclyl above are optionally substituted with one or more substituents independently selected from R⁷, halo, —CN, —C(O)N(R⁷)₂, —N(R⁷)₂, —N(R⁷)C(O)R⁷, —N(R⁷)C(O)₂R⁷, —N(R⁷)S(O)₂R⁷, and —OR⁷, and

R⁷ in each occurrence is independently selected from H, C₁₋₆alkyl, phenyl, C₃₋₆cycloalkyl, and 5 to 6-membered heterocyclyl, wherein each C₁₋₆alkyl, phenyl, C₃₋₆cycloalkyl, and 5 to 6-membered heterocyclyl are optionally substituted with one or more substituents independently selected from R^{7a}, halo, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, and —OR^{7a};

R^{7a} in each occurrence is independently selected from H and C₁₋₄alkyl; and

n is 0, 1, or 2. In one embodiment, R⁷ in each occurrence is independently selected from H, C₁₋₆alkyl, C₂₋₆alkenyl, C₂₋₆alkynyl, carbocyclyl, and heterocyclyl, wherein each C₁₋₆alkyl, C₂₋₆alkenyl, C₂₋₆alkynyl, carbocyclyl, and heterocyclyl are optionally substituted with one or more substituents independently selected from halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OR^{7a}, —OC(O)R^{7a}, —OC(O)N(R^{7a})₂, —S(O)R^{7a}, —S(O)₂R^{7a}, —S(O)N(R^{7a})₂, —S(O)₂N(R^{7a})₂, and —P(O)R^{7a}.

In a fifty-first embodiment, the compound is represented by the following formula:

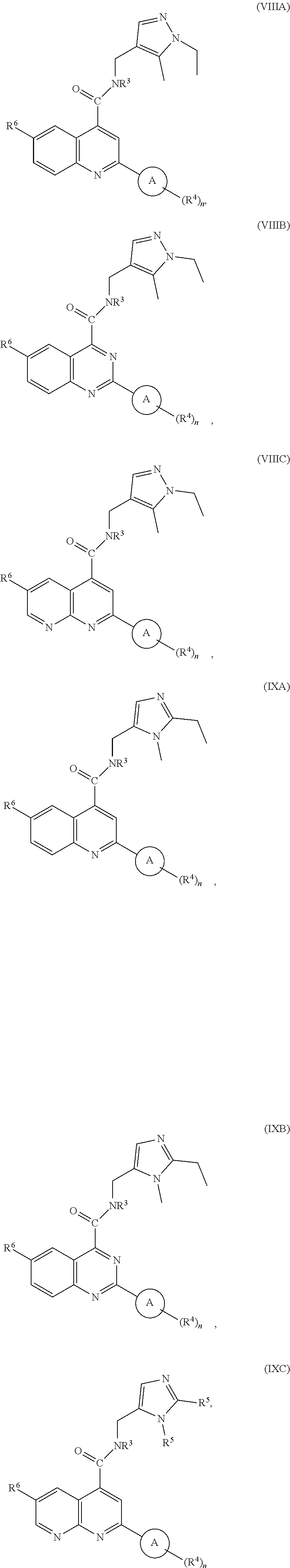

or a pharmaceutically acceptable salt thereof, and the remaining variables are as defined in the fiftieth embodiment.

In a fifty -second embodiment, for compounds of formula (IVA), (IVB), (IVC), (VA), (VB), (VC), (VIIIA), (VIIIC), (IXA), (IXB) or (IXC), or a pharmaceutically acceptable salt thereof, R³ is C₁₋₃alkyl; R⁵ in each occurrence is independently C₁₋₄alkyl; and R⁶ is halo, and the remaining values are as defined in fiftieth or fifty-first embodiment.

In a fifty-third embodiment, for compounds of formula (IVA), (IVB), (IVC), (VA), (VB), (VC), (VIIIA), (VIIIC), (IXA), (IXB) or (IXC), or a pharmaceutically acceptable salt thereof, R³ is methyl; R⁵ in each occurrence is independently methyl, ethyl or isopropyl; R⁶ is chloro, and the remaining values are as defined in fiftieth, fifty-first or fifty-second embodiment.

In a fifty-fourth embodiment, the present disclosure provides a pharmaceutically acceptable salt of compounds of any one of formulae (I), (IA), (IB), (IC), (IIA), (IIB), (IIC), (IIIA), (IIIC), (IVA), (IVB), (IVC), (VA), (VB), (VC), (VIA), (VIB), (VIC), (VITA), (VIIB) or (VIIC), (VIIIA), (VIIIB), (VIIIC), (IXA), (IXB) and (IXC), and the remaining values are as defined in any one of the first to fifty-third embodiments.

In a fifty-fifth embodiments, the present disclosure provides a compound as shown in Table 1, or a pharmaceutically acceptable salt thereof. In a fifty-sixth embodiment, the present disclosure provides a compound as shown in Table 2, or a pharmaceutically acceptable salt thereof In a fifty-seventh embodiment, the present disclosure provides a compound as shown in Table 3, or a pharmaceutically acceptable salt thereof.

### Methods of Treatment

In certain embodiments, the present disclosure provides methods and compositions useful in the treatment of cancer, e.g., for the treatment of a tumor in a subject.

In some embodiments, the cancer or tumor comprises a mutant EP300 sequence associated with a EP300 loss of function. In some embodiments, the cancer or tumor comprises a mutant CREBBP sequence associated with a CREBBP loss of function. In some embodiments, the cancer or tumor comprises a mutant CREBBP sequence and a mutant EP300 sequence associated with a CREBBP loss of function and EP300 loss of function. In some embodiments, the cancer or tumor comprises a mutant CREBBP sequence associated with a CREBBP loss of function and exhibits wild-type EP300 expression. In some embodiments, the cancer or tumor comprises a mutant EP300 sequence associated with a EP300 loss of function and exhibits wild-type CREBBP expression. In some embodiments, the cancer or tumor exhibits wild-type CREBBP expression and wild-type EP300 expression.

As will be known to those of ordinary skill in the art, CREB (cAMP responsive element binding protein) binding protein (CREBBP) and p300 (adenovirus E1A-associated 300-kD protein, also referred herein as EP300) are two closely related and evolutionary conserved histone acetyl transferases (HATs). CBP/EP300 function as transcriptional regulators by acetylating histone tails and other nuclear proteins. CREBBP and EP300 are also important regulators of RNA polymerase II-mediated transcription. Studies indicate that the ability of these multidomain proteins to acetylate histones and other proteins is critical for many biological processes. CREBBP and EP300 have been reported to interact with more than 400 different cellular proteins, including factors important to cancer development and progression such as hypoxia-inducible factors-1 (HIF-1), beta-catenin, c-Myc, c-Myb, CREB, E1, E6, p53, AR and estrogen receptor (ER). See, e.g., Kalkhoven et al., Biochemical Phamacology 2004, 68, 1145-1155; and Farria et al., Oncogene 2015, 34, 4901-4913. Genetic alterations in genes encoding CREBBP and EP300 and their functional inactivation have been linked to human disease. Furthermore, despite their high degree of homology, CREBBP and EP300 are not completely redundant but also have unique roles in cellular function. CREBBP and EP300 have been implicated in the process of DNA replication and DNA repair. CREBBP and EP300 have also been implicated in the regulation of cell cycle progression; ubiquitination and degradation of the transcription factor p53; and regulation of nuclear import. Due to these numerous roles, mutations in the gene or changes in the expression level, activity or localization of CREBBP or EP300 may result in a disease state. See, e.g., Vo et. al. J. Biol. Chem. 2001, 276(17), 13505-13508; and Chan et. al. Journal of Cell Science 2001, 114, 2363-2373, the entire contents of each of which are incorporated herein by reference. Diseases that may result from modulation of CREBBP or EP300 may include, but are not limited to, developmental disorders, for example Rubionstein-Taybi syndrome (RTS); progressive neurodegenerative diseases, e.g., Huntington Disease (HD), Kennedy Disease (spinal and bulbar muscular atrophy, SBMA); dentatorubral-pallidoluysian atrophy (DRPLA), Alzheimer's disease (AD) and 6 spinocerebellar ataxias (SCAs); and cancers. See, e.g., Iyer et al., Oncogene 2004, 23, 4225-4231; and Valor et al., Curr. Pharm. Des. 2013, 19(28), 5051-5064, the entire contents of each of which are incorporated herein by reference. High expression of EP300/CREBBP has been reported to be associated with various cancers. See WO 2018/022637, the entire contents of which are incorporated herein by reference.

In some embodiments, the compounds described herein may be used in the treatement of a cancer or tumor. In some embodiments, a cancer or tumor exhibiting a loss of function of EP300 is sensitive to compounds of the disclosure. In some embodiments, a cancer or tumor exhibiting a loss of function of CREBBP is sensitive to compounds of the disclosure. In some embodiments, a cancer or tumor exhibiting a loss of function of CREBBP and EP300 is sensitive to compounds of the disclosure. In some embodiments, the cancer or tumor is sensitive to treatment with a CREBBP inhibitor and the growth, proliferations, and/or survival of such mutant cancer cells can effectively be inhibited or abolished by contacting such cells with a CREBBP inhibitor in vitro or in vivo. In some embodiments, the cancer or tumor is sensitive to treatment with a EP300 inhibitor and the growth, proliferations, and/or survival of such mutant cancer cells can effectively be inhibited or abolished by contacting such cells with a EP300 inhibitor in vitro or in vivo. In some embodiments, the cancer or tumor is sensitive to treatment with a CREBBP and EP300 dual inhibitor and the growth, proliferations, and/or survival of such mutant cancer cells can effectively be inhibited or abolished by contacting such cells with a CREBBP and EP300 inhibitor in vitro or in vivo.

In some embodiments, a compound described herein is CREBBP inhibitor. In some embodiments, a compound described herein is a EP300 inhibitor. In some embodiments, a compound described herein is a CREBBP and EP300 inhibitor (“CREBBP and EP300 dual inhibitor”). Those of ordinary skill in the art will be able to determine whether a compound is a CREBBP inhibitor, an EP300 inhibitor, or CREBBP and EP300 dual inhibitor, for example, using the methods described in Example 3-6.

In some embodiments, administration of a compound described herein (e.g., a CREBBP inhibitor) decreases the activity of a CREBBP gene product. In some embodiments, methods are provided comprising administering a compound described herein (e.g., a CREBBP inhibitor) to a subject suffering from a cancer determined to harbor at least one mutation in EP300.

In some embodiments, administration of a compound described herein (e.g., a EP300 inhibitor) decreases the activity of a EP300 gene product. In some embodiments, administration of a compound described herein (e.g., a EP300 inhibitor) decreases the activity of a EP300 gene product. In some embodiments, methods are provided comprising administering a compound described herein (e.g., a EP300 inhibitor) to a subject suffering from a cancer determined to harbor at least one mutation in CREBBP.

In some embodiments, administration of a compound described herein (e.g., a CREBBP and EP300 inhibitor) decreases the activity of a CREBBP and EP300 gene products. In some embodiments, methods are provided comprising administering a compound described herein (e.g., a CREBBP and EP300 inhibitor) to a subject suffering from a cancer determined to harbor at least one mutation in CREBBP and/or EP300.

In some embodiments, the cancer or tumor exhibits an EP300 loss of function mutation. In some embodiments, the cancer or tumor exhibits a loss of function mutation as described herein. In some embodiments, the cancer or tumor exhibits an EP300 mutation that results in a EP300 truncated protein containing an EP300 HAT domain. In some embodiments, the cancer or tumor exhibits an EP300 mutation that results in an EP300 truncated protein without an EP300 HAT domain. In some embodiments, the cancer or tumor exhibits an EP300 mutation that results in a full length EP300 protein with a defective EP300 HAT domain. In all these cases, the mutations can also cause a significant reduction of protein expression or total loss of EP300 protein. In some embodiments, the cancer or tumor exhibits loss of wild-type EP300 expression. In some embodiments, the cancer or tumor comprises a mutant allele of EP300, e.g., an allele harboring a loss-of-function mutation of EP300, and exhibits loss of wild-type expression of EP300 protein. In some such embodiments, the cancer or tumor harbors a wild-type EP300 allele, but does not express wild-type EP300 from the wild-type allele. In some embodiments, the wild-type EP300 allele is silenced, e.g., via epigenetic mechanisms. In some embodiments, EP300 expression from the wild-type allele is decreased or abolished through transcriptional repression, or through post-transcriptional or post-translational mechanisms. In some embodiments, each EP300 allele of the cancer or tumor is affected by at least one EP300 loss of function mutation.

In some embodiments, the cancer or tumor exhibits a CREBBP loss of function mutation. In some embodiments, the cancer or tumor exhibits a loss of function mutation as described herein. In some embodiments, the cancer or tumor exhibits a CREBBP mutation that results in a CREBBP truncated protein containing a CREBBP HAT domain. In some embodiments, the cancer or tumor exhibits a CREBBP mutation that results in a CREBBP truncated protein without a CREBBP HAT domain. In some embodiments, the cancer or tumor exhibits a CREBBP mutation that results in a full length CREBBP protein with a defective CREBBP HAT domain. In all these cases, the mutations can also cause a significant reduction of protein expression or total loss of CREBBP protein. In some embodiments, the cancer or tumor exhibits loss of wild-type CREBBP expression. In some embodiments, the cancer or tumor comprises a mutant allele of CREBBP, e.g., an allele harboring a loss-of-function mutation of CREBBP, and exhibits loss of wild-type expression of CREBBP protein. In some such embodiments, the cancer or tumor harbors a wild-type CREBBP allele, but does not express wild-type CREBBP from the wild-type allele. In some embodiments, the wild-type CREBBP allele is silenced, e.g., via epigenetic mechanisms. In some embodiments, CREBBP expression from the wild-type allele is decreased or abolished through transcriptional repression, or through post-transcriptional or post-translational mechanisms. In some embodiments, each CREBBP allele of the cancer or tumor is affected by at least one CREBBP loss of function mutation.

In some embodiments, a cancer or tumor harboring a loss of function mutation in an EP300 gene is sensitive to treatment with CREBBP inhibitors. Accordingly, in some embodiments, the cancer or tumor treated with the compositions or according to the methods provided herein is an EP300 mutant cancer or tumor. In other embodiments, the cancer or tumor does not harbor an EP300 loss of function mutation. In some such embodiments, the cancer or tumor harbors an EP300 loss of function that is mediated by epigenetic mechanisms, e.g., by silencing of EP300, or by post-transcriptional and/or post-translational silencing.

In some embodiments, a cancer or tumor harboring a loss of function mutation in a CREBBP gene is sensitive to treatment with EP300 inhibitors. Accordingly, in some embodiments, the cancer or tumor treated with the compositions or according to the methods provided herein is an CREBBP mutant cancer or tumor. In other embodiments, the cancer or tumor does not harbor an CREBBP loss of function mutation. In some such embodiments, the cancer or tumor harbors a CREBBP loss of function that is mediated by epigenetic mechanisms, e.g., by silencing of CREBBP or by post-transcriptional and/or post-translational silencing.

In some particular embodiments, the present disclosure provides therapies for tumors with mutations in EP300, CREBBP, or EP300 and CREBBP. In some embodiments, methods and compositions of the present disclosure are not used in treatment of tumors harboring one or more particular CREBBP mutations, or EP300 mutations, or CREBBP and EP300 mutations. In some embodiments, methods and compositions of the present disclosure are not used in treatment of hematopoietic tumors deficient in CREBBP, in EP300, or EP300 and CREBBP. In some embodiments, methods and compositions of the present disclosure are used in treatment of hematopoietic tumors deficient in CREBBP, in EP300, or EP300 and CREBBP.

In some embodiments, the cancer or tumor exhibits an EP300 loss of function mutation, e.g., mediated by an EP300 loss of function mutation described herein, and may be sensitive to treatment with CREBBP inhibitors (or antagonist) of the present disclosure, and thus the cancer or tumor may be treated with the methods and compositions provided herein. In some embodiments, the cancer or tumor exhibits an EP300 loss of function mutation, e.g., mediated by an EP300 loss of function mutation described herein, and may be sensitive to treatment with a CREBBP and EP300 inhibitor (or antagonist) of the present disclosure, and thus the cancer or tumor may be treated with the methods and compositions provided herein.

In other embodiments, the cancer or tumor exhibits a CREBBP loss of function mutation, e.g., mediated by an CREBBP loss of function mutation, and may be sensitive to treatment with EP300 inhibitors (or antagonist) of the present disclosure, and thus the cancer or tumor may be treated with the methods and compositions provided herein. For example, see Cancer Discover, April 2016, page 431-445, herein incorporated by reference, which described loss-of-function mutations in the CREBBP gene, and use of an EP300 inhibitor to suppress the CREBBP cancer cells. In some embodiments, the cancer or tumor exhibits a CREBBP loss of function mutation, e.g., mediated by an CREBBP loss of function mutation, and may be sensitive to treatment with a CREBBP and EP300 inhibitor (or antagonist) of the present disclosure, and thus the cancer or tumor may be treated with the methods and compositions provided herein.

In yet other embodiments, the cancer or tumor exhibits a CREBBP loss of function mutation and EP300 loss of function mutation. In some embodiments, the cancer or tumor exhibits a CREBBP loss of function mutation and EP300 loss of function mutation, e.g., mediated by an CREBBP loss of function mutation and EP300 loss of function mutation, and may be sensitive to treatment with a CREBBP inhibitor (or antagonist), a EP300 inhibitor (or antagonist) or a CREBBP and EP300 inhibitor (or antagonist) of the present disclosure, and thus the cancer or tumor may be treated with the methods and compositions provided herein.

In some embodiments, the cancer or tumor exhibits wild-type CREBBP and/or EP300, and may be sensitive to treatment with a CREBBP inhibitor (or antagonist), a EP300 inhibitor (or antagonist) or a CREBBP and EP300 dual inhibitor (or antagonist) of the present disclosure, and thus the cancer or tumor may be treated with the methods and compositions provided herein.

Non-limiting examples of cancers include, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Other non-limiting examples of cancer include endometrial carcinoma, bladder urothelial carcinoma, cervical squamous cell carcinoma, endocervical adenocarcinoma, colon adenocarcinoma, head and neck squamous cell carcinoma, stomach adenocarcinoma, skin cutaneous melanoma, esophageal carcinoma, lymphoid neoplasm, diffuse large B-cell lymphoma, rectum adenocarcinoma, lung squamous cell carcinoma, kidney renal papillary cell carcinoma, cholangiocarcinoma, glioblastoma multiforme, liver hepatocellular carcinoma, ovarian serous cystadenocarcinoma, sarcoma, thymoma, breast invasive carcinoma, lung adenocarcinoma, pancreatic adenocarcinoma, kidney renal clear cell carcinoma, uterine carcinosarcoma, acute myeloid leukemia, uveal melanoma, mesothelioma, prostate adenocarcinoma, adrenocortical carcinoma, testicular germ cell tumors, or brain lower grade glioma.

In some embodiments, the present disclosure provides methods and compositions for treating a tumor in a subject. In some embodiments, the tumor is a solid tumor. In some embodiments, the tumor is a liquid or disperse tumor. In some embodiments, the tumor or a cell comprised in the tumor harbors a EP300 loss of function mutation. In some embodiments, the tumor or a cell comprised in the tumor harbors a CREBBP loss of function mutation. In some embodiments, the tumor or a cell comprised in the tumor harbors a CREBBP loss of function mutation and EP300 loss of function mutation. In some embodiments, the tumor or a cell comprised in the tumor harbors a EP300 loss of function mutation and the tumor or a cell comprised in the tumor does not harbor CREBBP loss of function mutation. In some embodiments, the tumor or a cell comprised in the tumor harbors a CREBBP loss of function mutation and the tumor or a cell comprised in the tumor does not harbor an EP300 loss of function mutation. In some embodiments, the cancer or tumor exhibits wild-type CREBBP and/or EP300. In some embodiments, the tumor is associated with a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, follicular lymphoma, diffuse large B-cell lymphoma, Mantle cell lymphoma, Langerhans cell histiocytosis, multiple myeloma, or myeloproliferative neoplasms.

In some embodiments, the tumor is associated with a hematologic malignancy, including but not limited to B-cell lymphomas. Non-limiting examples of B-cell Lymphoma include Hodgkin lymphoma, non-Hodgkin lymphoma, follicular lymphoma, diffuse large B-cell lymphoma, and Mantle cell lymphoma.

In some embodiments, the tumor is associated with a hematologic malignancy, including but not limited to T-cell lymphomas. Non-limiting examples of T-cell Lymphoma include cutaneous T-cell lymphoma, mycosis fungoides, Sézary disease, anaplastic large cell lymphoma, and precursor T-lymphoblastic lymphoma, and Angioimmunoblastic T-cell lymphoma.

In some embodiments, a tumor comprises a solid tumor. In some embodiments, solid tumors include but are not limited to tumors of the bladder, breast, central nervous system, cervix, colon, esophagus, endometrium, head and neck, kidney, liver, lung, ovary, pancreas, skin, stomach, uterus, or upper respiratory tract. In some embodiments, a tumor that may be treated by the compositions and methods of the present disclosure is a breast tumor. In some embodiments, a tumor that may be treated by the compositions and methods of the present disclosure is not a lung tumor.

In some embodiments, a tumor or cancer suitable for treatment with the methods and compositions provided herein includes, for example, Acute Lymphoblastic Leukemia (ALL), Acute Myeloid Leukemia (AML), Adrenal Cortex Cancer, Adrenocortical Carcinoma, AIDS-Related Cancer (e.g., Kaposi Sarcoma, AIDS-Related Lymphoma, Primary CNS Lymphoma), Anal Cancer, Appendix Cancer, Astrocytoma , Atypical Rhabdoid Tumor, Basal Cell Carcinoma, Bile Duct Cancer, Bladder Cancer, Bone Cancer , Brain Tumor, Breast Cancer, Bronchial Tumor, Burkitt Lymphoma, Carcinoid Tumor , Carcinoma, Cardiac (Heart) Tumor, Central Nervous System Tumor , Cervical Cancer, Cholangiocarcinoma, Chordoma, Chronic Lymphocytic Leukemia (CLL), Chronic Myelogenous Leukemia (CML), Chronic, Myeloproliferative Neoplasm, Colorectal Cancer, Craniopharyngioma, Cutaneous T-Cell Lymphoma, Ductal Carcinoma In Situ (DCIS), Embryonal Tumor , Endometrial Cancer, Endometrial Sarcoma, Ependymoma, Esophageal, Esthesioneuroblastoma, Ewing Sarcoma, Extracranial Germ Cell Tumor, Extragonadal Germ Cell Tumor, Eye Cancer, Fallopian Tube Cancer, Gallbladder Cancer, Gastric (Stomach) Cancer, Gastrointestinal Carcinoid Tumor, Gastrointestinal Stromal Tumor (GIST), Germ Cell Tumor, Gestational Trophoblastic Disease, Glioma, Hairy Cell Leukemia, Head and Neck Cancer, Hepatocellular (Liver) Cancer, Hodgkin Lymphoma, Hypopharyngeal Cancer, Intraocular Melanoma, Islet Cell Tumor , Kaposi Sarcoma, Kidney Tumor, Langerhans Cell Histiocytosis , Laryngeal Cancer, Leukemia, Lip and Oral Cavity Cancer, Liver Cancer, Lung Cancer, Lymphoma, Male Breast Cancer, Malignant Fibrous Histiocytoma, Melanoma, Merkel Cell Carcinoma, Mesothelioma, Mouth Cancer, Multiple Endocrine Neoplasia Syndrome, Multiple Myeloma, Plasma Cell Neoplasm, Mycosis Fungoides, Myelodysplastic Syndrome, Myelodysplastic/Myeloproliferative Neoplasm , Nasal Cavity Cancer, Nasopharyngeal Cancer, Neuroblastoma, Non-Hodgkin Lymphoma, Non-Small Cell Lung Cancer, Oral Cancer, Oral Cavity Cancer, Oropharyngeal Cancer, Osteosarcoma, Ovarian Cancer, Pancreatic Cancer, Pancreatic Neuroendocrine Tumor (Islet Cell Tumor), Paraganglioma, Paranasal Sinus Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Primary Peritoneal Cancer, Prostate Cancer, Rectal Cancer, Renal Cell (Kidney) Cancer, Retinoblastoma, Retinoblastoma, Rhabdomyosarcoma, Rhabdomyosarcoma, Salivary Gland Cancer, Sarcoma, Sézary Syndrome, Skin Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Testicular Cancer, Throat Cancer, Thymic Carcinoma, Thymoma, Thyroid Cancer, Urethral Cancer, Uterine Sarcoma, Uterine Sarcoma, Vaginal Cancer, Vascular Tumor, Vulvar Cancer, Waldenström Macroglobulinemia, Wilms' Tumor.

Non-limiting examples of leukemia include acute lymphocytic leukemia (ALL), acute myeloid leukemia (AML), chronic lymphocytic leukemia (CLL), hairy cell leukemia (HCL), acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, adult T cell leukemia, aggressive NK-cell leukemia, and mast cell leukemia.

Non-limiting examples of lymphoma include, small lymphocytic lymphoma (SLL), Hodgkin's lymphoma (HL), B-cell lymphoma, marginal zone B-cell lymphoma, splenic marginal zone lymphoma, diffuse large B-cell lymphoma (DLBCL), non-Hodgkin's lymphoma (NHL), mantle cell lymphoma (MCL), follicular lymphoma (FL), marginal zone lymphoma (MZL), Burkitt's lymphoma (BL), MALT lymphoma, precursor T-lymphoblastic lymphoma, T-cell lymphoma, adult T cell lymphoma and angioimmunoblastic T-cell lymphoma.

Non-limiting examples of B-cell Lymphoma include Hodgkin lymphoma, non-Hodgkin lymphoma, follicular lymphoma, diffuse large B-cell lymphoma, and Mantle cell lymphoma.

Non-limiting examples of T-cell Lymphoma include cutaneous T-cell lymphoma, mycosis fungoides, Sézary disease, anaplastic large cell lymphoma, and precursor T-lymphoblastic lymphoma, and Angioimmunoblastic T-cell lymphoma.

### Pharmaceutical Compositions

A compounds provided herein, can be administered to a subject, e.g., to a human patient, alone, or in a pharmaceutical composition, e.g., where the compound provided herein is admixed with a suitable carrier or excipient. A pharmaceutical composition typically comprises or can be administered at a dose sufficient to treat or ameliorate a disease or condition in the recipient subject, e.g., to treat or ameliorate a cancer as described herein. Accordingly, a pharmaceutical composition is formulated in a manner suitable for administration to a subject, e.g., in that it is free from pathogens and formulated according to the applicable regulatory standards for administration to a subject, e.g., for administration to a human subject. As an example, a formulation for injection is typically sterile and essentially pyrogen-free.

A suitable compound provided herein can also be administered to a subject as a mixture with other agents, e.g., in a suitably formulated pharmaceutical composition. For example, one aspect of the present disclosure relates to pharmaceutical compositions comprising a therapeutically effective dose of a compound provided herein, or a pharmaceutically acceptable salt, hydrate, enantiomer or stereoisomer thereof; and a pharmaceutically acceptable diluent or carrier.

Techniques for formulation and administration of compounds provided herein may be found in references well known to one of ordinary skill in the art, such as Remington's “The Science and Practice of Pharmacy,” 21st ed., Lippincott Williams & Wilkins 2005, the entire contents of which are incorporated herein by reference.

Pharmaceutical compositions as provided herein are typically formulated for a suitable route of administration. Suitable routes of administration may, for example, include enteral administration, e.g., oral, rectal, or intestinal administration; parenteral administration, e.g., intravenous, intramuscular, intraperitoneal, subcutaneous, or intramedullary injection, as well as intrathecal, direct intraventricular, or intraocular injections; topical delivery, including eyedrop and transdermal; and intranasal and other transmucosal delivery, or any suitable route provided herein or otherwise apparent to those of ordinary skill in the art.

The pharmaceutical compositions provided herein may be manufactured, e.g., by mixing, dissolving, granulating, dragee-making, levigating, emulsifying, encapsulating, entrapping, or lyophilizing processes, or by any other suitable processes known to those of ordinary skill in the art.

Pharmaceutical compositions for use in accordance with the present disclosure may be formulated using one or more physiologically acceptable carriers comprising excipients and auxiliaries which facilitate processing of the compounds provided herein into preparations which can be used pharmaceutically. Proper formulation is dependent upon the route of administration chosen.

For injection, the compounds of the disclosure may be formulated in aqueous solutions, preferably in physiologically compatible buffers such as Hanks' solution, Ringer's solution, or physiological saline buffer. For transmucosal administration, penetrants are used in the formulation appropriate to the barrier to be permeated. Such penetrants are generally known in the art.

For oral administration, a compounds provided herein can be formulated readily by combining a compound provided herein with pharmaceutically acceptable carriers known in the art. Such carriers enable the compound(s)provided herein to be formulated as tablets, pills, dragees, capsules, liquids, gels, syrups, slurries, suspensions and the like, for oral ingestion by a patient to be treated. Pharmaceutical preparations for oral use can be obtained by combining a compound(s) provided herein with a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of CREBBP antagonist(s) doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredient(s), e.g., one or more suitable compounds provided herein , in admixture with filler such as lactose, binders such as starches, and/or lubricants such as talc or magnesium stearate and, optionally, stabilizers. In soft capsules, a compound provided herein may be dissolved or suspended in suitable liquids, such as fatty oils, liquid paraffin, or liquid polyethylene glycols. In addition, stabilizers may be added.

For buccal administration, the compositions may take the form of tablets or lozenges formulated in conventional manner.

For administration by inhalation, a compound provided herein for use according to the present disclosure are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of a compound provided herein and a suitable powder base such as lactose or starch.

Suitable compounds provided herein can be formulated for parenteral administration by injection, e.g., bolus injection or continuous infusion. Formulations for injection may be presented in unit dosage form, e.g., in ampoules, or in multi-dose containers, and, in some embodiments, may contain an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of a compound provided herein in water-soluble form. Additionally, suspensions of a compound provided herein may be prepared as appropriate injection suspensions, e.g., a compound provided herein, e.g., aquaeous or oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility a compound provided herein to allow for the preparation of highly concentrated solutions.

Alternatively, the active ingredient(s), e.g., a compound provided herein, may be in powder form for reconstitution before use with a suitable vehicle, e.g., sterile pyrogen-free water.

A compound provided herein may also be formulated in rectal compositions such as suppositories or retention enemas, e.g., containing conventional suppository bases, such as cocoa butter or other glycerides.

In addition to the formulations described previously, a compound provided herein may also be formulated as a depot preparation. Such long acting formulations may be administered by implantation (for example, subcutaneously or intramuscularly or by intramuscular injection). Thus, for example, a compound provided herein may be formulated with suitable polymeric or hydrophobic materials (for example as an emulsion in an acceptable oil) or ion exchange resins, or as sparingly soluble derivatives (for example, as a sparingly soluble salt).

Alternatively, other delivery systems for compounds provided herein may be employed, for example, in embodiments where the compound is hydrophobic. Liposomes and emulsions are examples of delivery vehicles or carriers for hydrophobic drugs. Certain organic solvents such as dimethysulfoxide also may be employed. Additionally, a compound provided herein may be delivered using a sustained-release system, such as semi-permeable matrices of solid hydrophobic polymers containing the compound. Various sustained-release materials have been established and are well known by those skilled in the art. Sustained-release capsules may, depending on their chemical nature, release a compound provided herein for a few hours, a few days, a few weeks, or a few months, e.g., up to over 100 days.

The pharmaceutical compositions may also comprise suitable solid or gel phase carriers or excipients. Examples of such carriers or excipients include but are not limited to calcium carbonate, calcium phosphate, various sugars, starches, cellulose derivatives, gelatin, and polymers, such as polyethylene glycols.

Additional suitable pharmaceutical compositions and processes and strategies for formulating a suitable compound provided herein will be apparent to the skilled artisan based on the present disclosure. The disclosure is not limited in this respect.

### Administration

In some embodiments, a compound provided herein is formulated, dosed, and/or administered in a therapeutically effective amount using pharmaceutical compositions and dosing regimens that are consistent with good medical practice and appropriate for the relevant agent(s) and subject(s). In principle, therapeutic compositions can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transdermal, or other kinds of administration involving physical breaching of a tissue of a subject and administration of the therapeutic composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

Factors to be considered when optimizing routes and/or dosing schedule for a given therapeutic regimen may include, for example, the particular indication being treated, the clinical condition of a subject (e.g., age, overall health, prior therapy received and/or response thereto) the site of delivery of the agent, the nature of the agent (e.g. an antibody or other polypeptide-based compound), the mode and/or route of administration of the agent, the presence or absence of combination therapy, and other factors known to medical practitioners. For example, in the treatment of cancer, relevant features of the indication being treated may include, for example, one or more of cancer type, stage, location.

In some embodiments, one or more features of a particular pharmaceutical composition and/or of a utilized dosing regimen may be modified over time (e.g., increasing or decreasing the amount of active agent in any individual dose, increasing or decreasing time intervals between doses), for example in order to optimize a desired therapeutic effect or response (e.g., inhibition of a CREBBP gene or gene product).

In general, type, amount, and frequency of dosing of active agents in accordance with the present disclosure are governed by safety and efficacy requirements that apply when one or more relevant agent(s) is/are administered to a mammal, preferably a human. In general, such features of dosing are selected to provide a particular, and typically detectable, therapeutic response as compared to what is observed absent therapy.

In the context of the present disclosure, an exemplary desirable therapeutic response may involve, but is not limited to, inhibition of and/or decreased tumor growth, tumor size, metastasis, one or more of the symptoms and side effects that are associated with a tumor, as well as increased apoptosis of cancer cells, therapeutically relevant decrease or increase of one or more cell marker or circulating markers. Such criteria can be readily assessed by any of a variety of immunological, cytological, and other methods that are disclosed in the literature.

In some embodiments, an effective dose (and/or a unit dose) of an active agent, may be at least about 0.01 μg/kg body weight, at least about 0.05 μg/kg body weight; at least about 0.1 μg/kg body weight, at least about 1 μg/kg body weight, at least about 2.5 μg/kg body weight, at least about 5 μg/kg body weight, and not more than about 100 μg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of a compound provided herein.

In some embodiments, a “therapeutically effective amount” or “therapeutically effective dose” is an amount of a compound provided herein, or a combination of two or more compounds provided herein, which inhibits, totally or partially, the progression of the condition or alleviates, at least partially, one or more symptoms of the condition. In some embodiments, a therapeutically effective amount can be an amount which is prophylactically effective. In some embodiments, an amount which is therapeutically effective may depend upon a patient's size and/or gender, the condition to be treated, severity of the condition and/or the result sought. In some embodiments, a therapeutically effective amount refers to that amount of a compound provided herein that results in amelioration of at least one symptom in a patient. In some embodiments, for a given patient, a therapeutically effective amount may be determined by methods known to those of skill in the art.

In some embodiments, toxicity and/or therapeutic efficacy a compound provided herein can be determined by standard pharmaceutical procedures in cell cultures or experimental animals, e.g., for determining the maximum tolerated dose (MTD) and the ED50 (effective dose for 50% maximal response). Typically, the dose ratio between toxic and therapeutic effects is the therapeutic index; in some embodiments, this ratio can be expressed as the ratio between MTD and ED50. Data obtained from such cell culture assays and animal studies can be used in formulating a range of dosage for use in humans.

In some embodiments, dosage may be guided by monitoring effect of a compound provided herein on one or more pharmacodynamic markers of enzyme inhibition (e.g., histone acetylation or target gene expression) in diseased or surrogate tissue. For example, cell culture or animal experiments can be used to determine the relationship between doses required for changes in pharmacodynamic markers and doses required for therapeutic efficacy can be determined in cell culture or animal experiments or early stage clinical trials. In some embodiments, dosage of a compound provided herein lies preferably within a range of circulating concentrations that include the ED50 with little or no toxicity. In some embodiments, dosage may vary within such a range, for example depending upon the dosage form employed and/or the route of administration utilized. The exact formulation, route of administration and dosage can be chosen by the individual physician in view of the patient's condition. In the treatment of crises or severe conditions, administration of a dosage approaching the MTD may be required to obtain a rapid response.

In some embodiments, dosage amount and/or interval may be adjusted individually, for example to provide plasma levels of an active moiety which are sufficient to maintain, for example a desired effect, or a minimal effective concentration (MEC) for a period of time required to achieve therapeutic efficacy. In some embodiments, MEC for a particular compound provided herein can be estimated, for example, from in vitro data and/or animal experiments. Dosages necessary to achieve the MEC will depend on individual characteristics and route of administration. In some embodiments, high pressure liquid chromatography (HPLC) assays or bioassays can be used to determine plasma concentrations.

In some embodiments, dosage intervals can be determined using the MEC value. In certain embodiments, a compound provided herein should be administered using a regimen which maintains plasma levels above the MEC for 10-90% of the time, preferably between 30-90% and most preferably between 50-90% until the desired amelioration of a symptom is achieved. In other embodiments, different MEC plasma levels will be maintained for differing amounts of time. In cases of local administration or selective uptake, the effective local concentration of the drug may not be related to plasma concentration.

One of skill in the art can select from a variety of administration regimens and will understand that an effective amount of a particular compound provided herein may be dependent on the subject being treated, on the subject's weight, the severity of the affliction, the manner of administration and/or the judgment of the prescribing physician.

### EXEMPLIFICATION

The compounds described herein may be synthesized using methods known to those of ordinary skill in the art. For example, Scheme 1 and Scheme 2 provide non-limiting examples of synthetic methodologies. In some embodiments, the synthetic methods comprise providing an intermediate having the following structure, following by use of coupling methods known to those of ordinary skill in the art.

Intermediate:

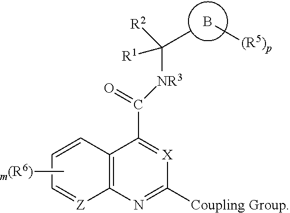

In some embodiments, the intermediate has the structure:

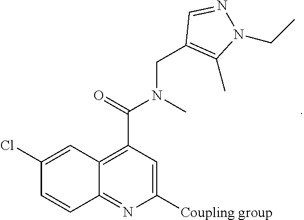

A non-limiting coupling group is Cl.

The synthesis of the compounds described herein may be carried out in any suitable solvent, including, but are not limited to, non-halogenated hydrocarbon solvents {e.g., pentane, hexane, heptane, cyclohexane), halogenated hydrocarbon solvents {e.g., dichloromethane, chloroform, fluorobenzene, trifluoromethylbenzene), aromatic hydrocarbon solvents {e.g., toluene, benzene, xylene), ester solvents {e.g., ethyl acetate), ether solvents {e.g. , tetrahydrofuran, dioxane, diethyl ether, dimethoxyethane.), and alcohol solvents {e.g., ethanol, methanol, propanol, isopropanol, tert-butanol). In certain embodiments, a protic solvent is used. In other embodiments, an aprotic solvent is used. Non-limiting examples of solvents useful include acetone, acetic acid, formic acid, dimethyl sulfoxide, dimethyl formamide, acetonitrile, cresol, glycol, petroleum ether, carbon tetrachloride, hexamethyl-phosphoric triamide, triethylamine, picoline, and pyridine.

The synthesis of the compounds may be carried out at any suitable temperature. In some cases, the synthesis is carried out at about room temperature {e.g., about 20° C., between about 20° C. and about 25° C., about 25° C., or the like). In some cases, however, the method synthesis carried out at a temperature below or above room temperature, for example, at about −78° C. at about −70° C., about −50° C., about −30° C., about −10° C., about −0° C., about 10° C., about 30° C., about 40° C., about 50° C., about 60° C., about 70° C., about 80° C., about 90° C., about 100° C., about 120° C., about 140° C., or the like. In some embodiments, the synthesis is carried out at temperatures above room temperature, for example, between about 25° C. and about 120° C., or between about 25° C. and about 100° C., or between about 40° C. and about 120° C., or between about 80° C. and about 120° C. The temperature may be maintained by reflux of the solution. In some cases, the synthesis is carried out at temperatures between about −78° C. and about 25° C., or between about 0° C. and about 25° C.

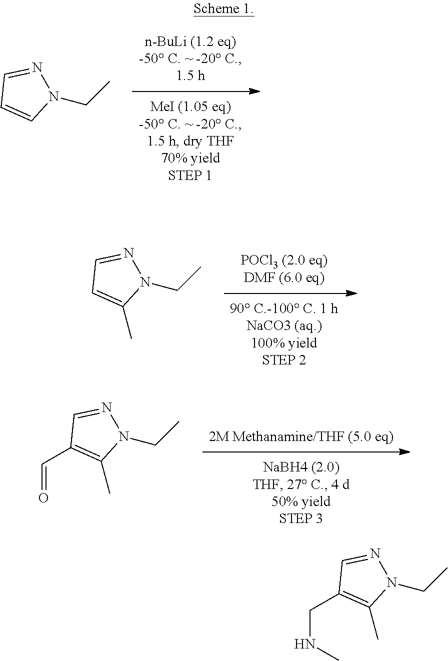

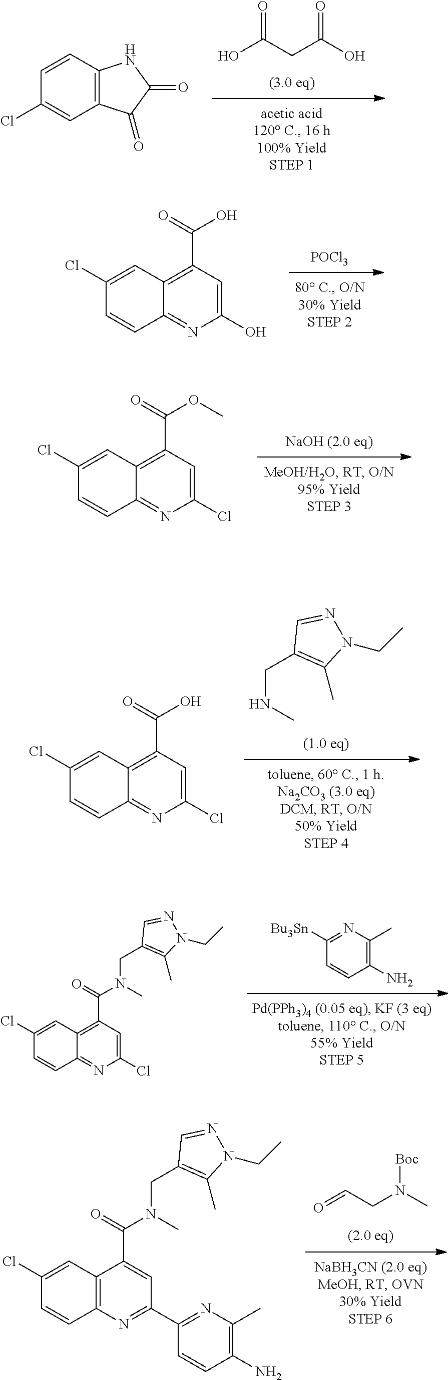

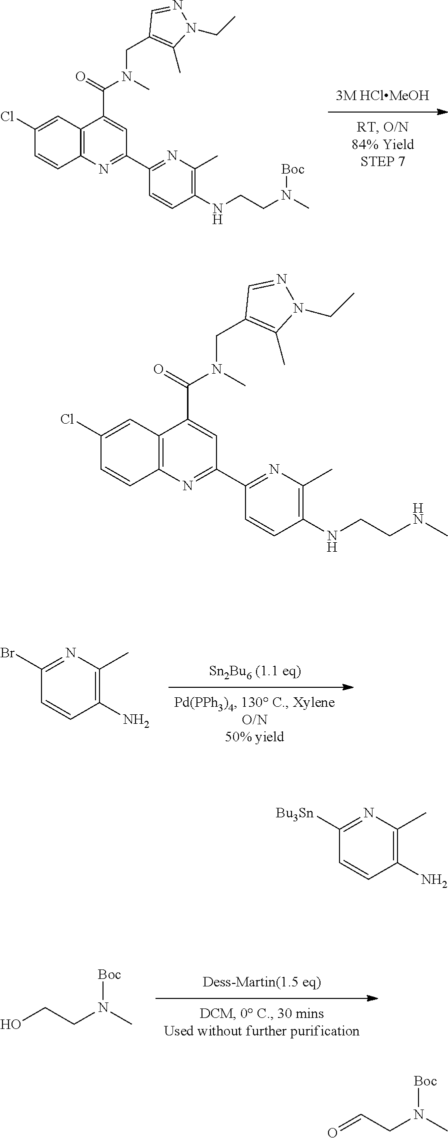

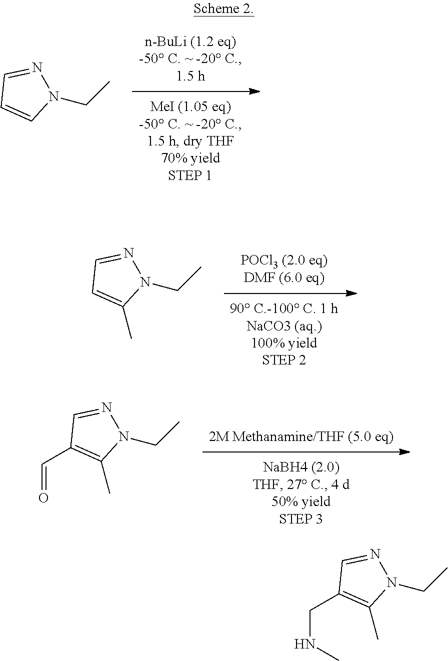

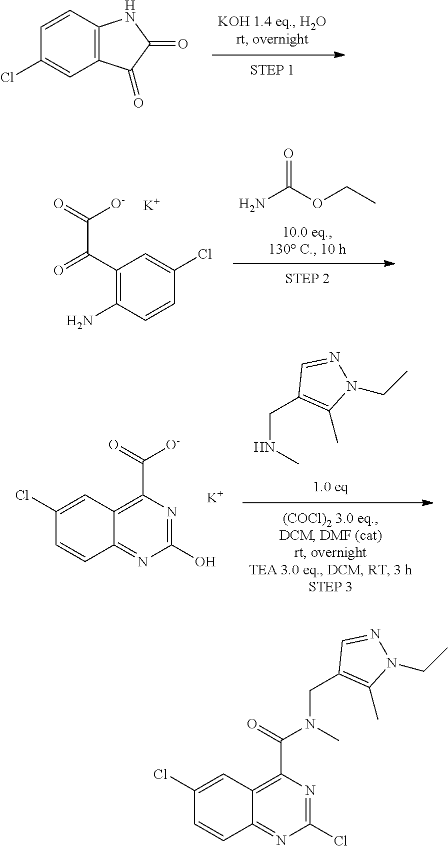

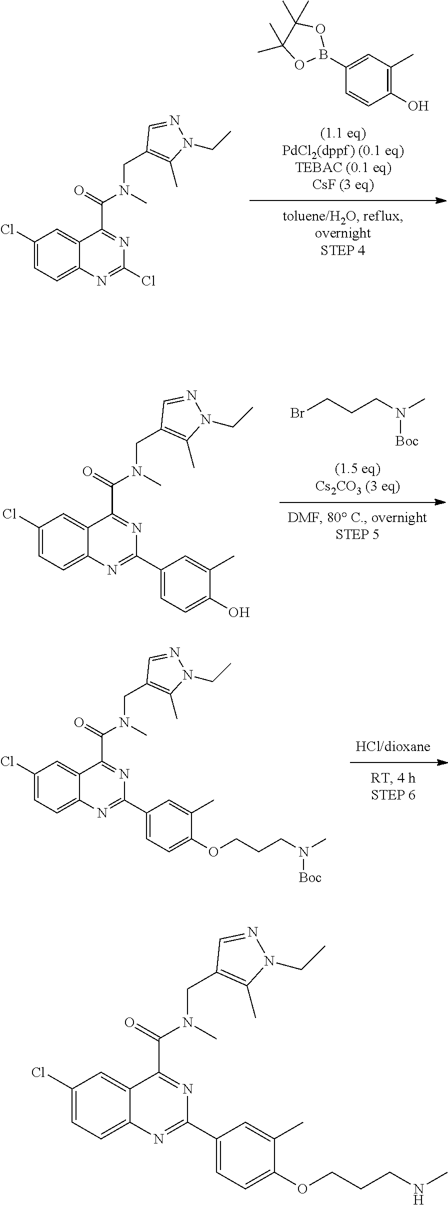

The synthesis of the compounds may be carried out at any suitable pH, for example, equal to or less than about 13, equal to or less than about 12, equal to or less than about 11, equal to or less than about 10, equal to or less than about 9, equal to or less than about 8, equal to or less than about 7, or equal to or less than about 6. In some cases, the pH may be greater than or equal to 1, greater than or equal to 2, greater than or equal to 3, greater than or equal to 4, greater than or equal to 5, greater than or equal to 6, greater than or equal to 7, or greater than or equal to 8. In some cases, the pH may be between about 2 and about 12, or between about 3 and about 11, or between about 4 and about 10, or between about 5 and about 9, or between about 6 and about 8, or about 7.

The percent yield of a compounds or intermediate may be greater than about 60%, greater than about 70%, greater than about 75%>, greater than about 80%>, greater than about 85%>, greater than about 90%, greater than about 92%, greater than about 95%, greater than about 96%o, greater than about 97%>, greater than about 98%>, greater than about 99%>, or greater.

### EXAMPLE 1

The following example describes the exemplary synthesis of 6-chloro-N-[(1-ethyl-5-methyl-1H-pyrazol-4-yl)methyl]-N-methyl-2-(6-methyl-5-{[2-(methylamino)ethyl]amino}pyridin-2-yl)quinoline-4-carboxamide (Compound 3). Scheme 3 shows the synthesis of 6-chloro-N-[(1-ethyl-5-methyl-1H-pyrazol-4-yl)methyl]-N-methyl-2-(6-methyl-5-{[2-(methylamino)ethyl]amino}pyridin-2-yl)quinoline-4-carboxamide (Compound 3).

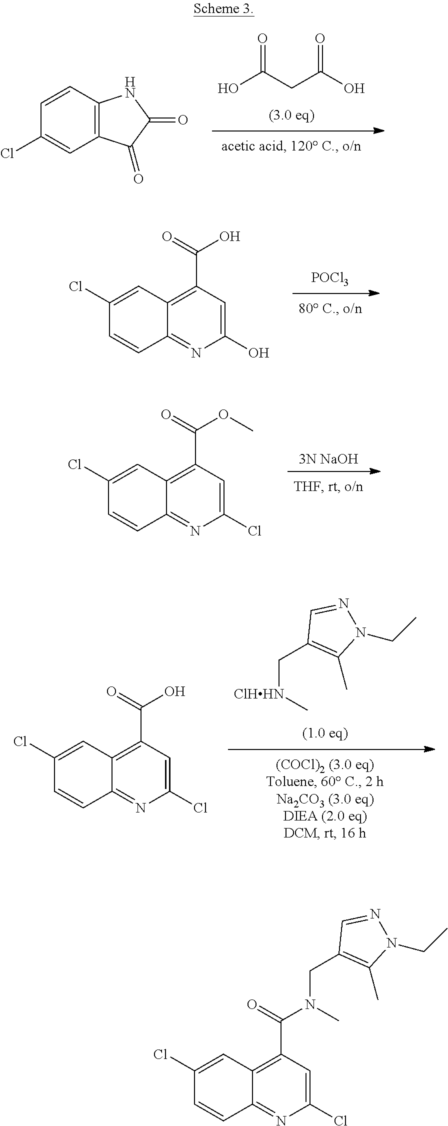

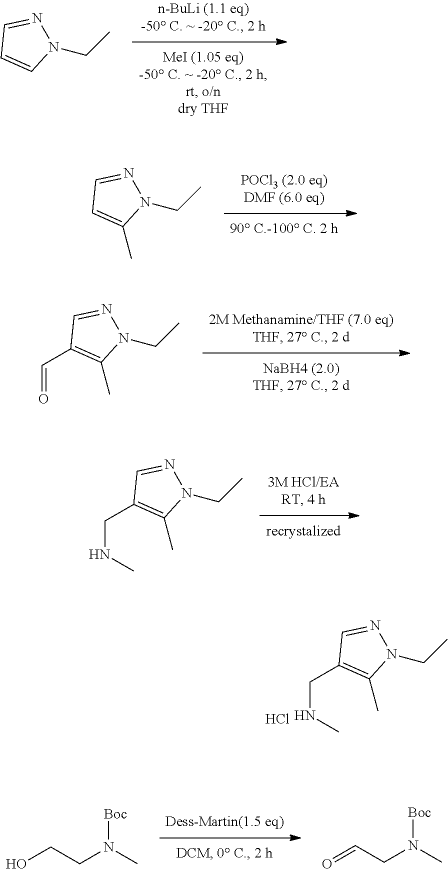

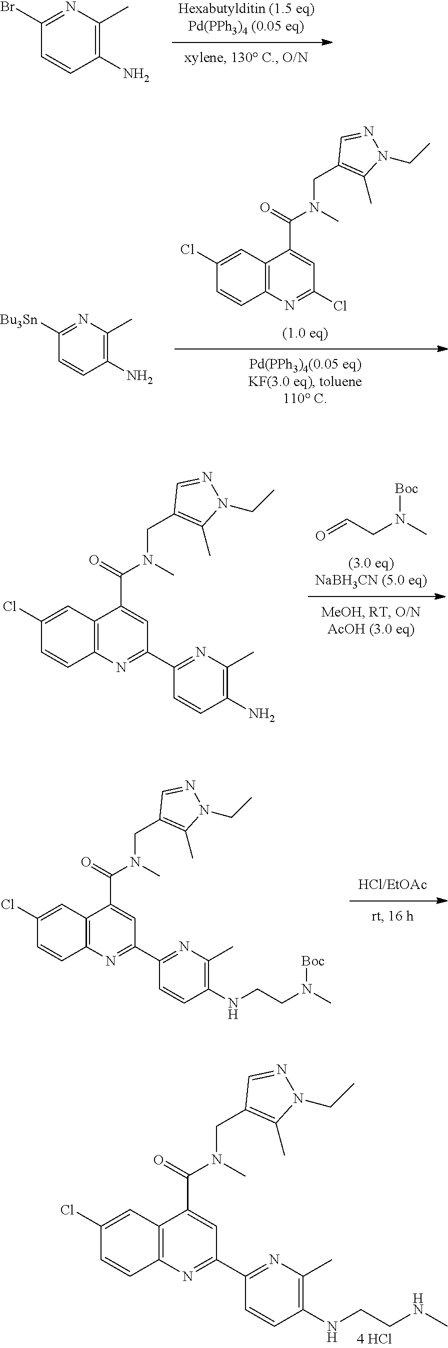

### Synthesis of 6-Chloro-2-hydroxyquinoline-4-carboxylic Acid

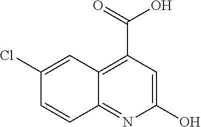 [SMILES: O=C(O)c1cc(O)nc2ccc(Cl)cc12]

To a solution of 5-chloro-2,3-dihydro-1H-indole-2,3-dione (300 g, 1.65 mol) in glacial acetic acid (3 L) was added malonic acid (515 g, 4.96 mol) and the mixture heated at reflux overnight. The reaction was repeated on an additional 700 g of starting material in 2 batches under the same conditions. The crude reaction mixtures were combined and worked up together. Acetic acid was removed under reduced pressure and the residue suspended in water (5 L). The solid was collected by filtration and the filter cake was washed with water to give a grey solid. The solid was suspended in water (5 L) again and filtered, the filter cake was washed with water and dried to give the desired product (1.23 kg, 70% based on 1 kg starting 5-chloro-2,3-dihydro-1H-indole-2,3-dione) as a pale yellow solid. This material was used for the next step without further purification. LC-MS (Agilent, Method: S12-5 mins): Rₜ 1.82 min; m/z calculated for C₁₀H₆ClNO₃ [M+H]⁺224.0, found 224.0/226.1

### Synthesis of Methyl 2,6-dichloroquinoline-4-carboxylate

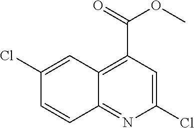 [SMILES: COC(=O)c1cc(Cl)nc2ccc(Cl)cc12]

A solution of 6-chloro-2-hydroxyquinoline-4-carboxylic acid (500 g, 2.24 mol) in POCl₃ (3.3 L) was heated at 80° C. overnight. The reaction mixture was then concentrated to dryness then dissolved in DCM (1.2 L) and cooled to 0° C. MeOH (2 L) was added and the precipitate that formed was collected by filtration. The filter cake was dried under vacuum to give the desired product (400 g, 70%) as a white solid. LC-MS (Agilent, Method: S12-5 mins): Rₜ 4.19 min; m/z calculated for C₁₁H₇C₁₂NO₂ [M+H]⁺255.9, found 256.0/258.0

### Synthesis of 2,6-Dichloroquinoline-4-carboxylic Acid

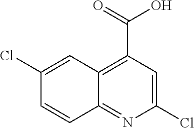 [SMILES: O=C(O)c1cc(Cl)nc2ccc(Cl)cc12]

To a solution of methyl 2,6-dichloroquinoline-4-carboxylate (800 g, 3.12 mol) in THF (8 L) was added 3 M aqueous NaOH solution (4.16 L, 12.50 mol) and the reaction stirred at room temperature overnight. The pH of the mixture was adjusted to 6.0 with HCl (6.0 M) and the precipitate that formed was collected by filtration. The filter cake was dried under vacuum to afford the desired product (700 g, 92%) as a white solid. LC-MS (Agilent, Method: S12-3.5 mins): Rₜ 1.86 min; m/z calculated for C₁₀H₅Cl₂NO₂ [M+H]⁺240.9, found 241.9/244.0

### Synthesis of 2,6-Dichloro-N-[(1-ethyl-5-methyl-1H-pyrazol-4-yl)methyl]-N-methylquinoline-4-carboxamide

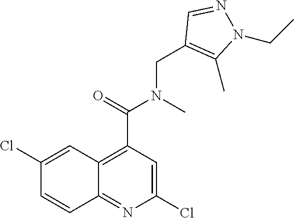 [SMILES: CCn1ncc(CN(C)C(=O)c2cc(Cl)nc3ccc(Cl)cc23)c1C]

To a solution of 2,6-dichloroquinoline-4-carboxylic acid (69.9 g, 289 mmol) in toluene (1.5 L) was added oxalyl chloride (100 g, 789 mmol) and DMF (0.2 mL). After heating at 60° C. for 16 h, the reaction mixture was concentrated in vacuo. A solution of [(1-ethyl-5-methyl-1H-pyrazol-4-yl)methyl](methyl)amine hydrochloride (50 g, 263 mmol) and N,N-Diisopropylethylamine (67.9 g, 526 mmol) in DCM (1.5 L) was stirred at room temperature for 20 mins, sodium carbonate (83.6 g, 789 mmol) was then added. The acid chloride made above was added to this suspension and the resulting mixture was stirred at room temperature for 16 h. The reaction mixture was filtered and the filtrate was concentrated, the residue was purified by silica gel column (MeOH/DCM=100/1) to give the desired product (70 g, 70%) as a white solid.

LC-MS (Agilent, Method: S12-5 mins): Rₜ 3.27 min; m/z calculated for C₁₈H₁₈Cl₂N₄O [M+H]⁺377.0, found 377.2/379.1

### Synthesis of 1-Ethyl-5-methyl-1H-pyrazole

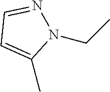

To a mixture of 1-ethyl-1H-pyrazole (200 g, 2.08 mol) in dry THF (2 L) at −50° C. under N₂ was added n-butyllithium (915 mL, 2.29 mol) dropwise. The reaction was stirred at −50° C. and slowly allowed to warm to −20° C. over 2 h. Methyl iodide (309 g, 2.18 mol) was added and the resulting mixture was stirred at −20° C. for 2 h. The reaction was allowed to warm to room temperature and stirred overnight. The reaction was repeated on an additional 800 g of starting material in 2 batches under the same conditions. The crude reaction mixtures were combined and worked up together. The mixture was quenched with water (8 L) and extracted with EtOAc (8 L×3). The combined organic layers were washed with brine (1 L) and concentrated. The residue obtained was purified by silica gel column (MeOH/DCM=1/100, v/v) to give the desired product (850 g, 74% based on 1 kg starting 1-ethyl-1H-pyrazole) as a red oil.

LC-MS (Agilent, Method: S12-5 mins): Rₜ 2.11 min; m/z calculated for C₆H₁₀N₂ [M+H]⁺111.1, found 111.1

### Synthesis of 1-Ethyl-5-methyl-1H-pyrazole-4-carboxylate

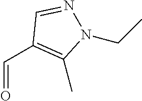

To a solution of 1-ethyl-5-methyl-1H-pyrazole (425 g, 3.86 mol) in dimethylformamide (1.69 kg, 23.15 mol) at 90° C. was added phosphorus oxychloride (1.18 kg, 7.72 mol) dropwise and the resulting mixture heated at 100° C. for 2 h. The reaction was repeated on the same scale and the crude reaction mixtures were combined and worked up together. The pH of the mixture was adjusted to 8 with saturated aqueous Na₂CO₃ solution and extracted with DCM (10 L×30). The combined organic phases were dried over Na₂SO₄ and concentrated. The residue was purified by silica gel column (DCM to DCM/MeOH=50/1, v/v) to give the crude product (2.34 kg, contains DMF, >100% yield) as a brown oil. 340 g of the crude product was used to the next step directly without further purification. Another 2 kg of the crude product was further purified by silica gel column (Pet. ether to DCM/MeOH=50/1, v/v) to give the crude product (1.45 kg, contains DMF, >100% yield) as a brown oil. LC-MS (Agilent, Method: S12-5 mins): Rₜ 1.76 min; m/z calculated for C₇H₁₀N₂O [M+H]⁺139.1, found 139.1.

### Synthesis of 1-(1-ethyl-5-methyl-1H-pyrazol-4-yl)-N-methylmethanamine hydrochloride

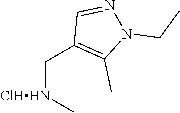

A solution of 1-ethyl-5-methyl-1H-pyrazole-4-carbaldehyde (340 g, 0.98 mol) in 2M Methylamine/THF (3.44 L, 6.89 mol) was stirred at RT for 2 days. NaBH₄ (74.7 g, 1.97 mol) was added and the reaction was stirred a further 2 days at room temperature before the reaction was quenched by addition of MeOH (150 mL) and NH₄Cl (80 g). The mixture was filtered, and the filtrate was concentrated to dryness. The residue obtained was purified by silica gel column (DCM/MeOH/NH₃.H₂O=50/1/0.2 to DCM/MeOH/NH₃.H₂O=5/1/0.05, v/v/v) to afford the crude product (83 g) as a yellow oil. The crude product was suspended in 3M HCl (gas)/EtOAc (600 mL) and the mixture was stirred at room temperature for 4 h. The precipitate that formed was collected by filtration then recrystallized from EtOH (500 mL) to give the desired product (40 g pure and 24 g with 2% impurity, 30% for 2 steps) as a white solid. LC-MS (Agilent, Method: S12-5 mins): Rₜ 0.56 min; m/z calculated for C₈H₁₅N₃ [M+H]⁺154.1, found 154.1.

### Synthesis of tert-butyl methyl(2-oxoethyl)carbamate

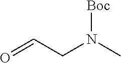

To a solution of tert-butyl N-(2-hydroxyethyl)-N-methylcarbamate (24 g, 136 mmol) in DCM (400 mL) was added Dess-Martin periodinane (86.5 g, 204 mmol), the resulting mixture was stirred at 0° C. for 2 h. The reaction mixture was filtered and the filtrate was washed with water and brine, dried over Na₂SO₄ and concentrated. The residue was purified by column (Pet.Ether/EtOAc=5/1, v/v) to give the desired product (18 g, 76%) as a colorless oil.

### Synthesis of 2-Methyl-6-(tributylstannyl)pyridin-3-amine

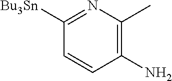

To a solution of 6-bromo-2-methylpyridin-3-amine (30 g, 160 mmol) and bis(tributyltin) (139 g, 240 mmol) in xylene (400 mL) was added tetrakis(triphenylphosphine) palladium (9.2 g, 8.0 mmol). The resulting mixture was heated at 130° C. for 17 h. The mixture was filtered through a silica gel pad and the filtrate was concentrated. The residue was purified by column (Pet.Ether/EtOAc=2/1, v/v) to give the desired product (27 g, 42%) as a yellow oil. LC-MS (Agilent, Method: S12-5 mins): Rₜ 0.90 min; m/z calculated for C₁₈H₃₄N₂Sn [M+H]+399.17, found 399.2

### Synthesis of 2-(5-Amino-6-methylpyridin-2-yl)-6-chloro-N-[(1-ethyl-5-methyl-1H-pyrazol-4-yl)methyl]-N-methylquinoline-4-carboxamide

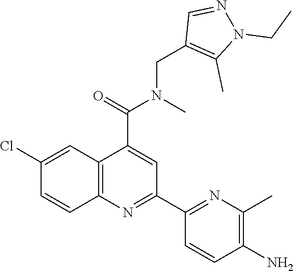 [SMILES: CCn1ncc(CN(C)C(=O)c2cc(-c3ccc(N)c(C)n3)nc3ccc(Cl)cc23)c1C]

To a solution of 2-methyl-6-(tributylstannyl)pyridin-3-amine (14.7 g, 37.1 mmol) in toluene (200 mL) were added 2,6-dichloro-N-[(1-ethyl-5-methyl-1H-pyrazol-4-yl)methyl]-N-methylquinoline-4-carboxamide (14 g, 37.1 mmol), potassium fluoride (6.44 g, 111 mmol) and tetrakis(triphenylphosphine) palladium (2.13 g, 1.85 mmol). The reaction was heated at 110° C. for 15 h, then cooled to room temperature and filtered. The filtrate was diluted with EtOAc (100 mL) and washed with water and brine. The organic solvent was removed under reduced pressure and the residue obtained purified by silica gel column (DCM/MeOH=10/1, v/v) to give the desired product (10 g, 60%) as a yellow solid. LC-MS (Agilent, Method: S12-3.5 mins): Rₜ 2.41 min; m/z calculated for C₂₄H₂₅ClN₆O [M+H]⁺449.2, found 449.2/451.2

### Synthesis of tert-butyl N-(2-{[6-(6-chloro-4-{[(1-ethyl-5-methyl-1H-pyrazol-4-yl)methyl](methyl)carbamoyl}quinolin-2-yl)-2-methylpyridin-3 -yl]amino}ethyl)-N-methylcarbamate

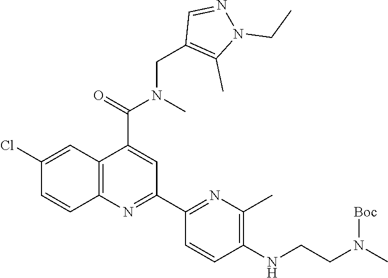 [SMILES: CCn1ncc(CN(C)C(=O)c2cc(-c3ccc(NCCN(C)C(=O)OC(C)(C)C)c(C)n3)nc3ccc(Cl)cc23)c1C]

To a solution of 2-(5-amino-6-methylpyridin-2-yl)-6-chloro-N-[(1-ethyl-5-methyl-1H-pyrazol-4-yl)methyl]-N-methylquinoline-4-carboxamide (10 g, 22.2 mmol) in MeOH (200 mL) was added tert-butyl N-methyl-N-(2-oxoethyl)carbamate (7.69 g, 44.4 mmol) and AcOH (3.99 g, 66.6 mmol). The mixture was stirred at room temperature overnight, LCMS showed the imine formation was not complete. Another portion of tert-butyl N-methyl-N-(2-oxoethyl)carbamate (3.85 g, 22.2 mmol) was added and the mixture was stirred for another 8 h. Sodium cyanoborohydride (6.97 g, 111 mmol) was added and the mixture was stirred at room temperature overnight. The mixture was poured into water (300 mL) and extracted with DCM (200 mL×2). The combined organic phases were washed with saturated aqueous Na₂CO₃ solution (200 mL×3), water (200 mL×3) and brine (200 mL), dried over Na₂SO₄ and concentrated. The residue was purified by silica gel column (DCM/MeOH=20/1, v/v) to give the crude product (10 g), which was purified by reverse phase column (38% MeCN in water) to give the desired product (8 g, 60%) as a yellow solid. LC-MS (Agilent, Method: S12-5 mins): Rₜ 2.19 min; m/z calculated for C₃₂H₄₀ClN₇O₃ [M+H]+605.3, found 605.3/607.3

### Synthesis of 6-Chloro-N-[(1-ethyl-5-methyl-1H-pyrazol-4-yl)methyl]-N-methyl-2-(6-methyl-5-{[2-(methylamino)ethyl]amino}pyridin-2-yl)quinoline-4-carboxamide

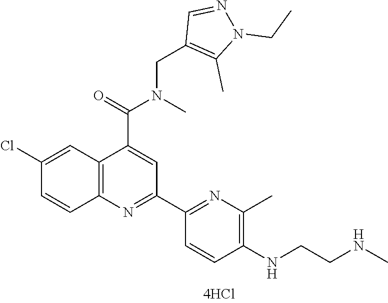

A solution of tert-butyl N-(2-{[6-(6-chloro-4-{[(1-ethyl-5-methyl-1H-pyrazol-4-yl)methyl](methyl)carbamoyl}quinolin-2-yl)-2-methylpyridin-3 -yl]amino}ethyl)-N-methylcarbamate (2.7 g, 4.45 mmol) in HCl (gas)/EtOAc (3.0 M, 25 mL) was stirred at room temperature overnight. The precipitate that formed was collected by filtration and the filter cake washed with EtOAc and dried under vacuum to give the desired product (2.4 g, 82%) as a red solid. LC-MS (Agilent, Method: S12-5 mins): Rₜ 2.16 min; m/z calculated for C₂₇H₃₂ClN₇O [M+H]506.2, found 506.2/508.2

### EXAMPLE 2

The following examples described materials and methods relating to the LC-MS detection of compound mass. Mass Spectrometry data for exemplary compounds is summarized in Table 1, Table 2, and Table 3 under column labelled: “Mass Detected M+1”.

LC-MS (Agilent) (S12-5 mins): LC: Agilent Technologies 1290 series, Binary Pump, Diode Array Detector. Agilent Poroshell 120 EC-C18, 2.7 μm, 4.6×50 mm column. Mobile phase: A: 0.05% Formate in water (v/v), B: 0.05% Formate in MeCN (v/v). Flow Rate: 1 mL/min at 25° C. Detector: 214 nm, 254 nm. Gradient stop time, 5 min.

TABLE A

| T (min) | A (%) | B (%) |
|---|---|---|
| 0.00 | 90 | 10 |
| 0.50 | 90 | 10 |
| 4.00 | 10 | 90 |
| 4.50 | 0 | 100 |
| 4.51 | 90 | 10 |
| 5.00 | 90 | 10 |

1. MS: G6120A, Quadrupole LC/MS, Ion Source: API-ES, TIC: 70˜1000 m/z, Fragmentor: 70, Drying gas flow: 12 L/min, Nebulizer pressure: 36 psi, Drying gas temperature: 350° C., Vcap: 3000V.
2. Sample preparation: samples were dissolved in methanol at 1˜10 μg/mL, then filtered through a 0.22 μm filter membrane. Injection volume: 1˜10 μL.

LC-MS (Agilent) (S12-3.5 mins): LC: Agilent Technologies 1290 series, Binary Pump, Diode Array Detector. Agilent Poroshell 120 EC-C18, 2.7 μm, 4.6×50 mm column. Mobile phase: A: 0.05% Formate in water (v/v), B: 0.05% Formate in MeCN (v/v). Flow Rate: 1.5 mL/min at 25° C. Detector: 214 nm, 254 nm. Gradient stop time, 3.5 min.

TABLE B

| T (min) | A (%) | B (%) |
|---|---|---|
| 0.00 | 80 | 20 |
| 3.00 | 20 | 80 |
| 3.50 | 20 | 80 |

1. MS: G6120A, Quadrupole LC/MS, Ion Source: API-ES, TIC: 70˜1000 m/z, Fragmentor: 70, Drying gas flow: 12 L/min, Nebulizer pressure: 36 psi, Drying gas temperature: 350° C., Vcap: 3000V.
2. Sample preparation: samples were dissolved in methanol at 1˜10 μg/mL, then filtered through a 0.22 μm filter membrane. Injection volume: 1˜10 μL.

### EXAMPLE 3

The following example describes methods and materials relating to an H3K18AC in-cell western assay. IC50 values (micromolar (μM)) are summarized in Table 1, Table 2, and Table 3 under the column labeled: “CREBBP ICW IC₅₀ (micromolar).”

MATERIALS: HB-CLS-2 cell line, DMEM: Ham's F12 medium (1:1 mixture), penicillin-streptomycin, heat inactivated fetal bovine serum, D-PBS, Odyssey blocking buffer, 800CW goat anti-rabbit IgG (H+L) antibody, Licor Odyssey CLx Infrared Scanner, H3K18Ac rabbit monoclonal antibody. DRAQS fluorescent probe solution (5 mM), and 100% methanol were commercially available. HB-CLS-2 adherent cells were maintained in complete growth medium (DMEM: Ham's F12 supplemented with 10% v/v heat inactivated fetal bovine serum) and cultured at 37° C. under 5% CO2.

METHOD: Cell Treatment, ICW for detection of H3K18Ac and DNA content. HB-CLS-2 cells were seeded in assay medium (DMEM: Ham's F12 supplemented with 10% v/v heat inactivated fetal bovine serum and 1% Penicillin/Streptomycin) at a concentration of 80,000 cells per mL in a Poly-D-Lysine coated 384-well culture plates at 50 μL per well. Plates were incubated at room temperature for 30 minutes and then incubated at 37° C., 5% CO2 for additional 16-24 hours. Compounds and DMSO normalization were then added directly to the plates using a D300 Digital Dispenser and returned to the incubator at 37° C., 5% CO2 for 2 hrs. After the incubation, the contents of the plates were discarded into the appropriate waste stream and blotted on laboratory tissue to remove residual liquid. 90 μL per well of 100% ice cold methanol was added to the plates and incubated at room temperature for 15 minutes. Then methanol was then discarded into the appropriate waste stream and the plates again blotted on laboratory tissue to remove residual liquid. Plates were transferred to a Biotek 405 plate washer and washed 3 times with 100 μL per well of wash buffer (1X PBS containing 0.1% Triton X-100 (v/v)). Next, 50 μL per well of Odyssey blocking buffer with 0.1% Tween 20 (v/v) was added to each plate and incubated for 1 hour at room temperature. Blocking buffer was removed and 20 μL of primary antibody were added (α-H3K18Ac diluted 1:800 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:400 800CW goat anti-rabbit IgG (H+L) antibody, 1:2000 DRAQ5 in Odyssey buffer with 0.1% Tween 20 (v/v)) and incubated for 1 hour at room temperature. The plates were washed 3 times with 100 μL per well wash buffer then 3 times with 100 μL per well of water. Plates were allowed to dry at room temperature then imaged on the Licor Odyssey CLx machine which measured integrated intensity at 700 nm and 800 nm wavelengths. Both 700 and 800 channels were scanned.

### EXAMPLE 4

The following example describes methods and materials relating to a high throughput proliferation (HTP) assay. IC50 values (micromolar) are summarized in Table 1, Table 2, and Table 3 under the column labeled: “CREBBP HTP IC₅₀ (micromolar).”

MATERIALS: 647V cell line, Dulbecco's MEM, penicillin-streptomycin, heat inactivated fetal bovine serum, D-PBS, and CellTiter-Glo were commercially available.

647V adherent cells were maintained in complete growth medium (Dulbecco's MEM supplemented with 15% v/v heat inactivated fetal bovine serum) and cultured at 37° C. under 5% CO₂.

METHOD: Measurement of the effect of compound in High Throughput Proliferation (HTP) assays was performed as follows: Exponentially growing 647V cells were plated, in triplicate, in 384-well plates at the appropriate cell density in a final volume of 50 μl. Cells were incubated in the presence of increasing concentrations of compound. Viable cell number was determined at day 7 by addition of 35 μl CellTiter-Glo to each well of the plate, incubated in the dark for 30 minutes, and read on a PerkinElmer EnVision instrument to enumerate the number of cells.

### EXAMPLE 5

The following example describes materials and methods relating to a biochemical assay for CREBBP (1084-1701). IC50 values (micromolar (μM)) are summarized in Table 1, Table 2, and Table 3 under the column labeled: “CREBBP Biochemistry IC₅₀ (micromolar).”

MATERIALS: Reagents 1M Tris pH 8.0, Tween 20 10%, DTT, bovine serum gelatin (BSG) 2%, Peptide #233 (biotin-H3 11-25, K14R, K23R), Acetyl-CoA, CREBBP (1084-1701), formic acid (100%), and sodium bicarbonate were commercially available.

METHODS: The effect of compounds was measured in the following biochemical assay using CREBBP (1084-1701). Enzyme mix 30 μL per well was added using a Multi-drop to wells of prepared Compound Stock plate. The enzyme was incubated in the Compound Stock plate for 30 minutes at room temperature. Substrate mix, 20 μL per well, was added to Compound Stock plate using a Multi-drop. The plate was covered and incubate 30 minutes at room temperature. The reaction was stopped with addition of 5 μL per well of 5% formic acid using a Multi-drop. The plate was Incubated for 30 minutes at room temperature. The reaction mixture was neutralized with addition of 5 μL per well of 10% sodium bicarbonate using a Multi-drop. The plate was Incubated for 35 minutes at room temperature. The reaction mixture was Transferred 2.5 μL per well to a SAMDI biochip. The plate was Incubated for 60 minutes at room temperature. The samples were washed, dried, and matrix applied to SAMDI biochip. The SAMDI biochip was then read on the mass spectrometer.

### EXAMPLE 6

The following example describes materials and methods relating to a biochemical assay for EP300. IC50 values (micromolar (μM)) are summarized in Table 1, Table 2, and Table 3 under the column labeled: “EP300 Biochemistry IC₅₀ (micromolar).”

Reagents:

| | Compound stock | | 384 well V-bottom | | |
|---|---|---|---|---|---|
| DAILY | plate: | Per well: | Plate | [Stock] | [Final] |
| 1 | Test compound IC₅₀ in | 1 μL | Serially diluted 3 | [top] at 10 mM | 200 μM |
| | 100% DMSO | | fold for 10 different | | |
| | | | points | | |
| 2 | 1X Assay Buffer (100 | 100000 μL | Tris pH 8.0 | 1M | 50 mM |
| | mL) | 5000 | Gelatin | 2% | 0.005% |
| | | 250 | DTT | 1M | 1 mM |
| | | 100 | Tween 20 | 10% | 0.002% |
| | | 20 | dH₂O | | |
| | | 94630 | | | |
| 3 | Enzyme Mix, 30 | 30 μL | 1X Assay Buffer | 18.9 μM | 0.125 nM |
| | μL/well | 30 | P300(1048-1664) | | |
| | | 0.00033 | | | |
| 4 | Substrate mix, 20 | 20 μL | 1X Assay Buffer | 10 mM | 3 μM |
| | μL/well | 19.98 | Peptide #233 | 10 mM | 1 μM |
| | | 0.015 | Ac-CoA | | |
| | | 0.005 | | | |
| 5 | Formic acid STOP | 5 μL | 500 Formic Acid | 5% | 0.5% |
| | mix, 5 μL/well | | solution | | |
| 6 | Sodium bicarbonate | 5 μL | 10% Sodium | 10% | 1% |
| | neutralizing solution | | Bicarbonate solution | | |

Methods:

| Step | |
|---|---|
| Number | Step Description |
| 1 | Add 30 μL per well of Enzyme mix using a Multi-drop |
| | to wells of prepared Compound Stock plate. |
| 2 | Incubate the enzyme in the Compound Stock plate for |
| | 30 minutes at room temperature. |
| 3 | Add 20 μL per well of Substrate mix to Compound |
| | Stock plate using a Multi-drop. |
| 4 | Cover plate and incubate 30 minutes at room temperature. |
| 5 | Stop reaction with addition of 5 μL per well of |
| | 5% formic acid using a Multi-drop. |
| 6 | Incubate for 30 minutes at room temperature. |
| 7 | Neutralize with addition of 5 μL per well of |
| | 10% sodium bicarbonate using a Multi-drop. |
| 8 | Incubate for 35 minutes at room temperature. |
| 9 | Transfer 2.5 μL per well to a SAMDI biochip. |
| 10 | Incubate for 60 minutes at room temperature. |
| 11 | Wash, dry, and apply matrix to SAMDI biochip. |
| 12 | Read SAMDI biochip on mass spectrometer |

### EXAMPLE 7

This example describes methods and materials for 7-day proliferation assay.

Materials

A total of 22 bladder cell lines were used (see table below). Cell lines were cultured in recommended growth media according to supplier.

| Cell Line | Vendor | Cat. No. | Media |
|---|---|---|---|
| 639V | DSMZ | ACC-413 | DMEM + 10% FBS |
| VMCUB1 | DSMZ | ACC-400 | DMEM + 10% FBS |
| 647V | DSMZ | ACC-414 | DMEM + 10% FBS |
| KU1919 | DSMZ | ACC-395 | EMEM + 10% FBS |
| 5637 | ATCC | HTB-9 | RPMI 1640 + 10% FBS |
| BC3C | DSMZ | ACC450 | DMEM + 10% FBS |
| BFTC905 | DSMZ | ACC-361 | DMEM + 2 mM Glutamine + 10% FBS |
| CAL29 | DSMZ | ACC-515 | EMEM + 10% FBS |
| HBCLS2 | CLS | 300191 | D/F + 10% FBS |
| HT-1197 | ATCC | CRL-1473 | EMEM + 10% FBS |
| HT-1376 | ATCC | CRL-1472 | EMEM + 10% FBS |
| JMSU1 | ATCC | TIB-152 | RPMI1640 + 10% FBS |
| KMBC2 | JCRB | JCRB1148 | F12 + 10% FBS |
| RT112 | CLS | 300324 | DMEM + 10% FBS |
| RT4 | ATCC | HTB-2 | McCoy's 5a + 10% FBS |
| SCABER | ATCC | HTB-3 | EMEM + 10% FBS + 1% SP |
| SW1710 | DSMZ | ACC-140 | DMEM + 10% FBS |
| SW780 | ATCC | CRL-2169 | L-15 + 10% FBS |
| TCCSUP | ATCC | HTB-5 | EMEM + 10% FBS |
| UBLC1 | ECACC | 6013102 | ⅔ DMEM:⅓ RPMI1640 + 2 |
| | | | mM Glutamine + 10% FBS |
| UMUC3 | ATCC | CRL-1749 | EMEM + 10% FBS |
| CLS439 | CLS | 300150 | McCoy's 5a + 10% FBS |

Method

Cells were in culture media at a density optimized for a 7-day culture in a final volume of 150 μL per well in white opaque 96-well plates. Cells were allowed to adhere for several hours (4-6 h) then compounds were added with HPD300 Digital Dispenser and placed into the incubator at 37° C., 5% CO2 for 7 days. After 7 days incubation, 100 μL of CellTiter-Glo® Luminescent Cell Viability Assay (Promega-G7573) reagents were added per well. After 20 minutes incubation luminescence was measured in plate reader. IC₅₀ were calculated from a non-linear logarithmic growth curve.

IC₅₀ values (nanomolar (nM)) are summarized in the table below, which depicts the inhibitory effect of certain compounds in bladder cell lines.

| | Compound 3 | Compound | Compound | Compound 577 |
|---|---|---|---|---|
| | (Table 1) | 537 | 480 | (Table 2) |
| | Average IC₅₀ | (Table 2) | (Table 2) | Average IC₅₀ |
| | (nM) N = 2 | Average | Average IC₅₀ | (nM) |
| Cell Line | unless | IC₅₀ | (nM) N = 2 | N = 1 unless |
| Name | specified | (nM) N = 2 | unless specified | specified |
| 639V | 40 | 99.5 | 57 | 100 |
| VMCUB1 | 69 | 116.5 | 116** | 51 |
| 647V | 127 | 125 | 52 | 53.5* |
| KU1919 | 145 | 217 | 90.5 | 101.5* |
| 5637 | 133 | 197 | 147 | 111 |
| BC3C | 5097.5 | 4059 | 10000 | 1189 |
| BFTC905 | 121.5 | 224 | 110 | 132.5* |
| CAL29 | 1987.5 | 704 | 523.5 | 1202 |
| HB-CLS-2 | 219.5 | 434 | 344.5 | 240 |
| HT1197 | 153.5 | 346 | 111.5 | 234 |
| HT1376 | 3888 | 2763.5 | 1247 | 2074 |
| JMSU1 | 377.5 | 748.5 | 546 | 997 |
| KMBC2 | 7638 | 1596.5 | 1809.5 | 896 |
| RT112 | 111 | 370 | 198 | 78 |
| RT4 | 158 | 343.5 | 174 | 199 |
| SCABER | 249 | 251.5 | 172** | 182 |
| SW1710 | 290 | 190 | 184** | 143 |
| SW780 | 227.7** | 322 | 125.5 | 168 |
| TCCSUP | 7595.5 | 10000 | 10000 | 10000 |
| UBLC1 | 141 | 494 | 234 | 427 |
| UM-UC-3 | 792.5 | 1377.5 | 584.5 | 1096 |
| CLS439 | 619** | 849 | 480** | 461 |

*N = 2, **N = 3

### Equivalents

The foregoing written specification is considered to be sufficient to enable one skilled in the art to practice the disclosure. The present disclosure is not to be limited in scope by examples provided, since the examples are intended as a single illustration of one aspect of the disclosure and other functionally equivalent embodiments are within the scope of the disclosure. Various modifications of the disclosure in addition to those shown and described herein will become apparent to those skilled in the art from the foregoing description and fall within the scope of the appended claims. The advantages and objects of the disclosure are not necessarily encompassed by each embodiment of the disclosure.

TABLE 1
Exemplary Compounds and Data

| | | | | CREBBP | | | EP300 |
|---|---|---|---|---|---|---|---|
| | | | Mass | Biochemistry | CREBBP ICW | CREBBP HTP | Biochemistry |
| Com- | | | Detected | IC₅₀ | IC₅₀ | IC₅₀ | IC₅₀ |
| pound Number | STRUCTURE | MW | M + 1 | (micromolar) | (micromolar) | (micromolar) | (micromolar) |
| 1 | 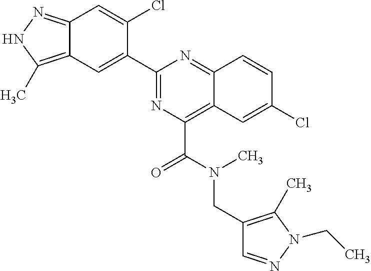 [SMILES: CCn1ncc(CN(C)C(=O)c2nc(-c3cc4c(C)[nH]nc4cc3Cl)nc3ccc(Cl)cc23)c1C] | 509.4 | 508.9 | 0.0033 | 0.027 | 0.020 | 0.0021 |
| 2 | 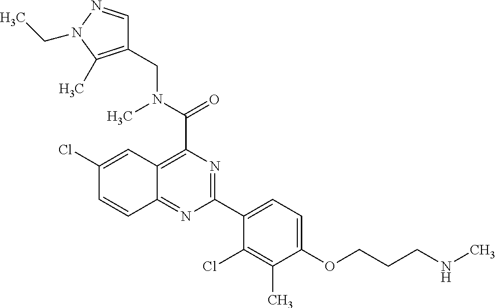 [SMILES: CCn1ncc(CN(C)C(=O)c2nc(-c3ccc(OCCCNC)c(C)c3Cl)nc3ccc(Cl)cc23)c1C] | 555.5 | 555.0 | 0.022 | 0.034 | 0.017 | 0.0050 |
| 3 | 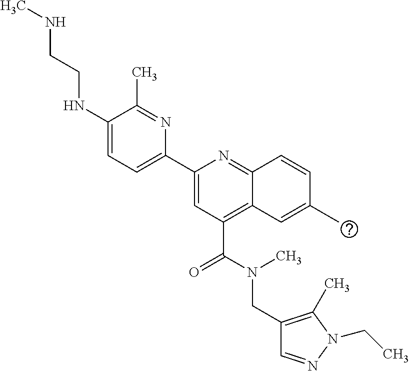 [SMILES: CCn1ncc(CN(C)C(=O)c2cc(-c3ccc(NCCNC)c(C)n3)nc3ccc(C)cc23)c1C] | 506.0 | 506.0 | 0.021 | 0.040 | 0.027 | 0.0044 |
| 4 | 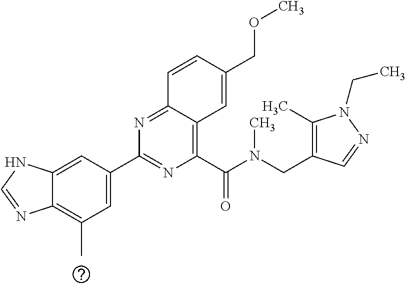 [SMILES: CCn1ncc(CN(C)C(=O)c2nc(-c3cc(C)c4nc[nH]c4c3)nc3ccc(COC)cc23)c1C] | 504.0 | 504.2 | 0.012 | 0.057 | 0.037 | 0.0037 |
| 5 | 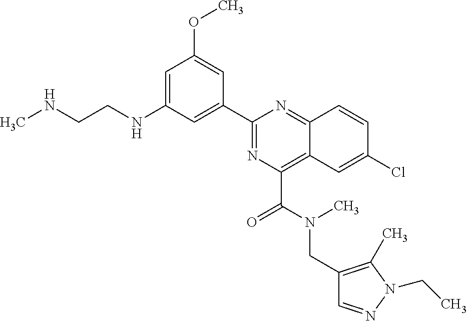 [SMILES: CCn1ncc(CN(C)C(=O)c2nc(-c3cc(NCCNC)cc(OC)c3)nc3ccc(Cl)cc23)c1C] | 522.0 | 522.3 | 0.016 | 0.060 | 0.035 | 0.011 |
| 6 | 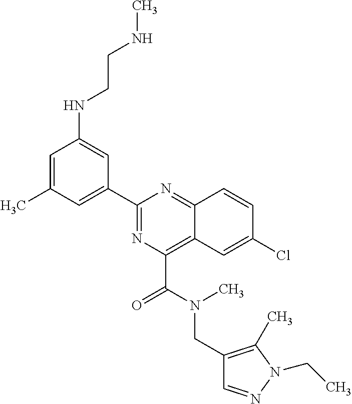 [SMILES: CCn1ncc(CN(C)C(=O)c2nc(-c3cc(C)cc(NCCNC)c3)nc3ccc(Cl)cc23)c1C] | 508.0 | 508.3 | 0.016 | 0.075 | 0.033 | 0.0087 |
| 7 | 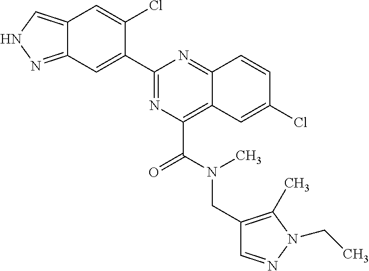 [SMILES: CCn1ncc(CN(C)C(=O)c2nc(-c3cc4n[nH]cc4cc3Cl)nc3ccc(Cl)cc23)c1C] | 494.4 | 494.0 | 0.0095 | 0.077 | 0.066 | 0.0053 |
| 8 | 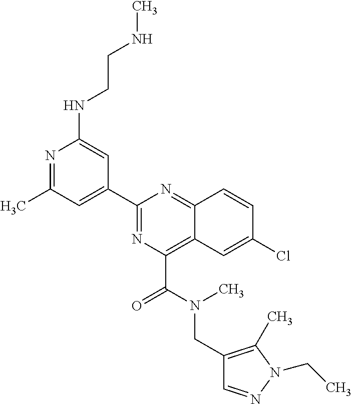 [SMILES: CCn1ncc(CN(C)C(=O)c2nc(-c3cc(C)nc(NCCNC)c3)nc3ccc(Cl)cc23)c1C] | 507.0 | 507.2 | 0.033 | 0.078 | 0.042 | 0.026 |
| 9 | 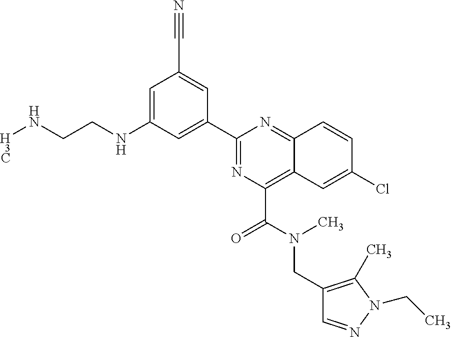 [SMILES: CCn1ncc(CN(C)C(=O)c2nc(-c3cc(C#N)cc(NCCNC)c3)nc3ccc(Cl)cc23)c1C] | 517.0 | 517.2 | 0.015 | 0.090 | 0.051 | 0.011 |
| 10 | 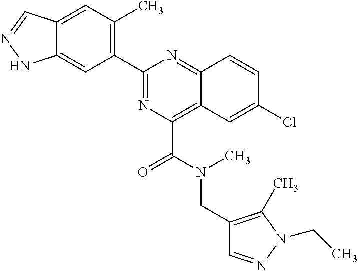 [SMILES: CCn1ncc(CN(C)C(=O)c2nc(-c3cc4[nH]ncc4cc3C)nc3ccc(Cl)cc23)c1C] | 474.0 | 474.2 | 0.022 | 0.093 | 0.077 | 0.0084 |
| 11 | 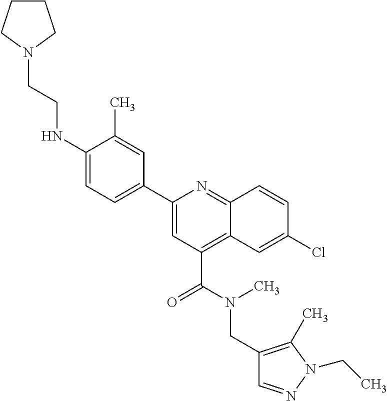 [SMILES: CCn1ncc(CN(C)C(=O)c2cc(-c3ccc(NCCN4CCCC4)c(C)c3)nc3ccc(Cl)cc23)c1C] | 546.1 | 548.3 | 0.025 | 0.10 | 0.067 | 0.0088 |
| 12 | 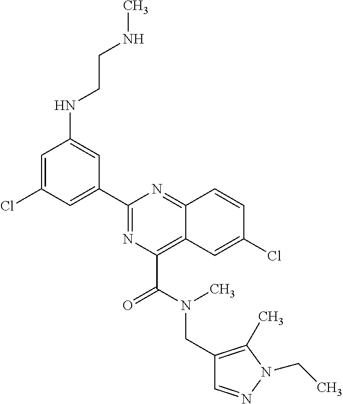 [SMILES: CCn1ncc(CN(C)C(=O)c2nc(-c3cc(Cl)cc(NCCNC)c3)nc3ccc(Cl)cc23)c1C] | 526.5 | 526.0 | 0.013 | 0.11 | 0.032 | 0.0035 |
| 13 | 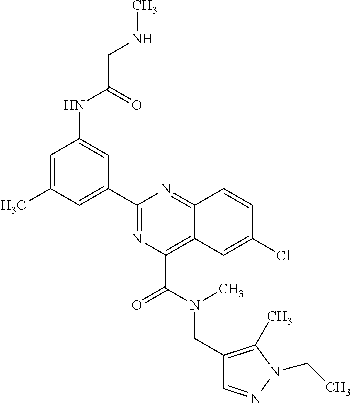 [SMILES: CCn1ncc(CN(C)C(=O)c2nc(-c3cc(C)cc(NC(=O)CNC)c3)nc3ccc(Cl)cc23)c1C] | 5520.0 | 520.1 | 0.010 | 0.12 | 0.11 | 0.0077 |
| 14 | 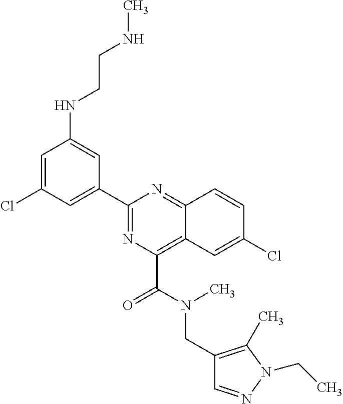 [SMILES: CCn1ncc(CN(C)C(=O)c2nc(-c3cc(Cl)cc(NCCNC)c3)nc3ccc(Cl)cc23)c1C] | 528.6 | 526.0 | 0.014 | 0.14 | 0.039 | 0.0029 |
| 15 | 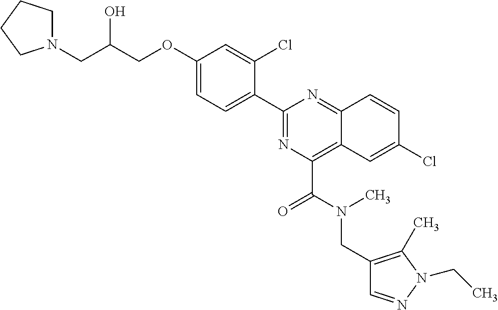 [SMILES: CCn1ncc(CN(C)C(=O)c2nc(-c3ccc(OCC(O)CN4CCCC4)cc3Cl)nc3ccc(Cl)cc23)c1C] | 597.5 | 597.0 | 0.028 | 0.15 | 0.064 | 0.012 |
| 16 | 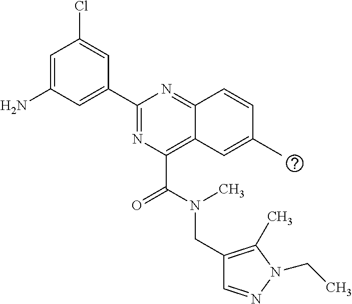 [SMILES: CCn1ncc(CN(C)C(=O)c2nc(-c3cc(N)cc(Cl)c3)nc3ccc(C)cc23)c1C] | 469.4 | 469.0 | 0.0046 | 0.15 | 0.16 | 0.0074 |
| 17 | 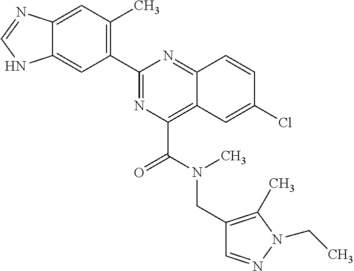 [SMILES: CCn1ncc(CN(C)C(=O)c2nc(-c3cc4[nH]cnc4cc3C)nc3ccc(Cl)cc23)c1C] | 474.0 | 474.2 | 0.060 | 0.17 | 0.13 | 0.12 |
| 18 | 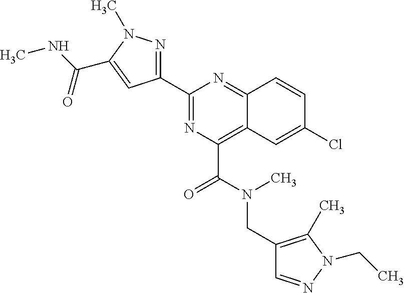 [SMILES: CCn1ncc(CN(C)C(=O)c2nc(-c3cc(C(=O)NC)n(C)n3)nc3ccc(Cl)cc23)c1C] | 481.0 | 481.2 | 0.023 | 0.17 | 0.22 | 0.010 |
| 19 | 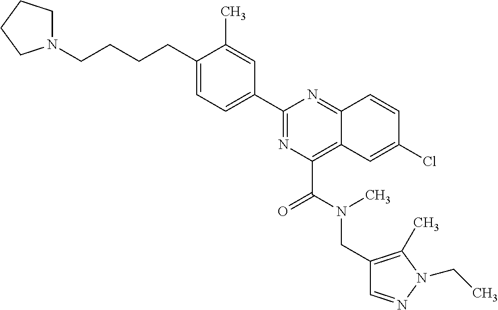 [SMILES: CCn1ncc(CN(C)C(=O)c2nc(-c3ccc(CCCCN4CCCC4)c(C)c3)nc3ccc(Cl)cc23)c1C] | 561.1 | 561.3 | 0.025 | 0.18 | 0.12 | 0.16 |
| 20 | 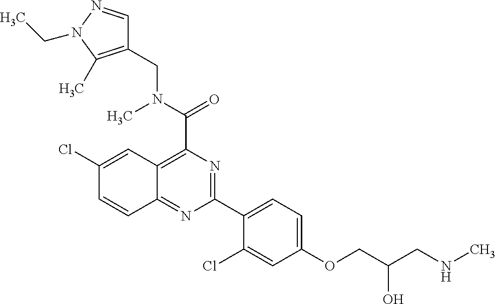 [SMILES: CCn1ncc(CN(C)C(=O)c2nc(-c3ccc(OCC(O)CNC)cc3Cl)nc3ccc(Cl)cc23)c1C] | 557.5 | 557.0 | 0.027 | 0.18 | 0.071 | 0.010 |
| 21 | 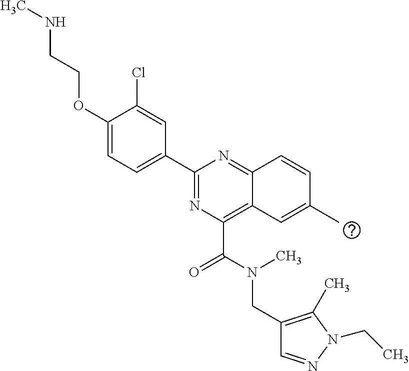 [SMILES: CCn1ncc(CN(C)C(=O)c2nc(-c3ccc(OCCNC)c(Cl)c3)nc3ccc(C)cc23)c1C] | 527.4 | 527.0 | 0.031 | 0.18 | 0.12 | 0.048 |
| 22 | 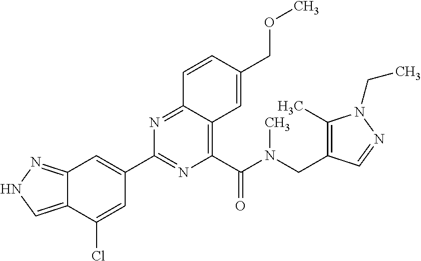 [SMILES: CCn1ncc(CN(C)C(=O)c2nc(-c3cc(Cl)c4c[nH]nc4c3)nc3ccc(COC)cc23)c1C] | 504.0 | 504.2 | 0.014 | 0.18 | 0.21 | 0.014 |
| 23 | 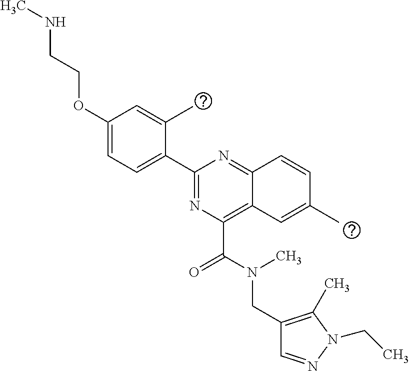 [SMILES: CCn1ncc(CN(C)C(=O)c2nc(-c3ccc(OCCNC)cc3C)nc3ccc(C)cc23)c1C] | 527.4 | 527.0 | 0.046 | 0.19 | 0.082 | 0.050 |
| 24 | 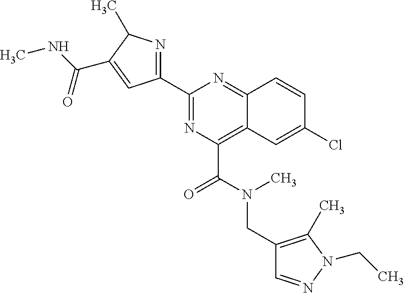 [SMILES: CCn1ncc(CN(C)C(=O)c2nc(C3=NC(C)C(C(=O)NC)=C3)nc3ccc(Cl)cc23)c1C] | 481.0 | 481.0 | 0.10 | 0.21 | 0.18 | 0.0022 |
| 25 | 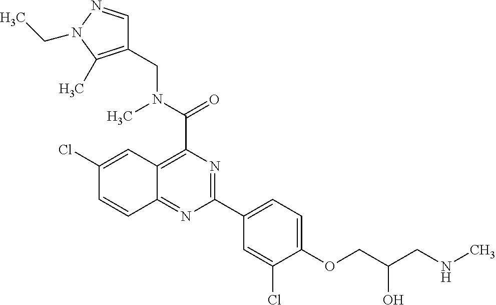 [SMILES: CCn1ncc(CN(C)C(=O)c2nc(-c3ccc(OCC(O)CNC)c(Cl)c3)nc3ccc(Cl)cc23)c1C] | 557.5 | 557.1 | 0.027 | 0.21 | 0.076 | 0.033 |
| 26 | 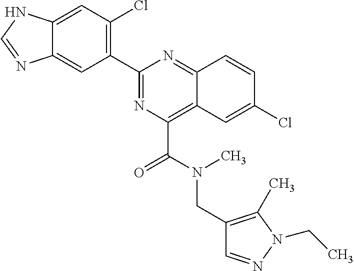 [SMILES: CCn1ncc(CN(C)C(=O)c2nc(-c3cc4nc[nH]c4cc3Cl)nc3ccc(Cl)cc23)c1C] | 494.4 | 494.0 | 0.084 | 0.21 | 0.13 | 0.17 |
| 27 | 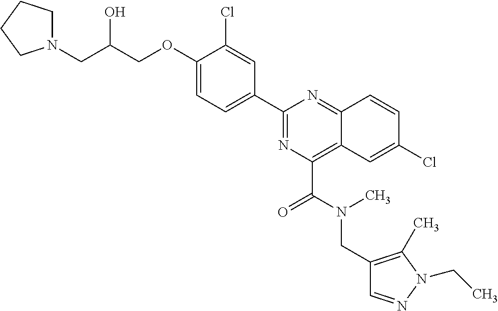 [SMILES: CCn1ncc(CN(C)C(=O)c2nc(-c3ccc(OCC(O)CN4CCCC4)c(Cl)c3)nc3ccc(Cl)cc23)c1C] | 597.5 | 597.1 | 0.039 | 0.22 | 0.11 | 0.020 |
| 28 | 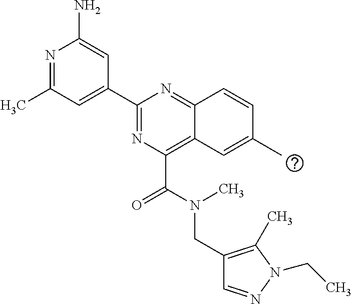 [SMILES: CCn1ncc(CN(C)C(=O)c2nc(-c3cc(C)nc(N)c3)nc3ccc(C)cc23)c1C] | 449.9 | 450.0 | 0.029 | 0.22 | 0.18 | 0.092 |
| 29 | 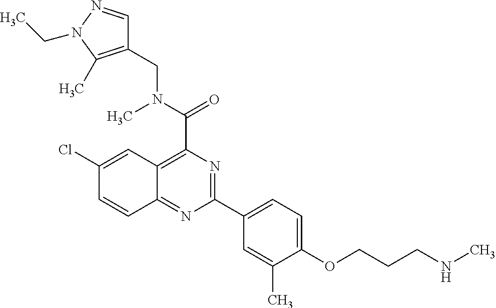 [SMILES: CCn1ncc(CN(C)C(=O)c2nc(-c3ccc(OCCCNC)c(C)c3)nc3ccc(Cl)cc23)c1C] | 521.1 | 521.0 | 0.028 | 0.23 | 0.10 | 0.098 |
| 30 | 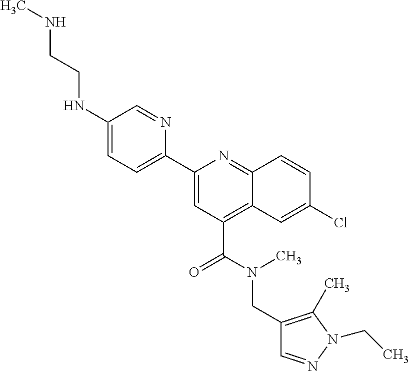 [SMILES: CCn1ncc(CN(C)C(=O)c2cc(-c3ccc(NCCNC)cn3)nc3ccc(Cl)cc23)c1C] | 492.0 | 492.3 | 0.056 | 0.23 | 0.31 | 0.012 |
| 31 | 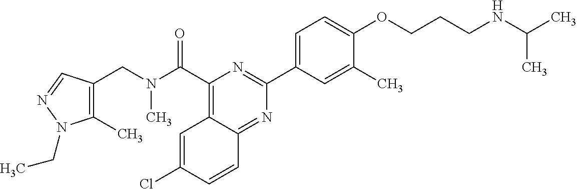 [SMILES: CCn1ncc(CN(C)C(=O)c2nc(-c3ccc(OCCCNC(C)C)c(C)c3)nc3ccc(Cl)cc23)c1C] | 549.1 | 549.3 | 0.028 | 0.26 | 0.13 | 0.14 |
| 32 | 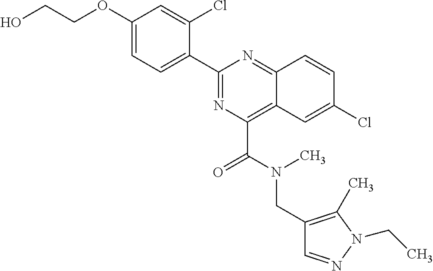 [SMILES: CCn1ncc(CN(C)C(=O)c2nc(-c3ccc(OCCO)cc3Cl)nc3ccc(Cl)cc23)c1C] | 514.4 | 514.0 | 0.15 | 0.27 | 0.15 | 0.33 |
| 33 | 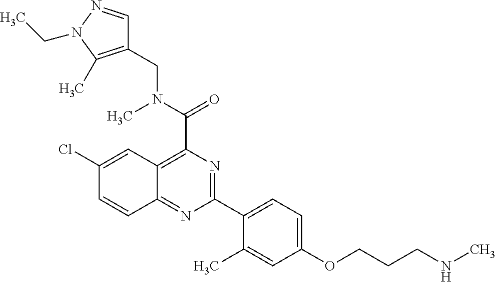 [SMILES: CCn1ncc(CN(C)C(=O)c2nc(-c3ccc(OCCCNC)cc3C)nc3ccc(Cl)cc23)c1C] | 521.1 | 521.0 | 0.068 | 0.27 | 0.18 | 0.053 |
| 34 | 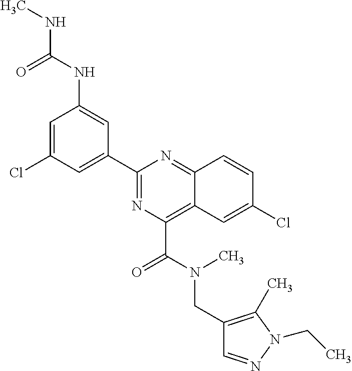 [SMILES: CCn1ncc(CN(C)C(=O)c2nc(-c3cc(Cl)cc(NC(=O)NC)c3)nc3ccc(Cl)cc23)c1C] | 526.4 | 526.0 | 0.0032 | 0.27 | 0.20 | 0.0032 |
| 35 | 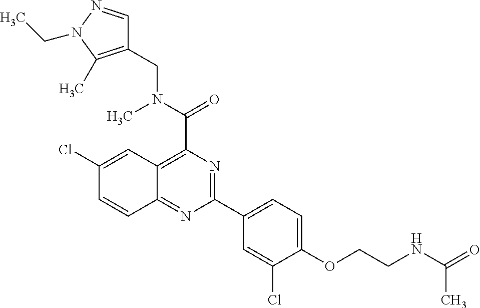 [SMILES: CCn1ncc(CN(C)C(=O)c2nc(-c3ccc(OCCNC(C)=O)c(Cl)c3)nc3ccc(Cl)cc23)c1C] | 555.5 | 555.0 | 0.044 | 0.29 | 0.22 | 0.18 |
| 36 | 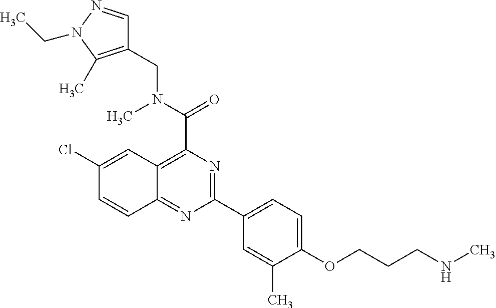 [SMILES: CCn1ncc(CN(C)C(=O)c2nc(-c3ccc(OCCCNC)c(C)c3)nc3ccc(Cl)cc23)c1C] | 521.1 | 521.0 | 0.052 | 0.30 | 0.18 | 0.36 |
| 37 | 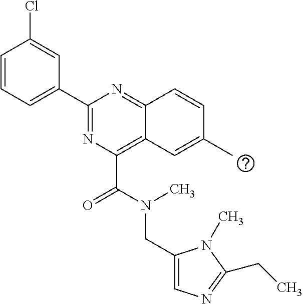 [SMILES: CCc1ncc(CN(C)C(=O)c2nc(-c3cccc(Cl)c3)nc3ccc(C)cc23)n1C] | 454.4 | 454.0 | 0.0034 | 0.30 | 0.29 | 0.0061 |
| 38 | 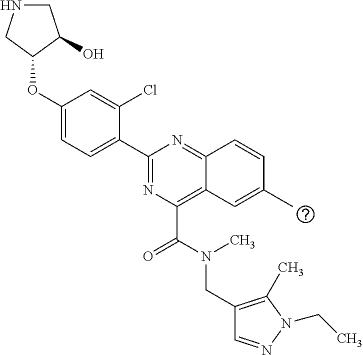 [SMILES: CCn1ncc(CN(C)C(=O)c2nc(-c3ccc(O[C@@H]4CNC[C@H]4O)cc3Cl)nc3ccc(C)cc23)c1C] | 555.5 | 555.2 | 0.034 | 0.33 | 0.23 | 0.012 |
| 39 | 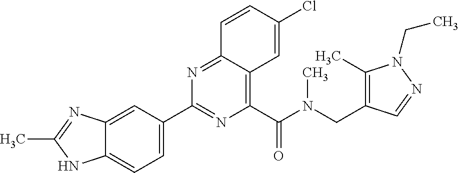 [SMILES: CCn1ncc(CN(C)C(=O)c2nc(-c3ccc4[nH]c(C)nc4c3)nc3ccc(Cl)cc23)c1C] | 474.0 | 474.0 | 0.65 | 0.34 | 0.25 | 0.68 |
| 40 | 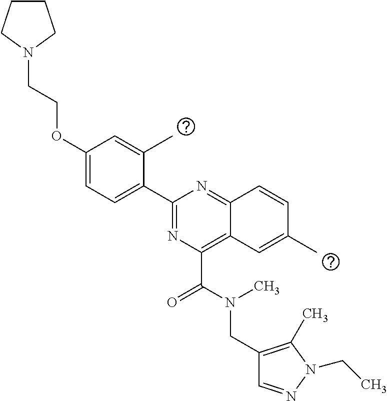 [SMILES: CCn1ncc(CN(C)C(=O)c2nc(-c3ccc(OCCN4CCCC4)cc3C)nc3ccc(C)cc23)c1C] | 567.5 | 567.0 | 0.11 | 0.35 | 0.22 | 0.13 |
| 41 | 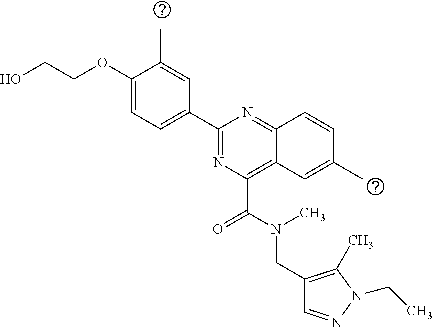 [SMILES: CCn1ncc(CN(C)C(=O)c2nc(-c3ccc(OCCO)c(C)c3)nc3ccc(C)cc23)c1C] | 514.4 | 514.0 | 0.062 | 0.37 | 0.16 | 0.25 |
| 42 | 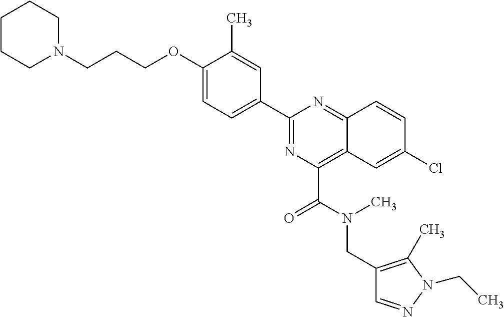 [SMILES: CCn1ncc(CN(C)C(=O)c2nc(-c3ccc(OCCCN4CCCCC4)c(C)c3)nc3ccc(Cl)cc23)c1C] | 575.1 | 575.3 | 0.069 | 0.38 | 0.18 | 0.088 |
| 43 | 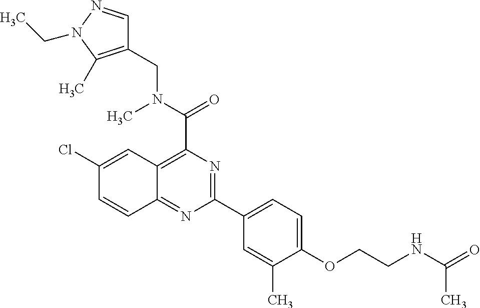 [SMILES: CCn1ncc(CN(C)C(=O)c2nc(-c3ccc(OCCNC(C)=O)c(C)c3)nc3ccc(Cl)cc23)c1C] | 535.0 | 535.2 | 0.069 | 0.38 | 0.46 | 0.34 |
| 44 | 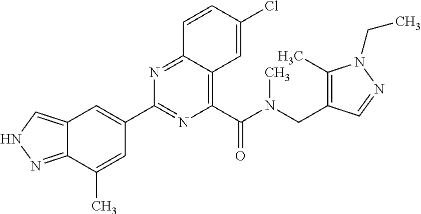 [SMILES: CCn1ncc(CN(C)C(=O)c2nc(-c3cc(C)c4n[nH]cc4c3)nc3ccc(Cl)cc23)c1C] | 474.0 | 474.3 | 0.027 | 0.38 | 0.27 | 0.083 |
| 45 | 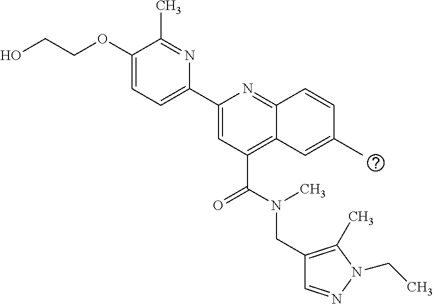 [SMILES: CCn1ncc(CN(C)C(=O)c2cc(-c3ccc(OCCO)c(C)n3)nc3ccc(C)cc23)c1C] | 494.0 | 494.0 | 0.074 | 0.39 | 0.38 | 0.075 |
| 46 | 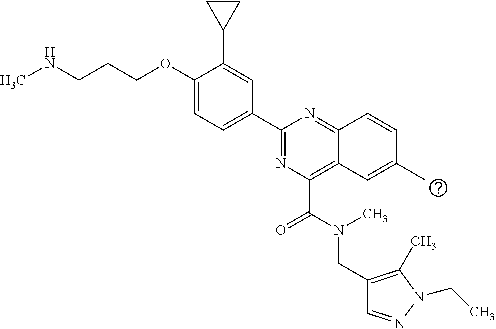 [SMILES: CCn1ncc(CN(C)C(=O)c2nc(-c3ccc(OCCCNC)c(C4CC4)c3)nc3ccc(C)cc23)c1C] | 547.1 | 547.1 | 0.041 | 0.41 | 0.20 | 0.32 |
| 47 | 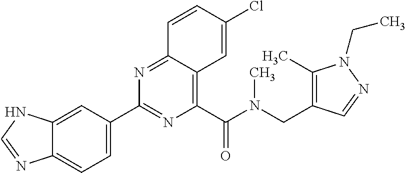 [SMILES: CCn1ncc(CN(C)C(=O)c2nc(-c3ccc4nc[nH]c4c3)nc3ccc(Cl)cc23)c1C] | 459.9 | 460.0 | 0.12 | 0.43 | 0.42 | 0.14 |
| 48 | 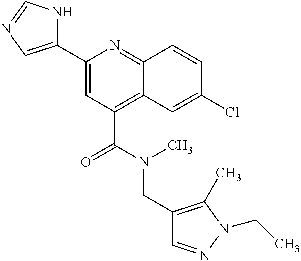 [SMILES: CCn1ncc(CN(C)C(=O)c2cc(-c3cnc[nH]3)nc3ccc(Cl)cc23)c1C] | 408.9 | 409.0 | 0.38 | 0.45 | 0.77 | 0.11 |
| 49 | 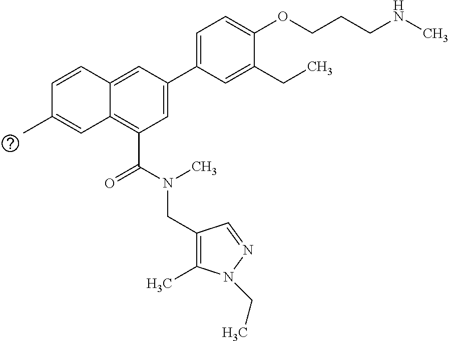 | 535.1 | 535.2 | 0.022 | 0.47 | 0.15 | 0.087 |
| 50 | 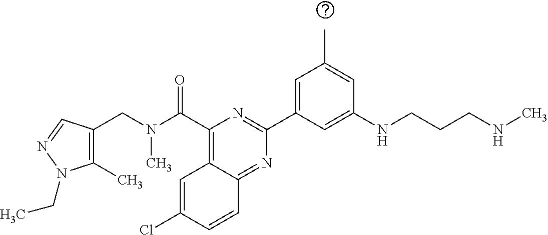 [SMILES: CCn1ncc(CN(C)C(=O)c2nc(-c3cc(C)cc(NCCCNC)c3)nc3ccc(Cl)cc23)c1C] | 540.5 | 540.0 | 0.016 | 0.48 | 0.15 | 0.0050 |
| 51 | 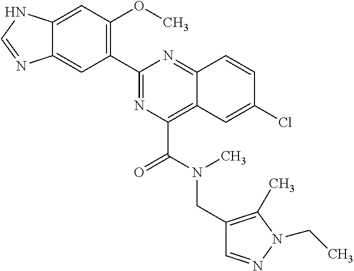 [SMILES: CCn1ncc(CN(C)C(=O)c2nc(-c3cc4nc[nH]c4cc3OC)nc3ccc(Cl)cc23)c1C] | 490.0 | 490.0 | 0.57 | 0.52 | 0.52 | 0.69 |
| 52 | 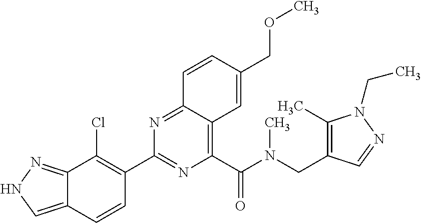 [SMILES: CCn1ncc(CN(C)C(=O)c2nc(-c3ccc4c[nH]nc4c3Cl)nc3ccc(COC)cc23)c1C] | 504.0 | 503.9 | 0.25 | 0.52 | 0.69 | 0.15 |
| 53 | 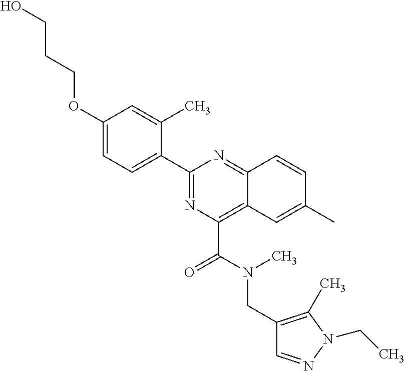 [SMILES: CCn1ncc(CN(C)C(=O)c2nc(-c3ccc(OCCCO)cc3C)nc3ccc(C)cc23)c1C] | 508.0 | 508.0 | 0.13 | 0.57 | 0.46 | 0.28 |
| 54 | 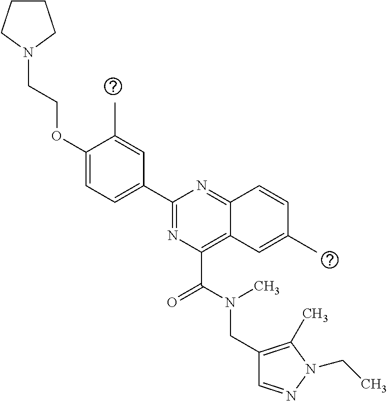 [SMILES: CCn1ncc(CN(C)C(=O)c2nc(-c3ccc(OCCN4CCCC4)c(C)c3)nc3ccc(C)cc23)c1C] | 567.5 | 567.1 | 0.063 | 0.57 | 0.28 | 0.33 |
| 55 | 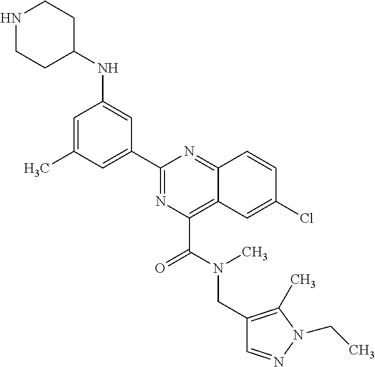 [SMILES: CCn1ncc(CN(C)C(=O)c2nc(-c3cc(C)cc(NC4CCNCC4)c3)nc3ccc(Cl)cc23)c1C] | 532.1 | 532.2 | 0.021 | 0.60 | 0.24 | 0.0070 |
| 56 | 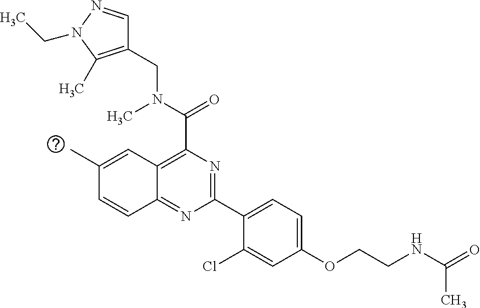 [SMILES: CCn1ncc(CN(C)C(=O)c2nc(-c3ccc(OCCNC(C)=O)cc3Cl)nc3ccc(C)cc23)c1C] | 555.5 | 555.0 | 0.28 | 0.63 | 0.52 | 0.58 |
| 57 | 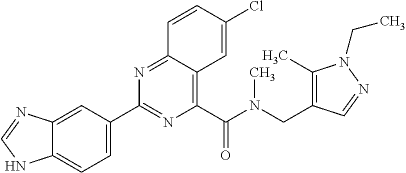 [SMILES: CCn1ncc(CN(C)C(=O)c2nc(-c3ccc4[nH]cnc4c3)nc3ccc(Cl)cc23)c1C] | 459.9 | 460.0 | 0.48 | 0.66 | 0.50 | 0.57 |
| 58 | 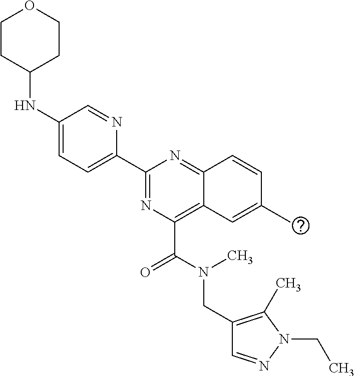 [SMILES: CCn1ncc(CN(C)C(=O)c2nc(-c3ccc(NC4CCOCC4)cn3)nc3ccc(C)cc23)c1C] | 520.0 | 520.1 | 1.5 | 0.67 | 1.4 | 0.61 |
| 59 | 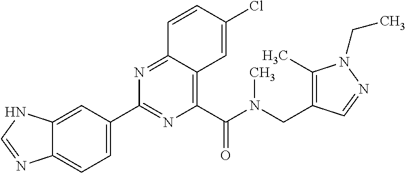 [SMILES: CCn1ncc(CN(C)C(=O)c2nc(-c3ccc4nc[nH]c4c3)nc3ccc(Cl)cc23)c1C] | 459.9 | 460.0 | 0.62 | 0.67 | 0.44 | 0.51 |
| 60 | 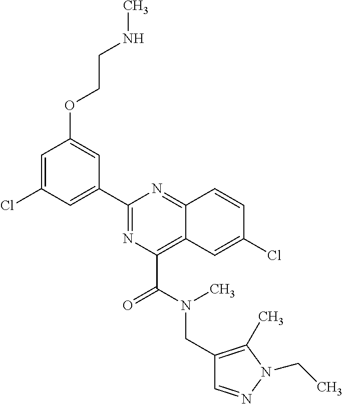 [SMILES: CCn1ncc(CN(C)C(=O)c2nc(-c3cc(Cl)cc(OCCNC)c3)nc3ccc(Cl)cc23)c1C] | 527.4 | 526.9 | 0.023 | 0.88 | 0.50 | 0.011 |
| 61 | 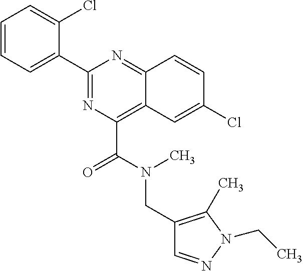 [SMILES: CCn1ncc(CN(C)C(=O)c2nc(-c3ccccc3Cl)nc3ccc(Cl)cc23)c1C] | 454.4 | 454.0 | 0.35 | 0.68 | 0.61 | 0.25 |
| 62 | 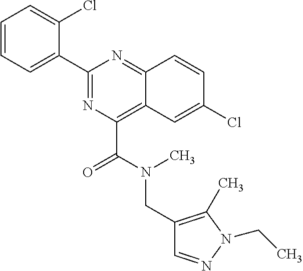 [SMILES: CCn1ncc(CN(C)C(=O)c2nc(-c3ccccc3Cl)nc3ccc(Cl)cc23)c1C] | 454.4 | 454.0 | 0.11 | 0.69 | ND | 0.22 |
| 63 | 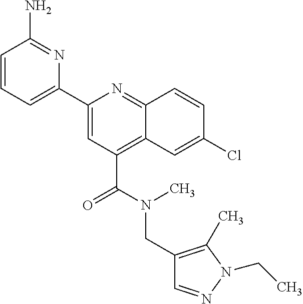 [SMILES: CCn1ncc(CN(C)C(=O)c2cc(-c3cccc(N)n3)nc3ccc(Cl)cc23)c1C] | 434.9 | 435.0 | 0.44 | 0.71 | 1.5 | 0.24 |
| 64 | 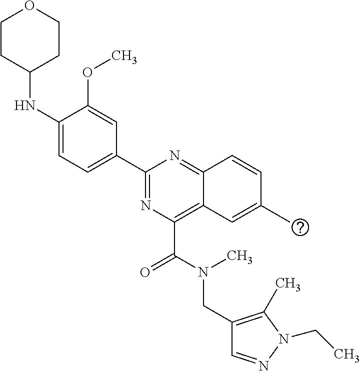 [SMILES: CCn1ncc(CN(C)C(=O)c2nc(-c3ccc(NC4CCOCC4)c(OC)c3)nc3ccc(C)cc23)c1C] | 549.1 | 549.0 | 0.079 | 0.71 | 0.58 | 0.30 |
| 65 | 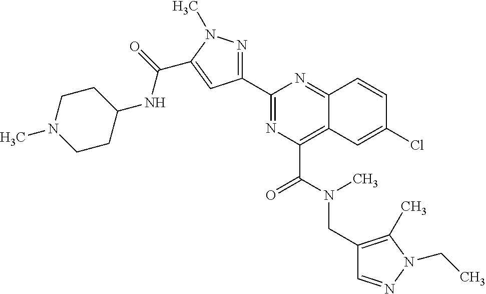 [SMILES: CCn1ncc(CN(C)C(=O)c2nc(-c3cc(C(=O)NC4CCN(C)CC4)n(C)n3)nc3ccc(Cl)cc23)c1C] | 564.1 | 564.3 | 0.41 | 0.72 | 0.74 | 0.79 |
| 66 | 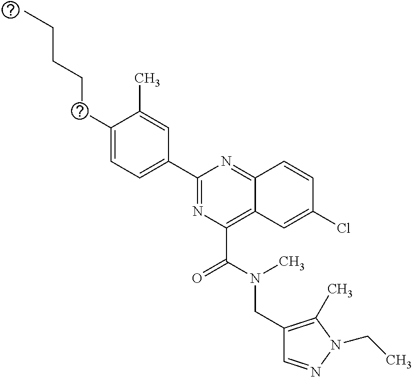 [SMILES: CCCCCc1ccc(-c2nc(C(=O)N(C)Cc3cnn(CC)c3C)c3cc(Cl)ccc3n2)cc1C] | 508.0 | 508.2 | 0.38 | 0.73 | 0.54 | 0.78 |
| 67 | 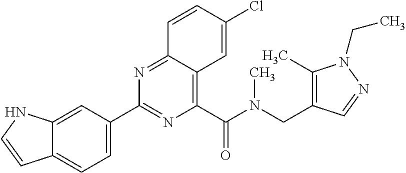 [SMILES: CCn1ncc(CN(C)C(=O)c2nc(-c3ccc4cc[nH]c4c3)nc3ccc(Cl)cc23)c1C] | 458.9 | 459.0 | 0.28 | 0.78 | 0.55 | 0.25 |
| 68 | 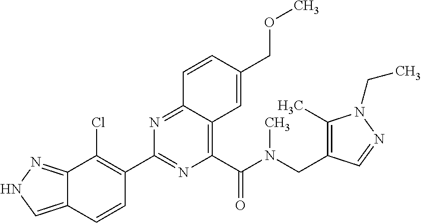 [SMILES: CCn1ncc(CN(C)C(=O)c2nc(-c3ccc4c[nH]nc4c3Cl)nc3ccc(COC)cc23)c1C] | 504.0 | 504.0 | 0.25 | 0.80 | 0.69 | 0.90 |
| 69 | 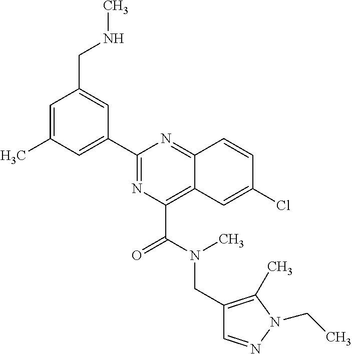 [SMILES: CCn1ncc(CN(C)C(=O)c2nc(-c3cc(C)cc(CNC)c3)nc3ccc(Cl)cc23)c1C] | 477.0 | 477.3 | 0.10 | 0.88 | 0.42 | 0.61 |
| 70 | 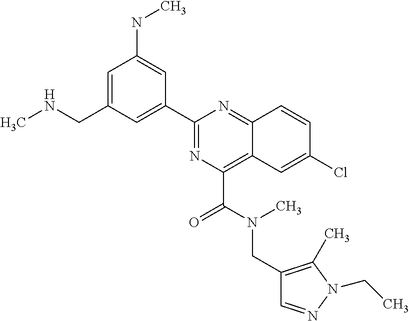 [SMILES: CCn1ncc(CN(C)C(=O)c2nc(-c3cc(CNC)cc(NC)c3)nc3ccc(Cl)cc23)c1C] | 493.0 | 492.9 | 0.15 | 0.89 | 0.51 | 0.99 |
| 71 | 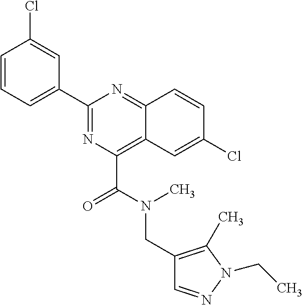 [SMILES: CCn1ncc(CN(C)C(=O)c2nc(-c3cccc(Cl)c3)nc3ccc(Cl)cc23)c1C] | 454.4 | 454.0 | 0.0094 | 0.92 | 1.2 | |
| 72 | 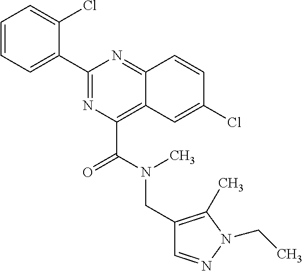 [SMILES: CCn1ncc(CN(C)C(=O)c2nc(-c3ccccc3Cl)nc3ccc(Cl)cc23)c1C] | 454.4 | 454.0 | 0.11 | 0.94 | 0.78 | |
| 73 | 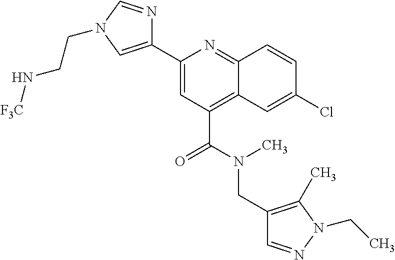 [SMILES: CCn1ncc(CN(C)C(=O)c2cc(-c3cn(CCNC(F)(F)F)cn3)nc3ccc(Cl)cc23)c1C] | 466.0 | 466.1 | 0.82 | 1.0 | 1.3 | 0.36 |
| 74 | 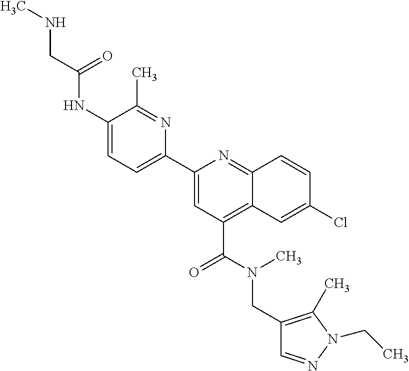 [SMILES: CCn1ncc(CN(C)C(=O)c2cc(-c3ccc(NC(=O)CNC)c(C)n3)nc3ccc(Cl)cc23)c1C] | 520.0 | 519.9 | 2.1 | 1.0 | 1.5 | 1.1 |
| 75 | 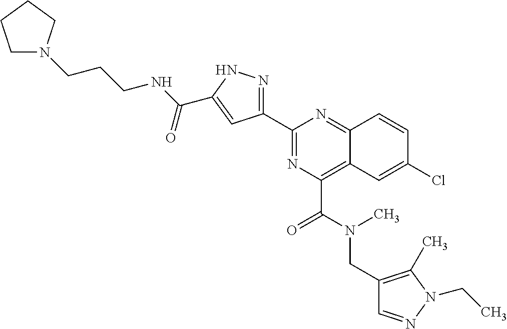 [SMILES: CCn1ncc(CN(C)C(=O)c2nc(-c3cc(C(=O)NCCCN4CCCC4)[nH]n3)nc3ccc(Cl)cc23)c1C] | 564.1 | 564.1 | 0.13 | 1.0 | 0.51 | 0.054 |
| 76 | 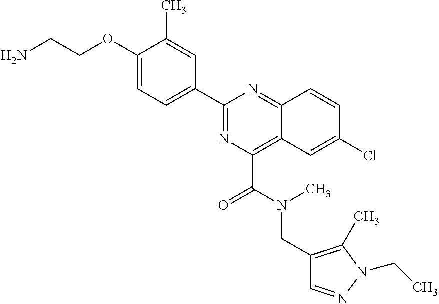 [SMILES: CCn1ncc(CN(C)C(=O)c2nc(-c3ccc(OCCN)c(C)c3)nc3ccc(Cl)cc23)c1C] | 493.0 | 492.9 | 0.028 | 1.1 | 0.84 | 0.18 |
| 77 | 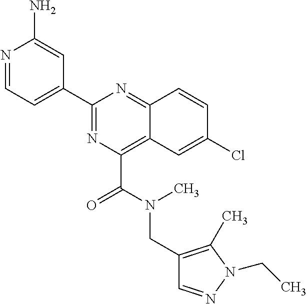 [SMILES: CCn1ncc(CN(C)C(=O)c2nc(-c3ccnc(N)c3)nc3ccc(Cl)cc23)c1C] | 435.9 | 436.0 | 1.6 | 1.1 | 0.76 | 1.3 |
| 78 | 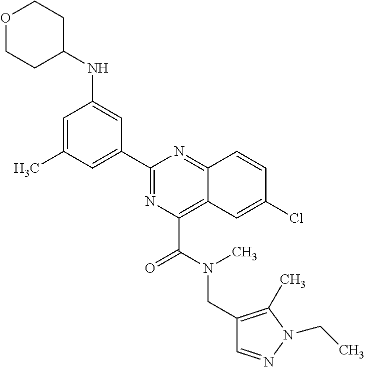 [SMILES: CCn1ncc(CN(C)C(=O)c2nc(-c3cc(C)cc(NC4CCOCC4)c3)nc3ccc(Cl)cc23)c1C] | 533.1 | 533.2 | 0.067 | 1.1 | 1.2 | 0.066 |
| 79 | 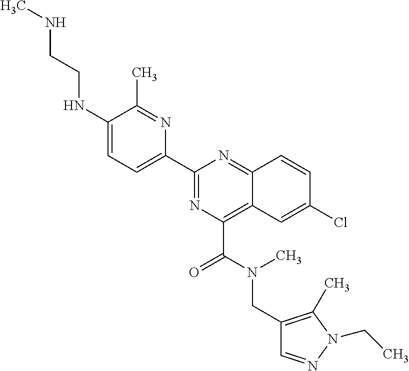 [SMILES: CCn1ncc(CN(C)C(=O)c2nc(-c3ccc(NCCNC)c(C)n3)nc3ccc(Cl)cc23)c1C] | 507.0 | 507.3 | 0.67 | 1.2 | 0.85 | 2.1 |
| 80 | 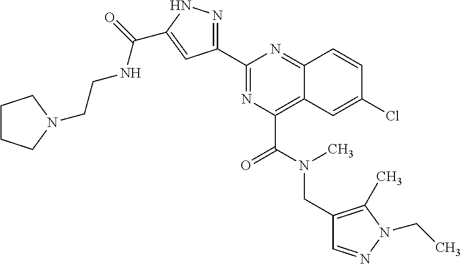 [SMILES: CCn1ncc(CN(C)C(=O)c2nc(-c3cc(C(=O)NCCN4CCCC4)[nH]n3)nc3ccc(Cl)cc23)c1C] | 550.1 | 550.1 | 1.2 | 1.2 | 1.4 | 0.69 |
| 81 | 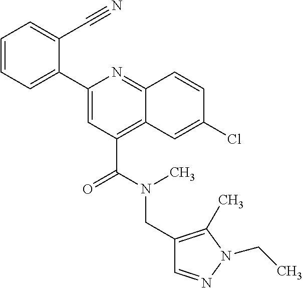 [SMILES: CCn1ncc(CN(C)C(=O)c2cc(-c3ccccc3C#N)nc3ccc(Cl)cc23)c1C] | 443.9 | 444.1 | 1.8 | 1.2 | 2.2 | |
| 82 | 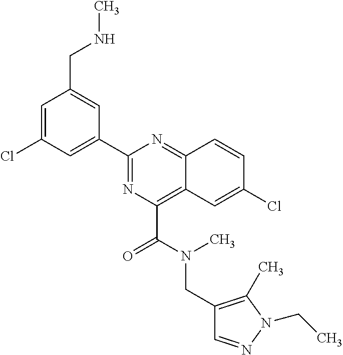 [SMILES: CCn1ncc(CN(C)C(=O)c2nc(-c3cc(Cl)cc(CNC)c3)nc3ccc(Cl)cc23)c1C] | 497.4 | 497.0 | 0.10 | 1.2 | 0.58 | 0.46 |
| 83 | 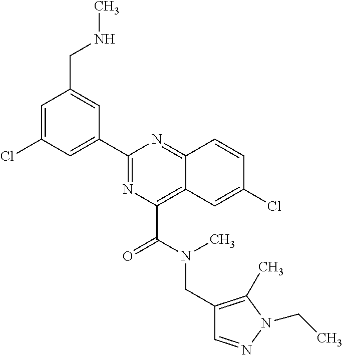 [SMILES: CCn1ncc(CN(C)C(=O)c2nc(-c3cc(Cl)cc(CNC)c3)nc3ccc(Cl)cc23)c1C] | 497.4 | 497.0 | 0.10 | 1.2 | 0.58 | 0.46 |
| 84 | 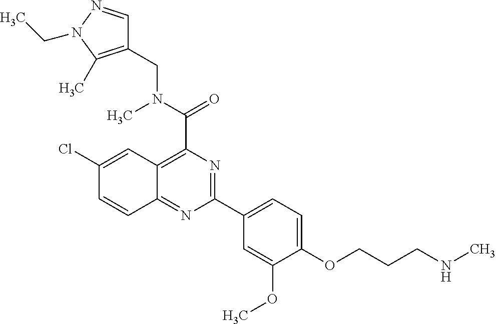 [SMILES: CCn1ncc(CN(C)C(=O)c2nc(-c3ccc(OCCCNC)c(OC)c3)nc3ccc(Cl)cc23)c1C] | 537.1 | 537.1 | 0.82 | 1.3 | 0.85 | 2.5 |
| 85 | 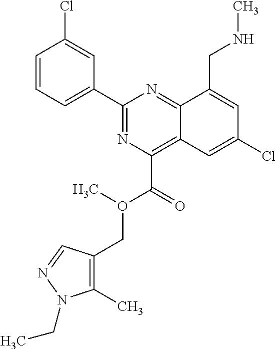 | 497.4 | 497.0 | 0.18 | 1.3 | 1.3 | 0.40 |
| 86 | 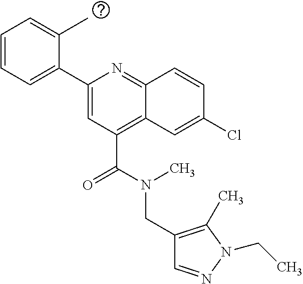 [SMILES: CCn1ncc(CN(C)C(=O)c2cc(-c3ccccc3C)nc3ccc(Cl)cc23)c1C] | 436.9 | 437.1 | 0.71 | 1.3 | 1.4 | |
| 87 | 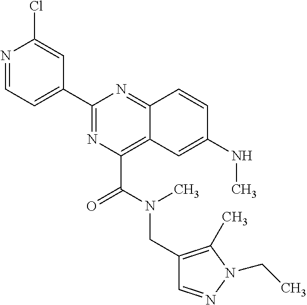 [SMILES: CCn1ncc(CN(C)C(=O)c2nc(-c3ccnc(Cl)c3)nc3ccc(NC)cc23)c1C] | 449.9 | 450.0 | 0.84 | 1.4 | 1.4 | 0.88 |
| 88 | 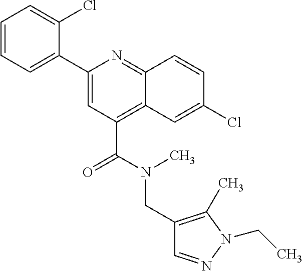 [SMILES: CCn1ncc(CN(C)C(=O)c2cc(-c3ccccc3Cl)nc3ccc(Cl)cc23)c1C] | 467.4 | 467.0 | 0.31 | 1.5 | ND | |
| 89 | 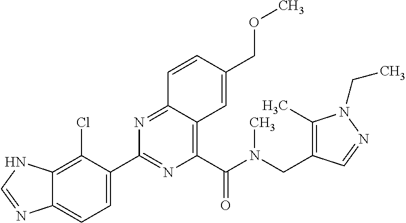 [SMILES: CCn1ncc(CN(C)C(=O)c2nc(-c3ccc4nc[nH]c4c3Cl)nc3ccc(COC)cc23)c1C] | 504.0 | 504.0 | 0.18 | 1.6 | 1.2 | 1.0 |
| 90 | 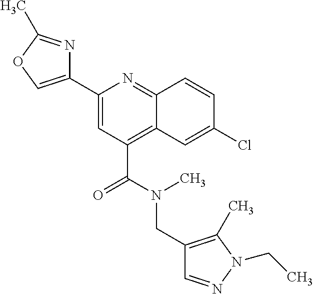 [SMILES: CCn1ncc(CN(C)C(=O)c2cc(-c3coc(C)n3)nc3ccc(Cl)cc23)c1C] | 440.0 | 440.0 | 0.34 | 1.6 | 1.5 | 0.21 |
| 91 | 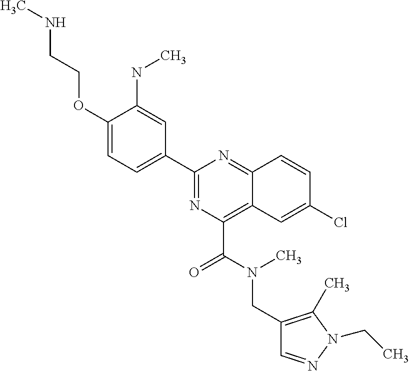 [SMILES: CCn1ncc(CN(C)C(=O)c2nc(-c3ccc(OCCNC)c(NC)c3)nc3ccc(Cl)cc23)c1C] | 523.0 | 523.1 | 0.91 | 1.6 | 0.89 | 1.2 |
| 92 | 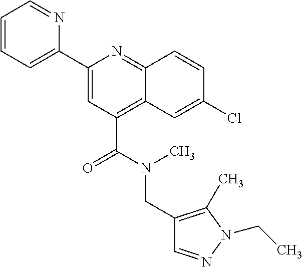 [SMILES: CCn1ncc(CN(C)C(=O)c2cc(-c3ccccn3)nc3ccc(Cl)cc23)c1C] | 419.9 | 420.0 | 0.31 | 1.7 | 0.41 | |
| 93 | 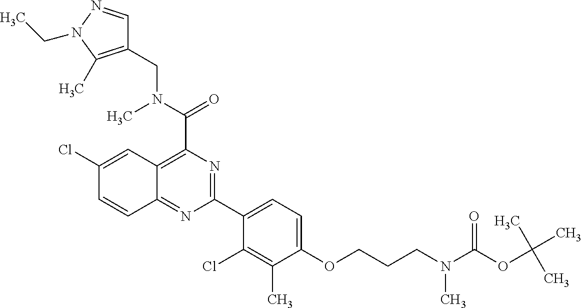 [SMILES: CCn1ncc(CN(C)C(=O)c2nc(-c3ccc(OCCCN(C)C(=O)OC(C)(C)C)c(C)c3Cl)nc3ccc(Cl)cc23)c1C] | 655.6 | 655.0 | 0.14 | 1.7 | 1.2 | 0.068 |
| 94 | 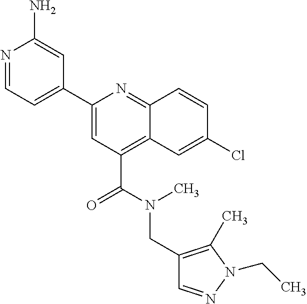 [SMILES: CCn1ncc(CN(C)C(=O)c2cc(-c3ccnc(N)c3)nc3ccc(Cl)cc23)c1C] | 434.9 | 435.0 | 2.1 | 1.7 | 1.5 | 1.5 |
| 95 | 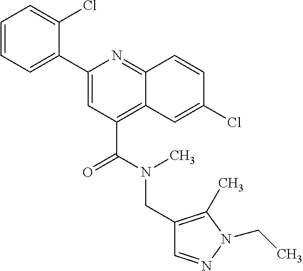 [SMILES: CCn1ncc(CN(C)C(=O)c2cc(-c3ccccc3Cl)nc3ccc(Cl)cc23)c1C] | 453.4 | 453.0 | 0.25 | 1.8 | | |
| 96 | 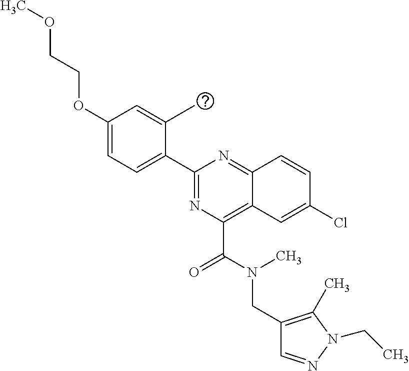 [SMILES: CCn1ncc(CN(C)C(=O)c2nc(-c3ccc(OCCOC)cc3C)nc3ccc(Cl)cc23)c1C] | 526.4 | 528.0 | 0.74 | 1.9 | 1.8 | 1.5 |
| 97 | 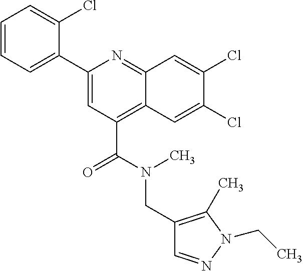 [SMILES: CCn1ncc(CN(C)C(=O)c2cc(-c3ccccc3Cl)nc3cc(Cl)c(Cl)cc23)c1C] | 487.8 | 487.0 | 0.47 | 1.9 | | |
| 98 | 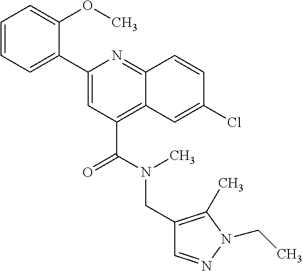 [SMILES: CCn1ncc(CN(C)C(=O)c2cc(-c3ccccc3OC)nc3ccc(Cl)cc23)c1C] | 448.9 | 449.1 | 0.40 | 2.0 | | |
| 99 | 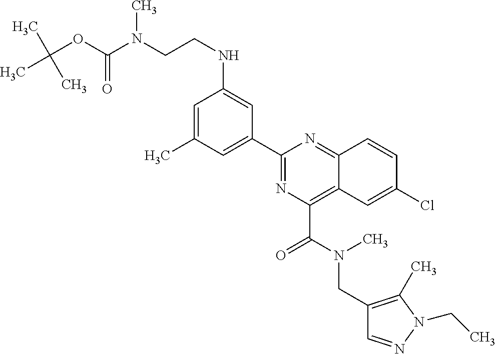 [SMILES: CCn1ncc(CN(C)C(=O)c2nc(-c3cc(C)cc(NCCN(C)C(=O)OC(C)(C)C)c3)nc3ccc(Cl)cc23)c1C] | 606.2 | 605.9 | 2.2 | 2.0 | 2.1 | 2.6 |
| 100 | 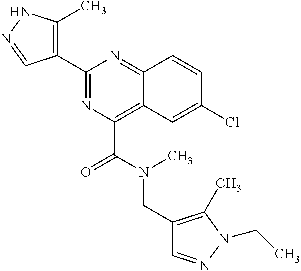 [SMILES: CCn1ncc(CN(C)C(=O)c2nc(-c3cn[nH]c3C)nc3ccc(Cl)cc23)c1C] | 423.9 | 424.1 | 11 | 2.1 | 1.6 | 6.3 |
| 101 | 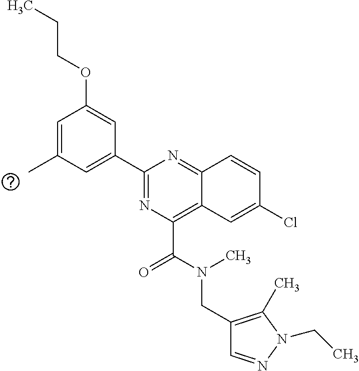 [SMILES: CCCOc1cc(C)cc(-c2nc(C(=O)N(C)Cc3cnn(CC)c3C)c3cc(Cl)ccc3n2)c1] | 514.4 | 513.9 | 1.9 | 2.1 | 2.9 | 1.0 |
| 102 | 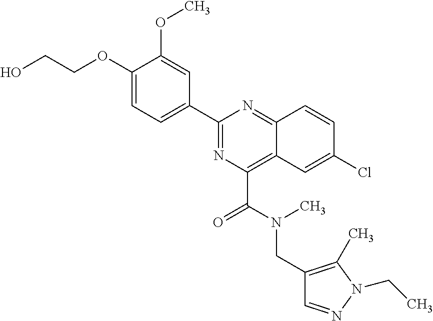 [SMILES: CCn1ncc(CN(C)C(=O)c2nc(-c3ccc(OCCO)c(OC)c3)nc3ccc(Cl)cc23)c1C] | 510.0 | 510.0 | 4.0 | 2.1 | 2.3 | 4.3 |
| 103 | 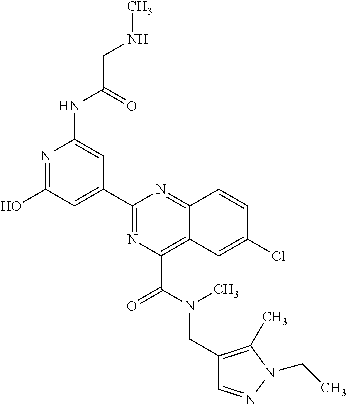 [SMILES: CCn1ncc(CN(C)C(=O)c2nc(-c3cc(O)nc(NC(=O)CNC)c3)nc3ccc(Cl)cc23)c1C] | 521.0 | 521.3 | 1.7 | 2.2 | 1.7 | 4.9 |
| 104 | 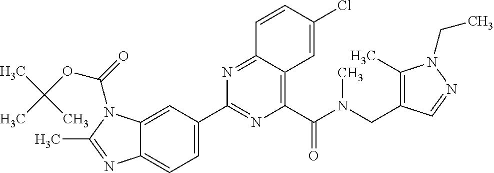 [SMILES: CCn1ncc(CN(C)C(=O)c2nc(-c3ccc4nc(C)n(C(=O)OC(C)(C)C)c4c3)nc3ccc(Cl)cc23)c1C] | 574.1 | 474.1 | 9.2 | 2.2 | 0.23 | 13 |
| 105 | 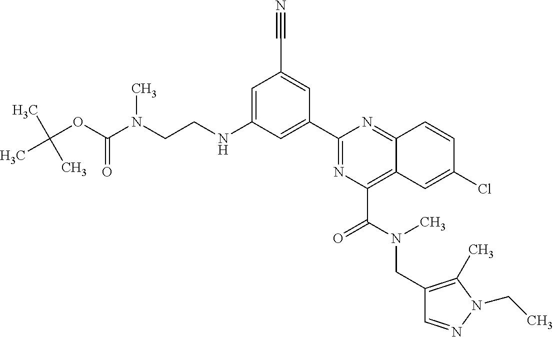 [SMILES: CCn1ncc(CN(C)C(=O)c2nc(-c3cc(C#N)cc(NCCN(C)C(=O)OC(C)(C)C)c3)nc3ccc(Cl)cc23)c1C] | 617.1 | 617.0 | 3.3 | 2.4 | 1.8 | 2.3 |
| 106 | 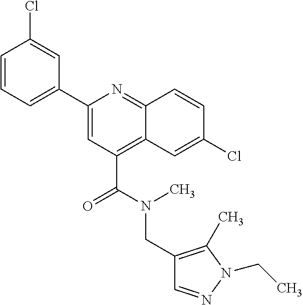 [SMILES: CCn1ncc(CN(C)C(=O)c2cc(-c3cccc(Cl)c3)nc3ccc(Cl)cc23)c1C] | 453.4 | 453.1 | 0.31 | 2.4 | 2.6 | |
| 107 | 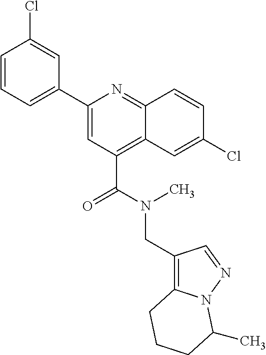 [SMILES: CC1CCCc2c(CN(C)C(=O)c3cc(-c4cccc(Cl)c4)nc4ccc(Cl)cc34)cnn21] | 479.4 | 479.0 | 1.1 | 2.5 | 6.2 | 0.59 |
| 108 | 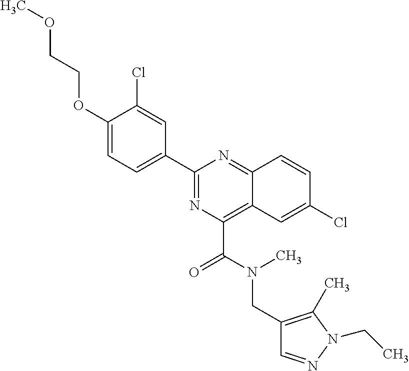 [SMILES: CCn1ncc(CN(C)C(=O)c2nc(-c3ccc(OCCOC)c(Cl)c3)nc3ccc(Cl)cc23)c1C] | 528.4 | 528.0 | 0.97 | 2.5 | 2.2 | 0.93 |
| 109 | 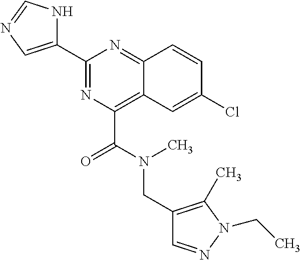 [SMILES: CCn1ncc(CN(C)C(=O)c2nc(-c3cnc[nH]3)nc3ccc(Cl)cc23)c1C] | 409.9 | 410.1 | 3.9 | 2.5 | 3.5 | 1.4 |
| 110 | 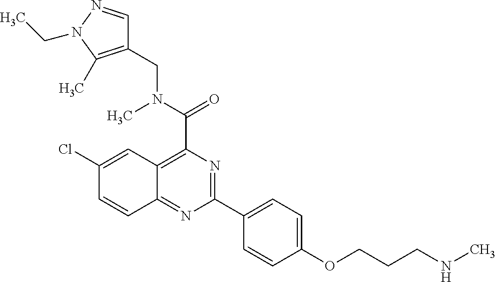 [SMILES: CCn1ncc(CN(C)C(=O)c2nc(-c3ccc(OCCCNC)cc3)nc3ccc(Cl)cc23)c1C] | 507.0 | 507.0 | 3.2 | 2.6 | 1.4 | 4.4 |
| 111 | 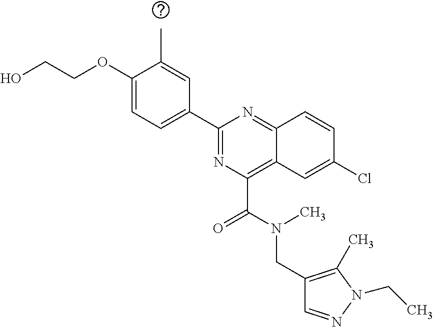 [SMILES: CCn1ncc(CN(C)C(=O)c2nc(-c3ccc(OCCO)c(C)c3)nc3ccc(Cl)cc23)c1C] | 498.0 | 498.0 | 6.4 | 2.6 | 2.4 | 4.7 |
| 112 | 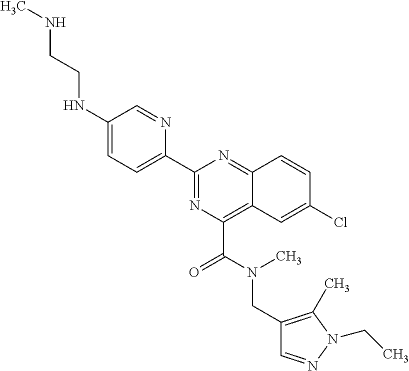 [SMILES: CCn1ncc(CN(C)C(=O)c2nc(-c3ccc(NCCNC)cn3)nc3ccc(Cl)cc23)c1C] | 493.0 | 493.0 | 1.3 | 2.7 | 2.1 | 0.73 |
| 113 | 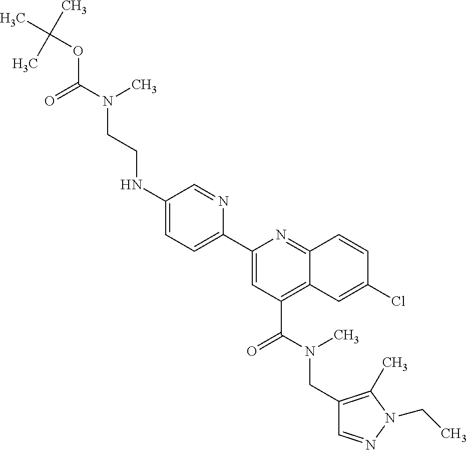 [SMILES: CCn1ncc(CN(C)C(=O)c2cc(-c3ccc(NCCN(C)C(=O)OC(C)(C)C)cn3)nc3ccc(Cl)cc23)c1C] | 592.1 | 592.2 | 4.8 | 2.8 | 6.7 | 1.5 |
| 114 | 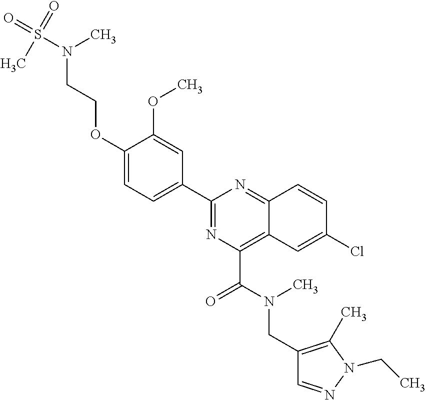 [SMILES: CCn1ncc(CN(C)C(=O)c2nc(-c3ccc(OCCN(C)S(C)(=O)=O)c(OC)c3)nc3ccc(Cl)cc23)c1C] | 601.1 | 601.1 | 5.2 | 2.8 | 2.8 | 4.3 |
| 115 | 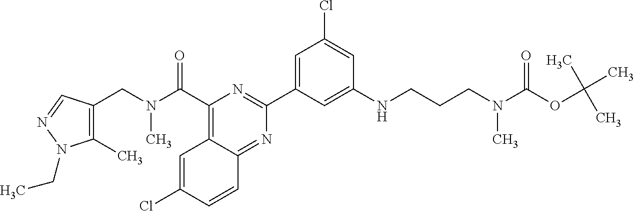 [SMILES: CCn1ncc(CN(C)C(=O)c2nc(-c3cc(Cl)cc(NCCCN(C)C(=O)OC(C)(C)C)c3)nc3ccc(Cl)cc23)c1C] | 640.6 | 640.1 | 0.16 | 2.8 | 4.4 | 0.11 |
| 116 | 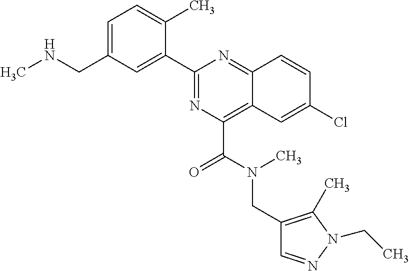 [SMILES: CCn1ncc(CN(C)C(=O)c2nc(-c3cc(CNC)ccc3C)nc3ccc(Cl)cc23)c1C] | 477.0 | 477.2 | 3.4 | 2.9 | 1.9 | 6.9 |
| 117 | 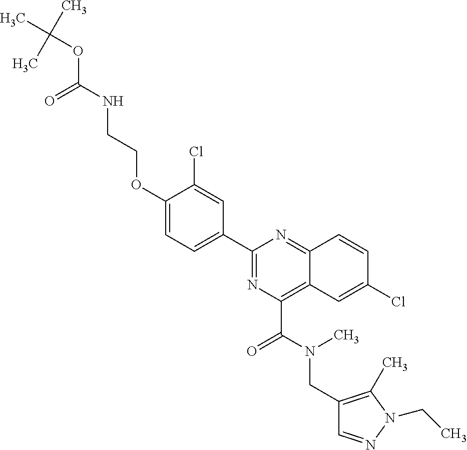 [SMILES: CCn1ncc(CN(C)C(=O)c2nc(-c3ccc(OCCNC(=O)OC(C)(C)C)c(Cl)c3)nc3ccc(Cl)cc23)c1C] | 613.5 | 613.0 | 0.27 | 3.0 | 2.4 | 4.3 |
| 118 | 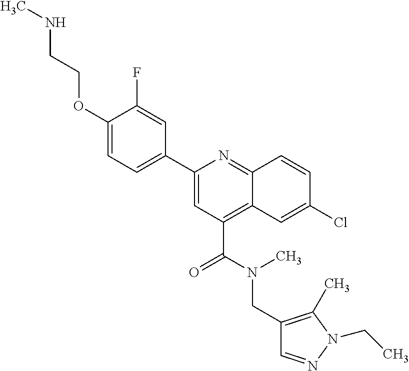 [SMILES: CCn1ncc(CN(C)C(=O)c2cc(-c3ccc(OCCNC)c(F)c3)nc3ccc(Cl)cc23)c1C] | 511.0 | 511.1 | 5.6 | 3.3 | 1.4 | 3.2 |
| 119 | 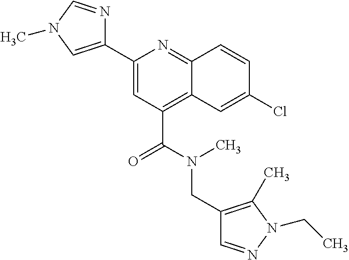 [SMILES: CCn1ncc(CN(C)C(=O)c2cc(-c3cn(C)cn3)nc3ccc(Cl)cc23)c1C] | 422.9 | 423.1 | 4.5 | 3.3 | 0.69 | 1.3 |
| 120 | 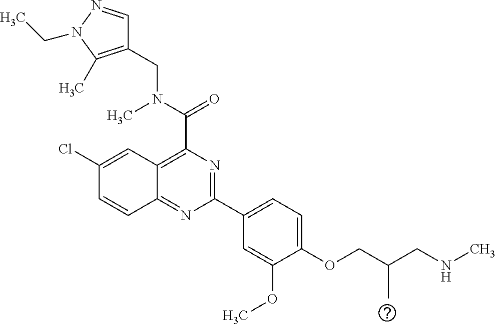 [SMILES: CCn1ncc(CN(C)C(=O)c2nc(-c3ccc(OCC(C)CNC)c(OC)c3)nc3ccc(Cl)cc23)c1C] | 553.1 | 553.0 | 0.55 | 3.4 | 0.63 | 1.4 |
| 121 | 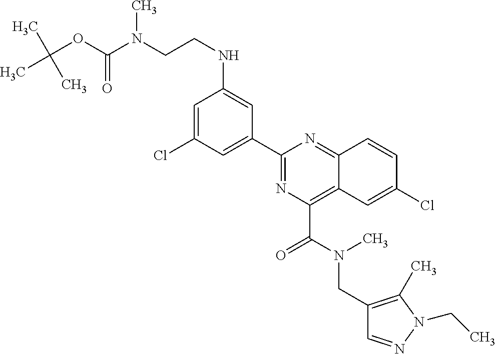 [SMILES: CCn1ncc(CN(C)C(=O)c2nc(-c3cc(Cl)cc(NCCN(C)C(=O)OC(C)(C)C)c3)nc3ccc(Cl)cc23)c1C] | 626.6 | 626.0 | 0.41 | 3.5 | 3.8 | 0.86 |
| 122 | 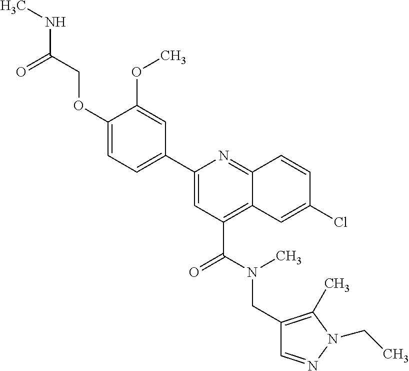 [SMILES: CCn1ncc(CN(C)C(=O)c2cc(-c3ccc(OCC(=O)NC)c(OC)c3)nc3ccc(Cl)cc23)c1C] | 537.0 | 537.0 | 4.5 | 3.6 | 2.1 | 4.3 |
| 123 | 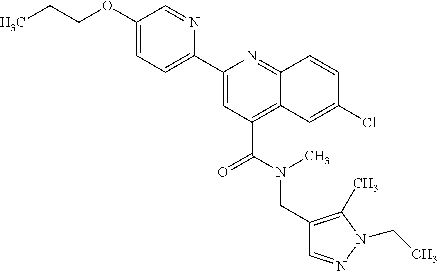 [SMILES: CCCOc1ccc(-c2cc(C(=O)N(C)Cc3cnn(CC)c3C)c3cc(Cl)ccc3n2)nc1] | 480.0 | 480.1 | 6.0 | 3.7 | 4.0 | 1.8 |
| 124 | 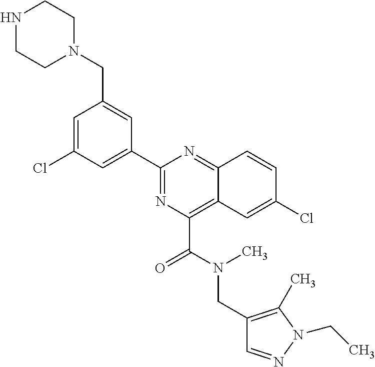 [SMILES: CCn1ncc(CN(C)C(=O)c2nc(-c3cc(Cl)cc(CN4CCNCC4)c3)nc3ccc(Cl)cc23)c1C] | 552.5 | 552.2 | 0.37 | 3.9 | 4.1 | 1.4 |
| 125 | 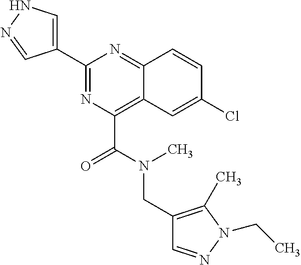 [SMILES: CCn1ncc(CN(C)C(=O)c2nc(-c3cn[nH]c3)nc3ccc(Cl)cc23)c1C] | 409.9 | 410.1 | 6.3 | 4.0 | 4.1 | 3.5 |
| 126 | 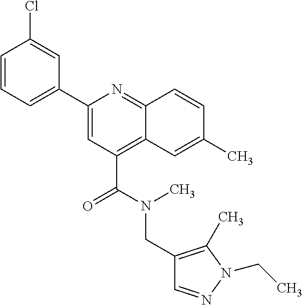 [SMILES: CCn1ncc(CN(C)C(=O)c2cc(-c3cccc(Cl)c3)nc3ccc(C)cc23)c1C] | 432.9 | 433.1 | 0.98 | 4.3 | | |
| 127 | 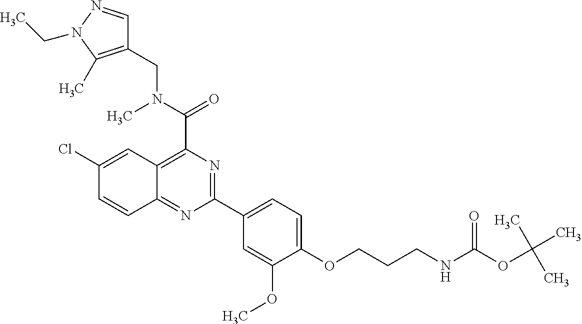 [SMILES: CCn1ncc(CN(C)C(=O)c2nc(-c3ccc(OCCCNC(=O)OC(C)(C)C)c(OC)c3)nc3ccc(Cl)cc23)c1C] | 623.1 | 623.1 | 12 | 4.4 | 3.5 | 15 |
| 128 | 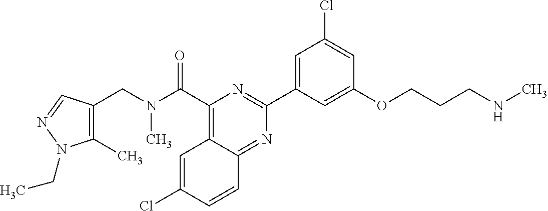 [SMILES: CCn1ncc(CN(C)C(=O)c2nc(-c3cc(Cl)cc(OCCCNC)c3)nc3ccc(Cl)cc23)c1C] | 541.5 | 541.0 | 0.35 | 4.5 | 1.9 | 0.80 |
| 129 | 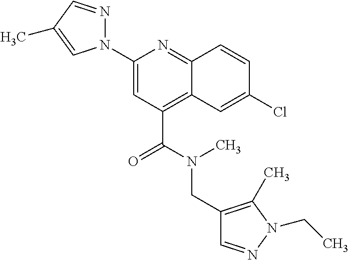 [SMILES: CCn1ncc(CN(C)C(=O)c2cc(-n3cc(C)cn3)nc3ccc(Cl)cc23)c1C] | 422.9 | 423.0 | 7.4 | 4.6 | 8.3 | 1.4 |
| 130 | 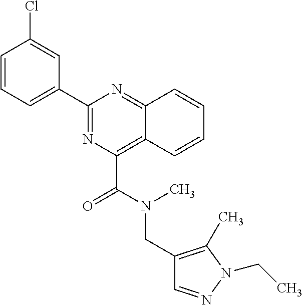 [SMILES: CCn1ncc(CN(C)C(=O)c2nc(-c3cccc(Cl)c3)nc3ccccc23)c1C] | 419.9 | 420.1 | 2.0 | 4.7 | 6.5 | |
| 131 | 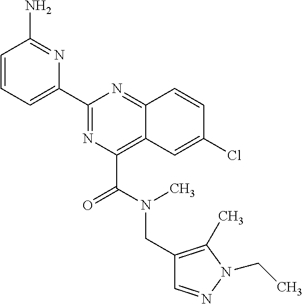 [SMILES: CCn1ncc(CN(C)C(=O)c2nc(-c3cccc(N)n3)nc3ccc(Cl)cc23)c1C] | 435.9 | 436.0 | 5.5 | 4.7 | 7.5 | 2.6 |
| 132 | 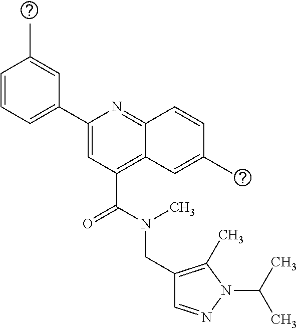 [SMILES: Cc1cccc(-c2cc(C(=O)N(C)Cc3cnn(C(C)C)c3C)c3cc(C)ccc3n2)c1] | 467.4 | 467.0 | 0.31 | 4.9 | 2.2 | |
| 133 | 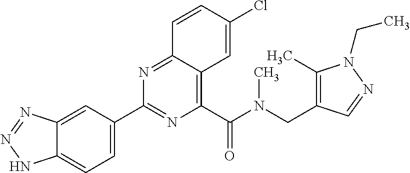 [SMILES: CCn1ncc(CN(C)C(=O)c2nc(-c3ccc4[nH]nnc4c3)nc3ccc(Cl)cc23)c1C] | 460.9 | 461.0 | 0.10 | 4.9 | 5.9 | 0.47 |
| 134 | 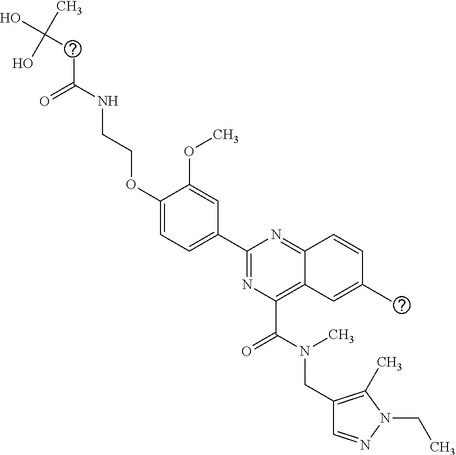 [SMILES: CCn1ncc(CN(C)C(=O)c2nc(-c3ccc(OCCNC(=O)CC(C)(O)O)c(OC)c3)nc3ccc(C)cc23)c1C] | 609.1 | 609.1 | 9.0 | 5.1 | 3.1 | 13 |
| 135 | 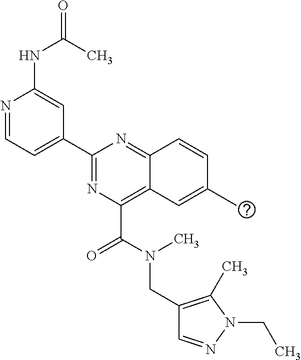 [SMILES: CCn1ncc(CN(C)C(=O)c2nc(-c3ccnc(NC(C)=O)c3)nc3ccc(C)cc23)c1C] | 478.0 | 478.0 | 7.5 | 5.1 | 6.7 | 7.1 |
| 136 | 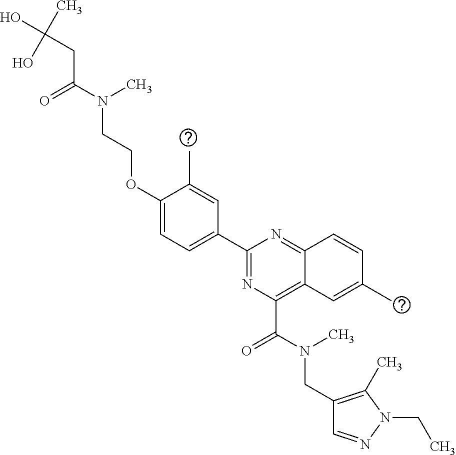 [SMILES: CCn1ncc(CN(C)C(=O)c2nc(-c3ccc(OCCN(C)C(=O)CC(C)(O)O)c(C)c3)nc3ccc(C)cc23)c1C] | 672.0 | 671.2 | 0.15 | 5.2 | 1.7 | 0.52 |
| 137 | 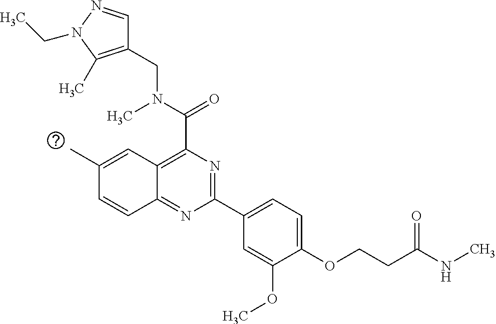 [SMILES: CCn1ncc(CN(C)C(=O)c2nc(-c3ccc(OCCC(=O)NC)c(OC)c3)nc3ccc(C)cc23)c1C] | 551.0 | 551.2 | 8.3 | 5.3 | 4.7 | 7.8 |
| 138 | 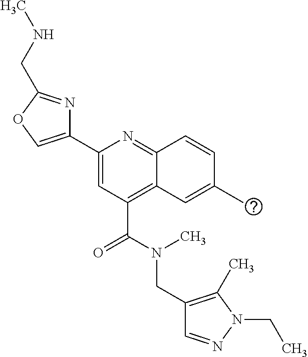 [SMILES: CCn1ncc(CN(C)C(=O)c2cc(-c3coc(CNC)n3)nc3ccc(C)cc23)c1C] | 469.0 | 469.0 | 12 | 5.9 | 8.3 | 5.2 |
| 139 | 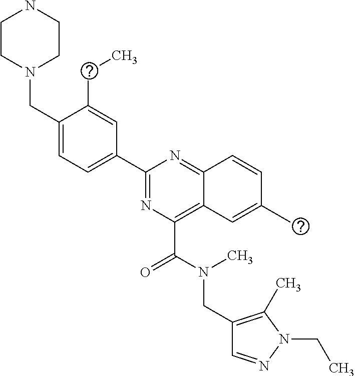 [SMILES: CCc1cc(-c2nc(C(=O)N(C)Cc3cnn(CC)c3C)c3cc(C)ccc3n2)ccc1CN1CCNCC1] | 549.1 | 549.0 | 6.8 | 5.9 | 7.5 | 7.4 |
| 140 | 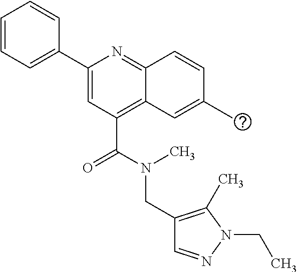 [SMILES: CCn1ncc(CN(C)C(=O)c2cc(-c3ccccc3)nc3ccc(C)cc23)c1C] | 4189 | 419.1 | 3.9 | 6.1 | | |
| 141 | 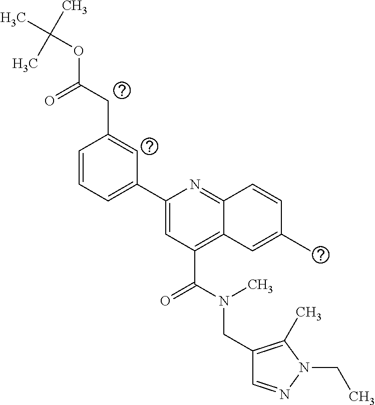 [SMILES: CCn1ncc(CN(C)C(=O)c2cc(-c3cccc(CC(=O)OC(C)(C)C)c3)nc3ccc(C)cc23)c1C] | 535.0 | 535.1 | 7.9 | 6.2 | 5.1 | 3.3 |
| 142 | 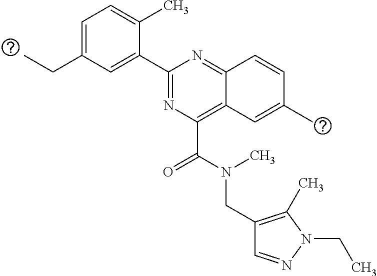 [SMILES: CCc1ccc(C)c(-c2nc(C(=O)N(C)Cc3cnn(CC)c3C)c3cc(C)ccc3n2)c1] | 464.0 | 464.2 | 18 | 6.3 | 7.6 | 10 |
| 143 | 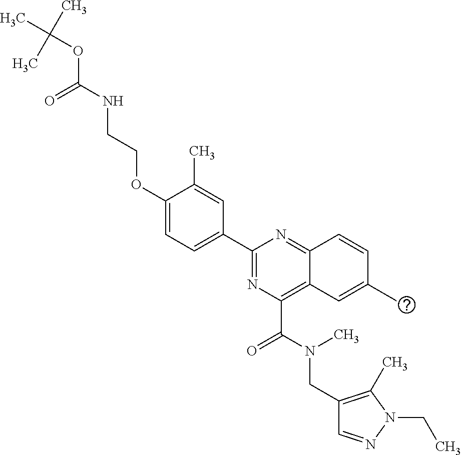 [SMILES: CCn1ncc(CN(C)C(=O)c2nc(-c3ccc(OCCNC(=O)OC(C)(C)C)c(C)c3)nc3ccc(C)cc23)c1C] | 593.1 | 593.3 | 0.13 | 6.4 | 4.3 | 8.1 |
| 144 | 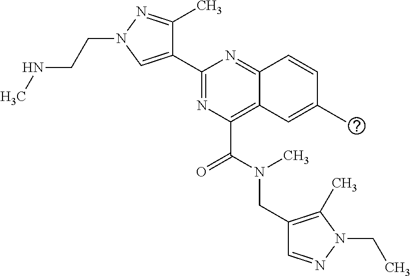 [SMILES: CCn1ncc(CN(C)C(=O)c2nc(-c3cn(CCNC)nc3C)nc3ccc(C)cc23)c1C] | 481.0 | 481.1 | 5.4 | 8.4 | 3.3 | 8.9 |
| 145 | 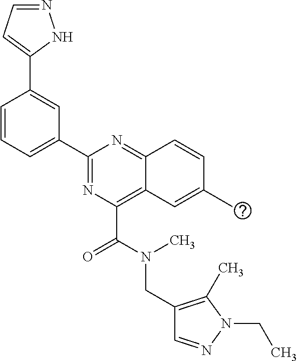 [SMILES: CCn1ncc(CN(C)C(=O)c2nc(-c3cccc(-c4ccn[nH]4)c3)nc3ccc(C)cc23)c1C] | 486.0 | 486.0 | 2.8 | 6.5 | 9.9 | 1.9 |
| 146 | 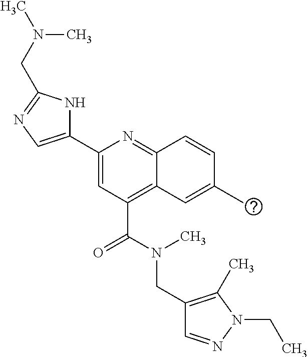 [SMILES: CCn1ncc(CN(C)C(=O)c2cc(-c3cnc(CN(C)C)[nH]3)nc3ccc(C)cc23)c1C] | 466.0 | 466.0 | 4.0 | 6.5 | 10.0 | 2.3 |
| 147 | 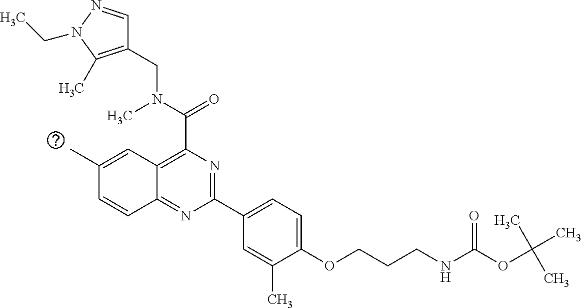 [SMILES: CCn1ncc(CN(C)C(=O)c2nc(-c3ccc(OCCCNC(=O)OC(C)(C)C)c(C)c3)nc3ccc(C)cc23)c1C] | 607.1 | 067.1 | 0.47 | 7.0 | 3.1 | 7.9 |
| 148 | 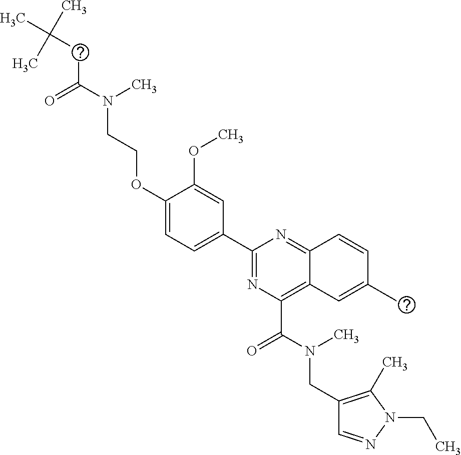 [SMILES: CCn1ncc(CN(C)C(=O)c2nc(-c3ccc(OCCN(C)C(=O)CC(C)(C)C)c(OC)c3)nc3ccc(C)cc23)c1C] | 623.1 | 623.1 | 7.5 | 7.2 | 3.9 | 11 |
| 149 | 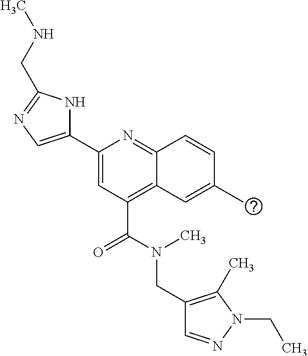 [SMILES: CCn1ncc(CN(C)C(=O)c2cc(-c3cnc(CNC)[nH]3)nc3ccc(C)cc23)c1C] | 452.0 | 452.0 | 14 | 7.8 | 9.2 | 6.7 |
| 150 | 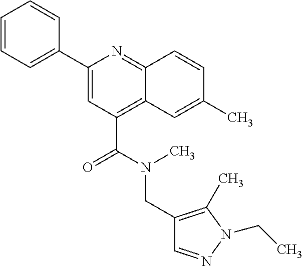 [SMILES: CCn1ncc(CN(C)C(=O)c2cc(-c3ccccc3)nc3ccc(C)cc23)c1C] | 398.5 | 399.1 | 6.6 | 8.2 | | |
| 151 | 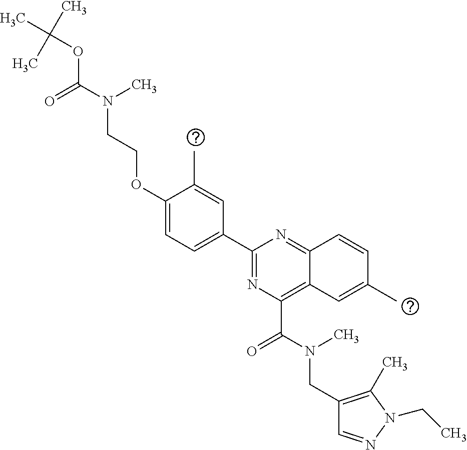 [SMILES: CCn1ncc(CN(C)C(=O)c2nc(-c3ccc(OCCN(C)C(=O)OC(C)(C)C)c(C)c3)nc3ccc(C)cc23)c1C] | 627.6 | 627.0 | 2.2 | 8.8 | 3.2 | 2.8 |
| 152 | 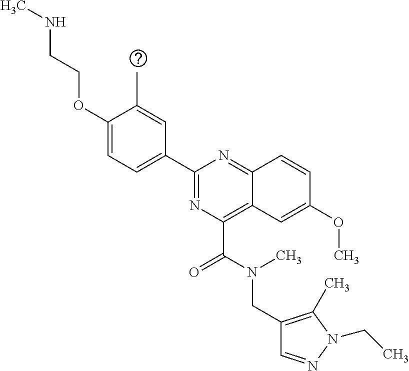 [SMILES: CCn1ncc(CN(C)C(=O)c2nc(-c3ccc(OCCNC)c(C)c3)nc3ccc(OC)cc23)c1C] | 523.0 | 523.0 | 3.6 | 8.9 | 1.6 | 3.6 |
| 153 | 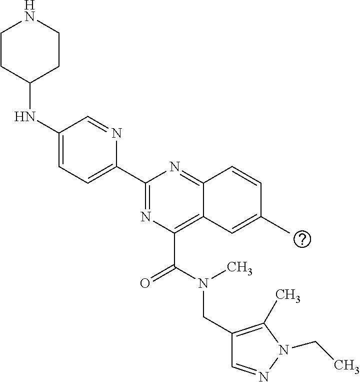 [SMILES: CCn1ncc(CN(C)C(=O)c2nc(-c3ccc(NC4CCNCC4)cn3)nc3ccc(C)cc23)c1C] | 519.0 | 591.2 | 0.39 | 9.0 | 4.9 | 0.35 |
| 154 | 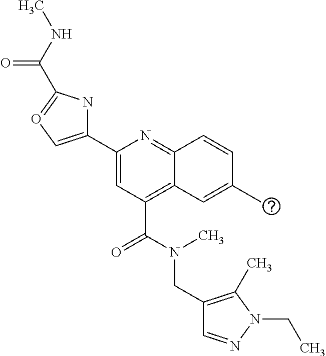 | 483.0 | 483.0 | 16 | 9.1 | 9.9 | 3.3 |
| 155 | 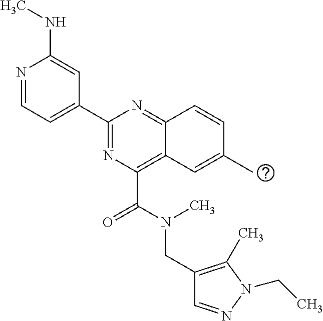 [SMILES: CCn1ncc(CN(C)C(=O)c2nc(-c3ccnc(NC)c3)nc3ccc(C)cc23)c1C] | 449.9 | 450.0 | 35 | 9.3 | 5.0 | 26 |
| 156 | 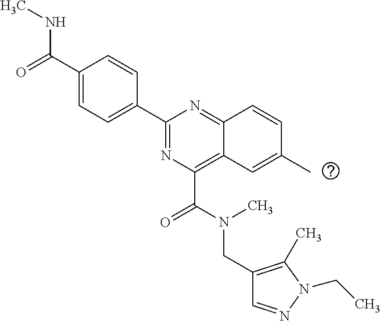 [SMILES: CCn1ncc(CN(C)C(=O)c2nc(-c3ccc(C(=O)NC)cc3)nc3ccc(C)cc23)c1C] | 477.0 | 477.0 | 34 | 9.6 | 9.6 | 13 |
| 157 | 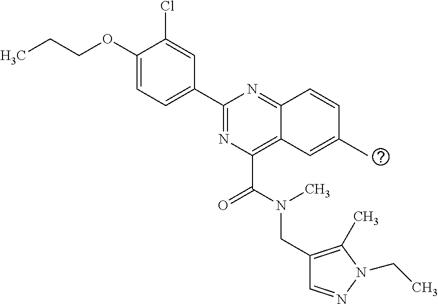 [SMILES: CCCOc1ccc(-c2nc(C(=O)N(C)Cc3cnn(CC)c3C)c3cc(C)ccc3n2)cc1Cl] | 513.4 | 512.9 | 3.2 | 9.6 | 5.6 | 2.8 |
| 158 | 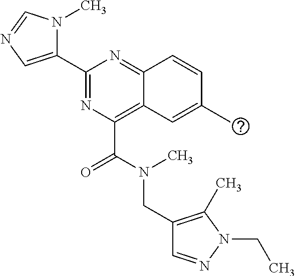 [SMILES: CCn1ncc(CN(C)C(=O)c2nc(-c3cncn3C)nc3ccc(C)cc23)c1C] | 423.9 | 424.0 | 14 | 9.8 | 6.6 | |
| 159 | 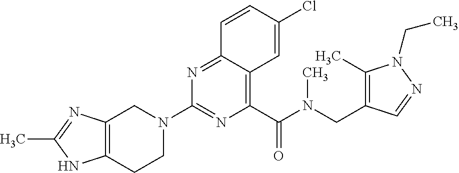 [SMILES: CCn1ncc(CN(C)C(=O)c2nc(N3CCc4[nH]c(C)nc4C3)nc3ccc(Cl)cc23)c1C] | 479.0 | 479.1 | 20 | 10.0 | 10 | 7.7 |
| 160 | 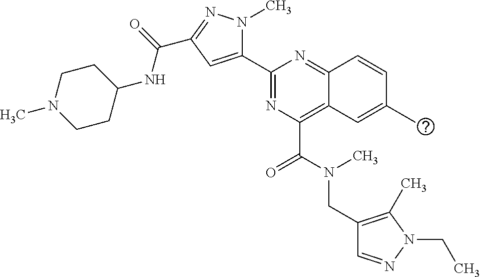 [SMILES: CCn1ncc(CN(C)C(=O)c2nc(-c3cc(C(=O)NC4CCN(C)CC4)nn3C)nc3ccc(C)cc23)c1C] | 564.1 | 564.1 | 32 | 11 | 5.1 | 20 |
| 161 | 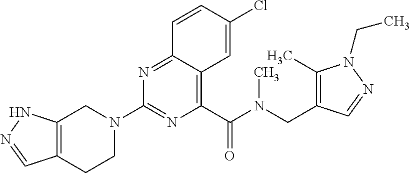 [SMILES: CCn1ncc(CN(C)C(=O)c2nc(N3CCc4cn[nH]c4C3)nc3ccc(Cl)cc23)c1C] | 465.0 | 465.0 | 17 | 11 | 11 | 6.2 |
| 162 | 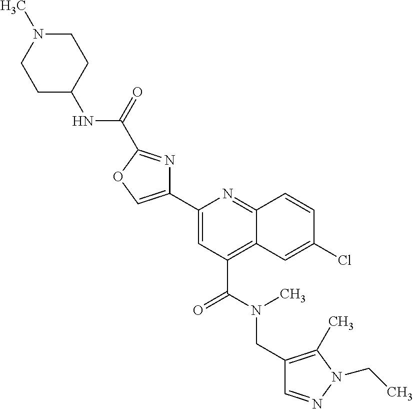 [SMILES: CCn1ncc(CN(C)C(=O)c2cc(-c3coc(C(=O)NC4CCN(C)CC4)n3)nc3ccc(Cl)cc23)c1C] | 566.1 | 569.0 | 200 | 12 | 12 | 200 |
| 163 | 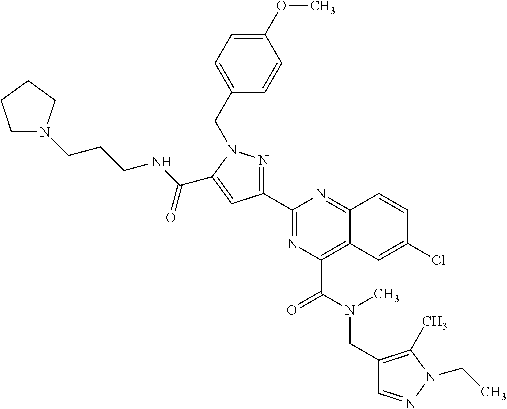 [SMILES: CCn1ncc(CN(C)C(=O)c2nc(-c3cc(C(=O)NCCCN4CCCC4)n(Cc4ccc(OC)cc4)n3)nc3ccc(Cl)cc23)c1C] | 684.2 | 684.1 | 62 | 12 | 2.7 | 35 |
| 164 | 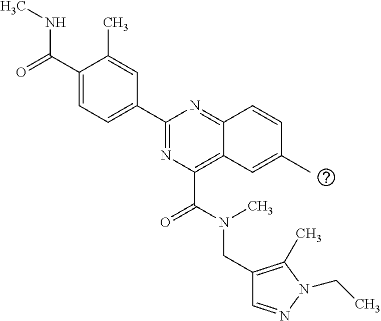 [SMILES: CCn1ncc(CN(C)C(=O)c2nc(-c3ccc(C(=O)NC)c(C)c3)nc3ccc(C)cc23)c1C] | 491.0 | 491.1 | 52 | 13 | 9.8 | 30 |
| 165 | 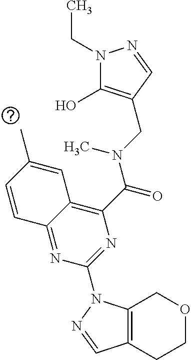 [SMILES: CCn1ncc(CN(C)C(=O)c2nc(-n3ncc4c3COCC4)nc3ccc(C)cc23)c1O] | 465.0 | 464.9 | 18 | 13 | 16 | 11 |
| 166 | 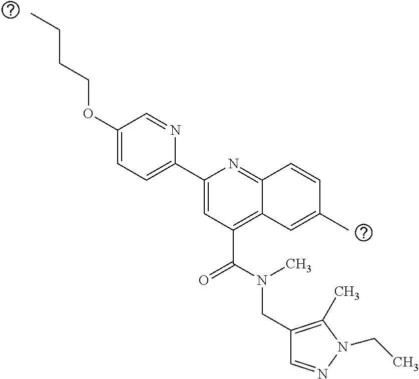 [SMILES: CCCCOc1ccc(-c2cc(C(=O)N(C)Cc3cnn(CC)c3C)c3cc(C)ccc3n2)nc1] | 494.0 | 494.1 | 28 | 13 | 11 | 15 |
| 167 | 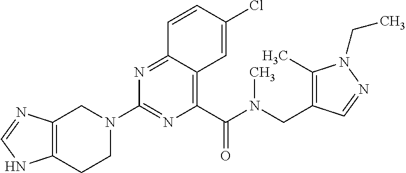 [SMILES: CCn1ncc(CN(C)C(=O)c2nc(N3CCc4[nH]cnc4C3)nc3ccc(Cl)cc23)c1C] | 465.0 | 465.1 | 18 | 13 | 11 | 8.0 |
| 168 | 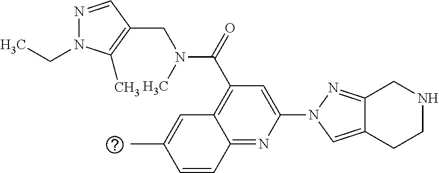 [SMILES: CCn1ncc(CN(C)C(=O)c2cc(-n3cc4c(n3)CNCC4)nc3ccc(C)cc23)c1C] | 464.0 | 464.0 | 18 | 13 | 18 | 5.8 |
| 169 | 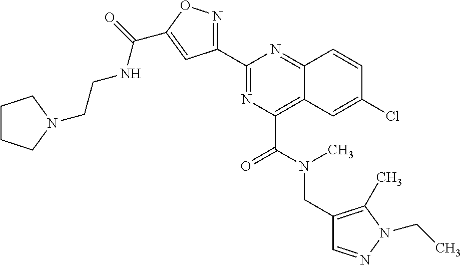 [SMILES: CCn1ncc(CN(C)C(=O)c2nc(-c3cc(C(=O)NCCN4CCCC4)on3)nc3ccc(Cl)cc23)c1C] | 551.0 | 551.2 | 8.4 | 13 | 6.7 | 15 |
| 170 | 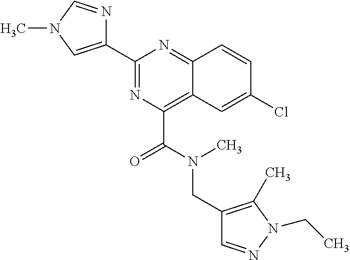 [SMILES: CCn1ncc(CN(C)C(=O)c2nc(-c3cn(C)cn3)nc3ccc(Cl)cc23)c1C] | 423.9 | 424.1 | 58 | 14 | 6.9 | 13 |
| 171 | 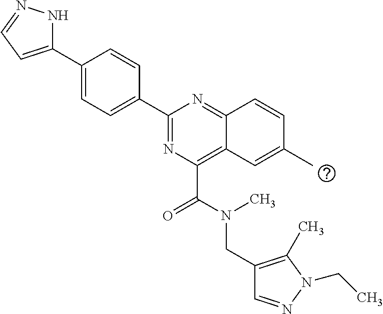 [SMILES: CCn1ncc(CN(C)C(=O)c2nc(-c3ccc(-c4ccn[nH]4)cc3)nc3ccc(C)cc23)c1C] | 486.0 | 486.0 | 8.5 | 14 | 7.8 | 3.6 |
| 172 | 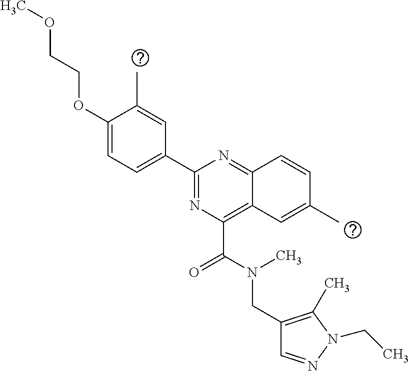 [SMILES: CCn1ncc(CN(C)C(=O)c2nc(-c3ccc(OCCOC)c(C)c3)nc3ccc(C)cc23)c1C] | 512.0 | 512.1 | 200 | 14 | 12 | |
| 173 | 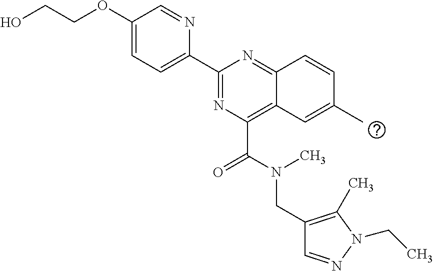 [SMILES: CCn1ncc(CN(C)C(=O)c2nc(-c3ccc(OCCO)cn3)nc3ccc(C)cc23)c1C] | 481.0 | 481.0 | 17 | 14 | 11 | 7.8 |
| 174 | 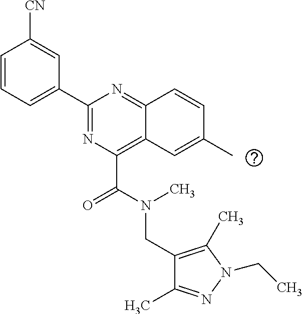 [SMILES: CCn1nc(C)c(CN(C)C(=O)c2nc(-c3cccc(C#N)c3)nc3ccc(C)cc23)c1C] | 468.4 | 467.9 | 27 | 15 | 11 | 35 |
| 175 | 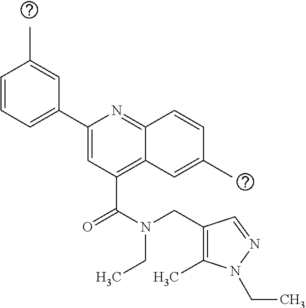 [SMILES: CCN(Cc1cnn(CC)c1C)C(=O)c1cc(-c2cccc(C)c2)nc2ccc(C)cc12] | 467.4 | 467.1 | 5.3 | 15 | 9.4 | |
| 176 | 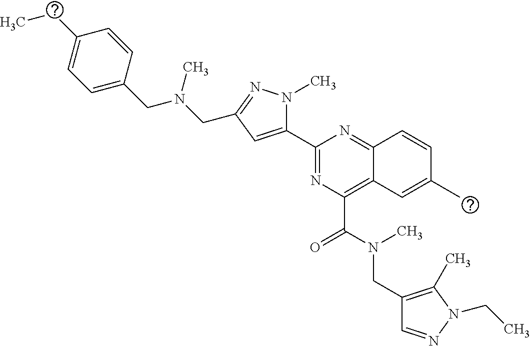 [SMILES: CCc1ccc(CN(C)Cc2cc(-c3nc(C(=O)N(C)Cc4cnn(CC)c4C)c4cc(C)ccc4n3)n(C)n2)cc1] | 587.1 | 587.1 | 86 | 16 | 8.2 | 44 |
| 177 | 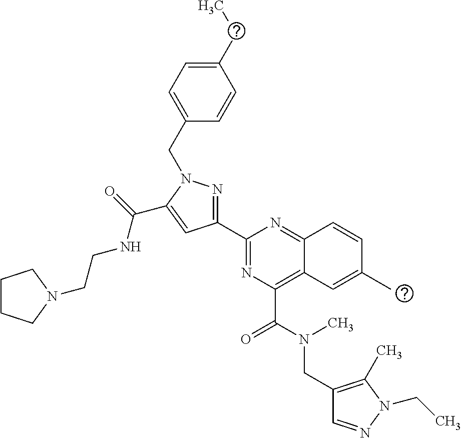 [SMILES: CCc1ccc(Cn2nc(-c3nc(C(=O)N(C)Cc4cnn(CC)c4C)c4cc(C)ccc4n3)cc2C(=O)NCCN2CCCC2)cc1] | 670.2 | 670.0 | 54 | 16 | 3.4 | 44 |
| 178 | 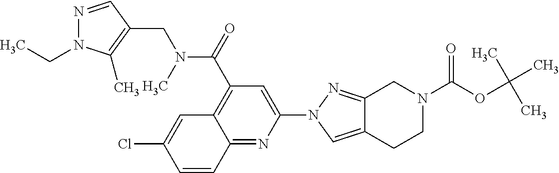 [SMILES: CCn1ncc(CN(C)C(=O)c2cc(-n3cc4c(n3)CN(C(=O)OC(C)(C)C)CC4)nc3ccc(Cl)cc23)c1C] | 564.1 | 564.1 | 200 | 17 | 4.8 | 200 |
| 179 | 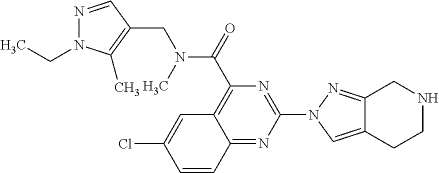 [SMILES: CCn1ncc(CN(C)C(=O)c2nc(-n3cc4c(n3)CNCC4)nc3ccc(Cl)cc23)c1C] | 465.0 | 465.0 | 115 | 18 | 20 | 55 |
| 180 | 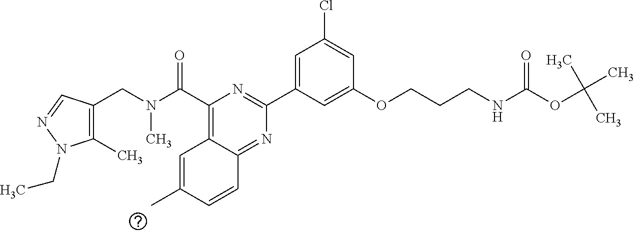 [SMILES: CCn1ncc(CN(C)C(=O)c2nc(-c3cc(Cl)cc(OCCCNC(=O)OC(C)(C)C)c3)nc3ccc(C)cc23)c1C] | 627.6 | 626.9 | 200 | 19 | 20 | 200 |
| 181 | 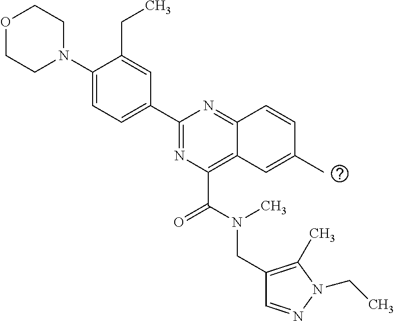 [SMILES: CCc1cc(-c2nc(C(=O)N(C)Cc3cnn(CC)c3C)c3cc(C)ccc3n2)ccc1N1CCOCC1] | 535.0 | 535.1 | 21 | 19 | 7.3 | 16 |
| 182 | 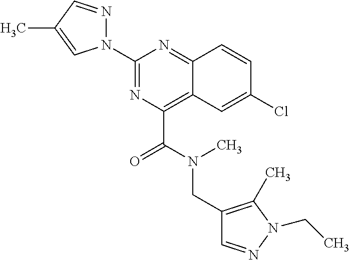 [SMILES: CCn1ncc(CN(C)C(=O)c2nc(-n3cc(C)cn3)nc3ccc(Cl)cc23)c1C] | 423.9 | 424.1 | 70 | 19 | 7.8 | 19 |
| 183 | 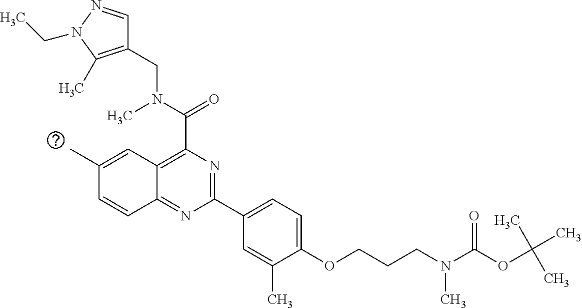 [SMILES: CCn1ncc(CN(C)C(=O)c2nc(-c3ccc(OCCCN(C)C(=O)OC(C)(C)C)c(C)c3)nc3ccc(C)cc23)c1C] | 621.2 | 621.0 | 18 | 20 | 8.7 | 42 |
| 184 | 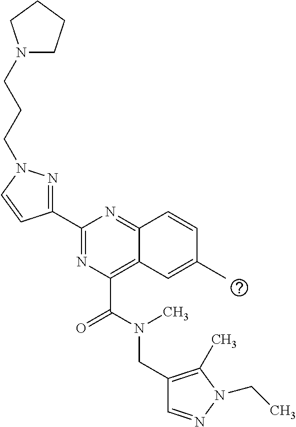 [SMILES: CCn1ncc(CN(C)C(=O)c2nc(-c3ccn(CCCN4CCCC4)n3)nc3ccc(C)cc23)c1C] | 521.1 | 521.0 | 200 | 20 | 15 | 163 |
| 185 | 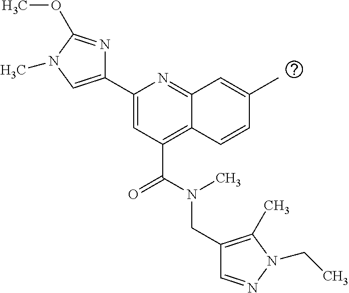 [SMILES: CCn1ncc(CN(C)C(=O)c2cc(-c3cn(C)c(OC)n3)nc3cc(C)ccc23)c1C] | 452.9 | 453.0 | 98 | 20 | 20 | 56 |
| 186 | 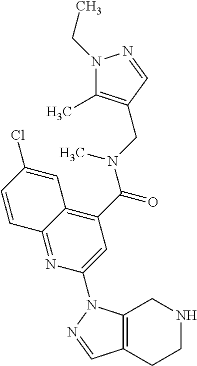 [SMILES: CCn1ncc(CN(C)C(=O)c2cc(-n3ncc4c3CNCC4)nc3ccc(Cl)cc23)c1C] | 464.0 | 464.1 | 114 | 20 | 7.8 | 64 |
| 187 | 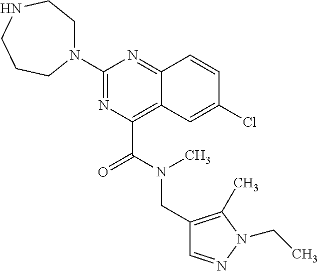 [SMILES: CCn1ncc(CN(C)C(=O)c2nc(N3CCCNCC3)nc3ccc(Cl)cc23)c1C] | 442.0 | 442.1 | 200 | 20 | | 200 |
| 188 | 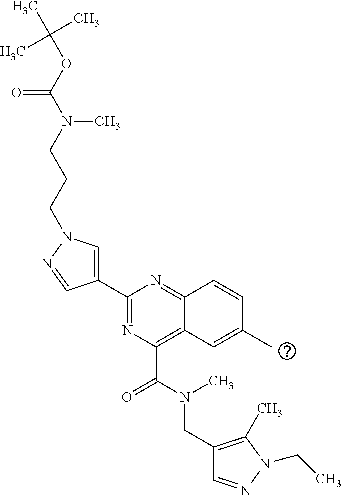 [SMILES: CCn1ncc(CN(C)C(=O)c2nc(-c3cnn(CCCN(C)C(=O)OC(C)(C)C)c3)nc3ccc(C)cc23)c1C] | 581.1 | 581.1 | 200 | 20 | | 200 |
| 189 | 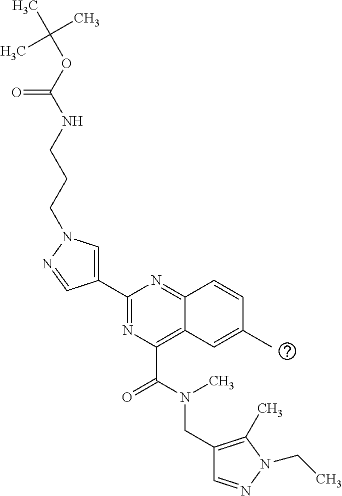 [SMILES: CCn1ncc(CN(C)C(=O)c2nc(-c3cnn(CCCNC(=O)OC(C)(C)C)c3)nc3ccc(C)cc23)c1C] | 567.1 | 567.1 | 200 | 20 | | 200 |
| 190 | 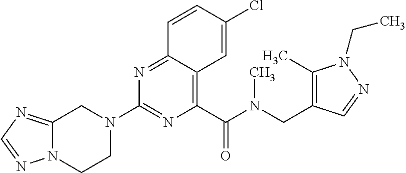 [SMILES: CCn1ncc(CN(C)C(=O)c2nc(N3CCn4ncnc4C3)nc3ccc(Cl)cc23)c1C] | 465.9 | 466.1 | 200 | 20 | 20 | 103 |
| 191 | 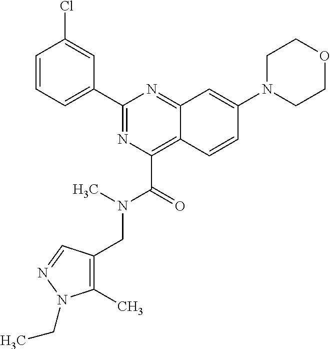 [SMILES: CCn1ncc(CN(C)C(=O)c2nc(-c3cccc(Cl)c3)nc3cc(N4CCOCC4)ccc23)c1C] | 505.0 | 505.0 | 200 | 20 | | 200 |
| 192 | 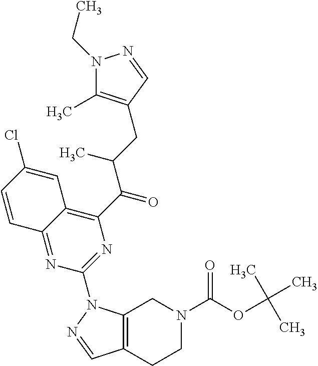 [SMILES: CCn1ncc(CC(C)C(=O)c2nc(-n3ncc4c3CN(C(=O)OC(C)(C)C)CC4)nc3ccc(Cl)cc23)c1C] | 565.1 | 565.0 | 200 | 20 | 9.4 | 200 |
| 193 | 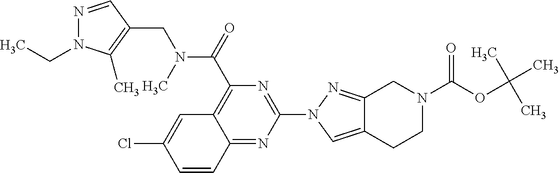 [SMILES: CCn1ncc(CN(C)C(=O)c2nc(-n3cc4c(n3)CN(C(=O)OC(C)(C)C)CC4)nc3ccc(Cl)cc23)c1C] | 565.1 | 565.0 | 200 | 20 | 8.2 | 200 |
| 194 | 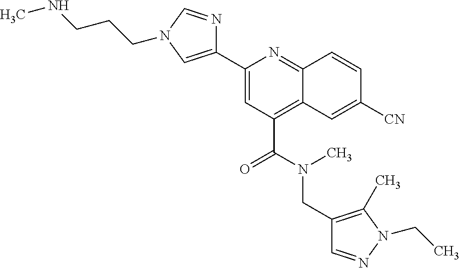 [SMILES: CCn1ncc(CN(C)C(=O)c2cc(-c3cn(CCCNC)cn3)nc3ccc(C#N)cc23)c1C] | 480.0 | 480.0 | 17 | 20 | 20 | 9.9 |
| 195 | 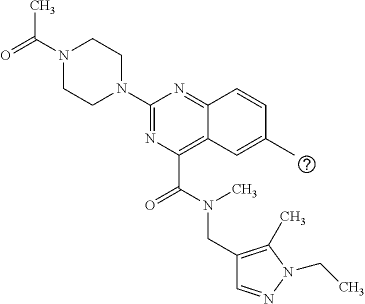 [SMILES: CCn1ncc(CN(C)C(=O)c2nc(N3CCN(C(C)=O)CC3)nc3ccc(C)cc23)c1C] | 470.0 | 470.1 | 200 | 20 | | 200 |
| 196 | 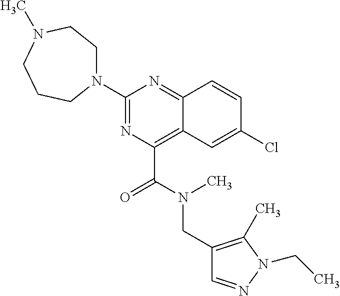 [SMILES: CCn1ncc(CN(C)C(=O)c2nc(N3CCCN(C)CC3)nc3ccc(Cl)cc23)c1C] | 456.0 | 456.1 | 200 | 20 | 17 | |
| 197 | 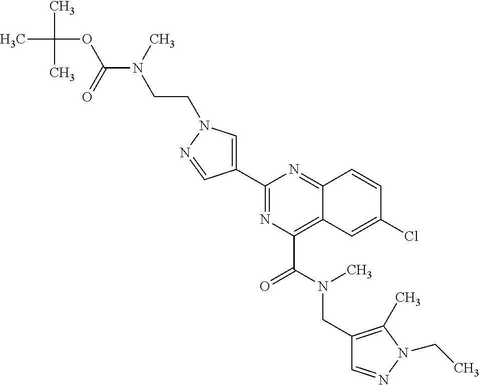 [SMILES: CCn1ncc(CN(C)C(=O)c2nc(-c3cnn(CCN(C)C(=O)OC(C)(C)C)c3)nc3ccc(Cl)cc23)c1C] | 567.1 | 567.1 | 200 | 20 | | |
| 198 | 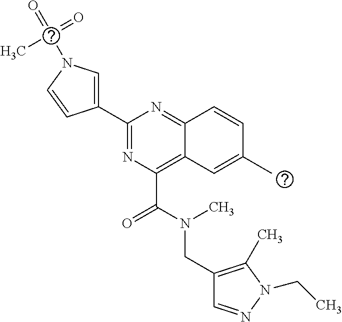 | 488.0 | 488.0 | 75 | 20 | | |
| 199 | 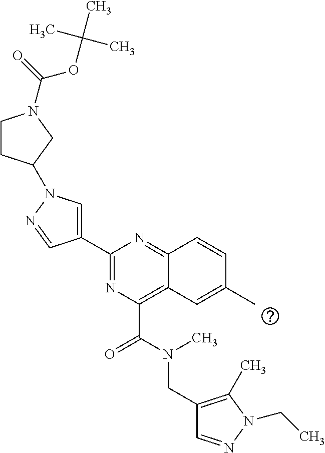 [SMILES: CCn1ncc(CN(C)C(=O)c2nc(-c3cnn(C4CCN(C(=O)OC(C)(C)C)C4)c3)nc3ccc(C)cc23)c1C] | 579.1 | 579.1 | 200 | 20 | | |
| 200 | 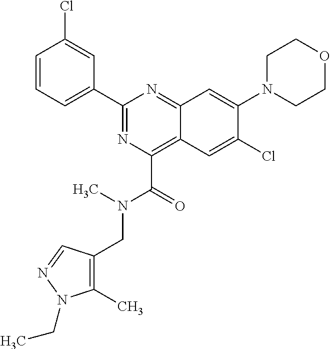 [SMILES: CCn1ncc(CN(C)C(=O)c2nc(-c3cccc(Cl)c3)nc3cc(N4CCOCC4)c(Cl)cc23)c1C] | 539.5 | 538.9 | 190 | 20 | | 126 |
| 201 | 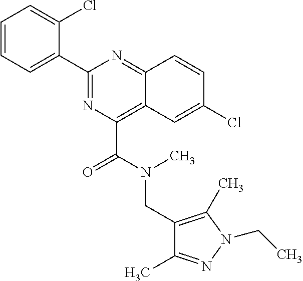 [SMILES: CCn1nc(C)c(CN(C)C(=O)c2nc(-c3ccccc3Cl)nc3ccc(Cl)cc23)c1C] | 468.4 | 467.9 | 200 | 20 | | 200 |
| 202 | 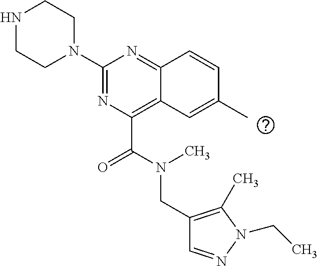 [SMILES: CCn1ncc(CN(C)C(=O)c2nc(N3CCNCC3)nc3ccc(C)cc23)c1C] | 427.9 | 428.1 | 200 | 20 | | 175 |
| 203 | 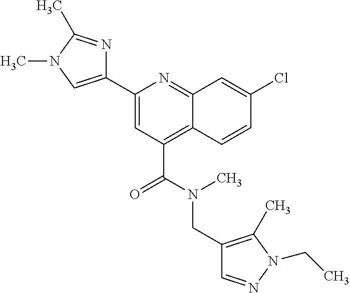 [SMILES: CCn1ncc(CN(C)C(=O)c2cc(-c3cn(C)c(C)n3)nc3cc(Cl)ccc23)c1C] | 436.9 | 437.0 | 200 | 20 | | |
| 204 | 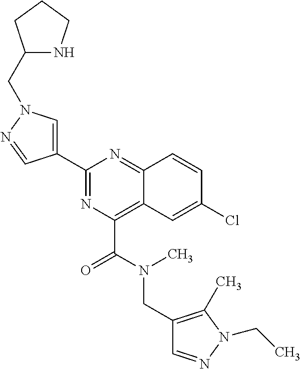 [SMILES: CCn1ncc(CN(C)C(=O)c2nc(-c3cnn(CC4CCCN4)c3)nc3ccc(Cl)cc23)c1C] | 493.0 | 493.1 | 200 | 20 | | |
| 205 | 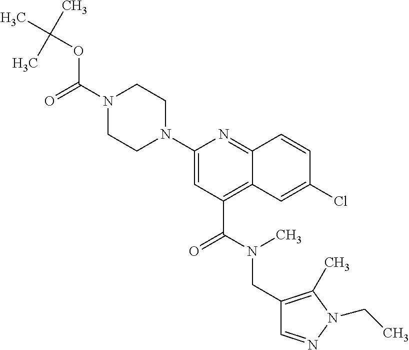 [SMILES: CCn1ncc(CN(C)C(=O)c2cc(N3CCN(C(=O)OC(C)(C)C)CC3)nc3ccc(Cl)cc23)c1C] | 527.1 | 527.1 | 200 | 20 | | |
| 206 | 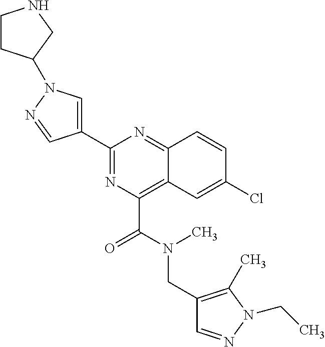 [SMILES: CCn1ncc(CN(C)C(=O)c2nc(-c3cnn(C4CCNC4)c3)nc3ccc(Cl)cc23)c1C] | 479.0 | 479.1 | 192 | 20 | | |
| 207 | 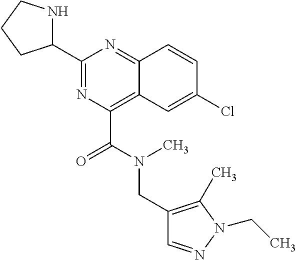 [SMILES: CCn1ncc(CN(C)C(=O)c2nc(C3CCCN3)nc3ccc(Cl)cc23)c1C] | 412.9 | 413.0 | 200 | 20 | | |
| 208 | 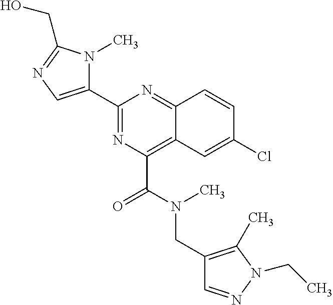 [SMILES: CCn1ncc(CN(C)C(=O)c2nc(-c3cnc(CO)n3C)nc3ccc(Cl)cc23)c1C] | 453.9 | 453.9 | 50 | 20 | 20 | 36 |
| 209 | 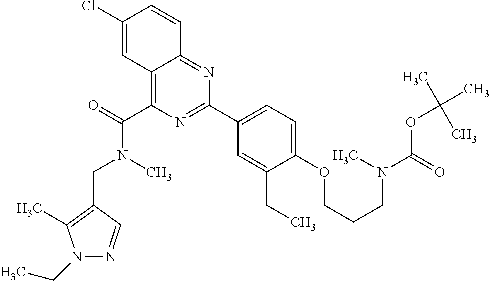 [SMILES: CCc1cc(-c2nc(C(=O)N(C)Cc3cnn(CC)c3C)c3cc(Cl)ccc3n2)ccc1OCCCN(C)C(=O)OC(C)(C)C] | 635.2 | 635.3 | 200 | 20 | 8.8 | 200 |
| 210 | 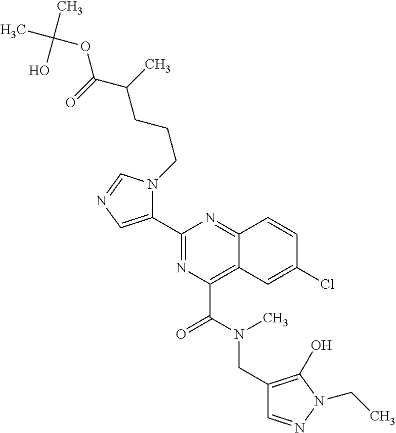 [SMILES: CCn1ncc(CN(C)C(=O)c2nc(-c3cncn3CCCC(C)C(=O)OC(C)(C)O)nc3ccc(Cl)cc23)c1O] | 581.1 | 581.1 | 151 | 20 | 20 | 98 |
| 211 | 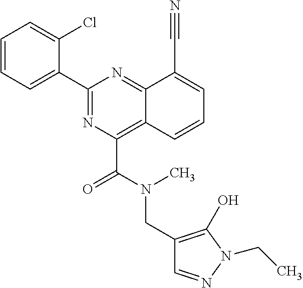 [SMILES: CCn1ncc(CN(C)C(=O)c2nc(-c3ccccc3Cl)nc3c(C#N)cccc23)c1O] | 444.9 | 445.0 | 32 | 20 | 15 | 17 |
| 212 | 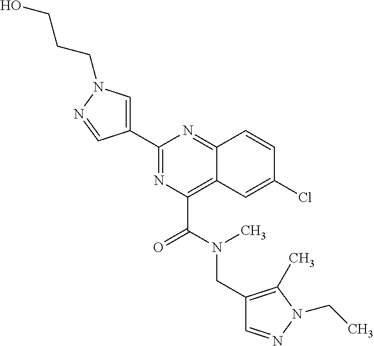 [SMILES: CCn1ncc(CN(C)C(=O)c2nc(-c3cnn(CCCO)c3)nc3ccc(Cl)cc23)c1C] | 468.0 | 468.0 | 96 | 20 | | |
| 213 | 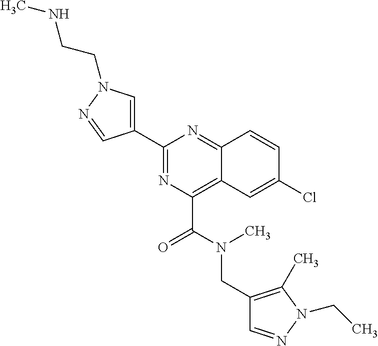 [SMILES: CCn1ncc(CN(C)C(=O)c2nc(-c3cnn(CCNC)c3)nc3ccc(Cl)cc23)c1C] | 467.0 | 467.1 | 135 | 20 | | |
| 214 | 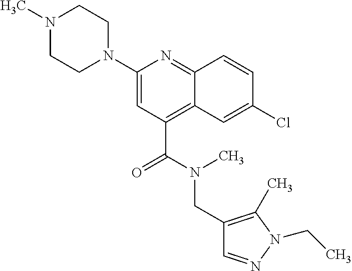 [SMILES: CCn1ncc(CN(C)C(=O)c2cc(N3CCN(C)CC3)nc3ccc(Cl)cc23)c1C] | 441.0 | 441.1 | 200 | 20 | | |
| 215 | 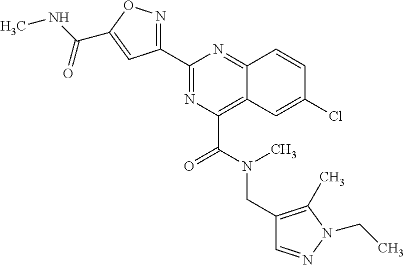 [SMILES: CCn1ncc(CN(C)C(=O)c2nc(-c3cc(C(=O)NC)on3)nc3ccc(Cl)cc23)c1C] | 467.9 | 468.2 | | 20 | | 41 |
| 216 | 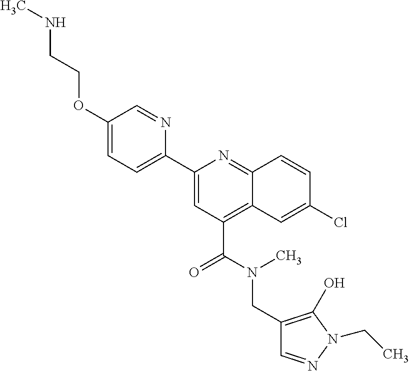 [SMILES: CCn1ncc(CN(C)C(=O)c2cc(-c3ccc(OCCNC)cn3)nc3ccc(Cl)cc23)c1O] | 493.0 | 493.0 | 200 | 20 | 20 | 200 |
| 217 | 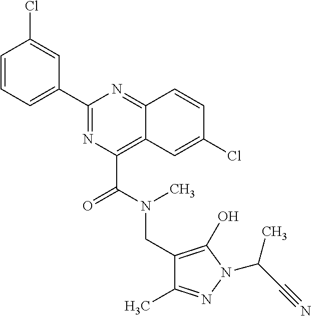 [SMILES: Cc1nn(C(C)C#N)c(O)c1CN(C)C(=O)c1nc(-c2cccc(Cl)c2)nc2ccc(Cl)cc12] | 493.4 | 493.0 | 200 | 20 | 20 | 200 |
| 218 | 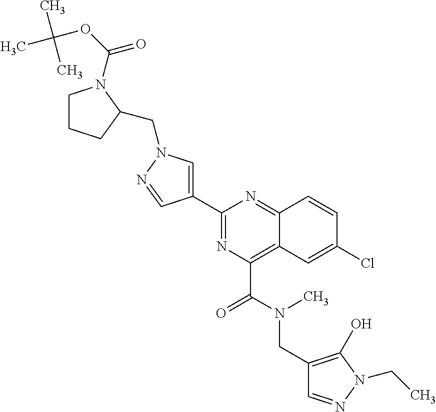 [SMILES: CCn1ncc(CN(C)C(=O)c2nc(-c3cnn(CC4CCCN4C(=O)OC(C)(C)C)c3)nc3ccc(Cl)cc23)c1O] | 593.1 | 593.1 | 200 | 20 | | |
| 219 | 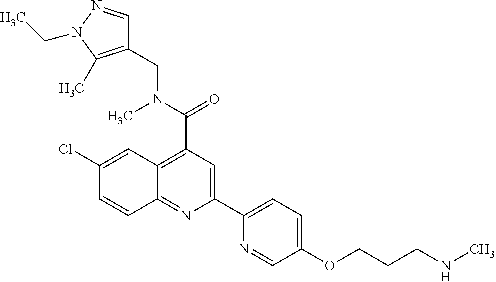 [SMILES: CCn1ncc(CN(C)C(=O)c2cc(-c3ccc(OCCCNC)cn3)nc3ccc(Cl)cc23)c1C] | 507.0 | 507.1 | 200 | 20 | 20 | 200 |
| 220 | 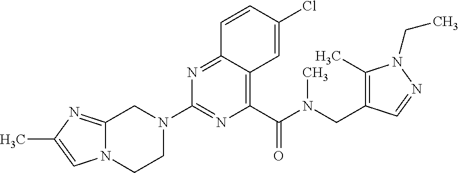 [SMILES: CCn1ncc(CN(C)C(=O)c2nc(N3CCn4cc(C)nc4C3)nc3ccc(Cl)cc23)c1C] | 479.0 | 479.0 | 141 | 20 | 20 | 100 |
| 221 | 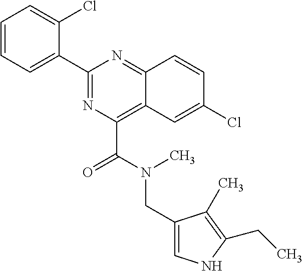 [SMILES: CCc1[nH]cc(CN(C)C(=O)c2nc(-c3ccccc3Cl)nc3ccc(Cl)cc23)c1C] | 453.4 | 453.1 | 200 | 20 | 10 | 200 |
| 222 | 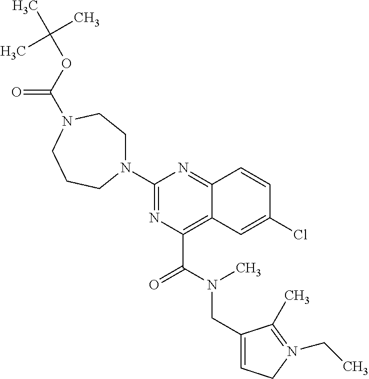 | 542.1 | 542.1 | 200 | 20 | 20 | 200 |
| 223 | 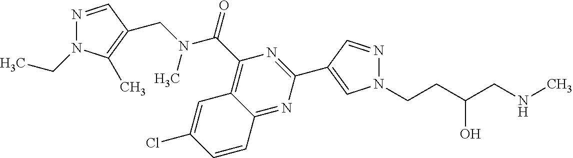 [SMILES: CCn1ncc(CN(C)C(=O)c2nc(-c3cnn(CCC(O)CNC)c3)nc3ccc(Cl)cc23)c1C] | 511.0 | 511.1 | 200 | 20 | | |
| 224 | 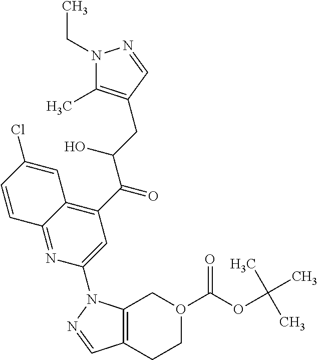 | 564.1 | 564.1 | 200 | 20 | | |
| 225 | 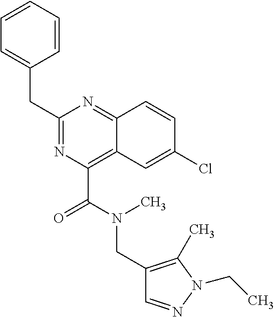 [SMILES: CCn1ncc(CN(C)C(=O)c2nc(Cc3ccccc3)nc3ccc(Cl)cc23)c1C] | 433.9 | 434.1 | 96 | 20 | | |
| 226 | 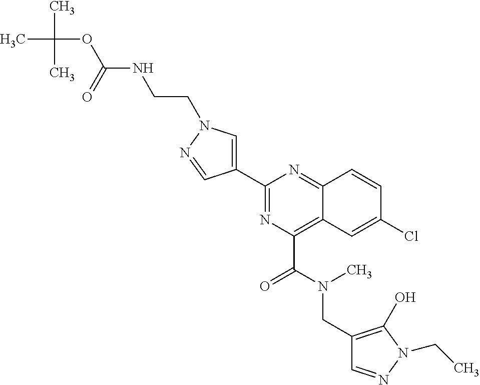 [SMILES: CCn1ncc(CN(C)C(=O)c2nc(-c3cnn(CCNC(=O)OC(C)(C)C)c3)nc3ccc(Cl)cc23)c1O] | 553.1 | 553.1 | 200 | 20 | | |
| 227 | 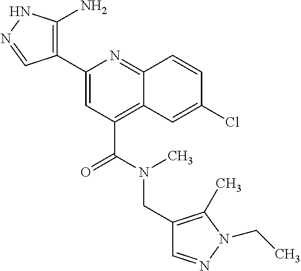 [SMILES: CCn1ncc(CN(C)C(=O)c2cc(-c3cn[nH]c3N)nc3ccc(Cl)cc23)c1C] | 423.9 | 424.1 | 50 | 20 | 20 | 41 |
| 228 | 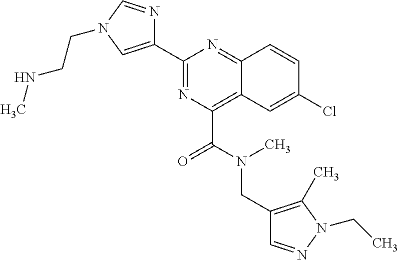 [SMILES: CCn1ncc(CN(C)C(=O)c2nc(-c3cn(CCNC)cn3)nc3ccc(Cl)cc23)c1C] | 467.0 | 467.0 | 200 | 20 | 20 | 140 |
| 229 | 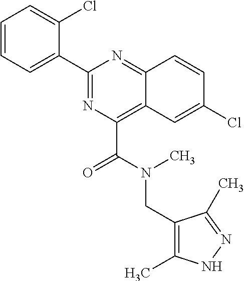 [SMILES: Cc1n[nH]c(C)c1CN(C)C(=O)c1nc(-c2ccccc2Cl)nc2ccc(Cl)cc12] | 440.3 | 439.9 | 200 | 20 | 20 | 200 |
| 230 | 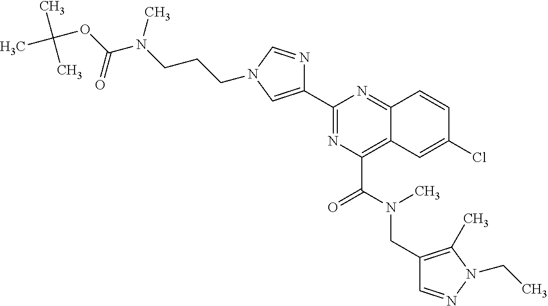 [SMILES: CCn1ncc(CN(C)C(=O)c2nc(-c3cn(CCCN(C)C(=O)OC(C)(C)C)cn3)nc3ccc(Cl)cc23)c1C] | 581.1 | 581.1 | 200 | 20 | 20 | 200 |
| 231 | 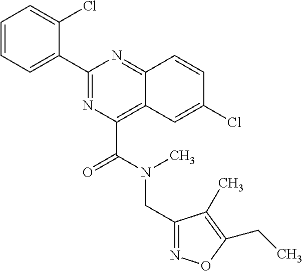 [SMILES: CCc1onc(CN(C)C(=O)c2nc(-c3ccccc3Cl)nc3ccc(Cl)cc23)c1C] | 455.3 | 454.9 | 76 | 20 | | |
| 232 | 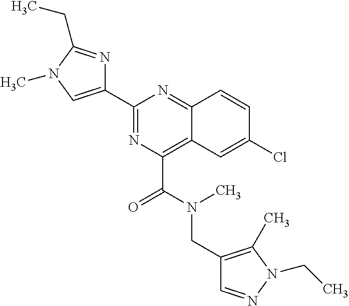 [SMILES: CCc1nc(-c2nc(C(=O)N(C)Cc3cnn(CC)c3C)c3cc(Cl)ccc3n2)cn1C] | 453.9 | 453.9 | 50 | 20 | 20 | 48 |
| 233 | 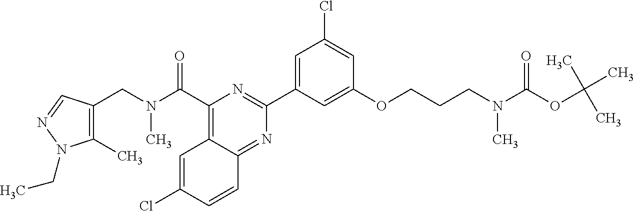 [SMILES: CCn1ncc(CN(C)C(=O)c2nc(-c3cc(Cl)cc(OCCCN(C)C(=O)OC(C)(C)C)c3)nc3ccc(Cl)cc23)c1C] | 641.6 | 641.0 | 200 | 20 | 20 | 200 |
| 234 | 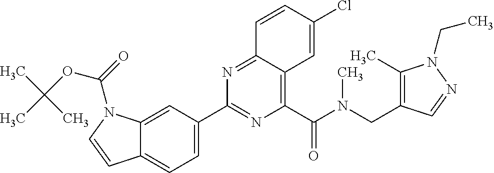 [SMILES: CCn1ncc(CN(C)C(=O)c2nc(-c3ccc4ccn(C(=O)OC(C)(C)C)c4c3)nc3ccc(Cl)cc23)c1C] | 559.1 | 559.0 | 200 | 20 | | 200 |
| 235 | 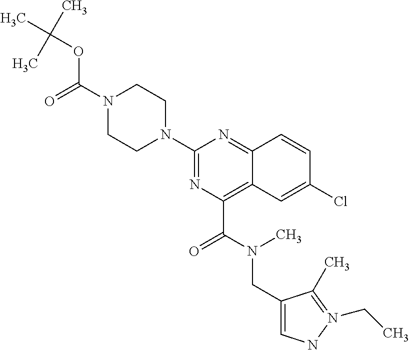 | 528.0 | 528.1 | 200 | 20 | | |
| 236 | 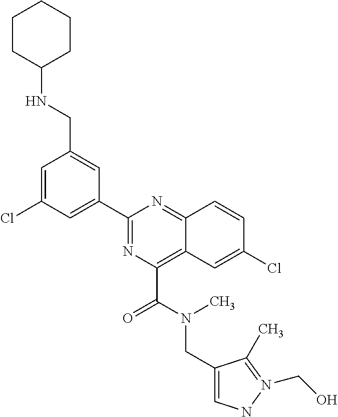 | 567.5 | 567.2 | #DIV/0! | 20 | | 13 |
| 237 | 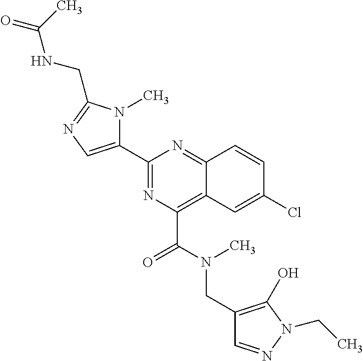 [SMILES: CCn1ncc(CN(C)C(=O)c2nc(-c3cnc(CNC(C)=O)n3C)nc3ccc(Cl)cc23)c1O] | 495.0 | 495.3 | #DIV/0! | 20 | | 50 |
| 238 | 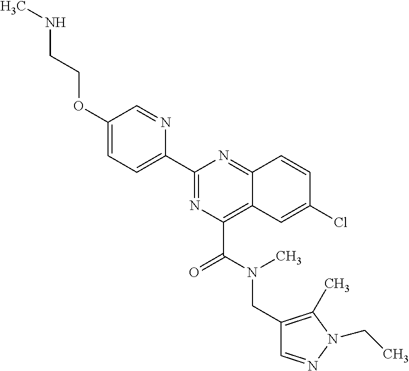 [SMILES: CCn1ncc(CN(C)C(=O)c2nc(-c3ccc(OCCNC)cn3)nc3ccc(Cl)cc23)c1C] | 494.0 | 494.0 | 200 | 20 | 20 | 200 |
| 239 | 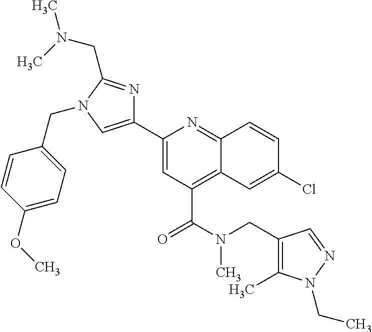 [SMILES: CCn1ncc(CN(C)C(=O)c2cc(-c3cn(Cc4ccc(OC)cc4)c(CN(C)C)n3)nc3ccc(Cl)cc23)c1C] | 586.1 | 586.1 | 200 | 20 | 20 | 200 |
| 240 | 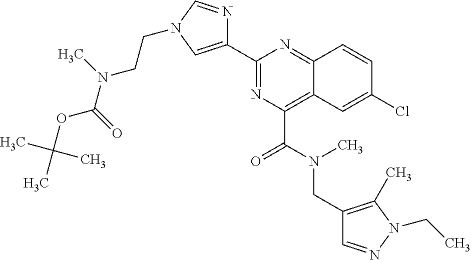 [SMILES: CCn1ncc(CN(C)C(=O)c2nc(-c3cn(CCN(C)C(=O)OC(C)(C)C)cn3)nc3ccc(Cl)cc23)c1C] | 567.1 | 567.1 | 200 | 20 | 20 | 200 |
| 241 | 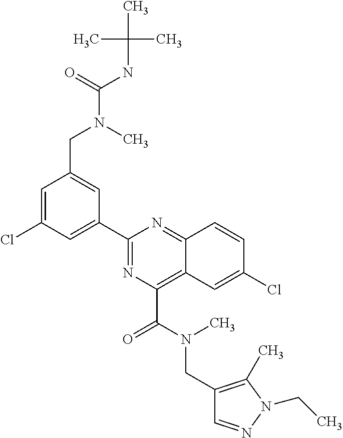 [SMILES: CCn1ncc(CN(C)C(=O)c2nc(-c3cc(Cl)cc(CN(C)C(=O)NC(C)(C)C)c3)nc3ccc(Cl)cc23)c1C] | 597.5 | 597.0 | 6.9 | 2.0 | 19 | 26 |
| 242 | 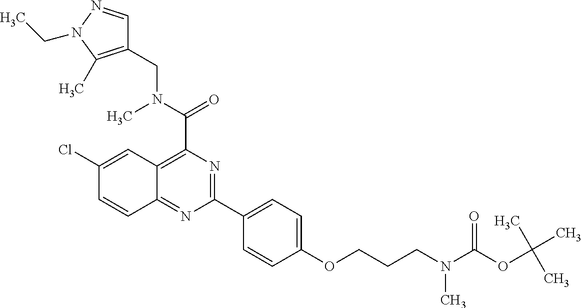 [SMILES: CCn1ncc(CN(C)C(=O)c2nc(-c3ccc(OCCCN(C)C(=O)OC(C)(C)C)cc3)nc3ccc(Cl)cc23)c1C] | 607.1 | 607.1 | 57 | 20 | | |
| 243 | 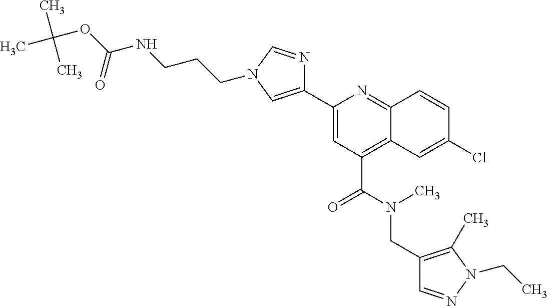 [SMILES: CCn1ncc(CN(C)C(=O)c2cc(-c3cn(CCCNC(=O)OC(C)(C)C)cn3)nc3ccc(Cl)cc23)c1C] | 566.1 | 566.1 | 99 | 20 | | |
| 244 | 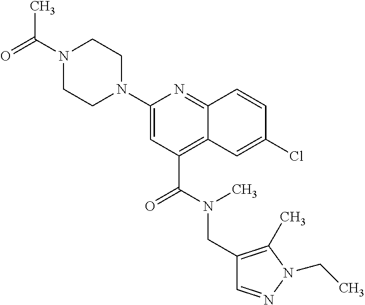 [SMILES: CCn1ncc(CN(C)C(=O)c2cc(N3CCN(C(C)=O)CC3)nc3ccc(Cl)cc23)c1C] | 469.0 | 469.1 | 200 | 20 | | |
| 245 | 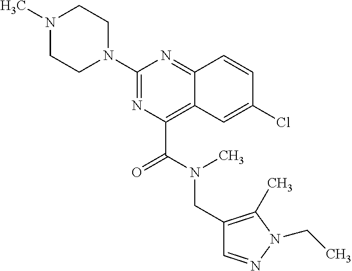 [SMILES: CCn1ncc(CN(C)C(=O)c2nc(N3CCN(C)CC3)nc3ccc(Cl)cc23)c1C] | 442.0 | 442.1 | 200 | 20 | 20 | |
| 246 | 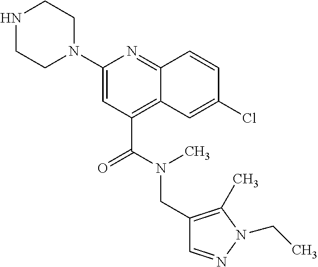 [SMILES: CCn1ncc(CN(C)C(=O)c2cc(N3CCNCC3)nc3ccc(Cl)cc23)c1C] | 426.9 | 427.1 | 200 | 20 | | |
| 247 | 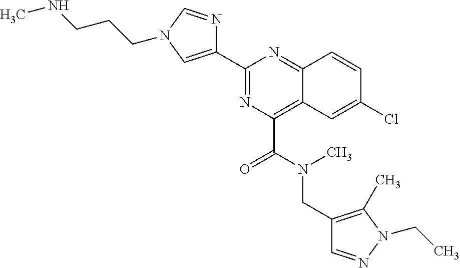 [SMILES: CCn1ncc(CN(C)C(=O)c2nc(-c3cn(CCCNC)cn3)nc3ccc(Cl)cc23)c1C] | 481.0 | 481.0 | 200 | 20 | 20 | 157 |
| 248 | 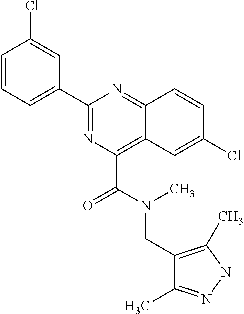 [SMILES: Cc1n[nH]c(C)c1CN(C)C(=O)c1nc(-c2cccc(Cl)c2)nc2ccc(Cl)cc12] | 440.3 | 440.0 | 200 | 20 | 19 | 114 |
| 249 | 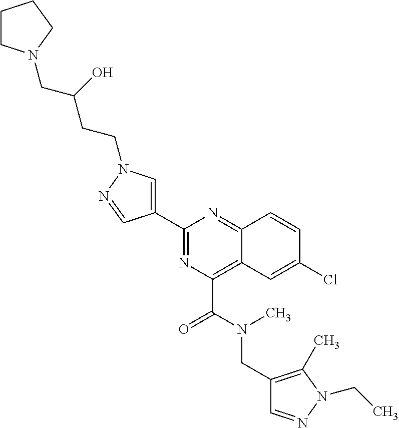 [SMILES: CCn1ncc(CN(C)C(=O)c2nc(-c3cnn(CCC(O)CN4CCCC4)c3)nc3ccc(Cl)cc23)c1C] | 551.1 | 551.0 | 200 | 20 | | 200 |
| 250 | 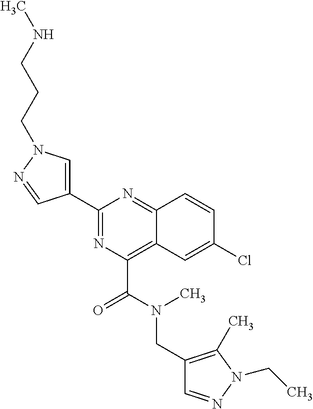 [SMILES: CCn1ncc(CN(C)C(=O)c2nc(-c3cnn(CCCNC)c3)nc3ccc(Cl)cc23)c1C] | 481.0 | 481.1 | 121 | 20 | | |
| 251 | 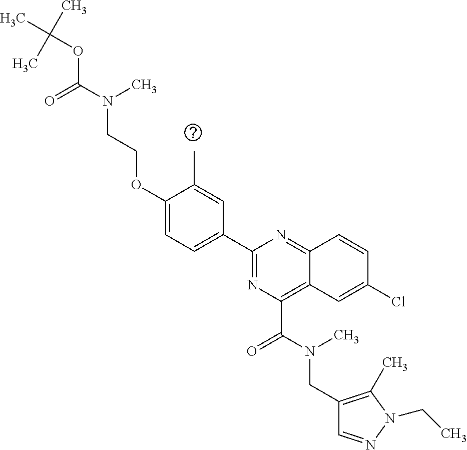 [SMILES: CCn1ncc(CN(C)C(=O)c2nc(-c3ccc(OCCN(C)C(=O)OC(C)(C)C)c(C)c3)nc3ccc(Cl)cc23)c1C] | 611.1 | 611.1 | 200 | 20 | | |
| 252 | 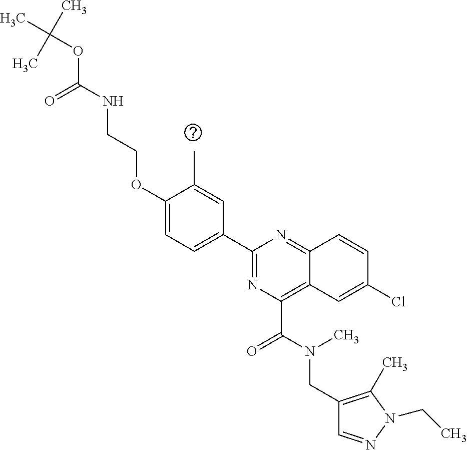 [SMILES: CCn1ncc(CN(C)C(=O)c2nc(-c3ccc(OCCNC(=O)OC(C)(C)C)c(C)c3)nc3ccc(Cl)cc23)c1C] | 597.1 | 597.1 | 200 | 20 | | |
| 253 | 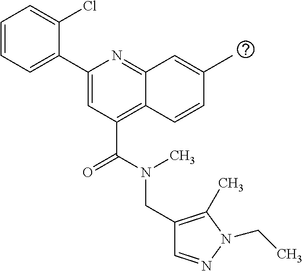 [SMILES: CCn1ncc(CN(C)C(=O)c2cc(-c3ccccc3Cl)nc3cc(C)ccc23)c1C] | 436.9 | 437.1 | 90 | | | |
| 254 | 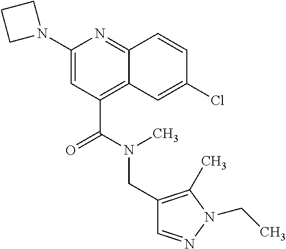 [SMILES: CCn1ncc(CN(C)C(=O)c2cc(N3CCC3)nc3ccc(Cl)cc23)c1C] | 397.9 | 398.1 | 200 | | | |
| 255 | 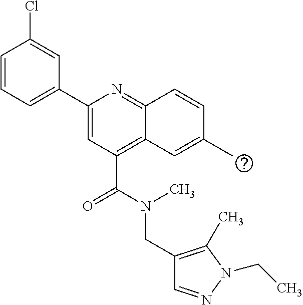 [SMILES: CCn1ncc(CN(C)C(=O)c2cc(-c3cccc(Cl)c3)nc3ccc(C)cc23)c1C] | 454.4 | 454.0 | 3.5 | 9.7 | | |
| 256 | 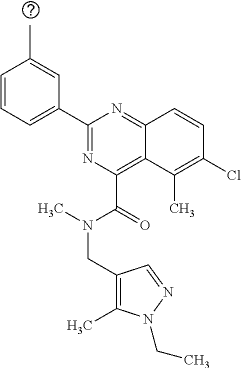 [SMILES: CCn1ncc(CN(C)C(=O)c2nc(-c3cccc(C)c3)nc3ccc(Cl)c(C)c23)c1C] | 488.4 | 468.1 | 1.4 | | | |
| 257 | 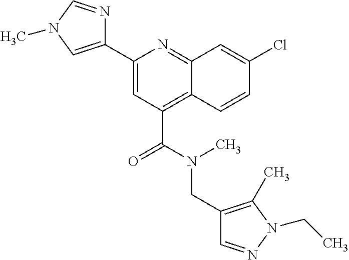 [SMILES: CCn1ncc(CN(C)C(=O)c2cc(-c3cn(C)cn3)nc3cc(Cl)ccc23)c1C] | 422.9 | 423.2 | 120 | | | |
| 258 | 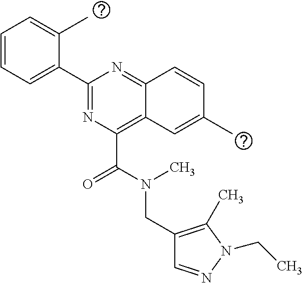 [SMILES: CCn1ncc(CN(C)C(=O)c2nc(-c3ccccc3C)nc3ccc(C)cc23)c1C] | 498.8 | 497.9 | 0.0070 | 0.37 | | |
| 259 | 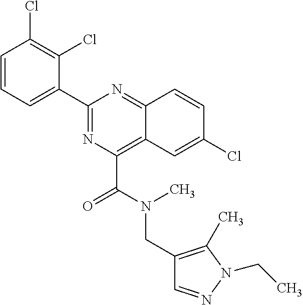 [SMILES: CCn1ncc(CN(C)C(=O)c2nc(-c3cccc(Cl)c3Cl)nc3ccc(Cl)cc23)c1C] | 488.8 | 488.0 | 0.0026 | 0.18 | | |
| 260 | 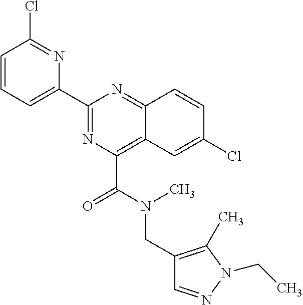 [SMILES: CCn1ncc(CN(C)C(=O)c2nc(-c3cccc(Cl)n3)nc3ccc(Cl)cc23)c1C] | 455.3 | 455.0 | 1.7 | 3.6 | | |
| 261 | 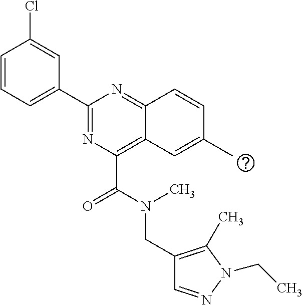 [SMILES: CCn1ncc(CN(C)C(=O)c2nc(-c3cccc(Cl)c3)nc3ccc(C)cc23)c1C] | 454.4 | 454.0 | 0.0056 | 0.71 | | |
| 262 | 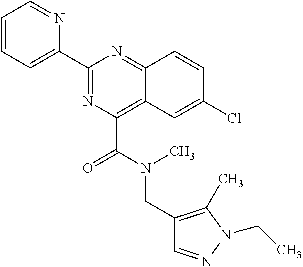 [SMILES: CCn1ncc(CN(C)C(=O)c2nc(-c3ccccn3)nc3ccc(Cl)cc23)c1C] | 420.9 | 421.1 | 5.4 | | | |
| 263 | 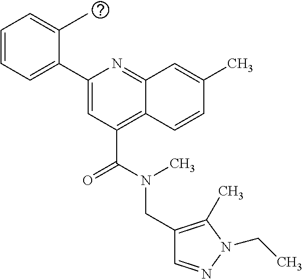 [SMILES: CCn1ncc(CN(C)C(=O)c2cc(-c3ccccc3C)nc3cc(C)ccc23)c1C] | 432.9 | 433.1 | 87 | | | |
| 264 | 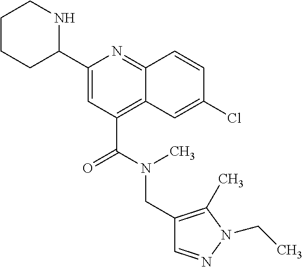 [SMILES: CCn1ncc(CN(C)C(=O)c2cc(C3CCCCN3)nc3ccc(Cl)cc23)c1C] | 426.0 | 426.1 | 200 | | | |
| 265 | 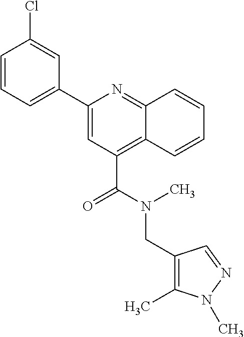 [SMILES: Cc1c(CN(C)C(=O)c2cc(-c3cccc(Cl)c3)nc3ccccc23)cnn1C] | 404.9 | 405.1 | 195 | | | |
| 266 | 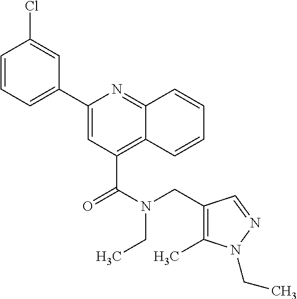 [SMILES: CCN(Cc1cnn(CC)c1C)C(=O)c1cc(-c2cccc(Cl)c2)nc2ccccc12] | 432.9 | 433.2 | 200 | | | |
| 267 | 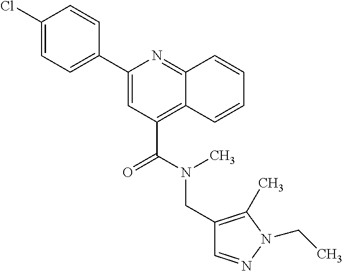 [SMILES: CCn1ncc(CN(C)C(=O)c2cc(-c3ccc(Cl)cc3)nc3ccccc23)c1C] | 402.5 | 403.2 | 113 | | | |
| 268 | 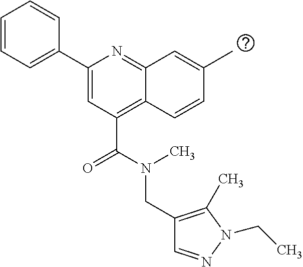 [SMILES: CCn1ncc(CN(C)C(=O)c2cc(-c3ccccc3)nc3cc(C)ccc23)c1C] | 402.5 | 403.1 | 200 | | | |
| 269 | 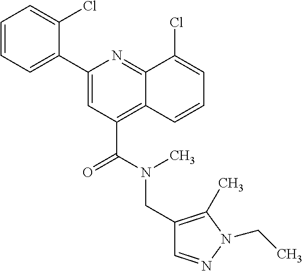 [SMILES: CCn1ncc(CN(C)C(=O)c2cc(-c3ccccc3Cl)nc3c(Cl)cccc23)c1C] | 453.4 | 453.0 | 26 | | | |
| 270 | 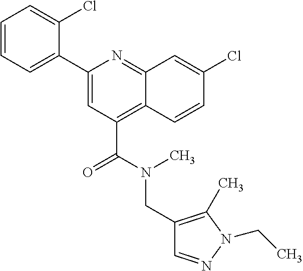 [SMILES: CCn1ncc(CN(C)C(=O)c2cc(-c3ccccc3Cl)nc3cc(Cl)ccc23)c1C] | 453.4 | 453.0 | 25 | | | |
| 271 | 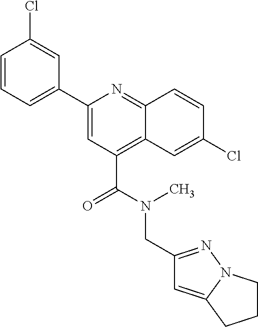 [SMILES: CN(Cc1cc2n(n1)CCC2)C(=O)c1cc(-c2cccc(Cl)c2)nc2ccc(Cl)cc12] | 451.4 | 451.0 | 14 | | | |
| 272 | 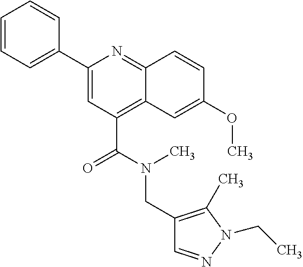 [SMILES: CCn1ncc(CN(C)C(=O)c2cc(-c3ccccc3)nc3ccc(OC)cc23)c1C] | 414.5 | 415.1 | 66 | | | |
| 273 | 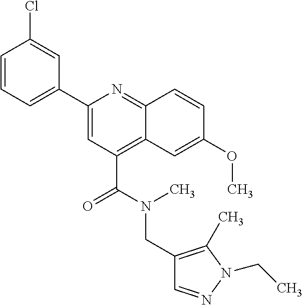 [SMILES: CCn1ncc(CN(C)C(=O)c2cc(-c3cccc(Cl)c3)nc3ccc(OC)cc23)c1C] | 448.9 | 449.0 | 19 | | | |
| 274 | 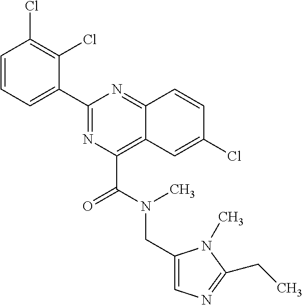 [SMILES: CCc1ncc(CN(C)C(=O)c2nc(-c3cccc(Cl)c3Cl)nc3ccc(Cl)cc23)n1C] | 488.8 | 488.1 | 0.0032 | | | |
| 275 | 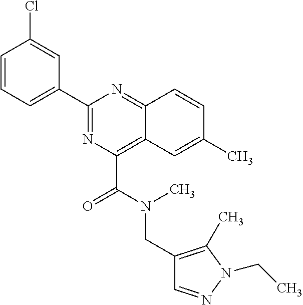 [SMILES: CCn1ncc(CN(C)C(=O)c2nc(-c3cccc(Cl)c3)nc3ccc(C)cc23)c1C] | 433.9 | 434.3 | 0.014 | | | |
| 276 | 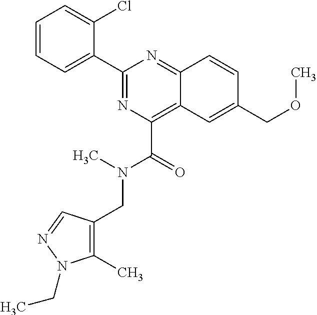 [SMILES: CCn1ncc(CN(C)C(=O)c2nc(-c3ccccc3Cl)nc3ccc(COC)cc23)c1C] | 464.0 | 464.3 | 0.62 | 1.5 | | |
| 277 | 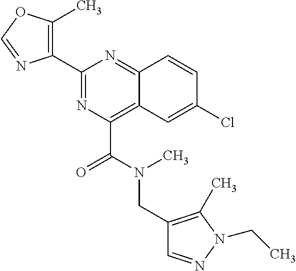 [SMILES: CCn1ncc(CN(C)C(=O)c2nc(-c3ncoc3C)nc3ccc(Cl)cc23)c1C] | 441.0 | 441.0 | 67 | | | |
| 278 | 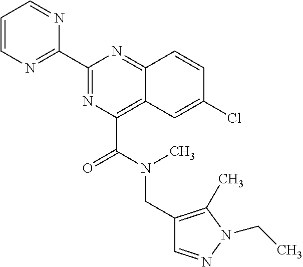 [SMILES: CCn1ncc(CN(C)C(=O)c2nc(-c3ncccn3)nc3ccc(Cl)cc23)c1C] | 421.9 | 422.0 | 132 | | | |
| 279 | 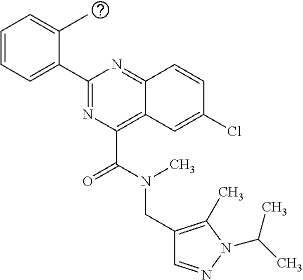 [SMILES: Cc1ccccc1-c1nc(C(=O)N(C)Cc2cnn(C(C)C)c2C)c2cc(Cl)ccc2n1] | 468.4 | 468.0 | 0.082 | 0.76 | | |
| 280 | 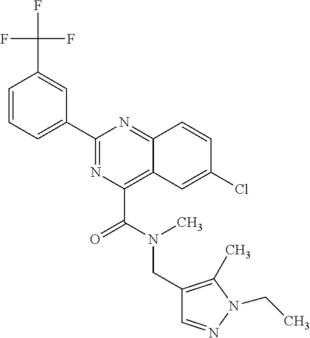 [SMILES: CCn1ncc(CN(C)C(=O)c2nc(-c3cccc(C(F)(F)F)c3)nc3ccc(Cl)cc23)c1C] | 487.9 | 488.0 | 0.27 | | | |
| 281 | 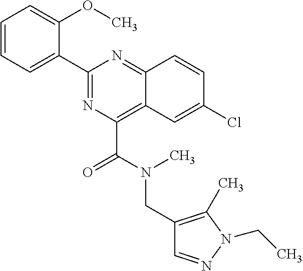 [SMILES: CCn1ncc(CN(C)C(=O)c2nc(-c3ccccc3OC)nc3ccc(Cl)cc23)c1C] | 449.9 | 450.1 | 0.83 | 1.3 | | |
| 282 | 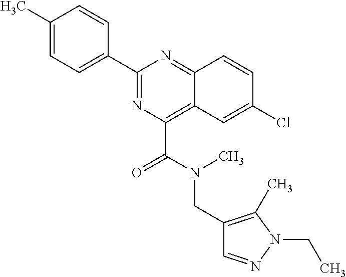 [SMILES: CCn1ncc(CN(C)C(=O)c2nc(-c3ccc(C)cc3)nc3ccc(Cl)cc23)c1C] | 433.9 | 434.1 | 5.5 | 11 | | |
| 283 | 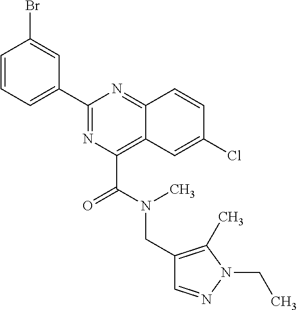 [SMILES: CCn1ncc(CN(C)C(=O)c2nc(-c3cccc(Br)c3)nc3ccc(Cl)cc23)c1C] | 498.8 | 497.9 | 0.00089 | 0.67 | | |
| 284 | 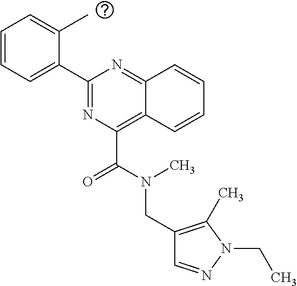 [SMILES: CCn1ncc(CN(C)C(=O)c2nc(-c3ccccc3C)nc3ccccc23)c1C] | 419.9 | 420.1 | 13 | 15 | 8.5 | |
| 285 | 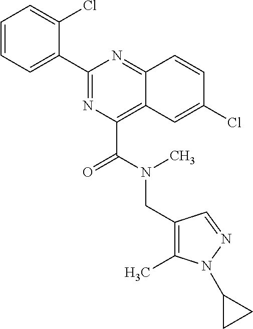 [SMILES: Cc1c(CN(C)C(=O)c2nc(-c3ccccc3Cl)nc3ccc(Cl)cc23)cnn1C1CC1] | 466.4 | 466.0 | 4.2 | 2.7 | | |
| 286 | 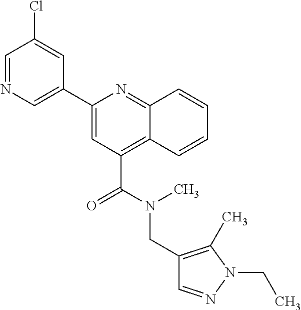 [SMILES: CCn1ncc(CN(C)C(=O)c2cc(-c3cncc(Cl)c3)nc3ccccc23)c1C] | 419.9 | 420.2 | 200 | | | |
| 287 | 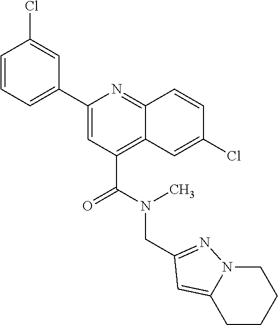 [SMILES: CN(Cc1cc2n(n1)CCCC2)C(=O)c1cc(-c2cccc(Cl)c2)nc2ccc(Cl)cc12] | 465.4 | 465.0 | 200 | | | |
| 288 | 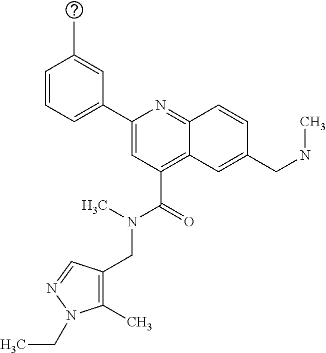 [SMILES: CCn1ncc(CN(C)C(=O)c2cc(-c3cccc(C)c3)nc3ccc(CNC)cc23)c1C] | 463.0 | 463.1 | 0.81 | | | |
| 289 | 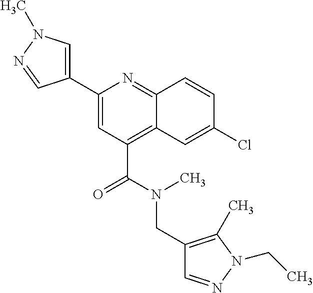 [SMILES: CCn1ncc(CN(C)C(=O)c2cc(-c3cnn(C)c3)nc3ccc(Cl)cc23)c1C] | 422.9 | 423.1 | 26 | | | |
| 290 | 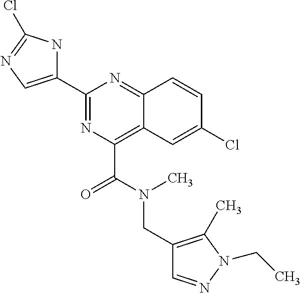 [SMILES: CCn1ncc(CN(C)C(=O)c2nc(-c3cnc(Cl)[nH]3)nc3ccc(Cl)cc23)c1C] | 461.4 | 461.0 | 7.6 | 14 | | |
| 291 | 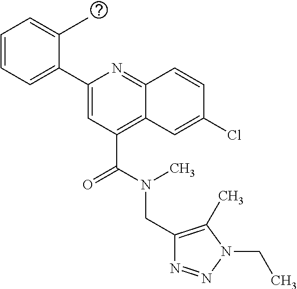 [SMILES: CCn1nnc(CN(C)C(=O)c2cc(-c3ccccc3C)nc3ccc(Cl)cc23)c1C] | 454.4 | 454.2 | 89 | | | |
| 292 | 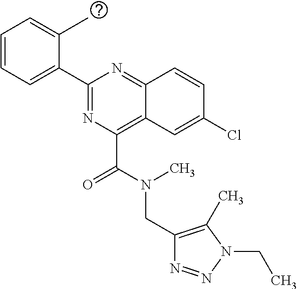 [SMILES: CCn1nnc(CN(C)C(=O)c2nc(-c3ccccc3C)nc3ccc(Cl)cc23)c1C] | 455.3 | 455.2 | 50 | | | |
| 293 | 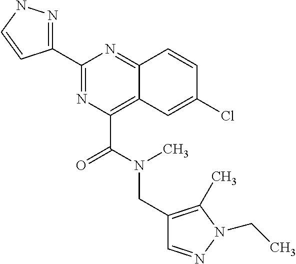 [SMILES: CCn1ncc(CN(C)C(=O)c2nc(-c3cc[nH]n3)nc3ccc(Cl)cc23)c1C] | 426.9 | 427.2 | 10 | 5.6 | | |
| 294 | 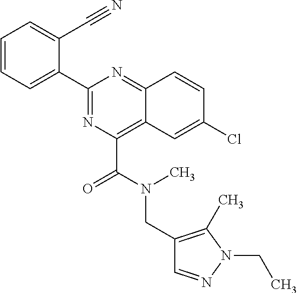 [SMILES: CCn1ncc(CN(C)C(=O)c2nc(-c3ccccc3C#N)nc3ccc(Cl)cc23)c1C] | 444.9 | 445.0 | 0.22 | 0.57 | | |
| 295 | 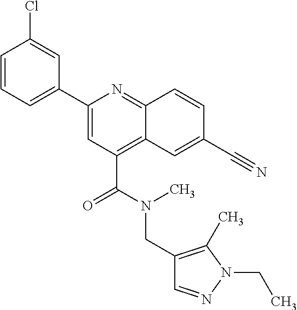 [SMILES: CCn1ncc(CN(C)C(=O)c2cc(-c3cccc(Cl)c3)nc3ccc(C#N)cc23)c1C] | 443.9 | 444.1 | 200 | | | |
| 296 | 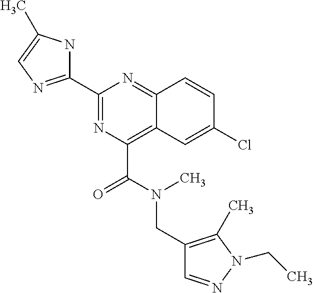 [SMILES: CCn1ncc(CN(C)C(=O)c2nc(-c3ncc(C)[nH]3)nc3ccc(Cl)cc23)c1C] | 441.0 | 441.0 | 18 | | | |
| 297 | 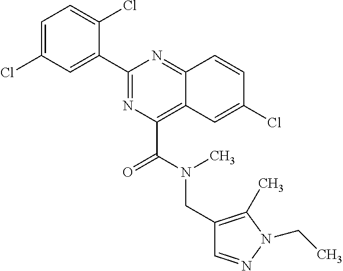 [SMILES: CCn1ncc(CN(C)C(=O)c2nc(-c3cc(Cl)ccc3Cl)nc3ccc(Cl)cc23)c1C] | 488.8 | 488.0 | 1.8 | 3.2 | | |
| 298 | 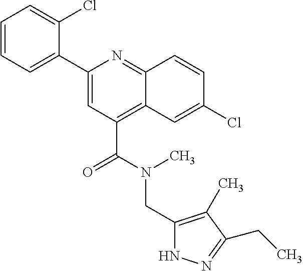 [SMILES: CCc1n[nH]c(CN(C)C(=O)c2cc(-c3ccccc3Cl)nc3ccc(Cl)cc23)c1C] | 453.4 | 453.0 | 31 | | | |
| 299 | 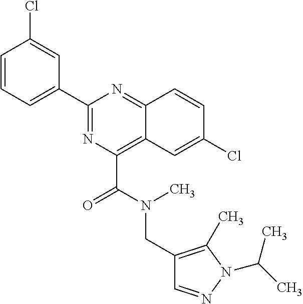 [SMILES: Cc1c(CN(C)C(=O)c2nc(-c3cccc(Cl)c3)nc3ccc(Cl)cc23)cnn1C(C)C] | 468.4 | 468.0 | 0.028 | 0.61 | | |
| 300 | 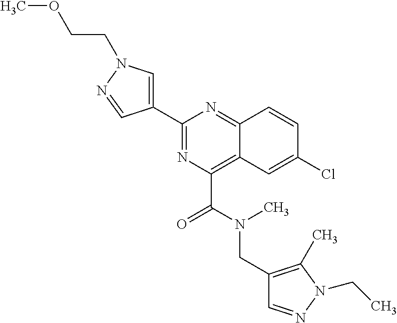 [SMILES: CCn1ncc(CN(C)C(=O)c2nc(-c3cnn(CCOC)c3)nc3ccc(Cl)cc23)c1C] | 468.0 | 468.1 | 200 | | | |
| 301 | 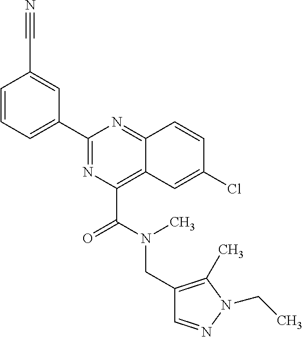 [SMILES: CCn1ncc(CN(C)C(=O)c2nc(-c3cccc(C#N)c3)nc3ccc(Cl)cc23)c1C] | 444.9 | 445.1 | 2.0 | 3.9 | | |
| 302 | 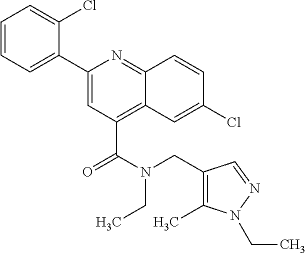 [SMILES: CCN(Cc1cnn(CC)c1C)C(=O)c1cc(-c2ccccc2Cl)nc2ccc(Cl)cc12] | 467.4 | 467.0 | 2.5 | 8.9 | | |
| 303 | 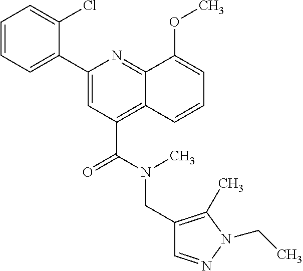 [SMILES: CCn1ncc(CN(C)C(=O)c2cc(-c3ccccc3Cl)nc3c(OC)cccc23)c1C] | 448.9 | 449.0 | 200 | | | |
| 304 | 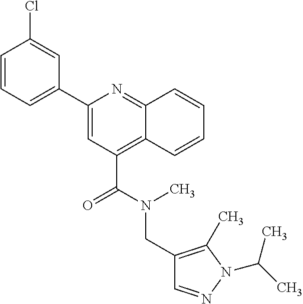 [SMILES: Cc1c(CN(C)C(=O)c2cc(-c3cccc(Cl)c3)nc3ccccc23)cnn1C(C)C] | 432.9 | 433.2 | 43 | | | |
| 305 | 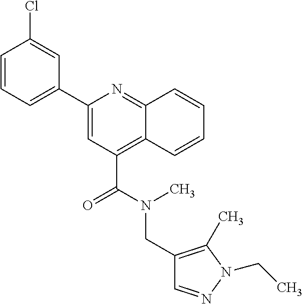 [SMILES: CCn1ncc(CN(C)C(=O)c2cc(-c3cccc(Cl)c3)nc3ccccc23)c1C] | 402.5 | 403.2 | 180 | | | |
| 306 | 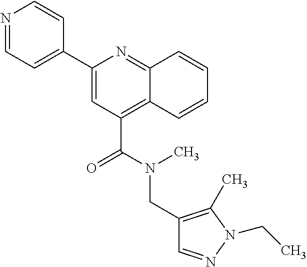 [SMILES: CCn1ncc(CN(C)C(=O)c2cc(-c3ccncc3)nc3ccccc23)c1C] | 385.5 | 386.2 | 200 | | | |
| 307 | 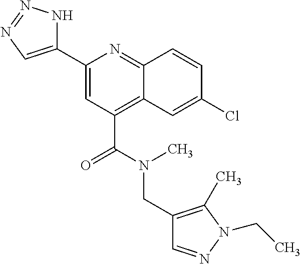 [SMILES: CCn1ncc(CN(C)C(=O)c2cc(-c3cnn[nH]3)nc3ccc(Cl)cc23)c1C] | 409.9 | 410.1 | | | | 8.0 |
| 308 | 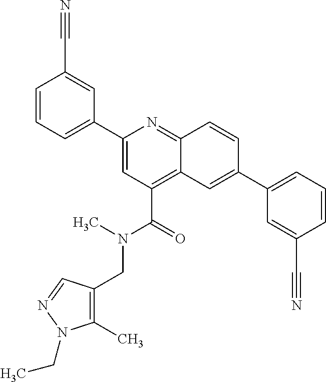 [SMILES: CCn1ncc(CN(C)C(=O)c2cc(-c3cccc(C#N)c3)nc3ccc(-c4cccc(C#N)c4)cc23)c1C] | 510.6 | 511.3 | 200 | | | |
| 309 | 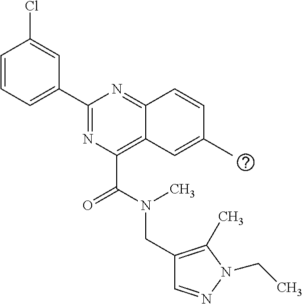 [SMILES: CCn1ncc(CN(C)C(=O)c2nc(-c3cccc(Cl)c3)nc3ccc(C)cc23)c1C] | 437.9 | 438.2 | 1.8 | | | |
| 310 | 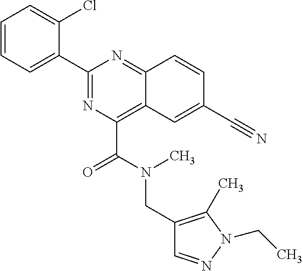 [SMILES: CCn1ncc(CN(C)C(=O)c2nc(-c3ccccc3Cl)nc3ccc(C#N)cc23)c1C] | 444.9 | 445.0 | 48 | | | |
| 311 | 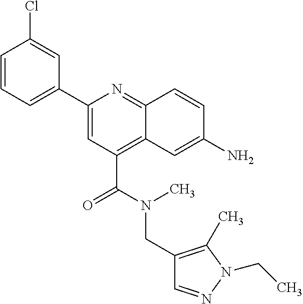 [SMILES: CCn1ncc(CN(C)C(=O)c2cc(-c3cccc(Cl)c3)nc3ccc(N)cc23)c1C] | 433.9 | 434.1 | 18 | | | |
| 312 | 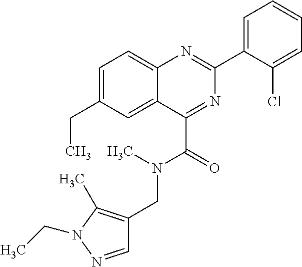 [SMILES: CCc1ccc2nc(-c3ccccc3Cl)nc(C(=O)N(C)Cc3cnn(CC)c3C)c2c1] | 448.0 | 448.1 | 0.072 | 0.58 | | |
| 313 | 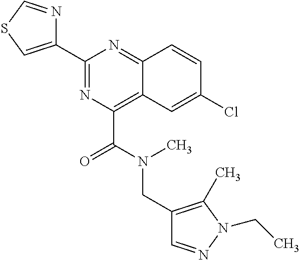 [SMILES: CCn1ncc(CN(C)C(=O)c2nc(-c3cscn3)nc3ccc(Cl)cc23)c1C] | 426.9 | 427.0 | 1.9 | 1.9 | | |
| 314 | 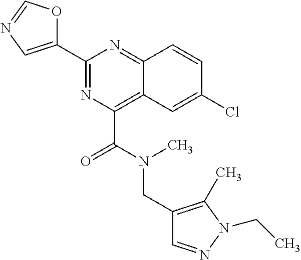 [SMILES: CCn1ncc(CN(C)C(=O)c2nc(-c3cnco3)nc3ccc(Cl)cc23)c1C] | 426.9 | 427.0 | 9.8 | 8.4 | | |
| 315 | 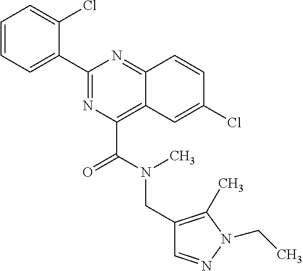 [SMILES: CCn1ncc(CN(C)C(=O)c2nc(-c3ccccc3Cl)nc3ccc(Cl)cc23)c1C] | 455.3 | 455.0 | 15 | | | |
| 316 | 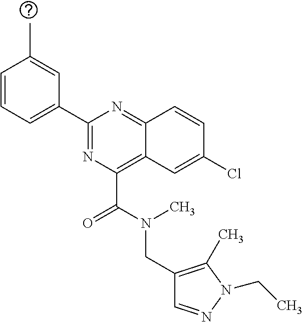 [SMILES: CCn1ncc(CN(C)C(=O)c2nc(-c3cccc(C)c3)nc3ccc(Cl)cc23)c1C] | 437.9 | 438.0 | 0.35 | 1.7 | | |
| 317 | 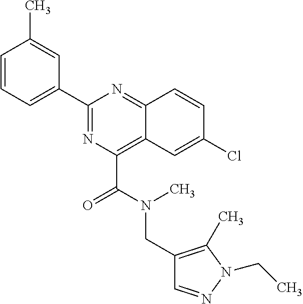 [SMILES: CCn1ncc(CN(C)C(=O)c2nc(-c3cccc(C)c3)nc3ccc(Cl)cc23)c1C] | 433.9 | 434.1 | 0.23 | 1.5 | | |
| 318 | 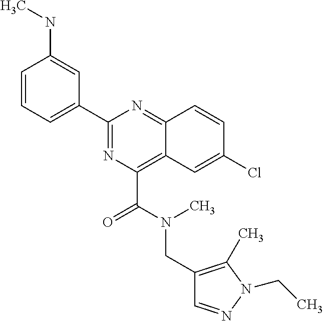 [SMILES: CCn1ncc(CN(C)C(=O)c2nc(-c3cccc(NC)c3)nc3ccc(Cl)cc23)c1C] | 449.9 | 450.1 | 0.48 | 2.0 | | |
| 319 | 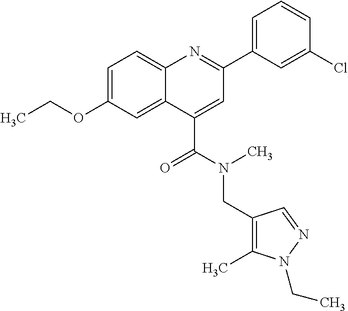 [SMILES: CCOc1ccc2nc(-c3cccc(Cl)c3)cc(C(=O)N(C)Cc3cnn(CC)c3C)c2c1] | 463.0 | 483.1 | 120 | | | |
| 320 | 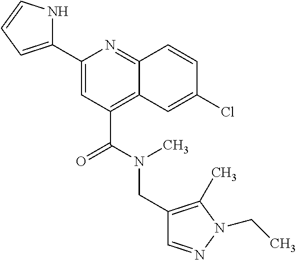 [SMILES: CCn1ncc(CN(C)C(=O)c2cc(-c3ccc[nH]3)nc3ccc(Cl)cc23)c1C] | 407.9 | 408.1 | 51 | | | |
| 321 | 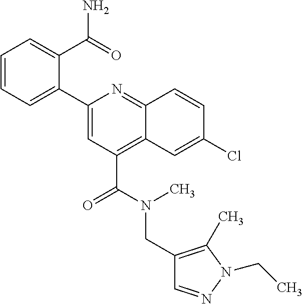 [SMILES: CCn1ncc(CN(C)C(=O)c2cc(-c3ccccc3C(N)=O)nc3ccc(Cl)cc23)c1C] | 461.9 | 482.1 | 200 | | | |
| 322 | 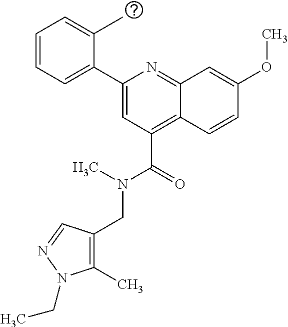 [SMILES: CCn1ncc(CN(C)C(=O)c2cc(-c3ccccc3C)nc3cc(OC)ccc23)c1C] | 448.9 | 449.0 | 134 | | | |
| 323 | 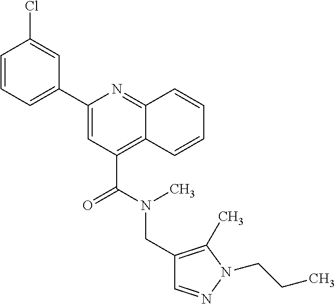 [SMILES: CCCn1ncc(CN(C)C(=O)c2cc(-c3cccc(Cl)c3)nc3ccccc23)c1C] | 432.9 | 433.2 | 200 | | | |
| 324 | 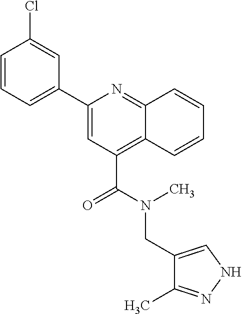 [SMILES: Cc1n[nH]cc1CN(C)C(=O)c1cc(-c2cccc(Cl)c2)nc2ccccc12] | 390.9 | 391.1 | 200 | | | |
| 325 | 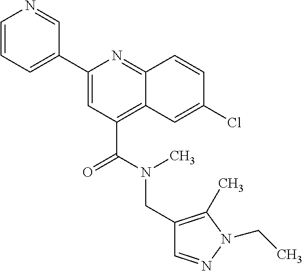 [SMILES: CCn1ncc(CN(C)C(=O)c2cc(-c3cccnc3)nc3ccc(Cl)cc23)c1C] | 419.9 | 420.1 | 55 | | | |
| 326 | 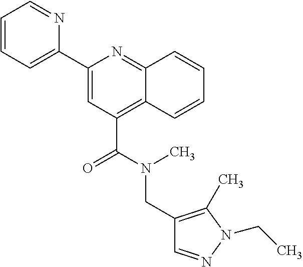 [SMILES: CCn1ncc(CN(C)C(=O)c2cc(-c3ccccn3)nc3ccccc23)c1C] | 385.5 | 386.2 | 40 | | | |
| 327 | 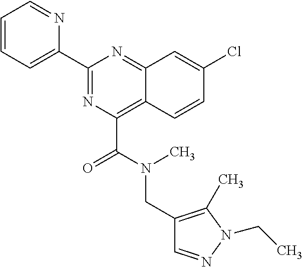 [SMILES: CCn1ncc(CN(C)C(=O)c2nc(-c3ccccn3)nc3cc(Cl)ccc23)c1C] | 420.9 | 421.0 | 152 | | | |
| 328 | 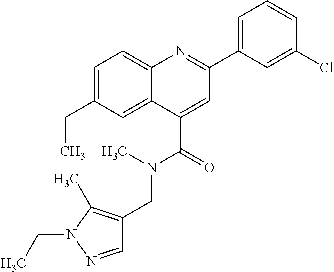 [SMILES: CCc1ccc2nc(-c3cccc(Cl)c3)cc(C(=O)N(C)Cc3cnn(CC)c3C)c2c1] | 447.0 | 447.1 | 0.29 | | | |
| 329 | 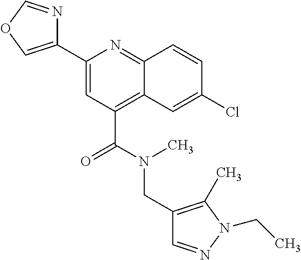 [SMILES: CCn1ncc(CN(C)C(=O)c2cc(-c3cocn3)nc3ccc(Cl)cc23)c1C] | 425.9 | 426.0 | 0.32 | 0.55 | | |
| 330 | 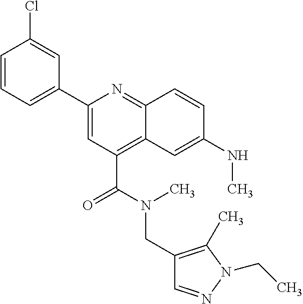 [SMILES: CCn1ncc(CN(C)C(=O)c2cc(-c3cccc(Cl)c3)nc3ccc(NC)cc23)c1C] | 448.0 | 448.3 | 9.0 | 17 | | |
| 331 | 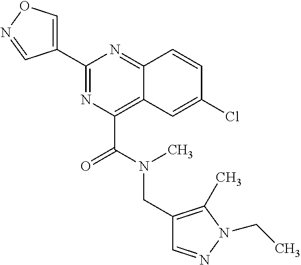 [SMILES: CCn1ncc(CN(C)C(=O)c2nc(-c3cnoc3)nc3ccc(Cl)cc23)c1C] | 428.9 | 427.2 | 17 | 12 | | |
| 332 | 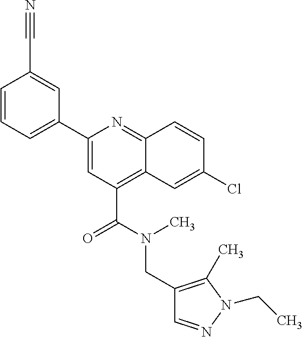 [SMILES: CCn1ncc(CN(C)C(=O)c2cc(-c3cccc(C#N)c3)nc3ccc(Cl)cc23)c1C] | 443.9 | 444.2 | 27 | | | |
| 333 | 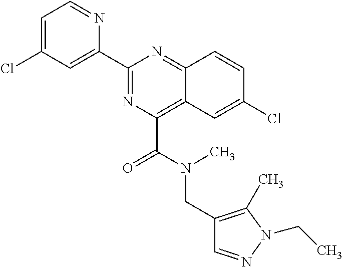 [SMILES: CCn1ncc(CN(C)C(=O)c2nc(-c3cc(Cl)ccn3)nc3ccc(Cl)cc23)c1C] | 455.3 | 455.0 | 3.9 | | | |
| 334 | 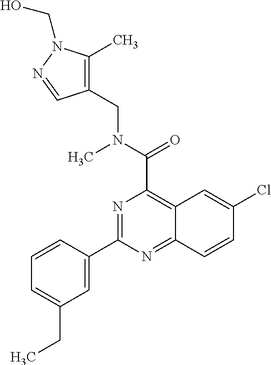 [SMILES: CCc1cccc(-c2nc(C(=O)N(C)Cc3cnn(CO)c3C)c3cc(Cl)ccc3n2)c1] | 448.0 | 448.1 | 0.51 | 4.0 | | |
| 335 | 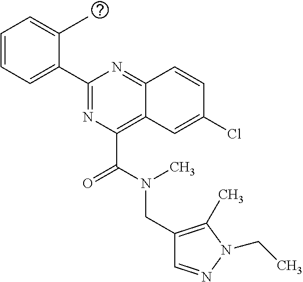 [SMILES: CCn1ncc(CN(C)C(=O)c2nc(-c3ccccc3C)nc3ccc(Cl)cc23)c1C] | 437.9 | 438.0 | 0.53 | 1.5 | | |
| 336 | 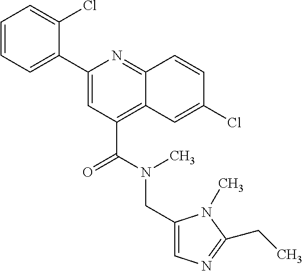 [SMILES: CCc1ncc(CN(C)C(=O)c2cc(-c3ccccc3Cl)nc3ccc(Cl)cc23)n1C] | 453.4 | 453.0 | 0.69 | 1.2 | | |
| 337 | 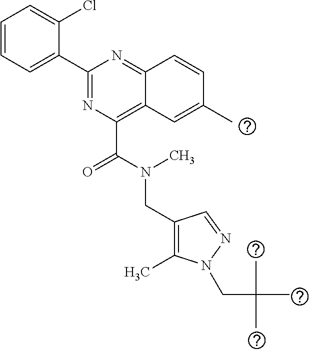 [SMILES: Cc1ccc2nc(-c3ccccc3Cl)nc(C(=O)N(C)Cc3cnn(CC(C)(C)C)c3C)c2c1] | 508.3 | 507.9 | 0.63 | 2.3 | | |
| 338 | 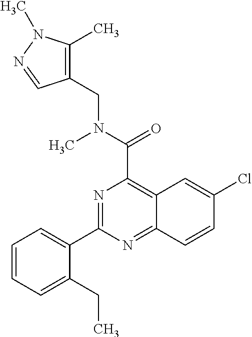 [SMILES: CCc1ccccc1-c1nc(C(=O)N(C)Cc2cnn(C)c2C)c2cc(Cl)ccc2n1] | 448.0 | 448.1 | 2.3 | 4.2 | | |
| 339 | 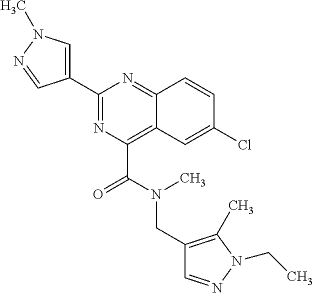 [SMILES: CCn1ncc(CN(C)C(=O)c2nc(-c3cnn(C)c3)nc3ccc(Cl)cc23)c1C] | 423.9 | 424.1 | 29 | | | |
| 340 | 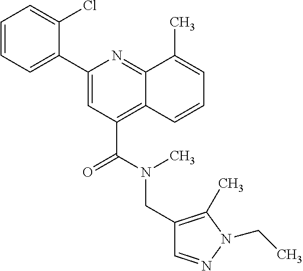 [SMILES: CCn1ncc(CN(C)C(=O)c2cc(-c3ccccc3Cl)nc3c(C)cccc23)c1C] | 432.9 | 433.0 | 40 | | | |
| 341 | 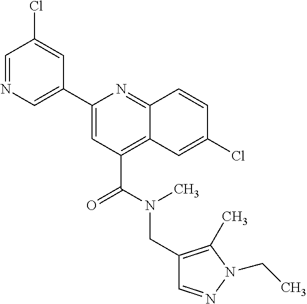 [SMILES: CCn1ncc(CN(C)C(=O)c2cc(-c3cncc(Cl)c3)nc3ccc(Cl)cc23)c1C] | 454.4 | 453.1 | 12 | | | |
| 342 | 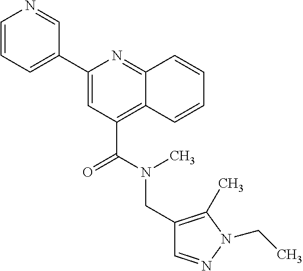 [SMILES: CCn1ncc(CN(C)C(=O)c2cc(-c3cccnc3)nc3ccccc23)c1C] | 385.5 | 386.2 | 200 | | | |
| 343 | 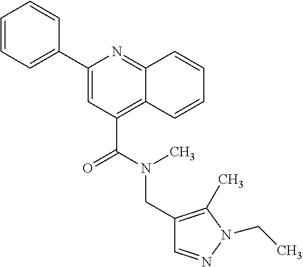 [SMILES: CCn1ncc(CN(C)C(=O)c2cc(-c3ccccc3)nc3ccccc23)c1C] | 384.5 | 385.2 | 176 | | | |
| 344 | 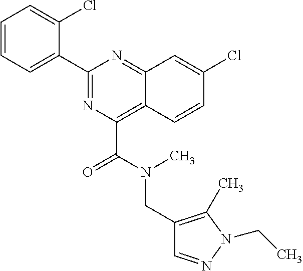 [SMILES: CCn1ncc(CN(C)C(=O)c2nc(-c3ccccc3Cl)nc3cc(Cl)ccc23)c1C] | 454.4 | 454.1 | 45 | | | |
| 345 | 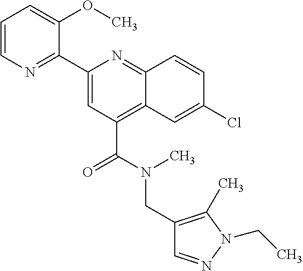 [SMILES: CCn1ncc(CN(C)C(=O)c2cc(-c3ncccc3OC)nc3ccc(Cl)cc23)c1C] | 449.9 | 450.0 | 72 | | | |
| 346 | 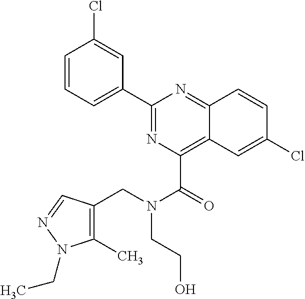 [SMILES: CCn1ncc(CN(CCO)C(=O)c2nc(-c3cccc(Cl)c3)nc3ccc(Cl)cc23)c1C] | 484.4 | 484.0 | 0.42 | 1.3 | | |
| 347 | 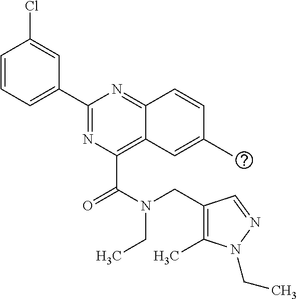 [SMILES: CCN(Cc1cnn(CC)c1C)C(=O)c1nc(-c2cccc(Cl)c2)nc2ccc(C)cc12] | 488.4 | 468.0 | 0.53 | 5.6 | | |
| 348 | 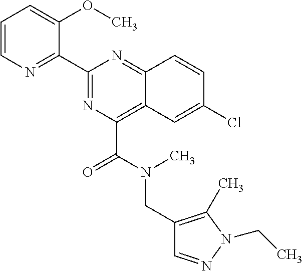 [SMILES: CCn1ncc(CN(C)C(=O)c2nc(-c3ncccc3OC)nc3ccc(Cl)cc23)c1C] | 450.9 | 451.1 | 21 | | | |
| 349 | 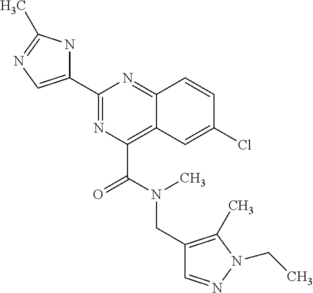 [SMILES: CCn1ncc(CN(C)C(=O)c2nc(-c3cnc(C)[nH]3)nc3ccc(Cl)cc23)c1C] | 441.0 | 441.1 | 16 | | | |
| 350 | 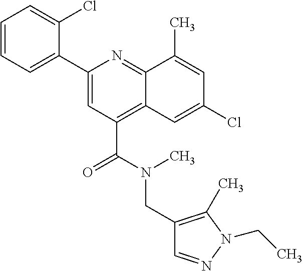 [SMILES: CCn1ncc(CN(C)C(=O)c2cc(-c3ccccc3Cl)nc3c(C)cc(Cl)cc23)c1C] | 467.4 | 467.0 | 0.63 | 6.4 | | |
| 351 | 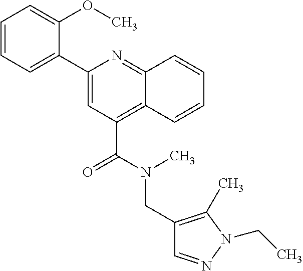 [SMILES: CCn1ncc(CN(C)C(=O)c2cc(-c3ccccc3OC)nc3ccccc23)c1C] | 414.5 | 415.2 | 32 | | | |
| 352 | 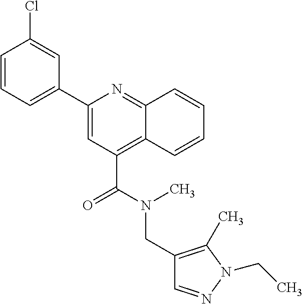 [SMILES: CCn1ncc(CN(C)C(=O)c2cc(-c3cccc(Cl)c3)nc3ccccc23)c1C] | 418.9 | 419.1 | 41 | | | |
| 353 | 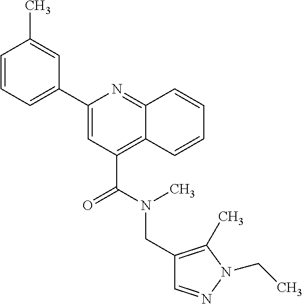 [SMILES: CCn1ncc(CN(C)C(=O)c2cc(-c3cccc(C)c3)nc3ccccc23)c1C] | 398.5 | 399.2 | 84 | | | |
| 354 | 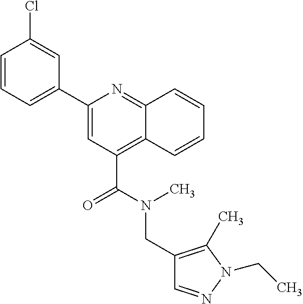 [SMILES: CCn1ncc(CN(C)C(=O)c2cc(-c3cccc(Cl)c3)nc3ccccc23)c1C] | 418.9 | #N/A | 38 | | | |
| 355 | 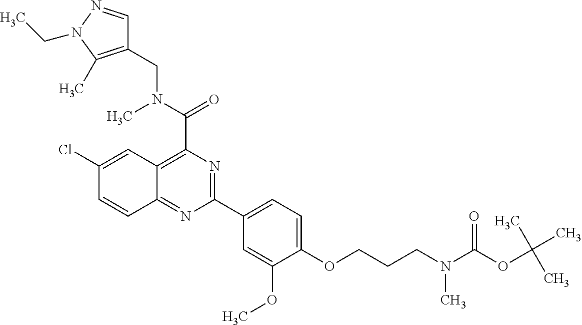 [SMILES: CCn1ncc(CN(C)C(=O)c2nc(-c3ccc(OCCCN(C)C(=O)OC(C)(C)C)c(OC)c3)nc3ccc(Cl)cc23)c1C] | 637.2 | 637.1 | 3.8 | | | |
| 356 | 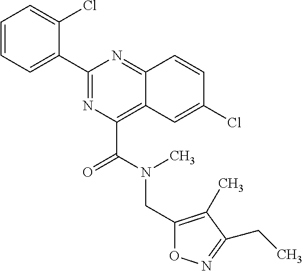 [SMILES: CCc1noc(CN(C)C(=O)c2nc(-c3ccccc3Cl)nc3ccc(Cl)cc23)c1C] | 455.3 | 455.0 | 0.40 | 1.9 | | |
| 357 | 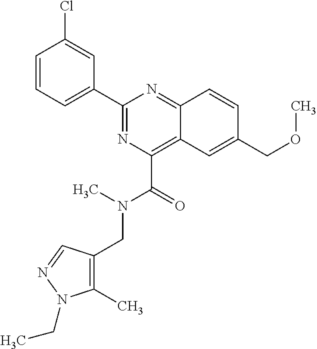 [SMILES: CCn1ncc(CN(C)C(=O)c2nc(-c3cccc(Cl)c3)nc3ccc(COC)cc23)c1C] | 464.0 | 464.1 | 0.028 | 0.72 | | |
| 358 | 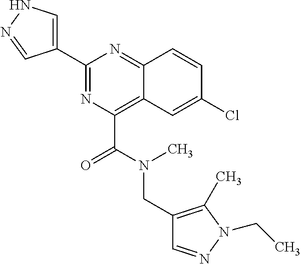 [SMILES: CCn1ncc(CN(C)C(=O)c2nc(-c3cn[nH]c3)nc3ccc(Cl)cc23)c1C] | 409.9 | 410.2 | 16 | 13 | | |
| 359 | 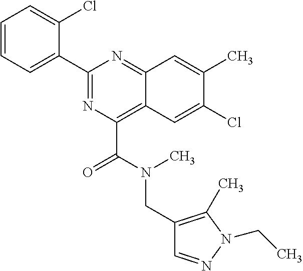 [SMILES: CCn1ncc(CN(C)C(=O)c2nc(-c3ccccc3Cl)nc3cc(C)c(Cl)cc23)c1C] | 468.4 | 468.0 | 0.40 | 1.0 | | |
| 360 | 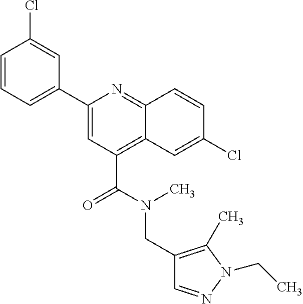 [SMILES: CCn1ncc(CN(C)C(=O)c2cc(-c3cccc(Cl)c3)nc3ccc(Cl)cc23)c1C] | 497.8 | 497.9 | 0.40 | 8.4 | | |
| 361 | 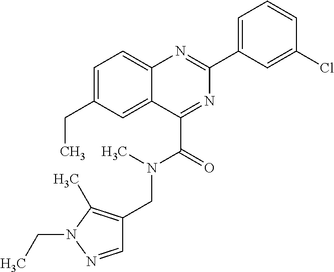 [SMILES: CCc1ccc2nc(-c3cccc(Cl)c3)nc(C(=O)N(C)Cc3cnn(CC)c3C)c2c1] | 448.0 | 448.1 | 0.0028 | 0.48 | | |
| 362 | 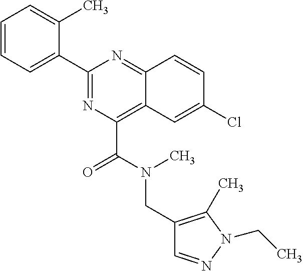 [SMILES: CCn1ncc(CN(C)C(=O)c2nc(-c3ccccc3C)nc3ccc(Cl)cc23)c1C] | 433.9 | 434.1 | 0.71 | 1.2 | | |
| 363 | 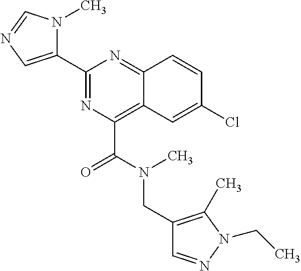 [SMILES: CCn1ncc(CN(C)C(=O)c2nc(-c3cncn3C)nc3ccc(Cl)cc23)c1C] | 423.9 | 424.1 | 9.2 | 7.4 | | |
| 364 | 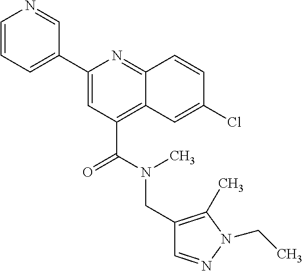 [SMILES: CCn1ncc(CN(C)C(=O)c2cc(-c3cccnc3)nc3ccc(Cl)cc23)c1C] | 419.9 | 420.1 | 34 | | | |
| 365 | 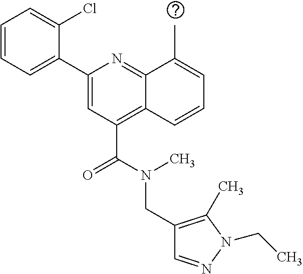 [SMILES: CCn1ncc(CN(C)C(=O)c2cc(-c3ccccc3Cl)nc3c(C)cccc23)c1C] | 436.9 | 437.1 | 14 | | | |
| 366 | 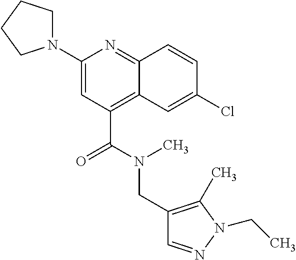 [SMILES: CCn1ncc(CN(C)C(=O)c2cc(N3CCCC3)nc3ccc(Cl)cc23)c1C] | 411.9 | 412.1 | 200 | | | |
| 367 | 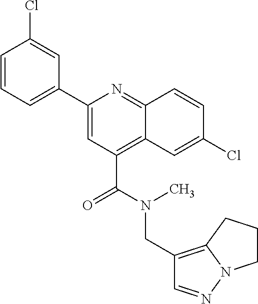 [SMILES: CN(Cc1cnn2c1CCC2)C(=O)c1cc(-c2cccc(Cl)c2)nc2ccc(Cl)cc12] | 451.4 | 451.0 | 72 | | | |
| 368 | 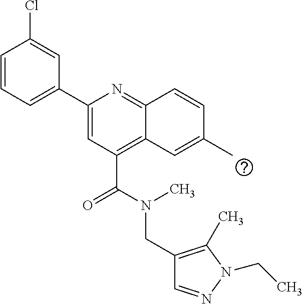 [SMILES: CCn1ncc(CN(C)C(=O)c2cc(-c3cccc(Cl)c3)nc3ccc(C)cc23)c1C] | 436.9 | 437.1 | 15 | | | |
| 369 | 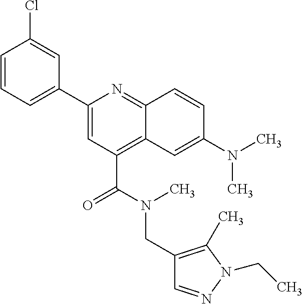 [SMILES: CCn1ncc(CN(C)C(=O)c2cc(-c3cccc(Cl)c3)nc3ccc(N(C)C)cc23)c1C] | 462.0 | 462.3 | 5.5 | 15 | | |
| 370 | 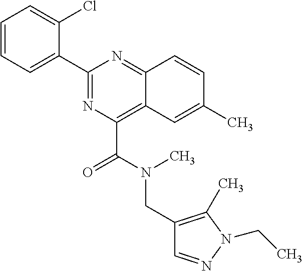 [SMILES: CCn1ncc(CN(C)C(=O)c2nc(-c3ccccc3Cl)nc3ccc(C)cc23)c1C] | 433.9 | 434.0 | 0.67 | | | |
| 371 | 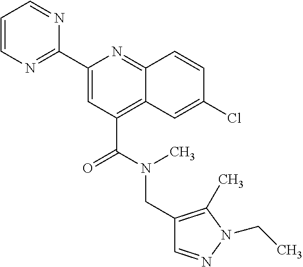 [SMILES: CCn1ncc(CN(C)C(=O)c2cc(-c3ncccn3)nc3ccc(Cl)cc23)c1C] | 420.9 | 421.1 | 25 | | | |
| 372 | 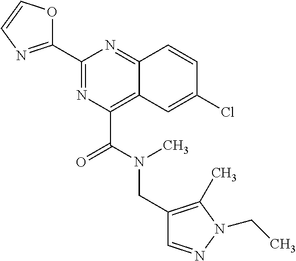 [SMILES: CCn1ncc(CN(C)C(=O)c2nc(-c3ncco3)nc3ccc(Cl)cc23)c1C] | 426.9 | 427.0 | 3.8 | 2.2 | | |
| 373 | 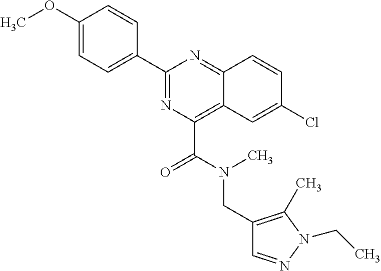 [SMILES: CCn1ncc(CN(C)C(=O)c2nc(-c3ccc(OC)cc3)nc3ccc(Cl)cc23)c1C] | 449.9 | 450.1 | 2.3 | 4.5 | | |
| 374 | 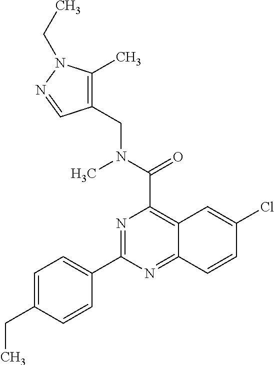 [SMILES: CCc1ccc(-c2nc(C(=O)N(C)Cc3cnn(CC)c3C)c3cc(Cl)ccc3n2)cc1] | 448.0 | 448.1 | 9.2 | 18 | | |
| 375 | 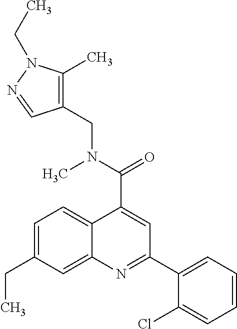 [SMILES: CCc1ccc2c(C(=O)N(C)Cc3cnn(CC)c3C)cc(-c3ccccc3Cl)nc2c1] | 447.0 | 447.1 | 102 | | | |
| 376 | 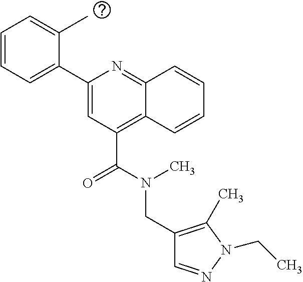 [SMILES: CCn1ncc(CN(C)C(=O)c2cc(-c3ccccc3C)nc3ccccc23)c1C] | 418.9 | 419.2 | 32 | | | |
| 377 | 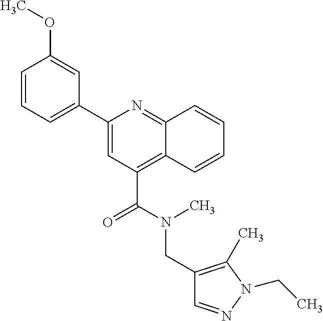 [SMILES: CCn1ncc(CN(C)C(=O)c2cc(-c3cccc(OC)c3)nc3ccccc23)c1C] | 414.5 | 415.2 | 200 | | | |
| 378 | 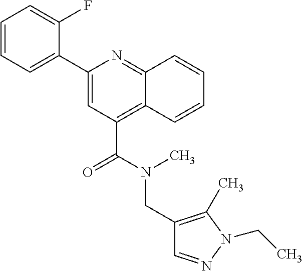 [SMILES: CCn1ncc(CN(C)C(=O)c2cc(-c3ccccc3F)nc3ccccc23)c1C] | 402.5 | 403.2 | 48 | | | |
| 379 | 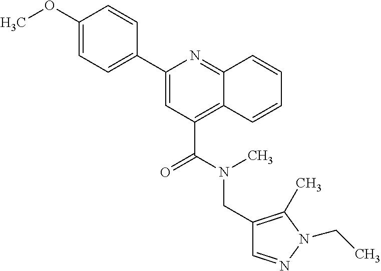 [SMILES: CCn1ncc(CN(C)C(=O)c2cc(-c3ccc(OC)cc3)nc3ccccc23)c1C] | 414.5 | 415.1 | 200 | | | |
| 380 | 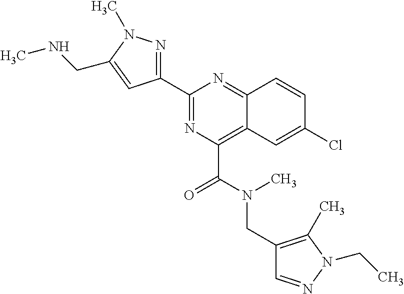 [SMILES: CCn1ncc(CN(C)C(=O)c2nc(-c3cc(CNC)n(C)n3)nc3ccc(Cl)cc23)c1C] | 467.0 | 467.2 | 25 | 20 | 20 | 14 |
| 381 | 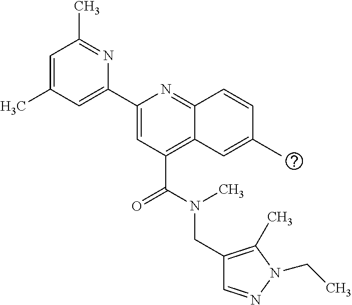 [SMILES: CCn1ncc(CN(C)C(=O)c2cc(-c3cc(C)cc(C)n3)nc3ccc(C)cc23)c1C] | 449.0 | 449.3 | 0.25 | 0.50 | 0.51 | 0.13 |
| 382 | 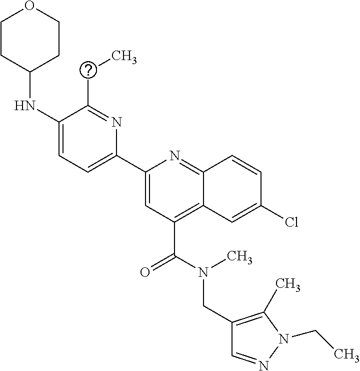 [SMILES: CCc1nc(-c2cc(C(=O)N(C)Cc3cnn(CC)c3C)c3cc(Cl)ccc3n2)ccc1NC1CCOCC1] | 549.1 | 549.3 | 0.023 | 0.61 | 0.58 | 0.020 |
| 383 | 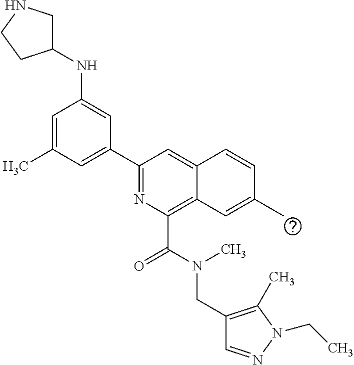 [SMILES: CCn1ncc(CN(C)C(=O)c2nc(-c3cc(C)cc(NC4CCNC4)c3)cc3ccc(C)cc23)c1C] | 518.1 | 518.2 | 0.017 | 0.19 | 0.063 | 0.0051 |
| 384 | 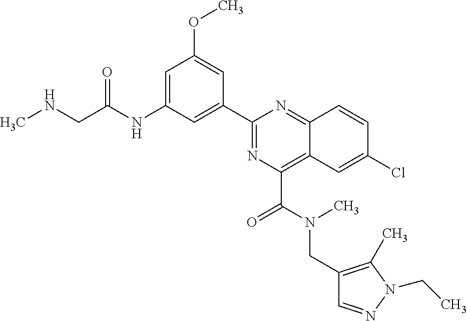 [SMILES: CCn1ncc(CN(C)C(=O)c2nc(-c3cc(NC(=O)CNC)cc(OC)c3)nc3ccc(Cl)cc23)c1C] | 536.0 | 536.2 | 0.033 | 0.13 | 0.10 | 0.012 |
| 385 | 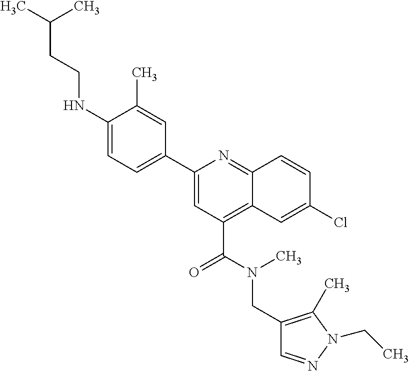 [SMILES: CCn1ncc(CN(C)C(=O)c2cc(-c3ccc(NCCC(C)C)c(C)c3)nc3ccc(Cl)cc23)c1C] | 520.1 | 520.3 | 0.033 | 0.089 | 0.069 | 0.012 |
| 386 | 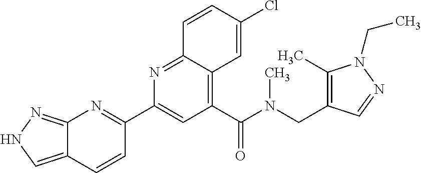 [SMILES: CCn1ncc(CN(C)C(=O)c2cc(-c3ccc4c[nH]nc4n3)nc3ccc(Cl)cc23)c1C] | 459.9 | 460.2 | 0.015 | 0.20 | 0.31 | 0.010 |
| 387 | 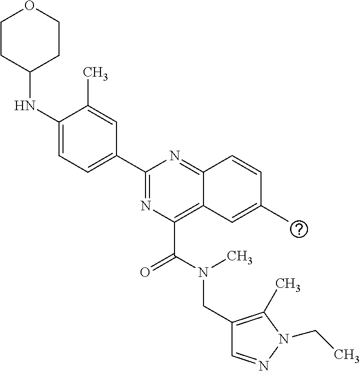 [SMILES: CCn1ncc(CN(C)C(=O)c2nc(-c3ccc(NC4CCOCC4)c(C)c3)nc3ccc(C)cc23)c1C] | 533.1 | 533.3 | 0.016 | 0.12 | 0.088 | 0.0075 |
| 388 | 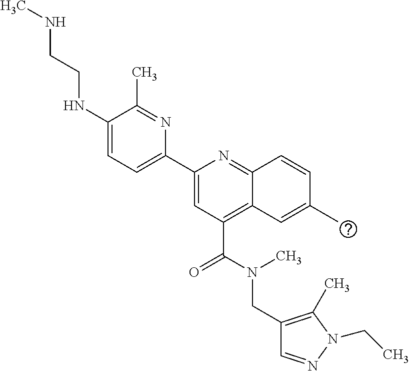 [SMILES: CCn1ncc(CN(C)C(=O)c2cc(-c3ccc(NCCNC)c(C)n3)nc3ccc(C)cc23)c1C] | 506.0 | 506.3 | 0.034 | 0.23 | 0.066 | 0.011 |
| 389 | 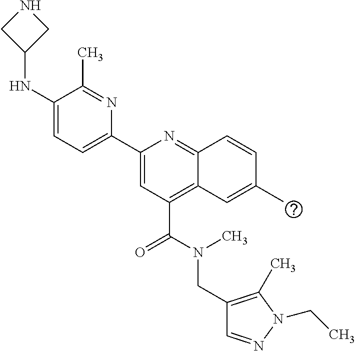 [SMILES: CCn1ncc(CN(C)C(=O)c2cc(-c3ccc(NC4CNC4)c(C)n3)nc3ccc(C)cc23)c1C] | 504.0 | 504.1 | 0.017 | 0.082 | 0.073 | 0.0074 |
| 390 | 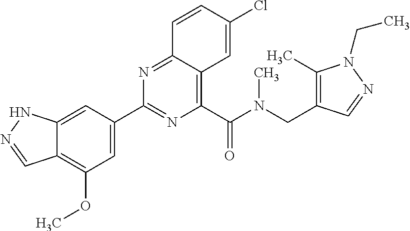 [SMILES: CCn1ncc(CN(C)C(=O)c2nc(-c3cc(OC)c4cn[nH]c4c3)nc3ccc(Cl)cc23)c1C] | 490.0 | 490.2 | 0.033 | 0.65 | 0.68 | 0.14 |
| 391 | 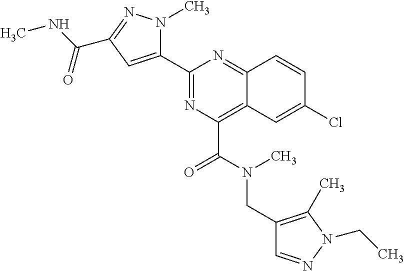 [SMILES: CCn1ncc(CN(C)C(=O)c2nc(-c3cc(C(=O)NC)nn3C)nc3ccc(Cl)cc23)c1C] | 481.0 | 481.2 | 8.5 | 3.2 | 3.1 | 4.1 |
| 392 | 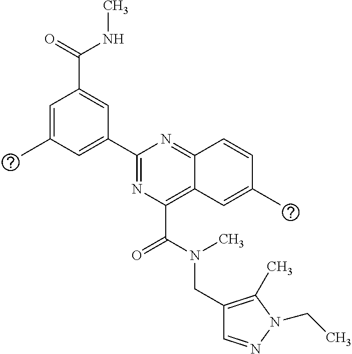 [SMILES: CCn1ncc(CN(C)C(=O)c2nc(-c3cc(C)cc(C(=O)NC)c3)nc3ccc(C)cc23)c1C] | 511.4 | 511.2 | 50 | 20 | 20 | 50 |
| 393 | 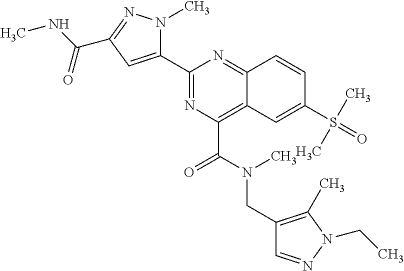 | 522.5 | 523.3 | 50 | 20 | 8.6 | 50 |
| 394 | 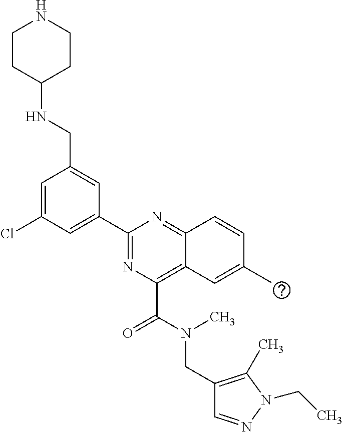 [SMILES: CCn1ncc(CN(C)C(=O)c2nc(-c3cc(Cl)cc(CNC4CCNCC4)c3)nc3ccc(C)cc23)c1C] | 566.5 | 566.3 | 2.0 | 5.4 | 4.2 | 3.8 |
| 395 | 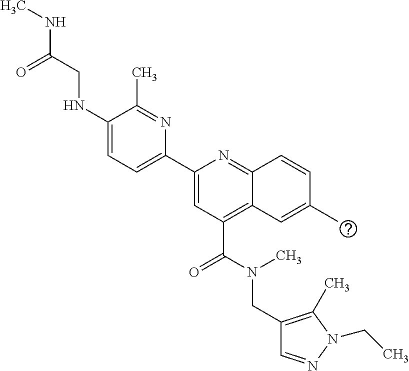 [SMILES: CCn1ncc(CN(C)C(=O)c2cc(-c3ccc(NCC(=O)NC)c(C)n3)nc3ccc(C)cc23)c1C] | 520.0 | 520.3 | 0.037 | 0.27 | 0.41 | 0.037 |
| 396 | 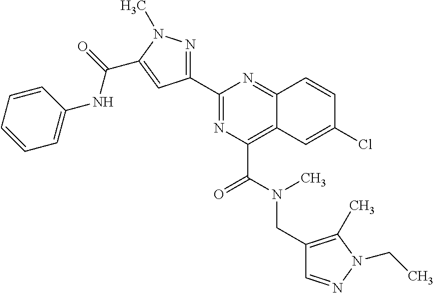 [SMILES: CCn1ncc(CN(C)C(=O)c2nc(-c3cc(C(=O)Nc4ccccc4)n(C)n3)nc3ccc(Cl)cc23)c1C] | 543.0 | 543.2 | 50 | 20 | 8.4 | 35 |
| 397 | 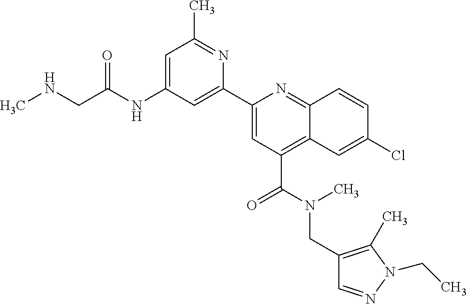 [SMILES: CCn1ncc(CN(C)C(=O)c2cc(-c3cc(NC(=O)CNC)cc(C)n3)nc3ccc(Cl)cc23)c1C] | 520.0 | 520.3 | 0.043 | 0.13 | 0.16 | 0.042 |
| 398 | 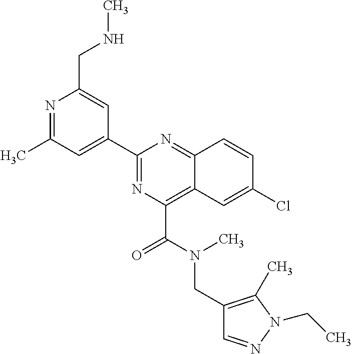 [SMILES: CCn1ncc(CN(C)C(=O)c2nc(-c3cc(C)nc(CNC)c3)nc3ccc(Cl)cc23)c1C] | 478.0 | 478.2 | 24 | 8.2 | 4.8 | 22 |
| 399 | 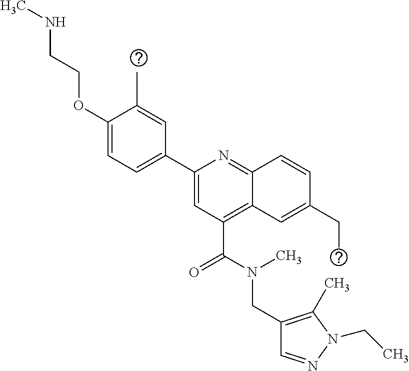 [SMILES: CCc1ccc2nc(-c3ccc(OCCNC)c(C)c3)cc(C(=O)N(C)Cc3cnn(CC)c3C)c2c1] | 523.0 | 523.3 | 11 | 4.0 | 2.2 | 9.2 |
| 400 | 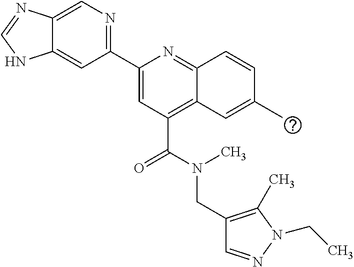 [SMILES: CCn1ncc(CN(C)C(=O)c2cc(-c3cc4[nH]cnc4cn3)nc3ccc(C)cc23)c1C] | 459.9 | 460.2 | 0.029 | 0.11 | 0.11 | 0.013 |
| 401 | 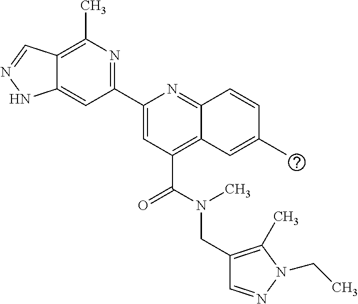 [SMILES: CCn1ncc(CN(C)C(=O)c2cc(-c3cc4[nH]ncc4c(C)n3)nc3ccc(C)cc23)c1C] | 474.0 | 474.2 | 0.056 | 0.12 | 0.13 | 0.018 |
| 402 | 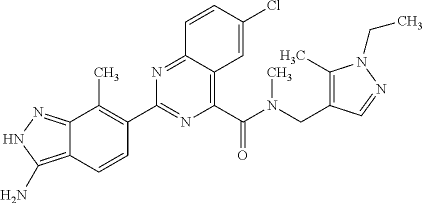 [SMILES: CCn1ncc(CN(C)C(=O)c2nc(-c3ccc4c(N)[nH]nc4c3C)nc3ccc(Cl)cc23)c1C] | 489.0 | 489.2 | 0.062 | 0.35 | 0.31 | 0.30 |
| 403 | 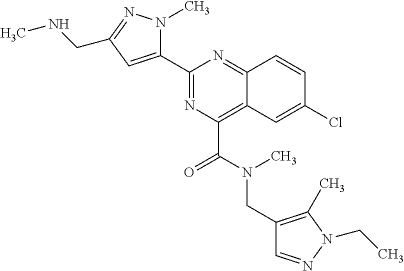 [SMILES: CCn1ncc(CN(C)C(=O)c2nc(-c3cc(CNC)nn3C)nc3ccc(Cl)cc23)c1C] | 467.0 | 467.2 | 50 | 12 | 10 | 22 |
| 404 | 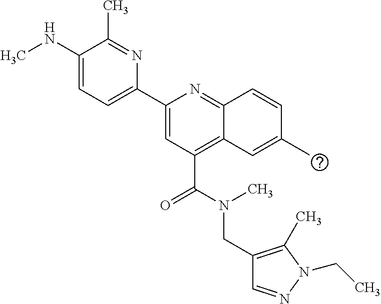 [SMILES: CCn1ncc(CN(C)C(=O)c2cc(-c3ccc(NC)c(C)n3)nc3ccc(C)cc23)c1C] | 463.0 | 463.2 | 0.0025 | 0.17 | 0.12 | 0.023 |
| 405 | 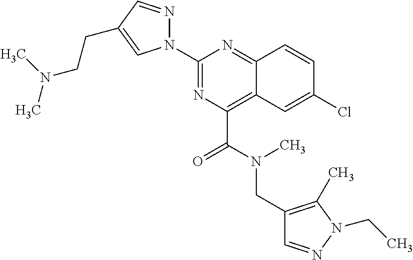 [SMILES: CCn1ncc(CN(C)C(=O)c2nc(-n3cc(CCN(C)C)cn3)nc3ccc(Cl)cc23)c1C] | 481.0 | 481.3 | 5.7 | 1.6 | 1.7 | 2.7 |
| 406 | 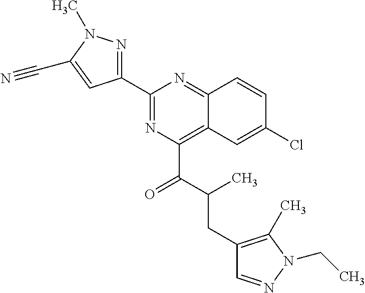 [SMILES: CCn1ncc(CC(C)C(=O)c2nc(-c3cc(C#N)n(C)n3)nc3ccc(Cl)cc23)c1C] | 448.9 | 449.1 | 50 | 20 | 20 | 50 |
| 407 | 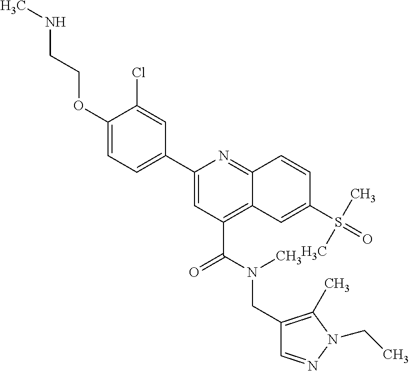 | 569.0 | 569.3 | 2.1 | 18 | 7.4 | 0.70 |
| 408 | 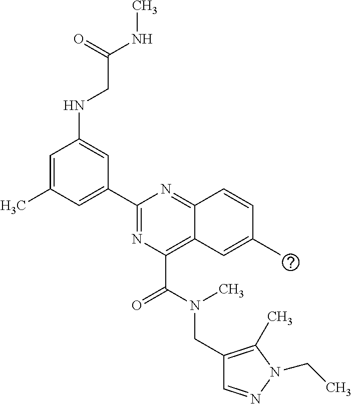 [SMILES: CCn1ncc(CN(C)C(=O)c2nc(-c3cc(C)cc(NCC(=O)NC)c3)nc3ccc(C)cc23)c1C] | 520.0 | 520.2 | 0.43 | 1.1 | 0.89 | 0.95 |
| 409 | 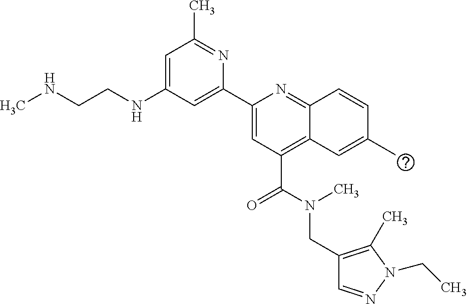 [SMILES: CCn1ncc(CN(C)C(=O)c2cc(-c3cc(NCCNC)cc(C)n3)nc3ccc(C)cc23)c1C] | 506.0 | 506.3 | 0.074 | 0.23 | 0.054 | 0.023 |
| 410 | 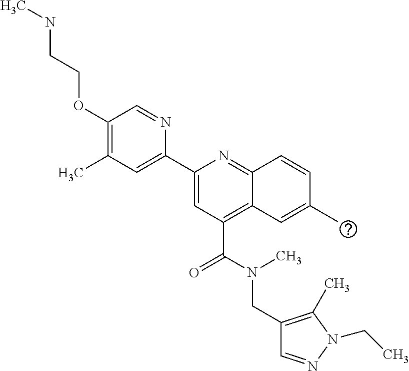 [SMILES: CCn1ncc(CN(C)C(=O)c2cc(-c3cc(C)c(OCCNC)cn3)nc3ccc(C)cc23)c1C] | 507.0 | 507.2 | 5.5 | 6.1 | 2.8 | 1.4 |
| 411 | 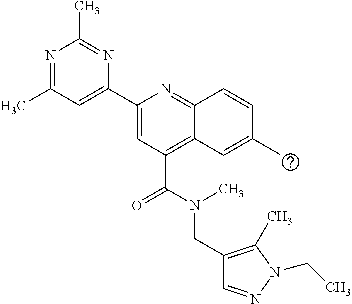 [SMILES: CCn1ncc(CN(C)C(=O)c2cc(-c3cc(C)nc(C)n3)nc3ccc(C)cc23)c1C] | 449.9 | 450.1 | 50 | 20 | 20 | 50 |
| 412 | 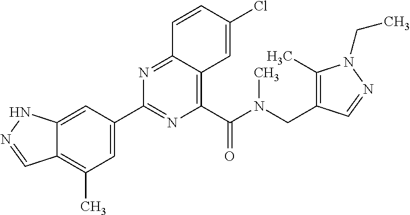 [SMILES: CCn1ncc(CN(C)C(=O)c2nc(-c3cc(C)c4cn[nH]c4c3)nc3ccc(Cl)cc23)c1C] | 474.0 | 474.2 | | 0.25 | 0.15 | 0.13 |
| 413 | 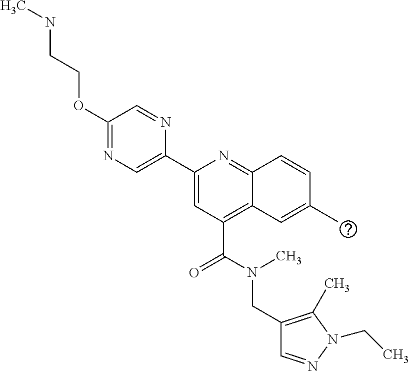 [SMILES: CCn1ncc(CN(C)C(=O)c2cc(-c3cnc(OCCNC)cn3)nc3ccc(C)cc23)c1C] | 494.0 | 494.3 | 21 | 6.4 | 5.0 | 7.4 |
| 414 | 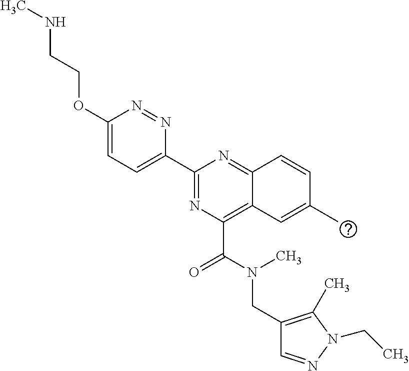 [SMILES: CCn1ncc(CN(C)C(=O)c2nc(-c3ccc(OCCNC)nn3)nc3ccc(C)cc23)c1C] | 495.0 | 495.3 | 30 | 20 | 20 | 44 |
| 415 | 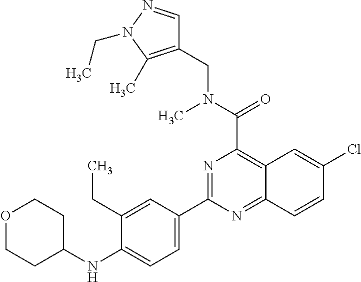 [SMILES: CCc1cc(-c2nc(C(=O)N(C)Cc3cnn(CC)c3C)c3cc(Cl)ccc3n2)ccc1NC1CCOCC1] | 547.1 | 547.3 | 0.032 | 0.24 | 0.24 | 0.023 |
| 416 | 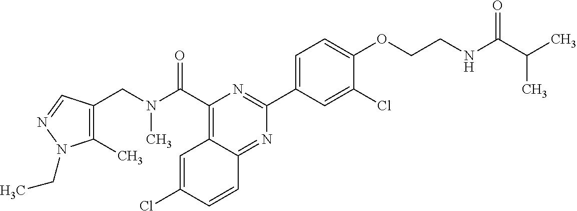 [SMILES: CCn1ncc(CN(C)C(=O)c2nc(-c3ccc(OCCNC(=O)C(C)C)c(Cl)c3)nc3ccc(Cl)cc23)c1C] | 583.5 | 583.3 | | 0.40 | 0.40 | 0.18 |
| 417 | 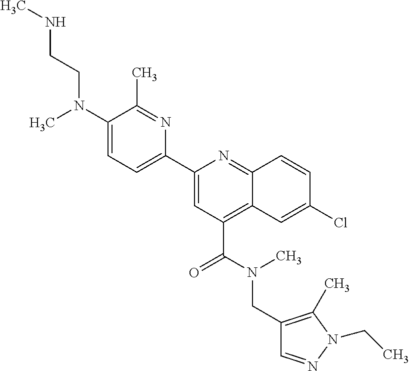 [SMILES: CCn1ncc(CN(C)C(=O)c2cc(-c3ccc(N(C)CCNC)c(C)n3)nc3ccc(Cl)cc23)c1C] | 520.1 | 520.3 | 22 | 4.9 | 3.1 | 16 |
| 418 | 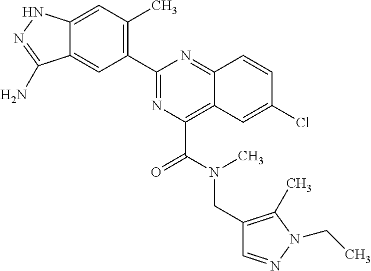 [SMILES: CCn1ncc(CN(C)C(=O)c2nc(-c3cc4c(N)n[nH]c4cc3C)nc3ccc(Cl)cc23)c1C] | 489.0 | 489.3 | 0.0025 | 0.078 | 0.062 | 0.0025 |
| 419 | 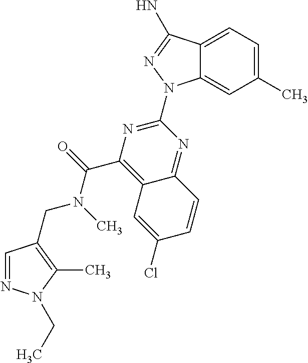 [SMILES: CCn1ncc(CN(C)C(=O)c2nc(-n3nc(N)c4ccc(C)cc43)nc3ccc(Cl)cc23)c1C] | 489.0 | 489.2 | 10 | 5.8 | 0.41 | 4.4 |
| 420 | 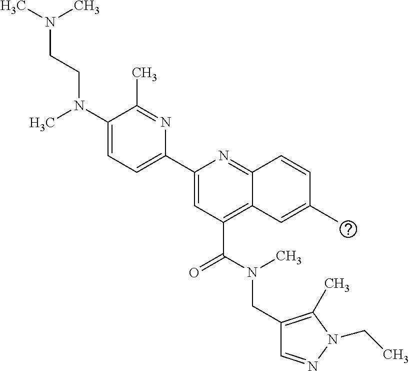 [SMILES: CCn1ncc(CN(C)C(=O)c2cc(-c3ccc(N(C)CCN(C)C)c(C)n3)nc3ccc(C)cc23)c1C] | 534.1 | 534.4 | | 0.74 | | |
| 421 | 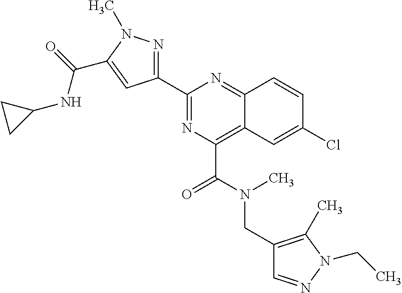 [SMILES: CCn1ncc(CN(C)C(=O)c2nc(-c3cc(C(=O)NC4CC4)n(C)n3)nc3ccc(Cl)cc23)c1C] | 507.0 | 507.3 | | 0.26 | | |
| 422 | 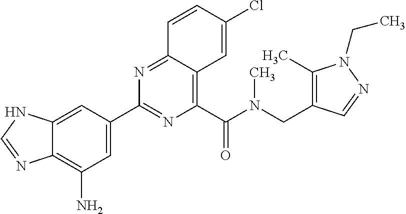 [SMILES: CCn1ncc(CN(C)C(=O)c2nc(-c3cc(N)c4nc[nH]c4c3)nc3ccc(Cl)cc23)c1C] | 474.9 | 474.2 | | 0.60 | | |
| 423 | 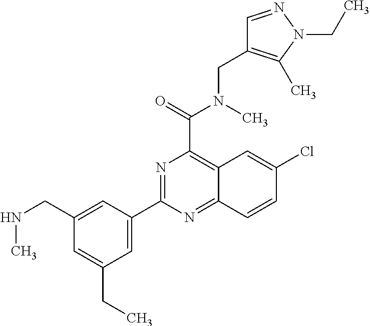 [SMILES: CCc1cc(CNC)cc(-c2nc(C(=O)N(C)Cc3cnn(CC)c3C)c3cc(Cl)ccc3n2)c1] | 491.0 | 491.3 | | 0.53 | | |
| 424 | 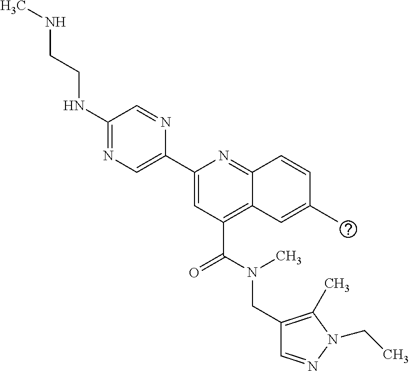 [SMILES: CCn1ncc(CN(C)C(=O)c2cc(-c3cnc(NCCNC)cn3)nc3ccc(C)cc23)c1C] | 493.0 | 493.3 | | 0.40 | | |
| 425 | 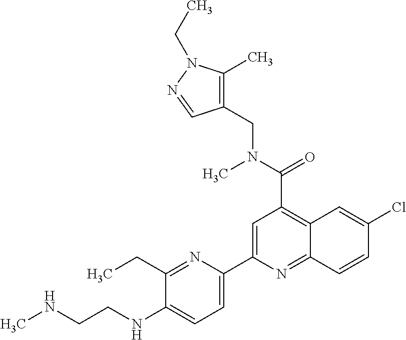 [SMILES: CCc1nc(-c2cc(C(=O)N(C)Cc3cnn(CC)c3C)c3cc(Cl)ccc3n2)ccc1NCCNC] | 520.1 | 520.3 | | 0.15 | | |
| 426 | 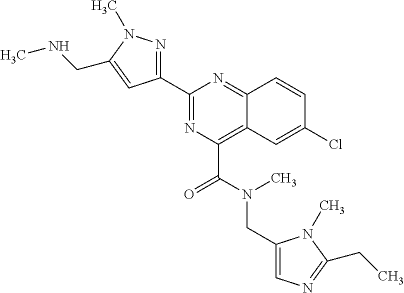 [SMILES: CCc1ncc(CN(C)C(=O)c2nc(-c3cc(CNC)n(C)n3)nc3ccc(Cl)cc23)n1C] | 481.0 | 481.2 | | 0.14 | | |
| 427 | 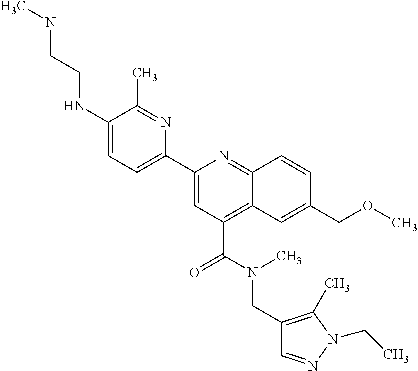 | 515.7 | 516.3 | | 0.19 | | |
| 428 | 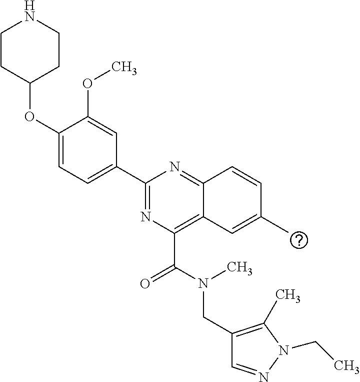 [SMILES: CCn1ncc(CN(C)C(=O)c2nc(-c3ccc(OC4CCNCC4)c(OC)c3)nc3ccc(C)cc23)c1C] | 549.1 | 549.2 | | 1.4 | | |
| 429 | 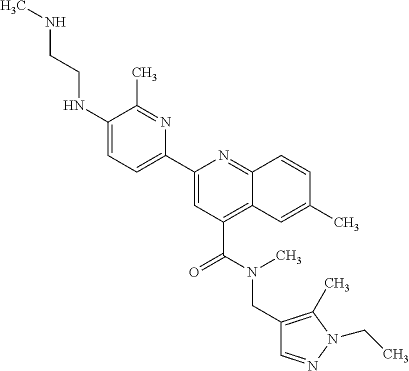 [SMILES: CCn1ncc(CN(C)C(=O)c2cc(-c3ccc(NCCNC)c(C)n3)nc3ccc(C)cc23)c1C] | 485.6 | 486.3 | | 0.16 | | |
| 430 | 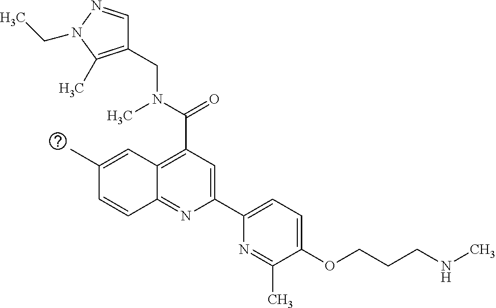 [SMILES: CCn1ncc(CN(C)C(=O)c2cc(-c3ccc(OCCCNC)c(C)n3)nc3ccc(C)cc23)c1C] | 521.1 | 521.3 | | 0.69 | | |
| 431 | 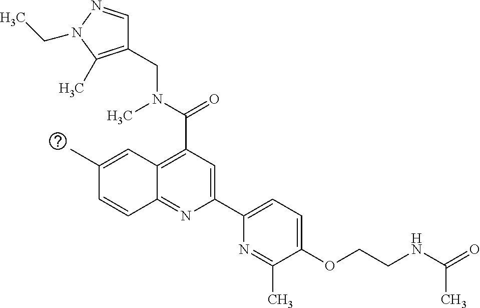 [SMILES: CCn1ncc(CN(C)C(=O)c2cc(-c3ccc(OCCNC(C)=O)c(C)n3)nc3ccc(C)cc23)c1C] | 535.0 | 535.2 | | 0.84 | | |

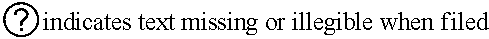

TABLE 2
Exemplary Compounds and Data

| | | | | CREBBP | | | EP300 |
|---|---|---|---|---|---|---|---|
| | | | Mass | Bioche- | CREBBP | CREBBP | Bioche- |
| Com- | | | De- | mistry | ICW | HTP | mistry |
| pound | | | tec- | IC₅₀ | IC₅₀ | IC₅₀ | IC₅₀ |
| Num- | | | ted | (micro- | (micro- | (micro- | (micro- |
| ber | STRUCTURE | MW | M + 1 | molar) | molar) | molar) | molar) |
| 432 | 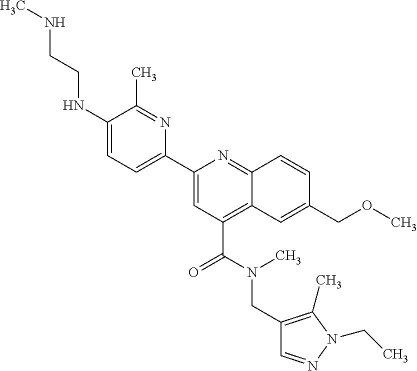 | 515.3 | 516.3 | 0.03965 | 0.1547 | 0.1061 | 0.0186 |
| 433 | 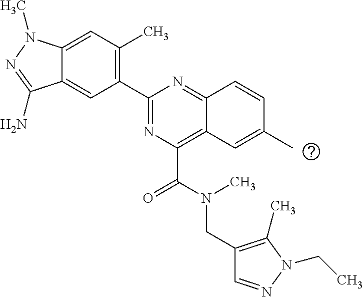 [SMILES: CCn1ncc(CN(C)C(=O)c2nc(-c3cc4c(N)nn(C)c4cc3C)nc3ccc(C)cc23)c1C] | 502.2 | 503.2 | 24.85 | 20 | 2.215 | 12.46 |
| 434 | 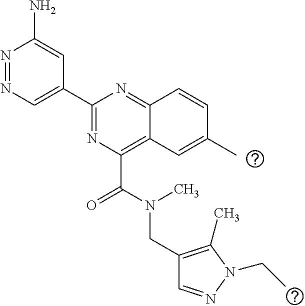 [SMILES: CCn1ncc(CN(C)C(=O)c2nc(-c3cnnc(N)c3)nc3ccc(C)cc23)c1C] | 436.2 | 437.2 | 3.507 | 1.716 | 2.253 | 2.702 |
| 435 | 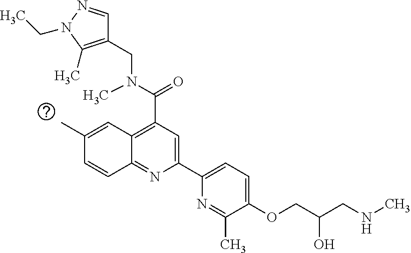 [SMILES: CCn1ncc(CN(C)C(=O)c2cc(-c3ccc(OCC(O)CNC)c(C)n3)nc3ccc(C)cc23)c1C] | 536.2 | 537.3 | 0.03682 | 0.1631 | 0.1023 | 0.0158 |
| 436 | 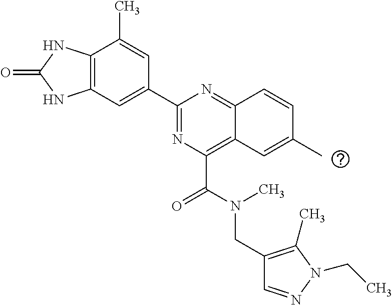 [SMILES: CCn1ncc(CN(C)C(=O)c2nc(-c3cc(C)c4[nH]c(=O)[nH]c4c3)nc3ccc(C)cc23)c1C] | 489.2 | 490.2 | 0.00721 | 0.1298 | 0.09424 | 0.0042 |
| 437 | 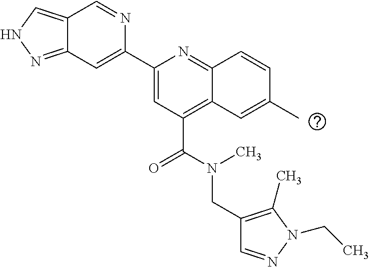 [SMILES: CCn1ncc(CN(C)C(=O)c2cc(-c3cc4n[nH]cc4cn3)nc3ccc(C)cc23)c1C] | 459.2 | 460.2 | 0.007821 | 0.07454 | 0.1187 | 0.0052 |
| 438 | 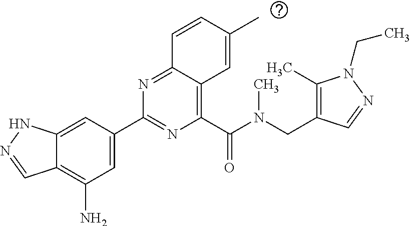 [SMILES: CCn1ncc(CN(C)C(=O)c2nc(-c3cc(N)c4cn[nH]c4c3)nc3ccc(C)cc23)c1C] | 474.2 | 475.2 | 0.0025 | 0.08485 | 0.07845 | 0.0378 |
| 439 | 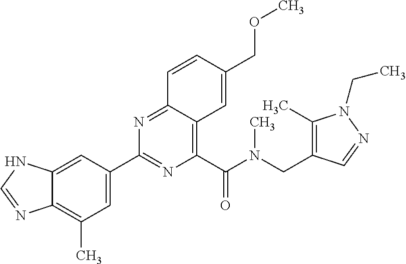 [SMILES: CCn1ncc(CN(C)C(=O)c2nc(-c3cc(C)c4nc[nH]c4c3)nc3ccc(COC)cc23)c1C] | 483.2 | 484.2 | 0.17 | 0.9192 | 0.7517 | 0.6282 |
| 440 | 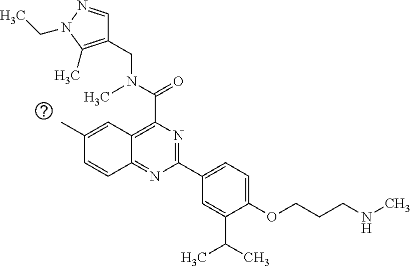 [SMILES: CCn1ncc(CN(C)C(=O)c2nc(-c3ccc(OCCCNC)c(C(C)C)c3)nc3ccc(C)cc23)c1C] | 548.3 | 549.3 | 0.08817 | 2.048 | 0.9908 | 0.9372 |
| 441 | 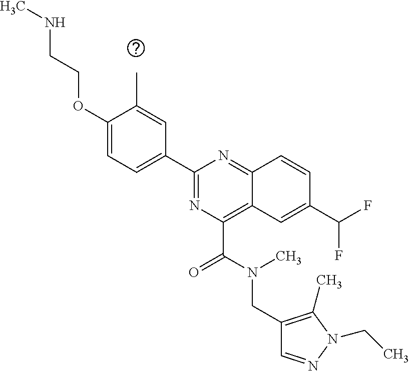 [SMILES: CCn1ncc(CN(C)C(=O)c2nc(-c3ccc(OCCNC)c(C)c3)nc3ccc(C(F)F)cc23)c1C] | 542.2 | 543.3 | 0.05026 | 0.2691 | 0.2163 | 0.0367 |
| 442 | 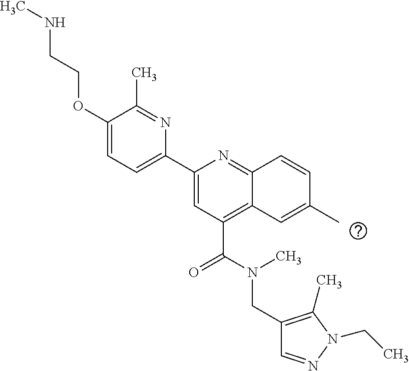 [SMILES: CCn1ncc(CN(C)C(=O)c2cc(-c3ccc(OCCNC)c(C)n3)nc3ccc(C)cc23)c1C] | 506.2 | 507.2 | 0.07686 | 0.1948 | 0.3503 | 0.414 |
| 443 | 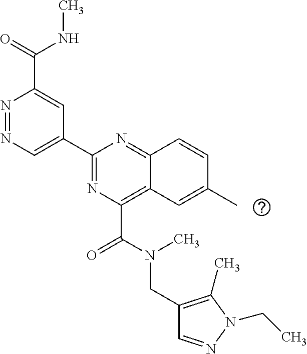 [SMILES: CCn1ncc(CN(C)C(=O)c2nc(-c3cnnc(C(=O)NC)c3)nc3ccc(C)cc23)c1C] | 478.2 | 479.2 | 50 | 20 | 20 | 50 |
| 444 | 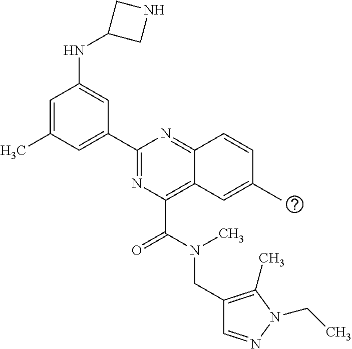 [SMILES: CCn1ncc(CN(C)C(=O)c2nc(-c3cc(C)cc(NC4CNC4)c3)nc3ccc(C)cc23)c1C] | 503.2 | 504.3 | 0.0123 | 0.1598 | 0.05816 | 0.0041 |
| 445 | 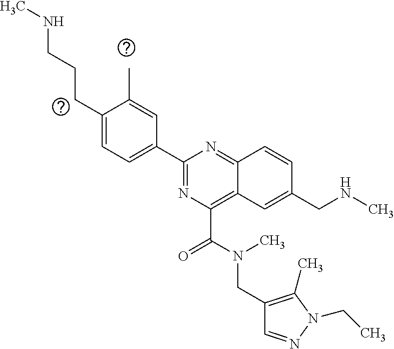 | 535.2 | 536.2 | 1.151 | 7.169 | 1.708 | 3.295 |
| 446 | 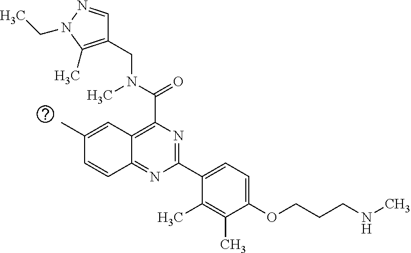 [SMILES: CCn1ncc(CN(C)C(=O)c2nc(-c3ccc(OCCCNC)c(C)c3C)nc3ccc(C)cc23)c1C] | 534.3 | 535.3 | 0.02479 | 0.1124 | 0.03423 | 0.0132 |
| 447 | 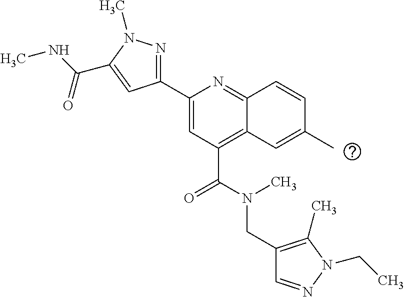 [SMILES: CCn1ncc(CN(C)C(=O)c2cc(-c3cc(C(=O)NC)n(C)n3)nc3ccc(C)cc23)c1C] | 479.2 | 480.2 | 0.01209 | 0.05144 | 0.06883 | 0.002 |
| 448 | 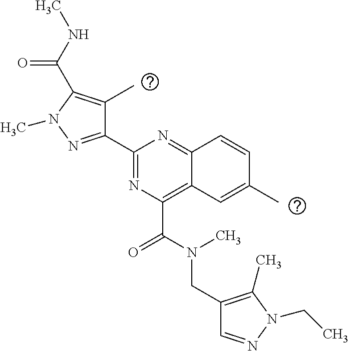 [SMILES: CCn1ncc(CN(C)C(=O)c2nc(-c3nn(C)c(C(=O)NC)c3C)nc3ccc(C)cc23)c1C] | 514.1 | 515.2 | 48.3 | 14.28 | 12.59 | 35.55 |
| 449 | 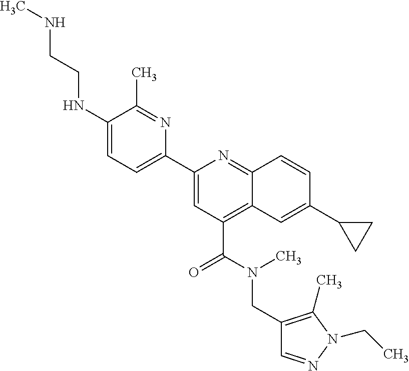 [SMILES: CCn1ncc(CN(C)C(=O)c2cc(-c3ccc(NCCNC)c(C)n3)nc3ccc(C4CC4)cc23)c1C] | 511.3 | 512.3 | 0.0498 | 0.2209 | 0.1347 | 0.0511 |
| 450 | 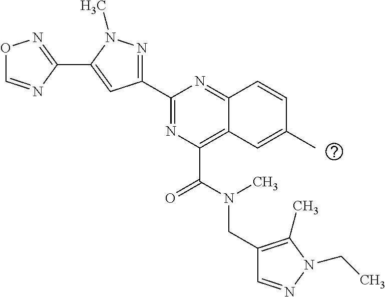 [SMILES: CCn1ncc(CN(C)C(=O)c2nc(-c3cc(-c4ncon4)n(C)n3)nc3ccc(C)cc23)c1C] | 491.2 | 492.2 | 50 | 20 | 20 | 24.55 |
| 451 | 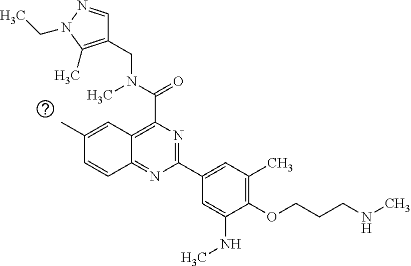 [SMILES: CCn1ncc(CN(C)C(=O)c2nc(-c3cc(C)c(OCCCNC)c(NC)c3)nc3ccc(C)cc23)c1C] | 549.3 | 550.3 | 3.011 | 12.99 | 2.285 | 3.883 |
| 452 | 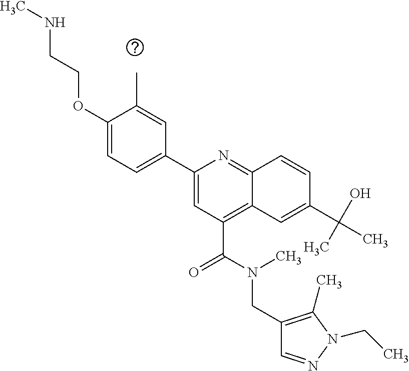 [SMILES: CCn1ncc(CN(C)C(=O)c2cc(-c3ccc(OCCNC)c(C)c3)nc3ccc(C(C)(C)O)cc23)c1C] | 550.2 | 551.3 | 34.5 | 20 | 7.122 | 8.753 |
| 453 | 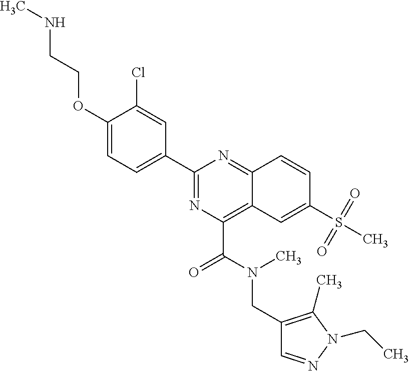 [SMILES: CCn1ncc(CN(C)C(=O)c2nc(-c3ccc(OCCNC)c(Cl)c3)nc3ccc(S(C)(=O)=O)cc23)c1C] | 570.2 | 571.3 | 3.912 | 4.829 | 1.204 | 2.365 |
| 454 | 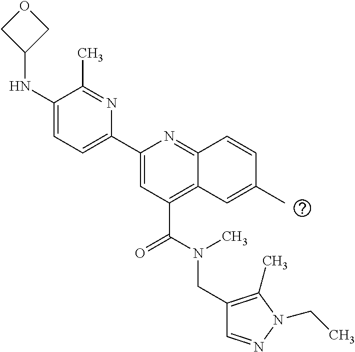 [SMILES: CCn1ncc(CN(C)C(=O)c2cc(-c3ccc(NC4COC4)c(C)n3)nc3ccc(C)cc23)c1C] | 504.2 | 505.3 | 0.02267 | 0.1409 | 0.1326 | 0.0055 |
| 455 | 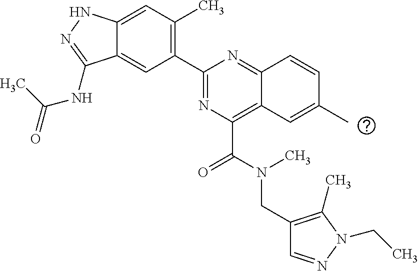 [SMILES: CCn1ncc(CN(C)C(=O)c2nc(-c3cc4c(NC(C)=O)n[nH]c4cc3C)nc3ccc(C)cc23)c1C] | 530.2 | 531.2 | 0.3534 | 1.205 | 0.5468 | 0.1136 |
| 456 | 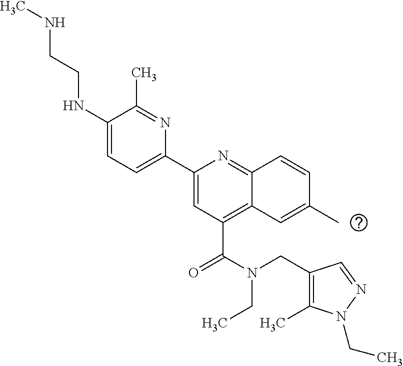 [SMILES: CCN(Cc1cnn(CC)c1C)C(=O)c1cc(-c2ccc(NCCNC)c(C)n2)nc2ccc(C)cc12] | 519.3 | 520.3 | 0.104 | 1.408 | 0.6264 | 0.8801 |
| 457 | 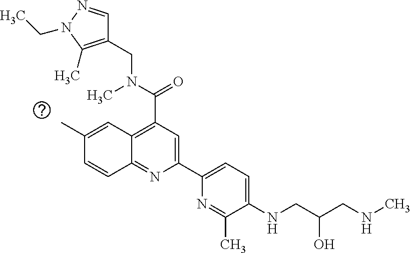 [SMILES: CCn1ncc(CN(C)C(=O)c2cc(-c3ccc(NCC(O)CNC)c(C)n3)nc3ccc(C)cc23)c1C] | 535.2 | 536.3 | 0.01548 | 0.1877 | 0.04169 | 0.0057 |
| 458 | 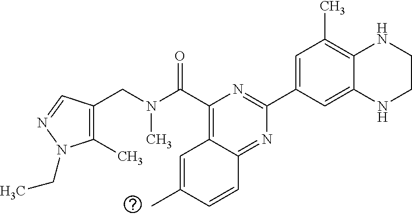 [SMILES: CCn1ncc(CN(C)C(=O)c2nc(-c3cc(C)c4c(c3)NCCN4)nc3ccc(C)cc23)c1C] | 489.2 | 490.2 | 1.433 | 1.054 | 0.5231 | 1.456 |
| 459 | 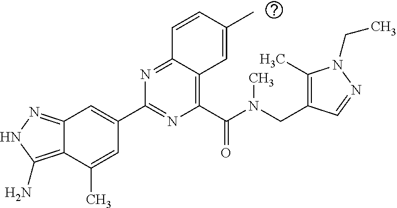 [SMILES: CCn1ncc(CN(C)C(=O)c2nc(-c3cc(C)c4c(N)[nH]nc4c3)nc3ccc(C)cc23)c1C] | 488.2 | 489.2 | 0.0139 | 0.06038 | 0.05436 | 0.0045 |
| 460 | 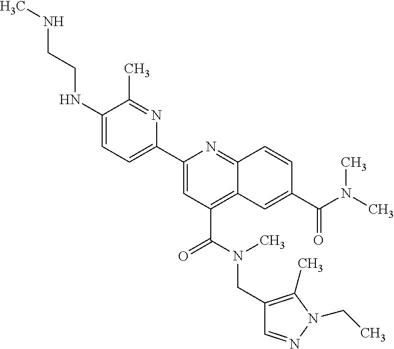 | 542.3 | 543.4 | 50 | 20 | 20 | 50 |
| 461 | 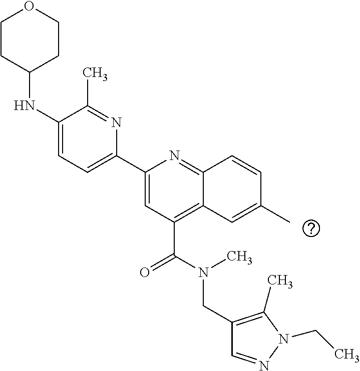 [SMILES: CCn1ncc(CN(C)C(=O)c2cc(-c3ccc(NC4CCOCC4)c(C)n3)nc3ccc(C)cc23)c1C] | 532.2 | 533.3 | 0.02745 | 0.1654 | 0.1267 | 0.0098 |
| 462 | 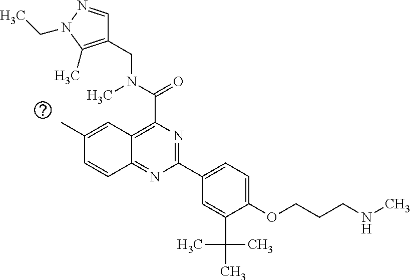 [SMILES: CCn1ncc(CN(C)C(=O)c2nc(-c3ccc(OCCCNC)c(C(C)(C)C)c3)nc3ccc(C)cc23)c1C] | 562.3 | 563.3 | 9.699 | 16.4 | 1.409 | 15.75 |
| 463 | 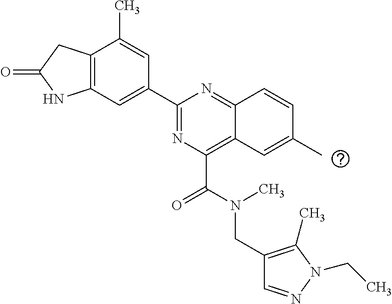 [SMILES: CCn1ncc(CN(C)C(=O)c2nc(-c3cc(C)c4c(c3)NC(=O)C4)nc3ccc(C)cc23)c1C] | 488.2 | 489.1 | 2.566 | 1.403 | 1.507 | 0.9927 |
| 464 | 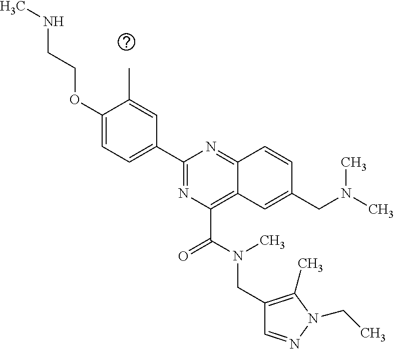 | 549.3 | 550.3 | 38 | 20 | 2.047 | 19.34 |
| 465 | 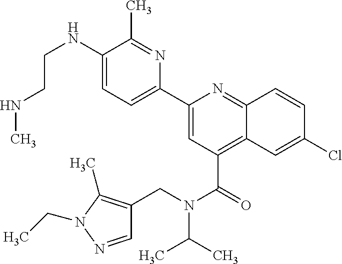 [SMILES: CCn1ncc(CN(C(=O)c2cc(-c3ccc(NCCNC)c(C)n3)nc3ccc(Cl)cc23)C(C)C)c1C] | 533.3 | 534.2 | 0.3637 | 4.867 | 1.934 | 1.479 |
| 466 | 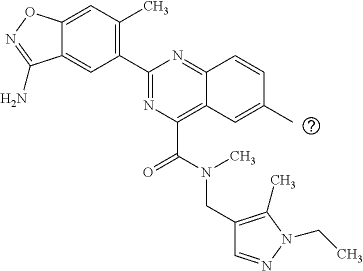 [SMILES: CCn1ncc(CN(C)C(=O)c2nc(-c3cc4c(N)noc4cc3C)nc3ccc(C)cc23)c1C] | 489.2 | 490.2 | 0.0295 | 0.2021 | 0.1887 | 0.0715 |
| 467 | 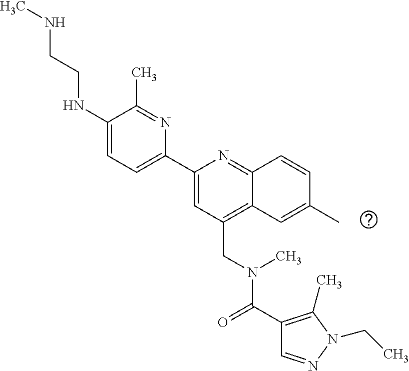 [SMILES: CCn1ncc(C(=O)N(C)Cc2cc(-c3ccc(NCCNC)c(C)n3)nc3ccc(C)cc23)c1C] | 505.2 | 506.3 | 50 | 20 | 5.585 | 34.2 |
| 468 | 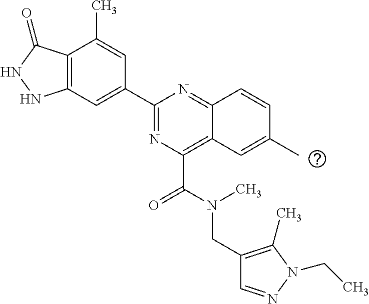 [SMILES: CCn1ncc(CN(C)C(=O)c2nc(-c3cc(C)c4c(=O)[nH][nH]c4c3)nc3ccc(C)cc23)c1C] | 489.2 | 490.1 | 0.01024 | 0.2131 | 0.1575 | 0.0135 |
| 469 | 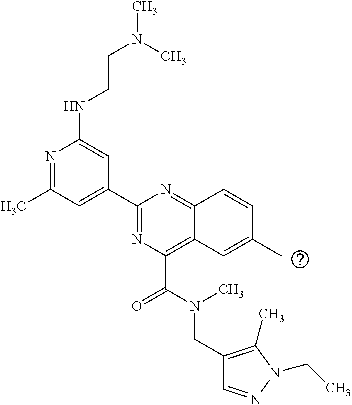 [SMILES: CCn1ncc(CN(C)C(=O)c2nc(-c3cc(C)nc(NCCN(C)C)c3)nc3ccc(C)cc23)c1C] | 520.2 | 521.2 | 0.01927 | 0.05609 | 0.02923 | 0.0109 |
| 470 | 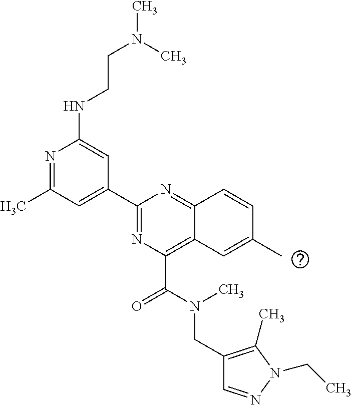 [SMILES: CCn1ncc(CN(C)C(=O)c2nc(-c3cc(C)nc(NCCN(C)C)c3)nc3ccc(C)cc23)c1C] | 520.2 | 521.3 | 0.01883 | 0.04903 | 0.02543 | 0.0099 |
| 471 | 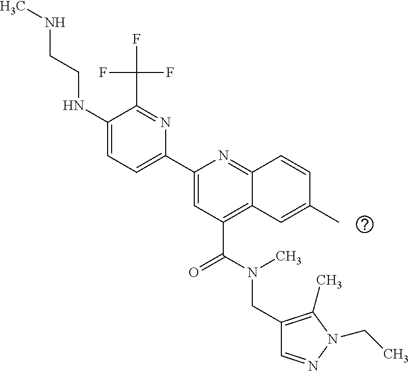 [SMILES: CCn1ncc(CN(C)C(=O)c2cc(-c3ccc(NCCNC)c(C(F)(F)F)n3)nc3ccc(C)cc23)c1C] | 559.2 | 560.2 | 0.03578 | 0.08859 | 0.02916 | 0.0095 |
| 472 | 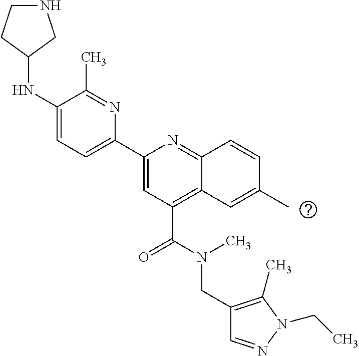 [SMILES: CCn1ncc(CN(C)C(=O)c2cc(-c3ccc(NC4CCNC4)c(C)n3)nc3ccc(C)cc23)c1C] | 517.2 | 518.3 | 0.02545 | 0.1325 | 0.048 | 0.01 |
| 473 | 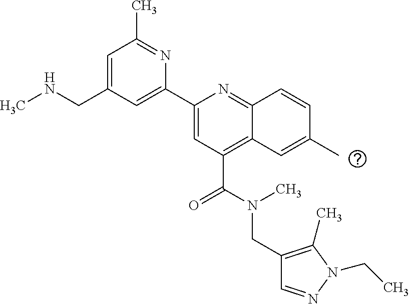 [SMILES: CCn1ncc(CN(C)C(=O)c2cc(-c3cc(CNC)cc(C)n3)nc3ccc(C)cc23)c1C] | 476.2 | 477.3 | 0.3216 | 0.7299 | 0.3803 | 0.3923 |
| 474 | 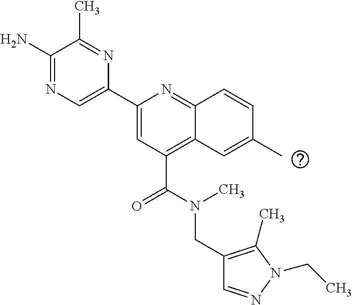 [SMILES: CCn1ncc(CN(C)C(=O)c2cc(-c3cnc(N)c(C)n3)nc3ccc(C)cc23)c1C] | 449.2 | 450.2 | 22.79 | 6.533 | 12.79 | 14.49 |
| 475 | 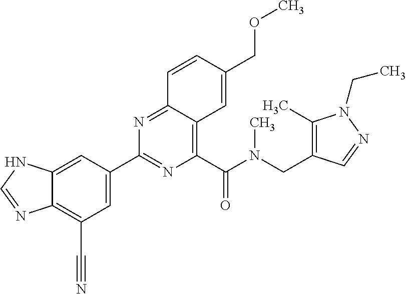 [SMILES: CCn1ncc(CN(C)C(=O)c2nc(-c3cc(C#N)c4nc[nH]c4c3)nc3ccc(COC)cc23)c1C] | 494.2 | 495.3 | 0.92 | 0.888 | 0.757 | 0.974 |
| 476 | 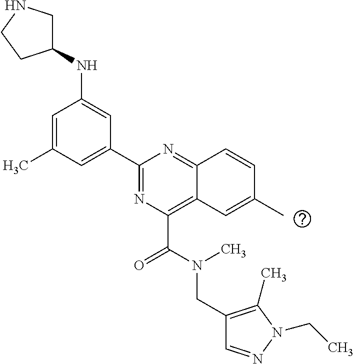 [SMILES: CCn1ncc(CN(C)C(=O)c2nc(-c3cc(C)cc(N[C@H]4CCNC4)c3)nc3ccc(C)cc23)c1C] | 517.2 | 518.3 | 0.0285 | 0.3784 | 0.2041 | 0.0064 |
| 477 | 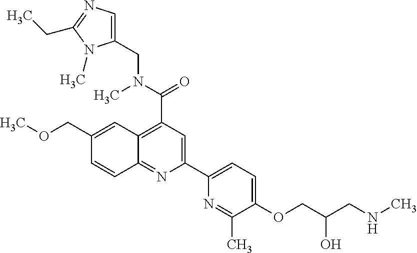 | 546.3 | | 0.1413 | 2.07 | 1.284 | 0.0476 |
| 478 | 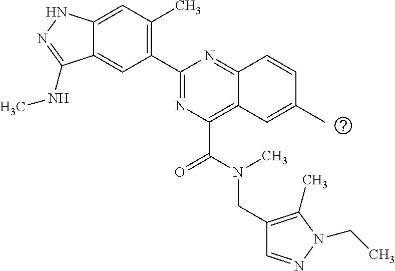 [SMILES: CCn1ncc(CN(C)C(=O)c2nc(-c3cc4c(NC)n[nH]c4cc3C)nc3ccc(C)cc23)c1C] | 502.2 | | 0.009401 | 0.03233 | 0.0242 | 0.0032 |
| 479 | 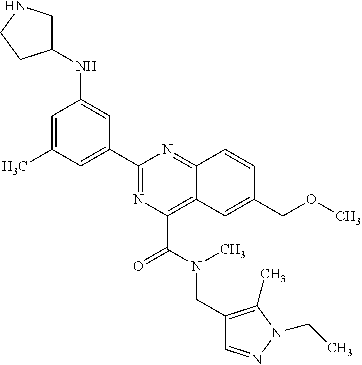 [SMILES: CCn1ncc(CN(C)C(=O)c2nc(-c3cc(C)cc(NC4CCNC4)c3)nc3ccc(COC)cc23)c1C] | 527.3 | | 0.01798 | 0.07084 | 0.04356 | 0.0101 |
| 480 | 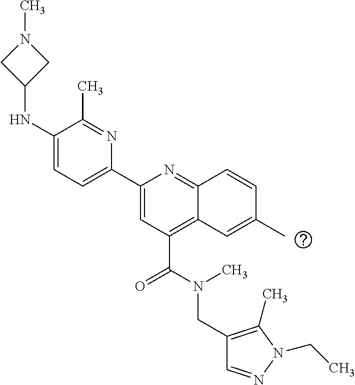 [SMILES: CCn1ncc(CN(C)C(=O)c2cc(-c3ccc(NC4CN(C)C4)c(C)n3)nc3ccc(C)cc23)c1C] | 517.2 | 518.2 | 0.01707 | 0.02306 | 0.01952 | 0.0065 |
| 481 | 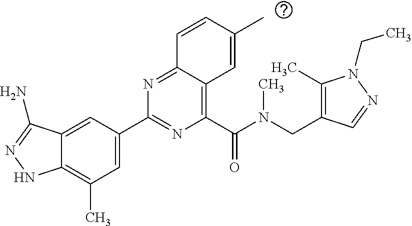 [SMILES: CCn1ncc(CN(C)C(=O)c2nc(-c3cc(C)c4[nH]nc(N)c4c3)nc3ccc(C)cc23)c1C] | 488.2 | | 0.01145 | 0.1133 | 0.1203 | 0.0235 |
| 482 | 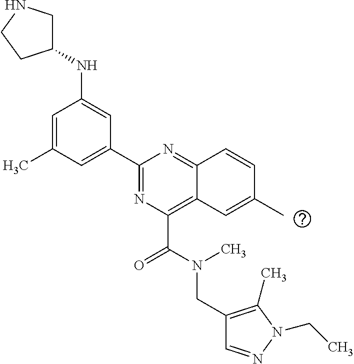 [SMILES: CCn1ncc(CN(C)C(=O)c2nc(-c3cc(C)cc(N[C@@H]4CCNC4)c3)nc3ccc(C)cc23)c1C] | 517.2 | | 0.01192 | 0.08802 | 0.03771 | 0.004 |
| 483 | 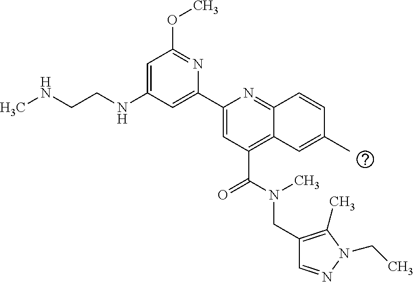 [SMILES: CCn1ncc(CN(C)C(=O)c2cc(-c3cc(NCCNC)cc(OC)n3)nc3ccc(C)cc23)c1C] | 521.2 | | 0.04183 | 0.03469 | 0.02524 | 0.0188 |
| 484 | 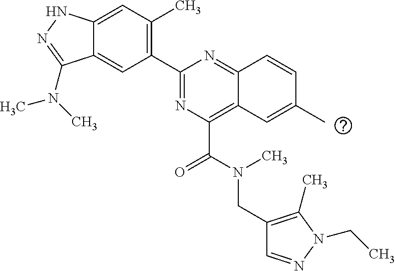 [SMILES: CCn1ncc(CN(C)C(=O)c2nc(-c3cc4c(N(C)C)n[nH]c4cc3C)nc3ccc(C)cc23)c1C] | 516.2 | | 5.118 | 3.782 | 2.367 | 2.285 |
| 485 | 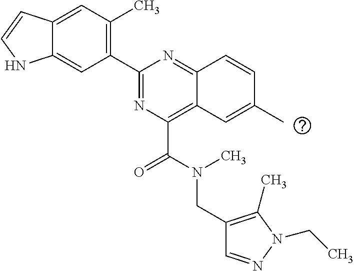 [SMILES: CCn1ncc(CN(C)C(=O)c2nc(-c3cc4[nH]ccc4cc3C)nc3ccc(C)cc23)c1C] | 472.2 | | 0.004868 | 0.1068 | 0.08774 | 0.0124 |
| 486 | 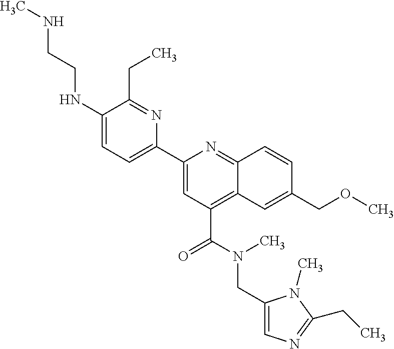 | 529.3 | | 0.05982 | 0.3145 | 0.08949 | 0.0301 |
| 487 | 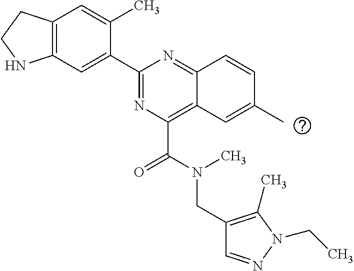 [SMILES: CCn1ncc(CN(C)C(=O)c2nc(-c3cc4c(cc3C)CCN4)nc3ccc(C)cc23)c1C] | 474.2 | | 10 | 11.75 | 6.725 | 4.077 |
| 488 | 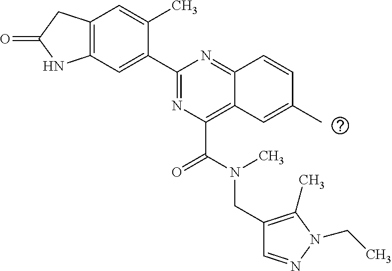 [SMILES: CCn1ncc(CN(C)C(=O)c2nc(-c3cc4c(cc3C)CC(=O)N4)nc3ccc(C)cc23)c1C] | 488.2 | | 0.2196 | 0.5436 | 1.009 | 0.466 |
| 489 | 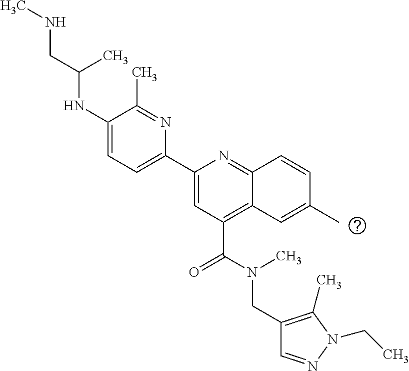 [SMILES: CCn1ncc(CN(C)C(=O)c2cc(-c3ccc(NC(C)CNC)c(C)n3)nc3ccc(C)cc23)c1C] | 519.3 | | 0.03391 | 0.1105 | 0.06743 | 0.0108 |
| 490 | 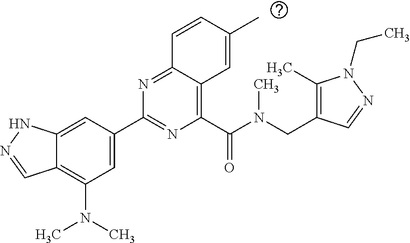 [SMILES: CCn1ncc(CN(C)C(=O)c2nc(-c3cc(N(C)C)c4cn[nH]c4c3)nc3ccc(C)cc23)c1C] | 502.2 | | 0.03279 | 0.4848 | 0.4922 | 0.1289 |
| 491 | 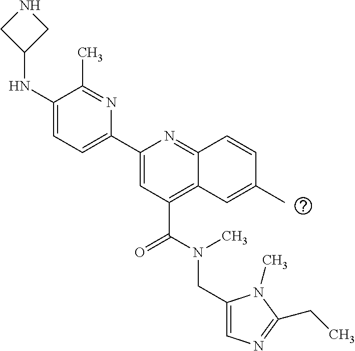 [SMILES: CCc1ncc(CN(C)C(=O)c2cc(-c3ccc(NC4CNC4)c(C)n3)nc3ccc(C)cc23)n1C] | 503.2 | | 0.01718 | 0.3664 | 0.08489 | 0.0096 |
| 492 | 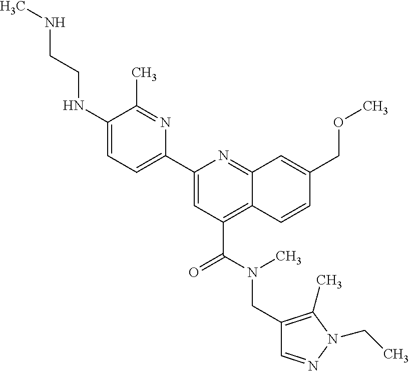 [SMILES: CCn1ncc(CN(C)C(=O)c2cc(-c3ccc(NCCNC)c(C)n3)nc3cc(COC)ccc23)c1C] | 515.3 | | 7.239 | 2.395 | 1.673 | 5.994 |
| 493 | 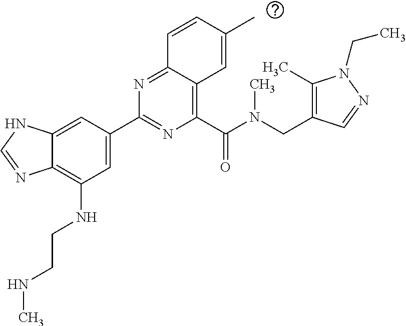 [SMILES: CCn1ncc(CN(C)C(=O)c2nc(-c3cc(NCCNC)c4nc[nH]c4c3)nc3ccc(C)cc23)c1C] | 531.2 | | 0.168 | 7.868 | 6.396 | 0.8543 |
| 494 | 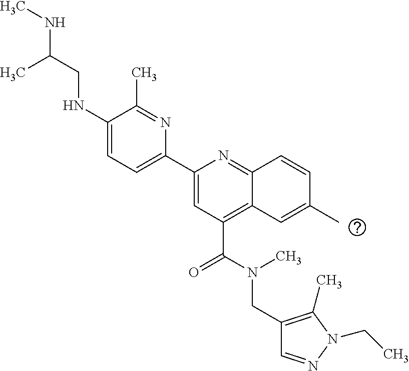 [SMILES: CCn1ncc(CN(C)C(=O)c2cc(-c3ccc(NCC(C)NC)c(C)n3)nc3ccc(C)cc23)c1C] | 519.3 | 520.3 | 0.01939 | 0.1028 | 0.06563 | 0.0079 |
| 495 | 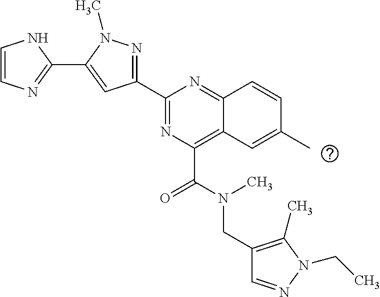 [SMILES: CCn1ncc(CN(C)C(=O)c2nc(-c3cc(-c4ncc[nH]4)n(C)n3)nc3ccc(C)cc23)c1C] | 489.2 | 490.2 | 0.02927 | 0.1526 | 0.1395 | 0.0163 |
| 496 | 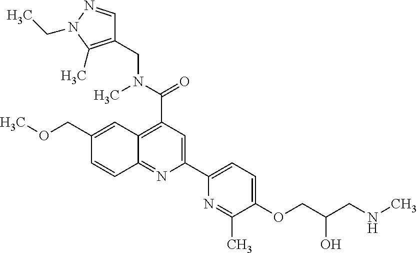 | 546.3 | 547.3 | 0.1672 | 0.937 | 0.6887 | 0.0599 |
| 497 | 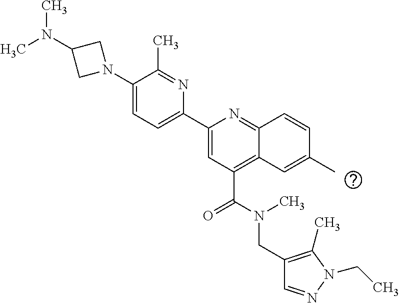 [SMILES: CCn1ncc(CN(C)C(=O)c2cc(-c3ccc(N4CC(N(C)C)C4)c(C)n3)nc3ccc(C)cc23)c1C] | 531.3 | 532.3 | 0.8458 | 1.626 | 2.564 | 0.6606 |
| 498 | 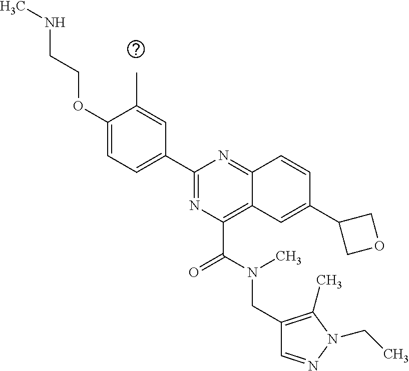 [SMILES: CCn1ncc(CN(C)C(=O)c2nc(-c3ccc(OCCNC)c(C)c3)nc3ccc(C4COC4)cc23)c1C] | 548.2 | 549.3 | 5.08 | 5.547 | 2.876 | 4.98 |
| 499 | 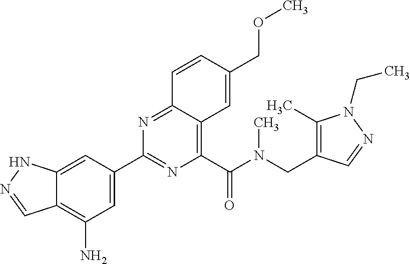 [SMILES: CCn1ncc(CN(C)C(=O)c2nc(-c3cc(N)c4cn[nH]c4c3)nc3ccc(COC)cc23)c1C] | 484.2 | 485.3 | 0.01491 | 0.1366 | 0.1828 | 0.0035 |
| 500 | 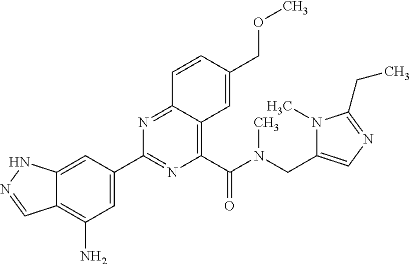 [SMILES: CCc1ncc(CN(C)C(=O)c2nc(-c3cc(N)c4cn[nH]c4c3)nc3ccc(COC)cc23)n1C] | 484.2 | 485.3 | 0.02534 | 0.371 | 0.4093 | 0.0285 |
| 501 | 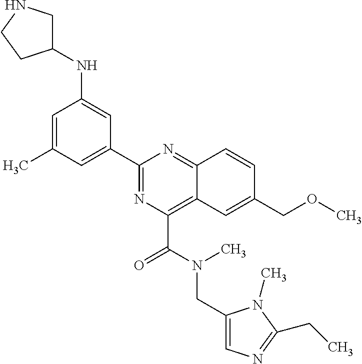 [SMILES: CCc1ncc(CN(C)C(=O)c2nc(-c3cc(C)cc(NC4CCNC4)c3)nc3ccc(COC)cc23)n1C] | 527.3 | 528.3 | 0.1485 | 3.888 | 1.153 | 0.0368 |
| 502 | 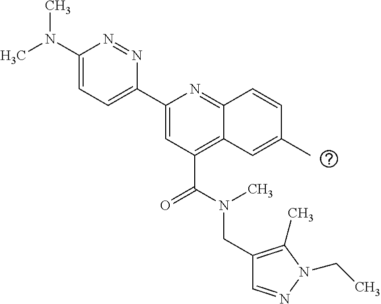 [SMILES: CCn1ncc(CN(C)C(=O)c2cc(-c3ccc(N(C)C)nn3)nc3ccc(C)cc23)c1C] | 463.2 | 464.2 | 10.62 | 2.527 | 9.01 | 4.849 |
| 503 | 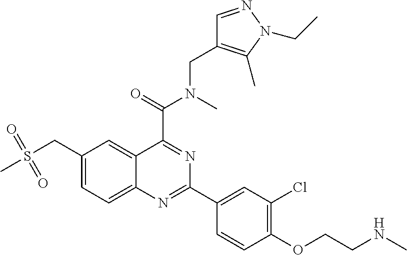 | 584.2 | 585.3 | 9.85 | 17.1 | 7.61 | 9.278 |
| 504 | 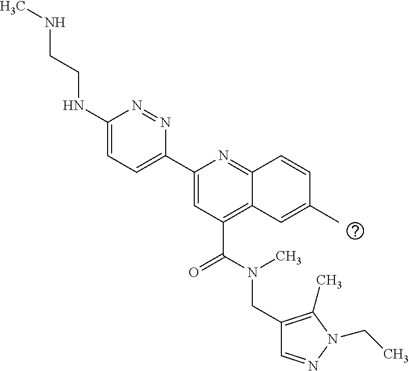 [SMILES: CCn1ncc(CN(C)C(=O)c2cc(-c3ccc(NCCNC)nn3)nc3ccc(C)cc23)c1C] | 492.2 | 493.3 | 5.775 | 3.991 | 3.734 | 1.995 |
| 505 | 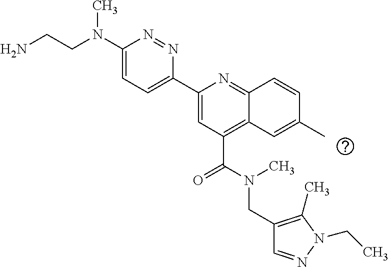 [SMILES: CCn1ncc(CN(C)C(=O)c2cc(-c3ccc(N(C)CCN)nn3)nc3ccc(C)cc23)c1C] | 492.2 | 493.2 | 4.524 | 2.335 | 5.963 | 3.277 |
| 506 | 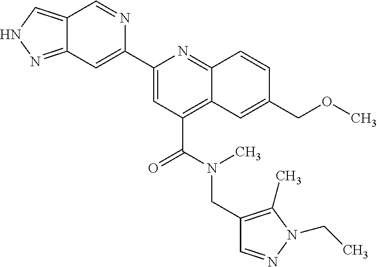 [SMILES: CCn1ncc(CN(C)C(=O)c2cc(-c3cc4n[nH]cc4cn3)nc3ccc(COC)cc23)c1C] | 469.2 | 470.3 | 0.06478 | 0.1851 | 0.5673 | 0.0076 |
| 507 | 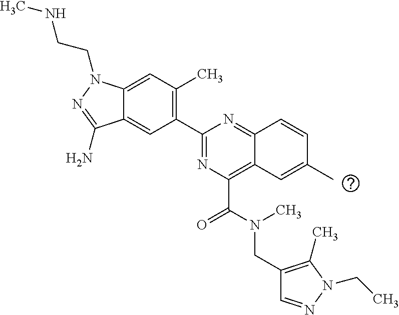 [SMILES: CCn1ncc(CN(C)C(=O)c2nc(-c3cc4c(N)nn(CCNC)c4cc3C)nc3ccc(C)cc23)c1C] | 545.2 | 546.3 | 0.5563 | 1.598 | 1.01 | 1.36 |
| 508 | 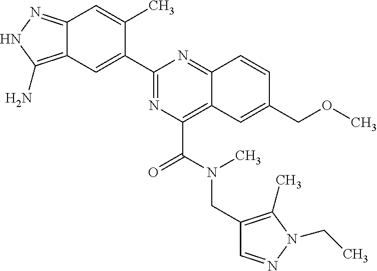 [SMILES: CCn1ncc(CN(C)C(=O)c2nc(-c3cc4c(N)[nH]nc4cc3C)nc3ccc(COC)cc23)c1C] | 498.2 | 499.2 | 0.04634 | 0.174 | 0.2115 | 0.0224 |
| 509 | 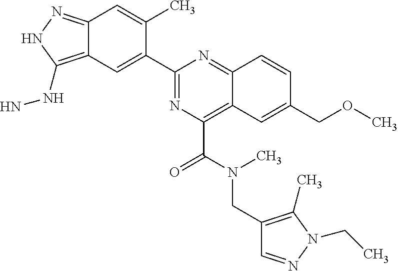 [SMILES: CCn1ncc(CN(C)C(=O)c2nc(-c3cc4c(NN)[nH]nc4cc3C)nc3ccc(COC)cc23)c1C] | 512.3 | 513.3 | 0.03377 | 0.07637 | 0.06647 | 0.0132 |
| 510 | 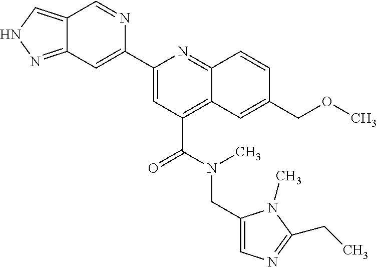 [SMILES: CCc1ncc(CN(C)C(=O)c2cc(-c3cc4n[nH]cc4cn3)nc3ccc(COC)cc23)n1C] | 469.2 | 470.2 | 0.06289 | 0.2249 | 0.4654 | 0.0093 |
| 511 | 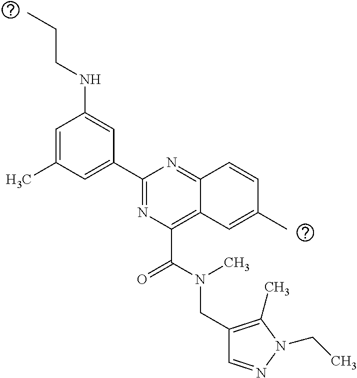 [SMILES: CCCNc1cc(C)cc(-c2nc(C(=O)N(C)Cc3cnn(CC)c3C)c3cc(C)ccc3n2)c1] | 492.2 | 493.2 | 0.1978 | 0.9286 | 0.9977 | 0.457 |
| 512 | 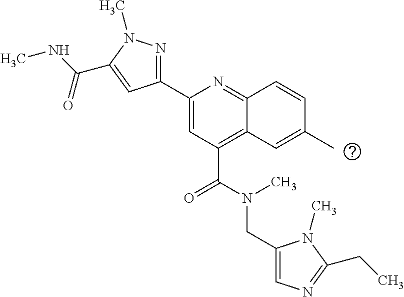 [SMILES: CCc1ncc(CN(C)C(=O)c2cc(-c3cc(C(=O)NC)n(C)n3)nc3ccc(C)cc23)n1C] | 479.2 | 480.2 | 0.01521 | 0.1042 | 0.1105 | 0.004 |
| 513 | 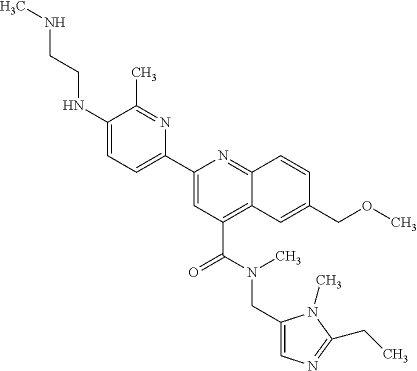 | 515.3 | 516.3 | 0.0684 | 0.4702 | 0.203 | 0.035 |
| 514 | 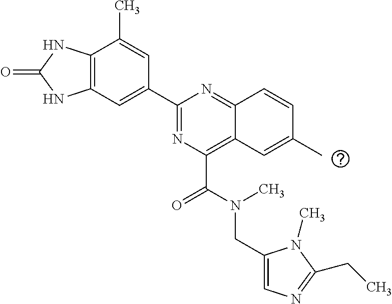 [SMILES: CCc1ncc(CN(C)C(=O)c2nc(-c3cc(C)c4[nH]c(=O)[nH]c4c3)nc3ccc(C)cc23)n1C] | 489.2 | 490.1 | 0.008958 | 0.4705 | 0.3628 | 0.0027 |
| 515 | 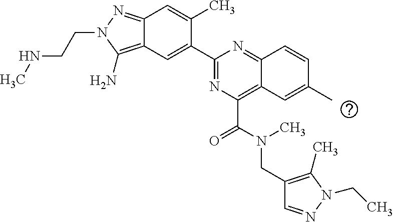 [SMILES: CCn1ncc(CN(C)C(=O)c2nc(-c3cc4c(N)n(CCNC)nc4cc3C)nc3ccc(C)cc23)c1C] | 545.2 | 546.2 | 0.0705 | 0.1658 | 0.07851 | 0.0494 |
| 516 | 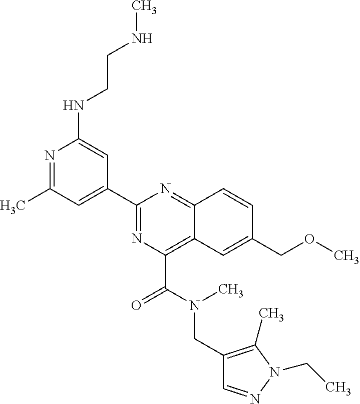 | 516.3 | 517.3 | 0.05835 | 0.1943 | 0.1247 | 0.0465 |
| 517 | 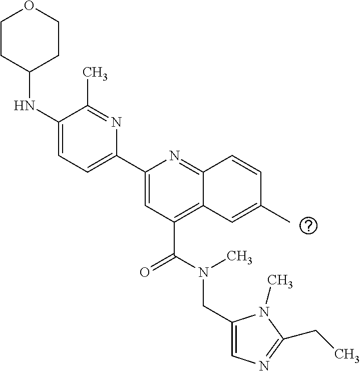 [SMILES: CCc1ncc(CN(C)C(=O)c2cc(-c3ccc(NC4CCOCC4)c(C)n3)nc3ccc(C)cc23)n1C] | 532.2 | 533.2 | 0.03609 | 0.1594 | 0.1074 | 0.0121 |
| 518 | 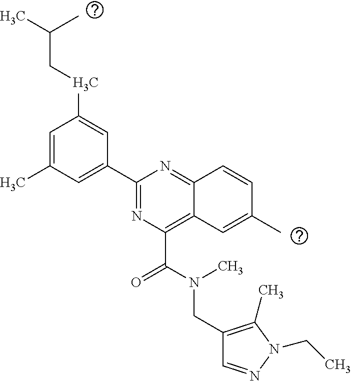 [SMILES: CCn1ncc(CN(C)C(=O)c2nc(-c3cc(C)cc(CCC(C)C)c3)nc3ccc(C)cc23)c1C] | 506.2 | 507.2 | 0.03817 | 1.339 | 1.528 | 0.2596 |
| 519 | 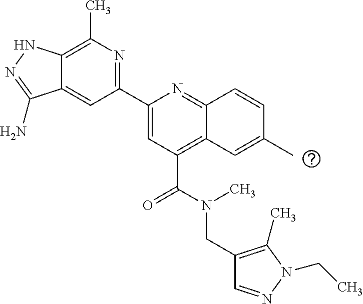 [SMILES: CCn1ncc(CN(C)C(=O)c2cc(-c3cc4c(N)n[nH]c4c(C)n3)nc3ccc(C)cc23)c1C] | 488.2 | 489.2 | 0.03142 | 0.253 | 0.3255 | 0.107 |
| 520 | 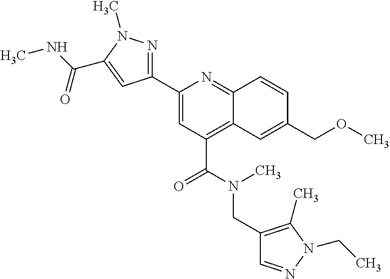 | 489.2 | 490.3 | 0.0807 | 0.3343 | 0.6502 | 0.0238 |
| 521 | 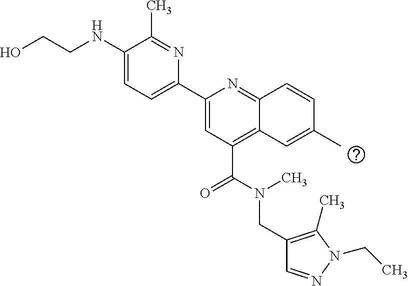 [SMILES: CCn1ncc(CN(C)C(=O)c2cc(-c3ccc(NCCO)c(C)n3)nc3ccc(C)cc23)c1C] | 492.2 | 493.3 | 0.01004 | 0.0804 | 0.11 | 0.0071 |
| 522 | 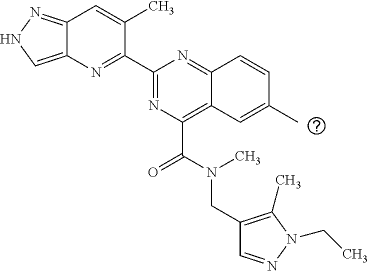 [SMILES: CCn1ncc(CN(C)C(=O)c2nc(-c3nc4c[nH]nc4cc3C)nc3ccc(C)cc23)c1C] | 474.2 | 475.2 | 26.04 | 16.65 | 8.065 | 16.6 |
| 523 | 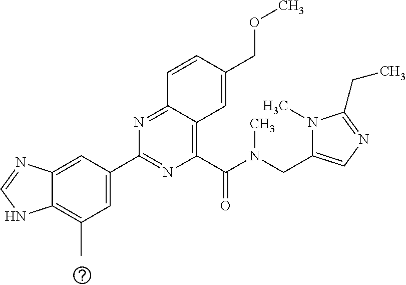 [SMILES: CCc1ncc(CN(C)C(=O)c2nc(-c3cc(C)c4[nH]cnc4c3)nc3ccc(COC)cc23)n1C] | 503.2 | 504.1 | 0.006218 | 0.07409 | 0.03473 | 0.0027 |
| 524 | 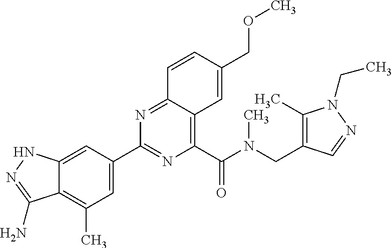 [SMILES: CCn1ncc(CN(C)C(=O)c2nc(-c3cc(C)c4c(N)n[nH]c4c3)nc3ccc(COC)cc23)c1C] | 498.2 | 499.3 | 0.01599 | 0.08142 | 0.1634 | 0.004 |
| 525 | 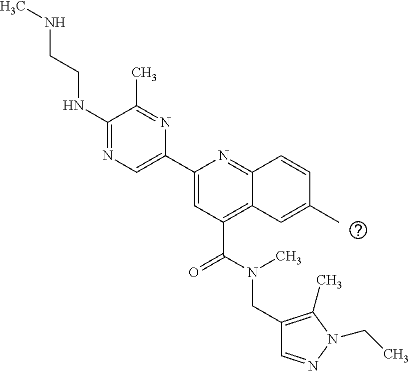 [SMILES: CCn1ncc(CN(C)C(=O)c2cc(-c3cnc(NCCNC)c(C)n3)nc3ccc(C)cc23)c1C] | 506.2 | 507.3 | 0.05402 | 0.2605 | 0.3133 | 0.1416 |
| 526 | 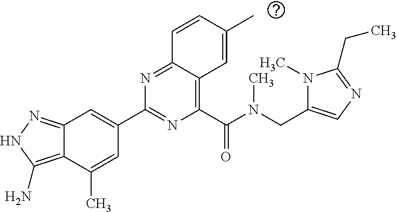 [SMILES: CCc1ncc(CN(C)C(=O)c2nc(-c3cc(C)c4c(N)[nH]nc4c3)nc3ccc(C)cc23)n1C] | 488.2 | 489.2 | 0.006424 | 0.04648 | 0.07195 | 0.002 |
| 527 | 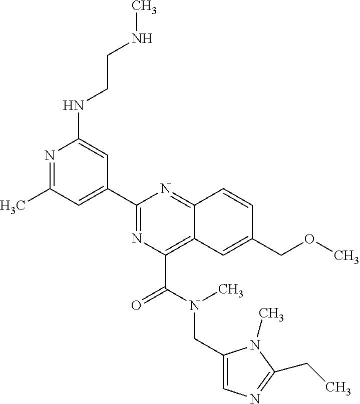 | 516.3 | 517.3 | 0.06725 | 0.7976 | 0.295 | 0.0212 |
| 528 | 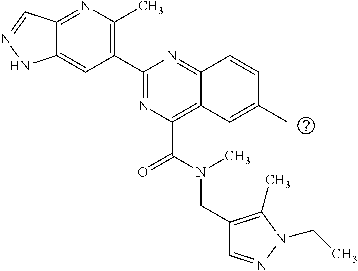 [SMILES: CCn1ncc(CN(C)C(=O)c2nc(-c3cc4[nH]ncc4nc3C)nc3ccc(C)cc23)c1C] | 474.2 | 475.1 | 1.065 | 0.6033 | 1.107 | 1.052 |
| 529 | 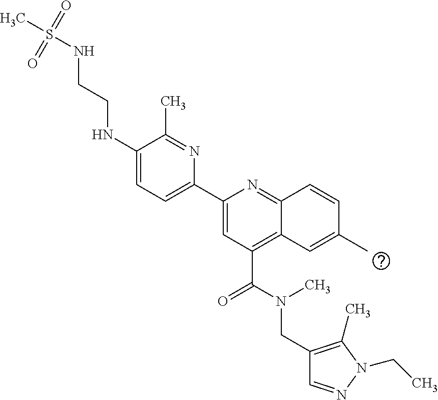 [SMILES: CCn1ncc(CN(C)C(=O)c2cc(-c3ccc(NCCNS(C)(=O)=O)c(C)n3)nc3ccc(C)cc23)c1C] | 569.2 | 570.2 | 0.02075 | 0.1374 | 0.2375 | 0.0104 |
| 530 | 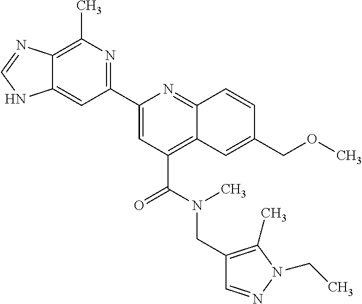 [SMILES: CCn1ncc(CN(C)C(=O)c2cc(-c3cc4[nH]cnc4c(C)n3)nc3ccc(COC)cc23)c1C] | 483.2 | 484.2 | 0.05027 | 0.1538 | 0.2476 | 0.0125 |
| 531 | 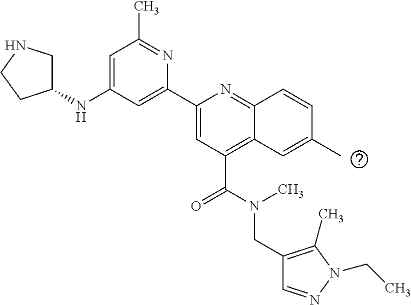 [SMILES: CCn1ncc(CN(C)C(=O)c2cc(-c3cc(N[C@@H]4CCNC4)cc(C)n3)nc3ccc(C)cc23)c1C] | 517.2 | 518.3 | 0.02988 | 0.3496 | 0.08889 | 0.0063 |
| 532 | 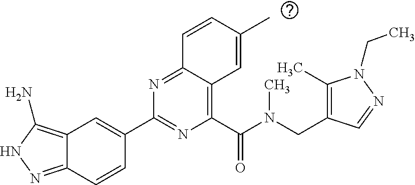 [SMILES: CCn1ncc(CN(C)C(=O)c2nc(-c3ccc4n[nH]c(N)c4c3)nc3ccc(C)cc23)c1C] | 474.2 | 475.1 | 0.01726 | 0.1585 | 0.2883 | 0.0154 |
| 533 | 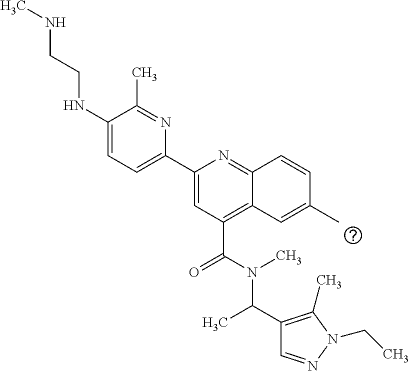 [SMILES: CCn1ncc(C(C)N(C)C(=O)c2cc(-c3ccc(NCCNC)c(C)n3)nc3ccc(C)cc23)c1C] | 519.3 | 520.2 | 0.1829 | 0.1593 | 0.07962 | 0.0324 |
| 534 | 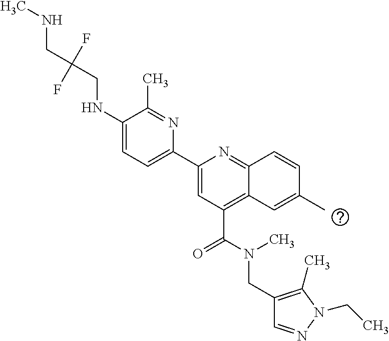 [SMILES: CCn1ncc(CN(C)C(=O)c2cc(-c3ccc(NCC(F)(F)CNC)c(C)n3)nc3ccc(C)cc23)c1C] | 555.2 | 556.2 | 0.03241 | 0.1708 | 0.2276 | 0.0091 |
| 535 | 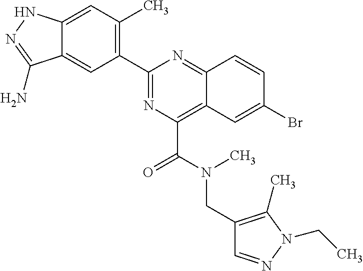 [SMILES: CCn1ncc(CN(C)C(=O)c2nc(-c3cc4c(N)n[nH]c4cc3C)nc3ccc(Br)cc23)c1C] | 532.1 | 533.2 | 0.01198 | 0.05832 | 0.05813 | 0.0041 |
| 536 | 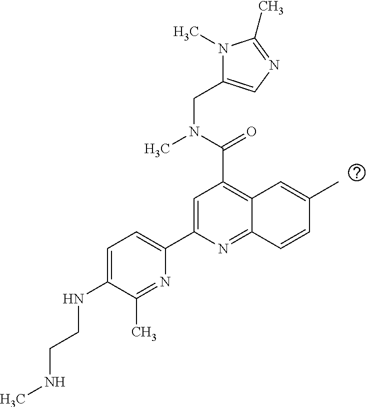 | 491.2 | 492.3 | 0.06978 | 0.3006 | 0.207 | 0.073 |
| 537 | 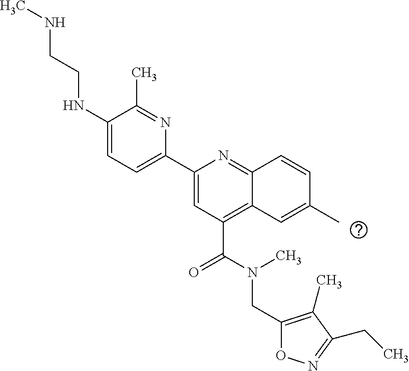 [SMILES: CCc1noc(CN(C)C(=O)c2cc(-c3ccc(NCCNC)c(C)n3)nc3ccc(C)cc23)c1C] | 506.2 | 507.2 | 0.0328 | 0.04832 | 0.05043 | 0.022 |
| 538 | 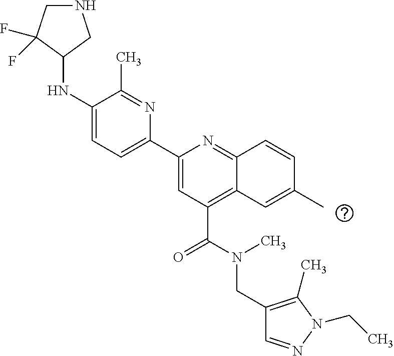 [SMILES: CCn1ncc(CN(C)C(=O)c2cc(-c3ccc(NC4CNCC4(F)F)c(C)n3)nc3ccc(C)cc23)c1C] | 553.2 | 554.2 | 0.08871 | 1.06 | 1.734 | 0.0886 |
| 539 | 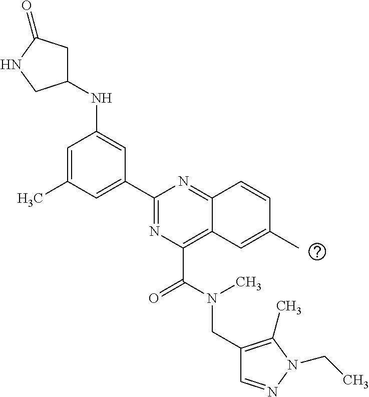 [SMILES: CCn1ncc(CN(C)C(=O)c2nc(-c3cc(C)cc(NC4CNC(=O)C4)c3)nc3ccc(C)cc23)c1C] | 531.2 | 532.2 | 0.03042 | 0.7138 | 1.342 | 0.0262 |
| 540 | 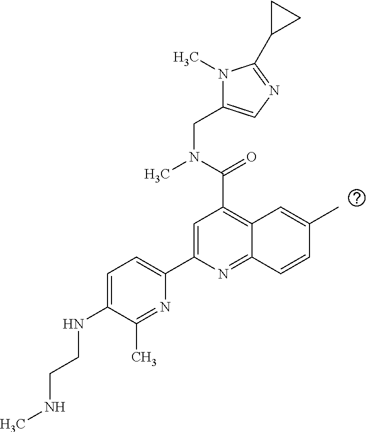 | 517.2 | 518.2 | 0.02046 | 0.08281 | 0.02329 | 0.0061 |
| 541 | 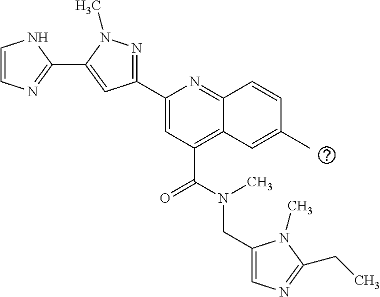 [SMILES: CCc1ncc(CN(C)C(=O)c2cc(-c3cc(-c4ncc[nH]4)n(C)n3)nc3ccc(C)cc23)n1C] | 488.2 | 489.1 | 0.01117 | 0.1045 | 0.08542 | 0.0027 |
| 542 | 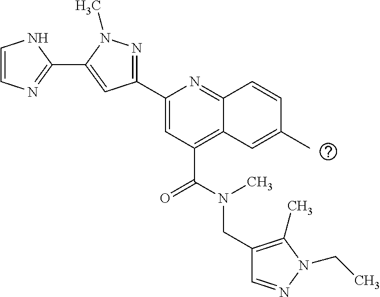 [SMILES: CCn1ncc(CN(C)C(=O)c2cc(-c3cc(-c4ncc[nH]4)n(C)n3)nc3ccc(C)cc23)c1C] | 488.2 | 489.1 | 0.006915 | 0.0535 | 0.05665 | 0.0024 |
| 543 | 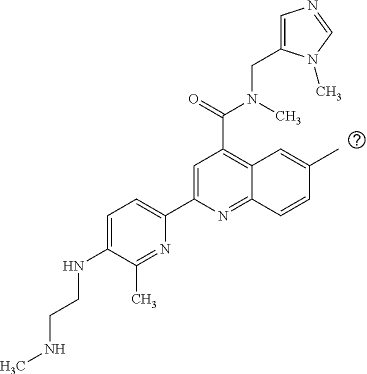 | 477.2 | 478.2 | 8.544 | 6.817 | 2.264 | 8.959 |
| 544 | 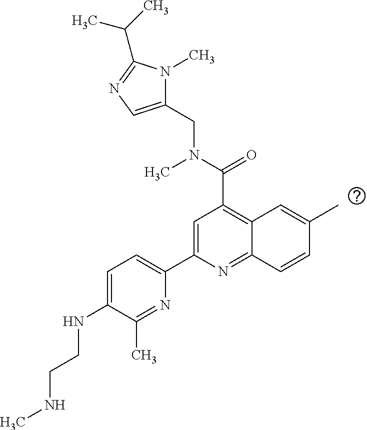 | 519.3 | 520.3 | 0.01462 | 0.1244 | 0.04253 | 0.006 |
| 545 | 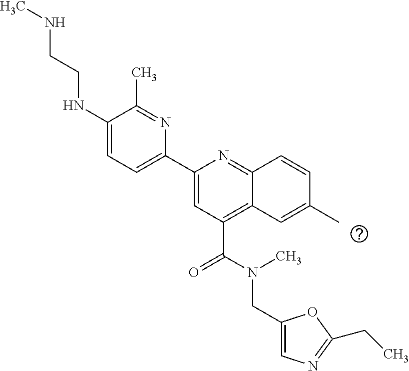 [SMILES: CCc1ncc(CN(C)C(=O)c2cc(-c3ccc(NCCNC)c(C)n3)nc3ccc(C)cc23)o1] | 508.2 | 509.1 | 0.01724 | 0.1486 | 0.1847 | 0.0141 |
| 546 | 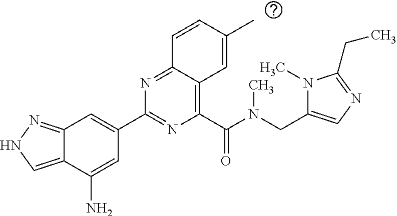 [SMILES: CCc1ncc(CN(C)C(=O)c2nc(-c3cc(N)c4c[nH]nc4c3)nc3ccc(C)cc23)n1C] | 474.2 | 475.1 | 0.0137 | 0.113 | 0.09644 | 0.0337 |
| 547 | 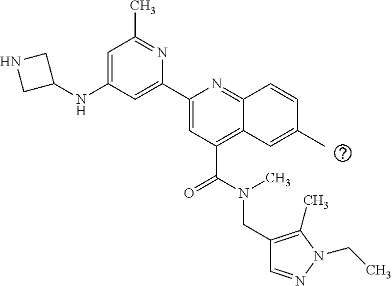 [SMILES: CCn1ncc(CN(C)C(=O)c2cc(-c3cc(NC4CNC4)cc(C)n3)nc3ccc(C)cc23)c1C] | 503.2 | 504.2 | 0.02376 | 0.5499 | 0.105 | 0.0125 |
| 548 | 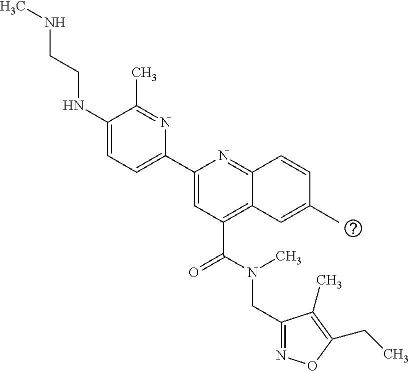 [SMILES: CCc1onc(CN(C)C(=O)c2cc(-c3ccc(NCCNC)c(C)n3)nc3ccc(C)cc23)c1C] | 506.2 | 507.2 | 2.024 | 2.151 | 1.865 | 2.164 |
| 549 | 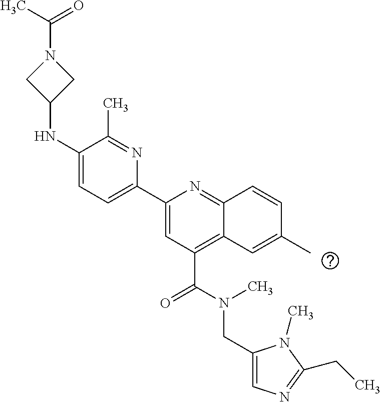 [SMILES: CCc1ncc(CN(C)C(=O)c2cc(-c3ccc(NC4CN(C(C)=O)C4)c(C)n3)nc3ccc(C)cc23)n1C] | 545.2 | 546.2 | 0.03494 | 0.2397 | 0.1503 | 0.0124 |
| 550 | 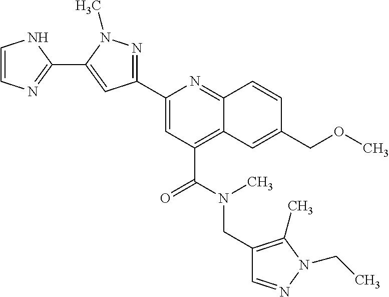 [SMILES: CCn1ncc(CN(C)C(=O)c2cc(-c3cc(-c4ncc[nH]4)n(C)n3)nc3ccc(COC)cc23)c1C] | 498.2 | 499.3 | 0.03219 | 0.1281 | 0.2472 | 0.0141 |
| 551 | 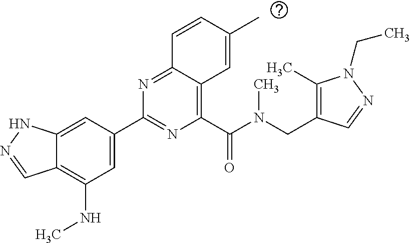 [SMILES: CCn1ncc(CN(C)C(=O)c2nc(-c3cc(NC)c4cn[nH]c4c3)nc3ccc(C)cc23)c1C] | 488.2 | 489.3 | 0.02699 | 0.1529 | 0.2329 | 0.1366 |
| 552 | 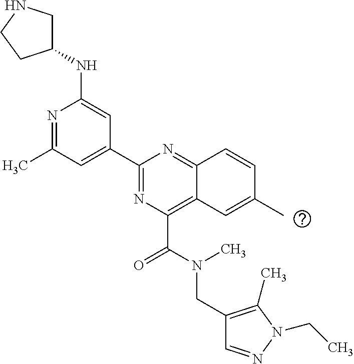 [SMILES: CCn1ncc(CN(C)C(=O)c2nc(-c3cc(C)nc(N[C@@H]4CCNC4)c3)nc3ccc(C)cc23)c1C] | 518.2 | 519.2 | 0.01335 | 0.04974 | 0.07224 | 0.0063 |
| 553 | 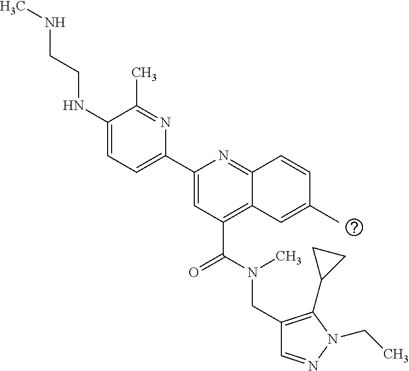 [SMILES: CCn1ncc(CN(C)C(=O)c2cc(-c3ccc(NCCNC)c(C)n3)nc3ccc(C)cc23)c1C1CC1] | 531.3 | 532.3 | 0.05251 | 0.4665 | 0.7626 | 0.5348 |
| 554 | 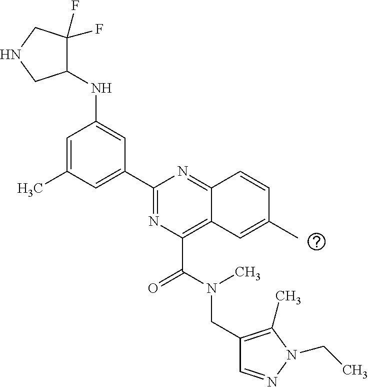 [SMILES: CCn1ncc(CN(C)C(=O)c2nc(-c3cc(C)cc(NC4CNCC4(F)F)c3)nc3ccc(C)cc23)c1C] | 553.2 | 554.2 | 0.05348 | 1.069 | 1.667 | 0.1732 |
| 555 | 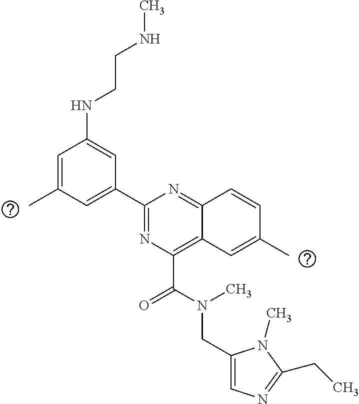 [SMILES: CCc1ncc(CN(C)C(=O)c2nc(-c3cc(C)cc(NCCNC)c3)nc3ccc(C)cc23)n1C] | 525.2 | 526.1 | 0.02281 | 0.2256 | 0.1022 | 0.0071 |
| 556 | 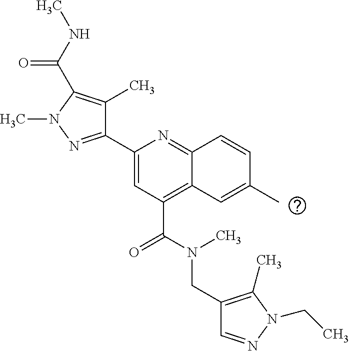 [SMILES: CCn1ncc(CN(C)C(=O)c2cc(-c3nn(C)c(C(=O)NC)c3C)nc3ccc(C)cc23)c1C] | 493.2 | 494.1 | 50 | 20 | 6.286 | 50 |
| 557 | 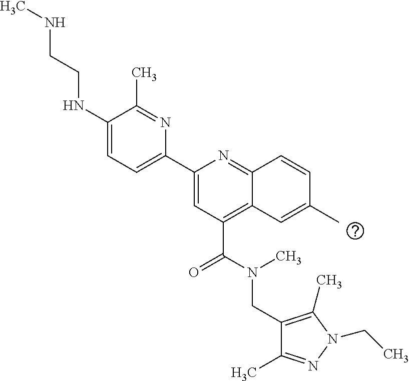 [SMILES: CCn1nc(C)c(CN(C)C(=O)c2cc(-c3ccc(NCCNC)c(C)n3)nc3ccc(C)cc23)c1C] | 519.3 | 520.3 | 14.12 | 18.35 | 6.803 | 5.201 |
| 558 | 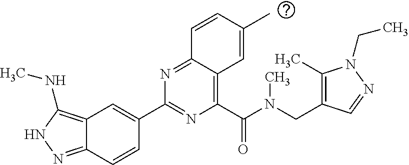 [SMILES: CCn1ncc(CN(C)C(=O)c2nc(-c3ccc4n[nH]c(NC)c4c3)nc3ccc(C)cc23)c1C] | 488.2 | 489.2 | 0.004272 | 0.03926 | 0.07626 | 0.0015 |
| 559 | 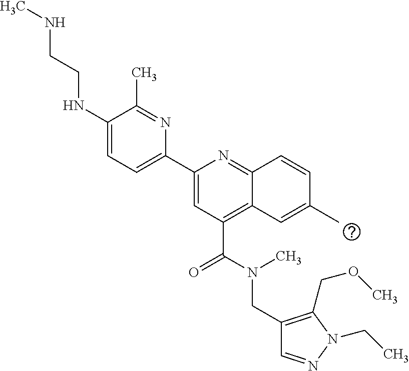 [SMILES: CCn1ncc(CN(C)C(=O)c2cc(-c3ccc(NCCNC)c(C)n3)nc3ccc(C)cc23)c1COC] | 535.2 | 536.2 | 0.593 | 0.8377 | 1.244 | 2.053 |
| 560 | 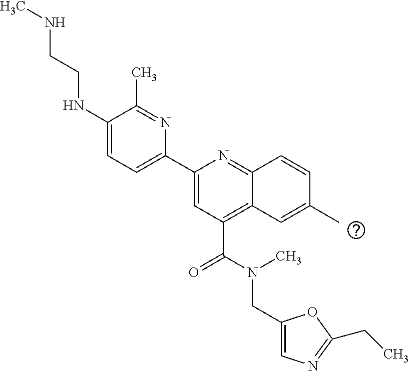 [SMILES: CCc1ncc(CN(C)C(=O)c2cc(-c3ccc(NCCNC)c(C)n3)nc3ccc(C)cc23)o1] | 492.2 | 493.2 | 0.1425 | 0.2424 | 0.4483 | 0.412 |
| 561 | 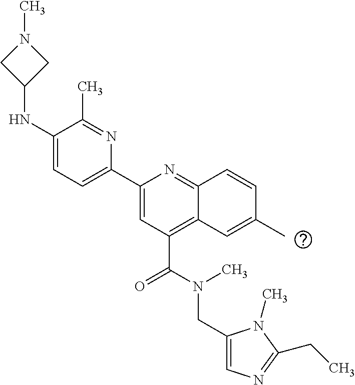 [SMILES: CCc1ncc(CN(C)C(=O)c2cc(-c3ccc(NC4CN(C)C4)c(C)n3)nc3ccc(C)cc23)n1C] | 517.2 | 518.2 | 0.01909 | 0.05822 | 0.02279 | 0.0065 |
| 562 | 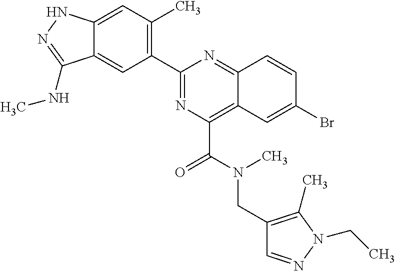 [SMILES: CCn1ncc(CN(C)C(=O)c2nc(-c3cc4c(NC)n[nH]c4cc3C)nc3ccc(Br)cc23)c1C] | 546.1 | 547.1 | 0.04944 | 0.1545 | 0.08787 | 0.0149 |
| 563 | 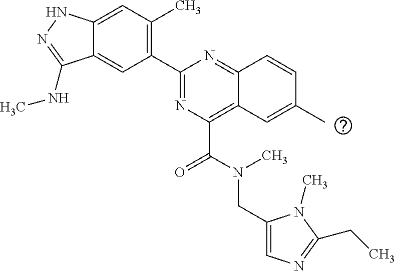 [SMILES: CCc1ncc(CN(C)C(=O)c2nc(-c3cc4c(NC)n[nH]c4cc3C)nc3ccc(C)cc23)n1C] | 502.2 | 503.2 | 0.01346 | 0.05538 | 0.03754 | 0.0057 |
| 564 | 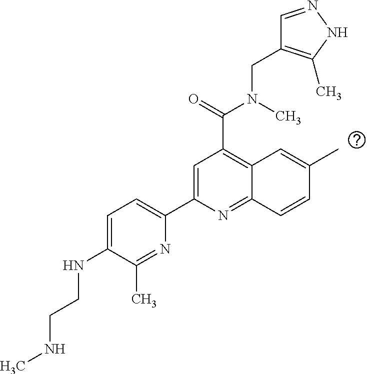 | 477.2 | 478.2 | 50 | 20 | 9.527 | 50 |
| 565 | 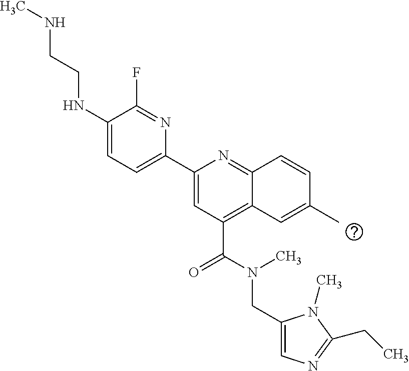 [SMILES: CCc1ncc(CN(C)C(=O)c2cc(-c3ccc(NCCNC)c(F)n3)nc3ccc(C)cc23)n1C] | 509.2 | 510.2 | 0.05115 | 0.1534 | 0.1929 | 0.0338 |
| 566 | 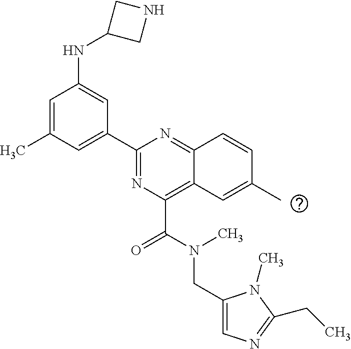 [SMILES: CCc1ncc(CN(C)C(=O)c2nc(-c3cc(C)cc(NC4CNC4)c3)nc3ccc(C)cc23)n1C] | 503.2 | 504.2 | 0.03753 | 0.8159 | 0.3379 | 0.0113 |
| 567 | 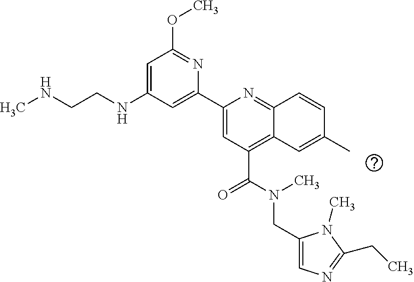 [SMILES: CCc1ncc(CN(C)C(=O)c2cc(-c3cc(NCCNC)cc(OC)n3)nc3ccc(C)cc23)n1C] | 521.2 | 522.2 | 0.07966 | 0.07785 | 0.03199 | 0.0222 |
| 568 | 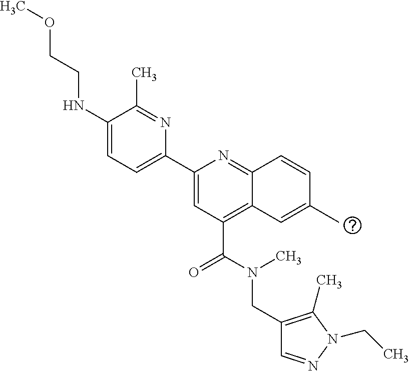 [SMILES: CCn1ncc(CN(C)C(=O)c2cc(-c3ccc(NCCOC)c(C)n3)nc3ccc(C)cc23)c1C] | 506.2 | 507.2 | 0.02364 | 0.1272 | 0.2599 | 0.0311 |
| 569 | 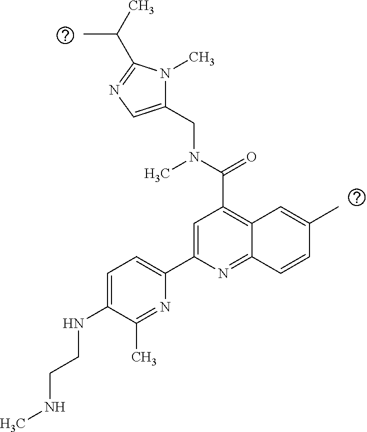 | 521.2 | 522.1 | 8.647 | 14.19 | 9.327 | 9.478 |
| 570 | 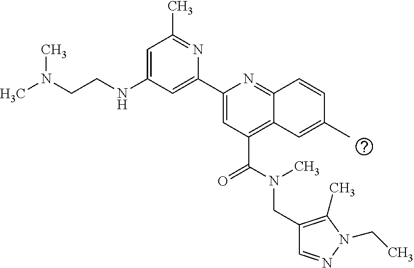 [SMILES: CCn1ncc(CN(C)C(=O)c2cc(-c3cc(NCCN(C)C)cc(C)n3)nc3ccc(C)cc23)c1C] | 519.3 | 520.3 | 0.04501 | 0.07626 | 0.03012 | 0.0137 |
| 571 | 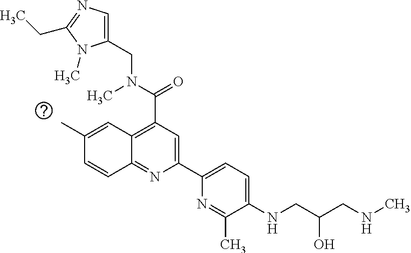 [SMILES: CCc1ncc(CN(C)C(=O)c2cc(-c3ccc(NCC(O)CNC)c(C)n3)nc3ccc(C)cc23)n1C] | 535.2 | 536.3 | 0.02542 | 0.4282 | 0.1175 | 0.0111 |
| 572 | 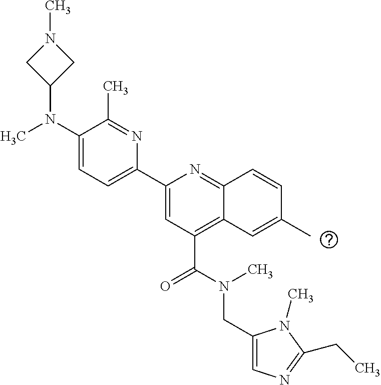 [SMILES: CCc1ncc(CN(C)C(=O)c2cc(-c3ccc(N(C)C4CN(C)C4)c(C)n3)nc3ccc(C)cc23)n1C] | 531.3 | 532.2 | 0.1161 | 0.1966 | 0.1331 | 0.0233 |
| 573 | 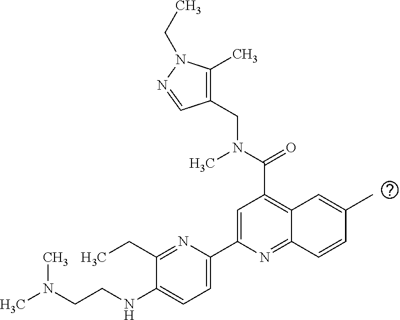 | 533.3 | 534.3 | 0.04652 | 0.05265 | 0.04931 | 0.0454 |
| 574 | 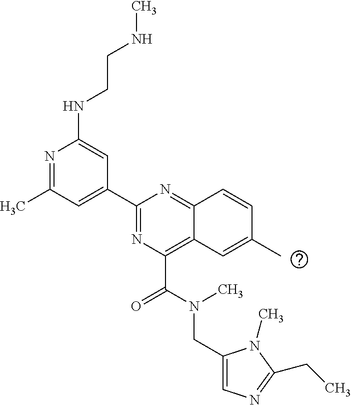 [SMILES: CCc1ncc(CN(C)C(=O)c2nc(-c3cc(C)nc(NCCNC)c3)nc3ccc(C)cc23)n1C] | 506.2 | 507.2 | 0.04795 | 0.149 | 0.06133 | 0.0169 |
| 575 | 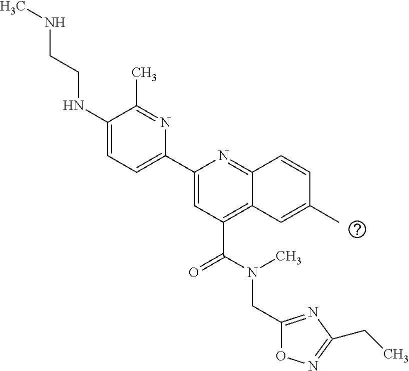 [SMILES: CCc1noc(CN(C)C(=O)c2cc(-c3ccc(NCCNC)c(C)n3)nc3ccc(C)cc23)n1] | 493.2 | 494.2 | 3.381 | 1.523 | 1.529 | 3.866 |
| 576 | 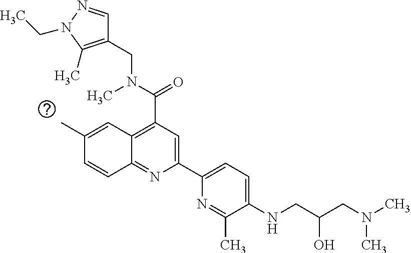 [SMILES: CCn1ncc(CN(C)C(=O)c2cc(-c3ccc(NCC(O)CN(C)C)c(C)n3)nc3ccc(C)cc23)c1C] | 549.3 | 550.2 | 0.02705 | 0.03177 | 0.02113 | 0.0106 |
| 577 | 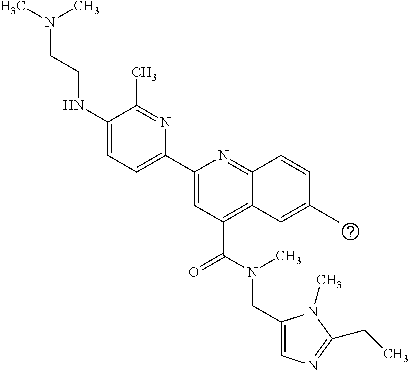 [SMILES: CCc1ncc(CN(C)C(=O)c2cc(-c3ccc(NCCN(C)C)c(C)n3)nc3ccc(C)cc23)n1C] | 519.3 | 520.2 | 0.04292 | 0.08543 | 0.03123 | 0.0187 |
| 578 | 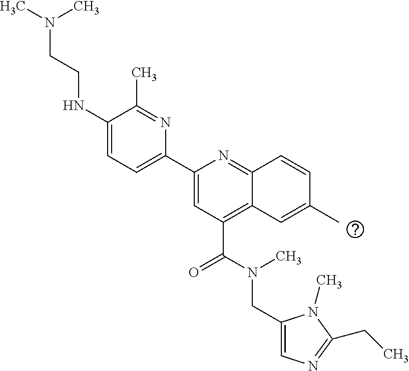 [SMILES: CCc1ncc(CN(C)C(=O)c2cc(-c3ccc(NCCN(C)C)c(C)n3)nc3ccc(C)cc23)n1C] | 519.3 | 520.3 | 0.04203 | 0.1037 | 0.04322 | 0.0155 |
| 579 | 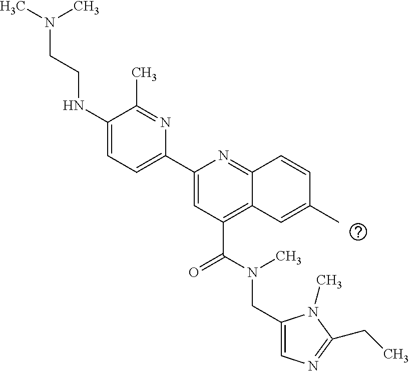 [SMILES: CCc1ncc(CN(C)C(=O)c2cc(-c3ccc(NCCN(C)C)c(C)n3)nc3ccc(C)cc23)n1C] | 519.3 | 520.1 | 0.02327 | 0.1357 | | 0.009 |
| 580 | 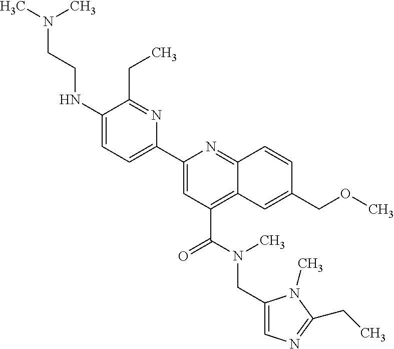 | 543.3 | 544.3 | 0.06587 | 0.1755 | 0.1299 | 0.0439 |
| 581 | 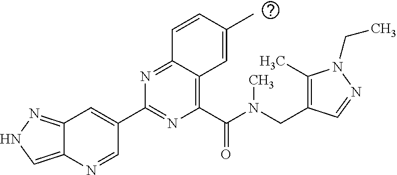 [SMILES: CCn1ncc(CN(C)C(=O)c2nc(-c3cnc4c[nH]nc4c3)nc3ccc(C)cc23)c1C] | 460.2 | 461.2 | 3.315 | 0.5133 | 0.8394 | 1.432 |
| 582 | 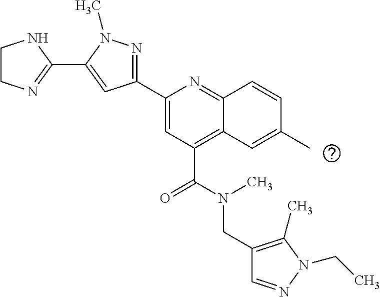 [SMILES: CCn1ncc(CN(C)C(=O)c2cc(-c3cc(C4=NCCN4)n(C)n3)nc3ccc(C)cc23)c1C] | 490.2 | 491.2 | 0.02578 | 0.0655 | 0.1224 | 0.0093 |
| 583 | 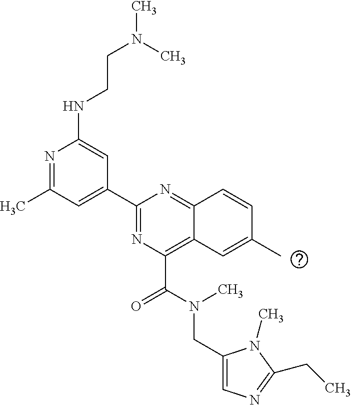 [SMILES: CCc1ncc(CN(C)C(=O)c2nc(-c3cc(C)nc(NCCN(C)C)c3)nc3ccc(C)cc23)n1C] | 520.2 | 521.3 | 0.03405 | 0.02972 | 0.01078 | 0.0142 |
| 584 | 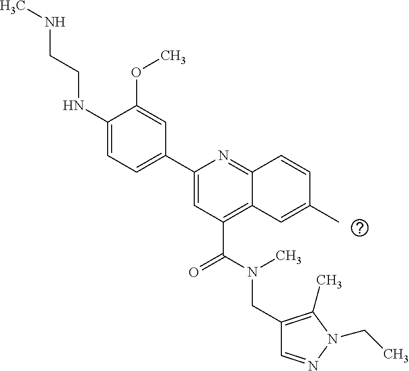 [SMILES: CCn1ncc(CN(C)C(=O)c2cc(-c3ccc(NCCNC)c(OC)c3)nc3ccc(C)cc23)c1C] | 520.2 | 521.3 | 0.04453 | 0.273 | 0.165 | 0.2212 |
| 585 | 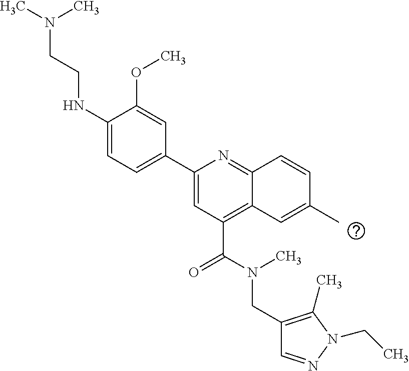 [SMILES: CCn1ncc(CN(C)C(=O)c2cc(-c3ccc(NCCN(C)C)c(OC)c3)nc3ccc(C)cc23)c1C] | 534.3 | 535.3 | 0.1037 | 0.3318 | 0.1774 | 0.9032 |
| 586 | 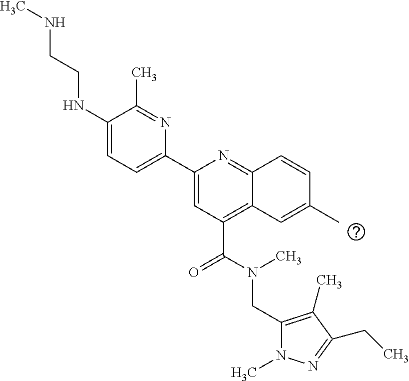 [SMILES: CCc1nn(C)c(CN(C)C(=O)c2cc(-c3ccc(NCCNC)c(C)n3)nc3ccc(C)cc23)c1C] | 519.3 | 520.3 | 19.37 | 7.239 | 2.129 | 23.28 |
| 587 | 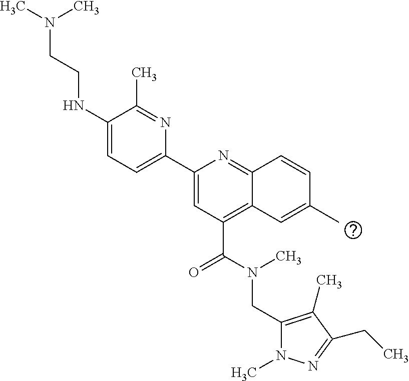 [SMILES: CCc1nn(C)c(CN(C)C(=O)c2cc(-c3ccc(NCCN(C)C)c(C)n3)nc3ccc(C)cc23)c1C] | 533.3 | 534.2 | 14.16 | 8.025 | 1.608 | 17.34 |
| 588 | 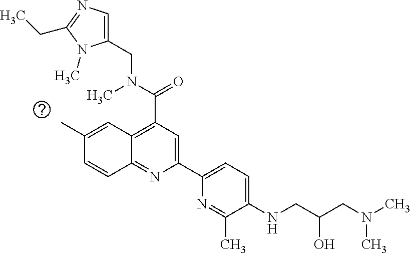 [SMILES: CCc1ncc(CN(C)C(=O)c2cc(-c3ccc(NCC(O)CN(C)C)c(C)n3)nc3ccc(C)cc23)n1C] | 549.3 | 550.2 | 0.02857 | 0.07326 | 0.02785 | 0.0089 |
| 589 | 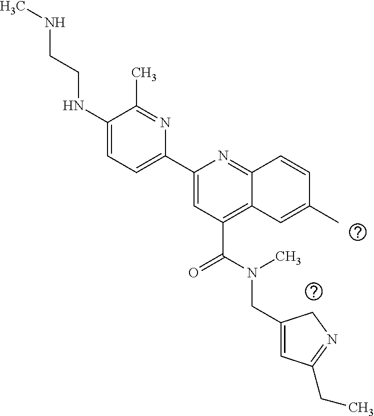 [SMILES: CCC1=NCC(CN(C)C(=O)c2cc(-c3ccc(NCCNC)c(C)n3)nc3ccc(C)cc23)=C1] | 492.2 | 493.2 | 0.04672 | 0.2436 | 0.2107 | 0.3053 |
| 590 | 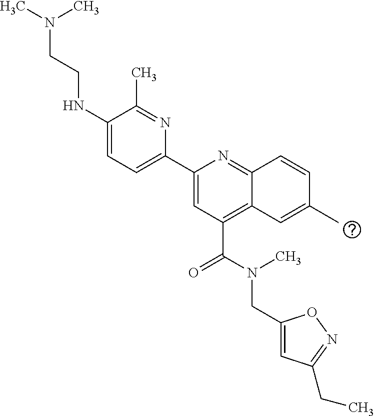 [SMILES: CCc1cc(CN(C)C(=O)c2cc(-c3ccc(NCCN(C)C)c(C)n3)nc3ccc(C)cc23)on1] | 506.2 | 507.3 | 0.3364 | 0.4026 | 0.3895 | 0.7341 |
| 591 | 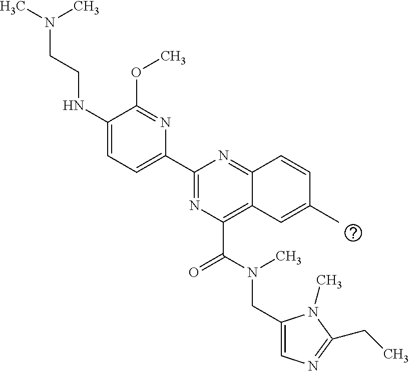 [SMILES: CCc1ncc(CN(C)C(=O)c2nc(-c3ccc(NCCN(C)C)c(OC)n3)nc3ccc(C)cc23)n1C] | 535.2 | 536.2 | 0.02885 | 0.09948 | 0.03509 | 0.029 |
| 592 | 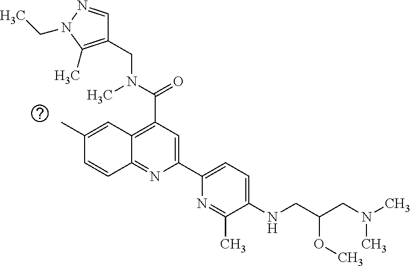 [SMILES: CCn1ncc(CN(C)C(=O)c2cc(-c3ccc(NCC(CN(C)C)OC)c(C)n3)nc3ccc(C)cc23)c1C] | 563.3 | 564.2 | 0.03552 | 4.5 | 7.228 | 0.0109 |
| 593 | 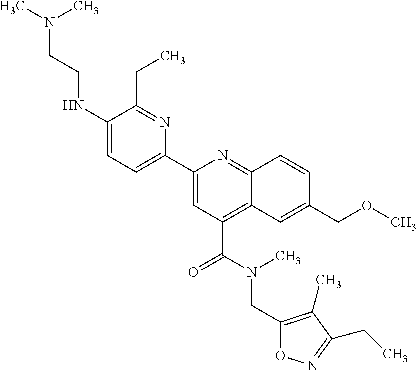 | 544.3 | 545.2 | 0.05386 | 0.1698 | 0.1029 | 0.1748 |
| 594 | 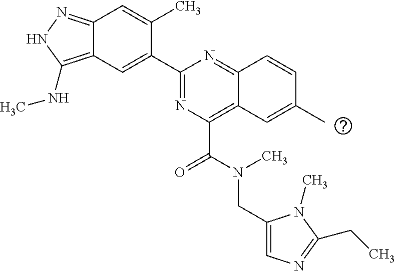 [SMILES: CCc1ncc(CN(C)C(=O)c2nc(-c3cc4c(NC)[nH]nc4cc3C)nc3ccc(C)cc23)n1C] | 512.3 | 513.3 | 0.03656 | 0.1135 | 0.05835 | 0.0136 |
| 595 | 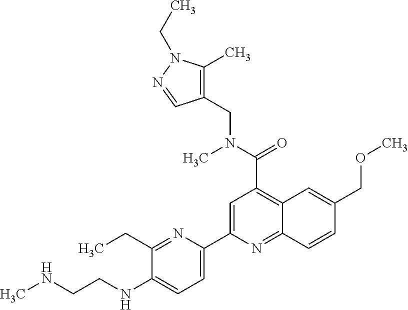 | 529.3 | 530.3 | 0.05161 | 0.1446 | 0.09907 | 0.0599 |
| 596 | 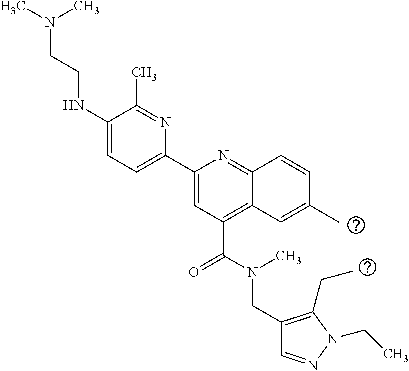 [SMILES: CCc1c(CN(C)C(=O)c2cc(-c3ccc(NCCN(C)C)c(C)n3)nc3ccc(C)cc23)cnn1CC] | 537.2 | 538.2 | 0.1797 | 0.5866 | 0.9979 | 0.6927 |
| 597 | 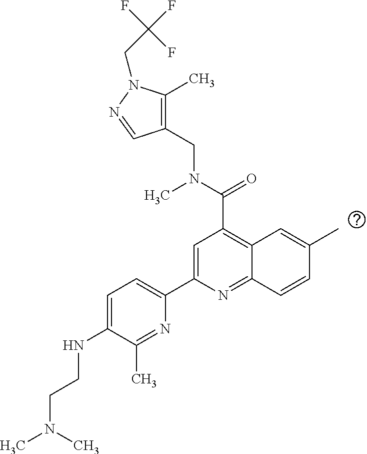 | 573.2 | 574.3 | 0.03616 | 0.07259 | 0.07871 | 0.0285 |
| 598 | 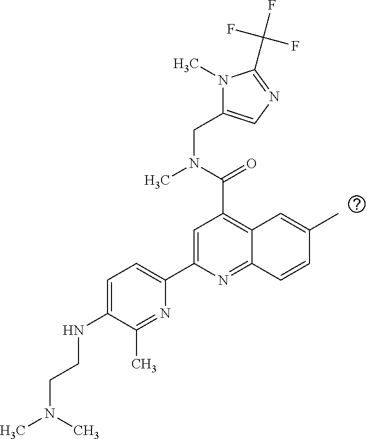 | 559.2 | 560.2 | 0.1055 | 0.3161 | 0.2828 | 0.2818 |
| 599 | 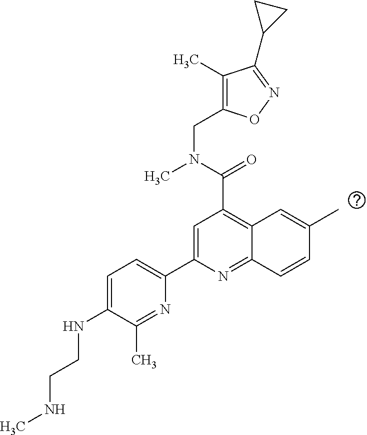 | 518.2 | 519.3 | 0.5177 | 1.603 | 1.259 | 1.75 |
| 600 | 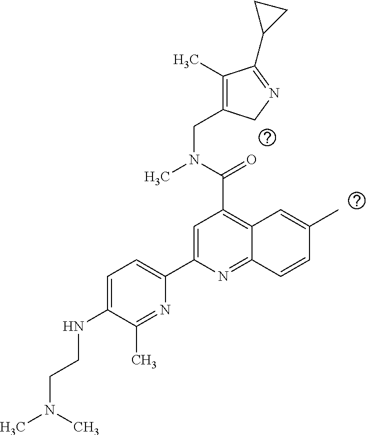 | 532.2 | 533.3 | 2.588 | 1.543 | 1.155 | 3.835 |
| 601 | 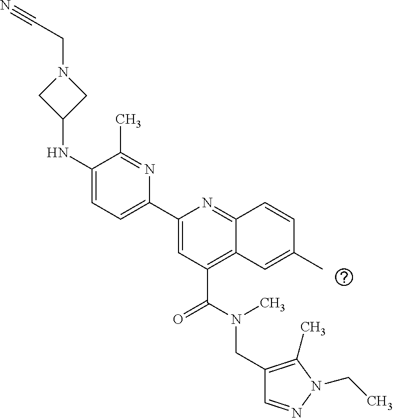 [SMILES: CCn1ncc(CN(C)C(=O)c2cc(-c3ccc(NC4CN(CC#N)C4)c(C)n3)nc3ccc(C)cc23)c1C] | 542.2 | 543.3 | 0.04489 | 0.1428 | 0.07258 | 0.0302 |
| 602 | 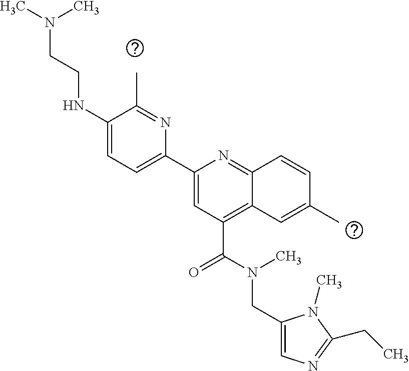 [SMILES: CCc1ncc(CN(C)C(=O)c2cc(-c3ccc(NCCN(C)C)c(C)n3)nc3ccc(C)cc23)n1C] | 523.2 | 524.2 | 0.04278 | 0.303 | 0.3001 | 0.0513 |
| 603 | 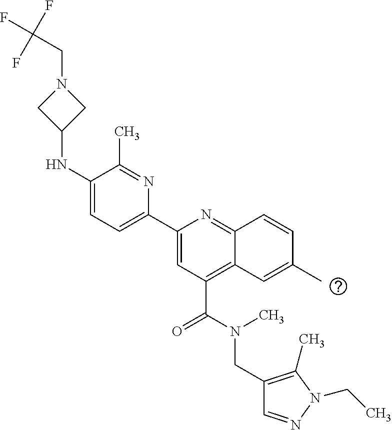 [SMILES: CCn1ncc(CN(C)C(=O)c2cc(-c3ccc(NC4CN(CC(F)(F)F)C4)c(C)n3)nc3ccc(C)cc23)c1C] | 585.2 | 586.3 | 0.04867 | 0.1745 | 0.253 | 0.0203 |
| 604 | 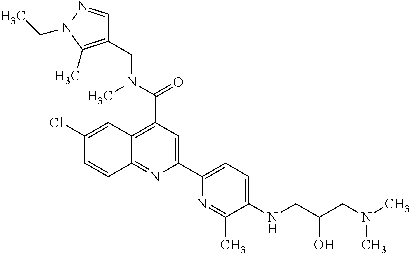 [SMILES: CCn1ncc(CN(C)C(=O)c2cc(-c3ccc(NCC(O)CN(C)C)c(C)n3)nc3ccc(Cl)cc23)c1C] | 551.3 | 552.3 | 0.0285 | 0.06481 | 0.02838 | 0.0114 |
| 605 | 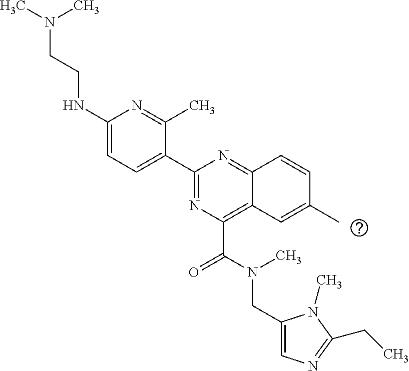 [SMILES: CCc1ncc(CN(C)C(=O)c2nc(-c3ccc(NCCN(C)C)nc3C)nc3ccc(C)cc23)n1C] | 520.2 | 521.2 | 0.5328 | 0.9431 | 0.4473 | 3.088 |
| 606 | 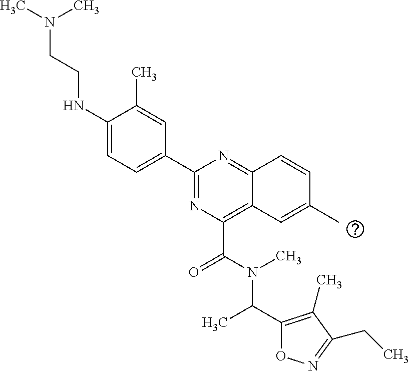 [SMILES: CCc1noc(C(C)N(C)C(=O)c2nc(-c3ccc(NCCN(C)C)c(C)c3)nc3ccc(C)cc23)c1C] | 534.3 | 535.2 | 0.03033 | 0.2944 | 0.08072 | 0.014 |
| 607 | 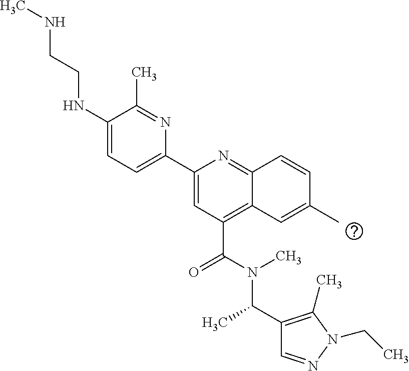 [SMILES: CCn1ncc([C@H](C)N(C)C(=O)c2cc(-c3ccc(NCCNC)c(C)n3)nc3ccc(C)cc23)c1C] | 519.3 | 520.3 | 6.51 | 3.267 | 1.967 | 3.377 |
| 608 | 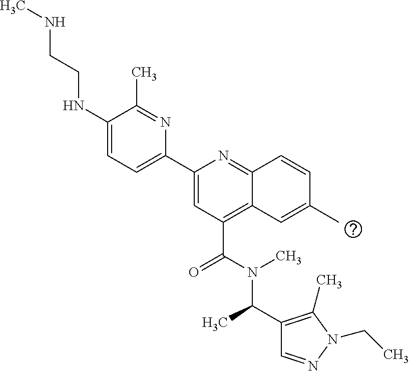 [SMILES: CCn1ncc([C@@H](C)N(C)C(=O)c2cc(-c3ccc(NCCNC)c(C)n3)nc3ccc(C)cc23)c1C] | 519.3 | 520.3 | 0.0928 | 0.1071 | 0.03897 | 0.0264 |
| 609 | 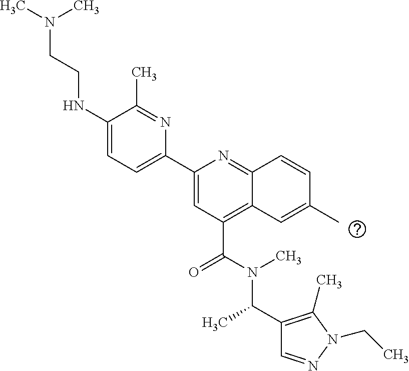 [SMILES: CCn1ncc([C@H](C)N(C)C(=O)c2cc(-c3ccc(NCCN(C)C)c(C)n3)nc3ccc(C)cc23)c1C] | 533.3 | 534.3 | 15.68 | 2.727 | 1.514 | 5.529 |
| 610 | 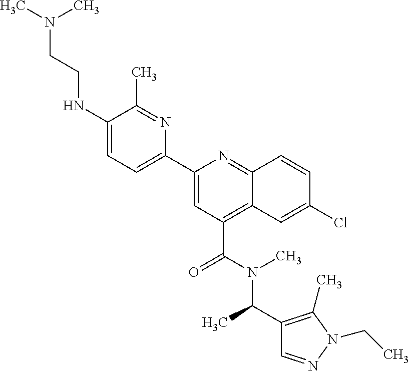 [SMILES: CCn1ncc([C@@H](C)N(C)C(=O)c2cc(-c3ccc(NCCN(C)C)c(C)n3)nc3ccc(Cl)cc23)c1C] | 533.3 | 534.3 | 0.05957 | 0.08504 | 0.03775 | 0.0209 |
| 611 | 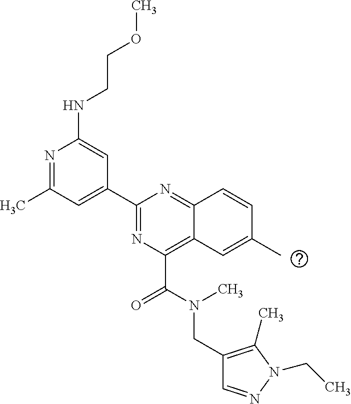 [SMILES: CCn1ncc(CN(C)C(=O)c2nc(-c3cc(C)nc(NCCOC)c3)nc3ccc(C)cc23)c1C] | 507.2 | 508.2 | 0.07361 | 0.3446 | 0.3119 | 0.4926 |
| 612 | 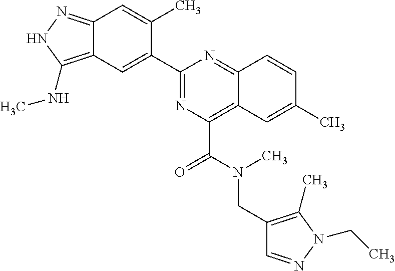 [SMILES: CCn1ncc(CN(C)C(=O)c2nc(-c3cc4c(NC)[nH]nc4cc3C)nc3ccc(C)cc23)c1C] | 482.3 | 483.3 | 0.01805 | 0.04631 | 0.04086 | 0.0066 |
| 613 | 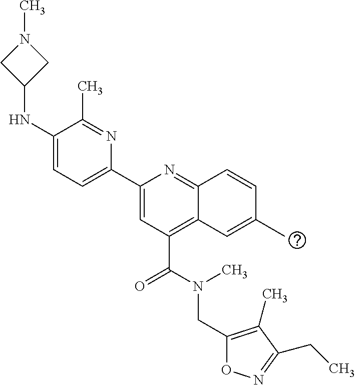 [SMILES: CCc1noc(CN(C)C(=O)c2cc(-c3ccc(NC4CN(C)C4)c(C)n3)nc3ccc(C)cc23)c1C] | 518.2 | 519.2 | 0.01908 | 0.05377 | 0.05175 | 0.0112 |
| 614 | 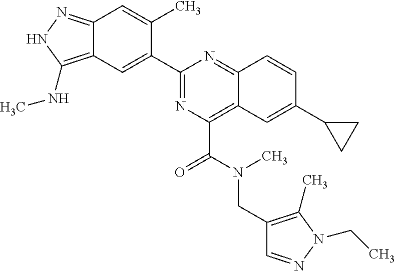 [SMILES: CCn1ncc(CN(C)C(=O)c2nc(-c3cc4c(NC)[nH]nc4cc3C)nc3ccc(C4CC4)cc23)c1C] | 508.3 | 509.2 | 0.02294 | 0.03209 | 0.03463 | 0.0087 |
| 615 | 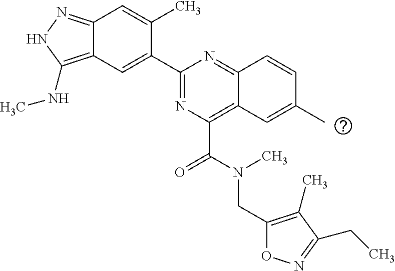 [SMILES: CCc1noc(CN(C)C(=O)c2nc(-c3cc4c(NC)[nH]nc4cc3C)nc3ccc(C)cc23)c1C] | 503.2 | 504.2 | 0.009745 | 0.02915 | 0.0195 | 0.0031 |
| 616 | 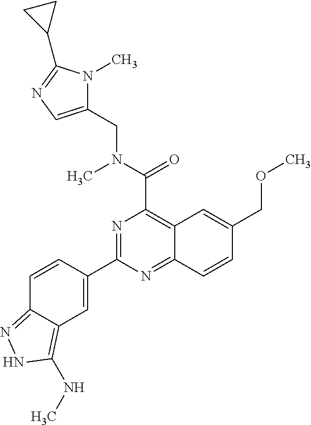 [SMILES: CNc1[nH]nc2ccc(-c3nc(C(=O)N(C)Cc4cnc(C5CC5)n4C)c4cc(COC)ccc4n3)cc12] | 510.2 | 511.3 | 0.0251 | 0.1023 | 0.1597 | 0.0144 |
| 617 | 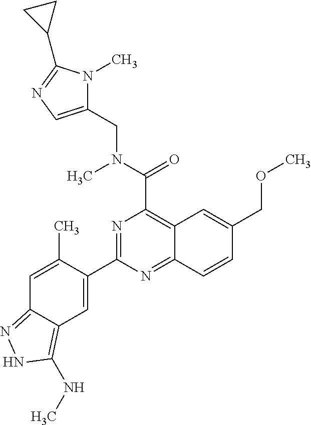 [SMILES: CNc1[nH]nc2cc(C)c(-c3nc(C(=O)N(C)Cc4cnc(C5CC5)n4C)c4cc(COC)ccc4n3)cc12] | 524.3 | 525.2 | 0.04084 | 0.08762 | 0.08413 | 0.0133 |
| 618 | 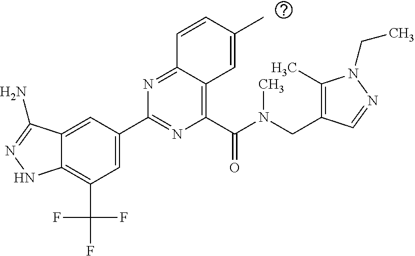 [SMILES: CCn1ncc(CN(C)C(=O)c2nc(-c3cc(C(F)(F)F)c4[nH]nc(N)c4c3)nc3ccc(C)cc23)c1C] | 542.2 | 543.2 | 0.05375 | 0.2482 | 0.7006 | 0.2848 |
| 619 | 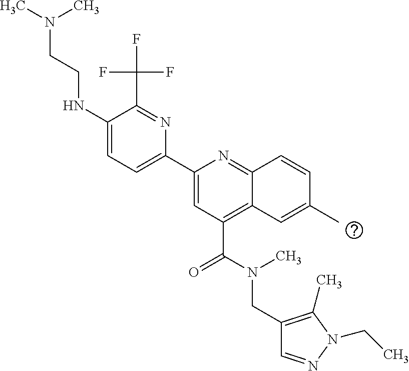 [SMILES: CCn1ncc(CN(C)C(=O)c2cc(-c3ccc(NCCN(C)C)c(C(F)(F)F)n3)nc3ccc(C)cc23)c1C] | 573.2 | 574.2 | 0.02379 | 0.05991 | 0.03249 | 0.0145 |
| 620 | 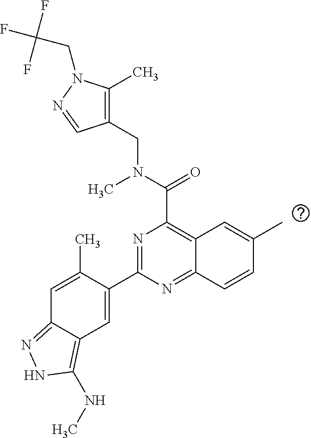 [SMILES: CNc1[nH]nc2cc(C)c(-c3nc(C(=O)N(C)Cc4cnn(CC(F)(F)F)c4C)c4cc(C)ccc4n3)cc12] | 556.2 | 557.2 | 0.02515 | 0.06721 | 0.0777 | 0.0123 |
| 621 | 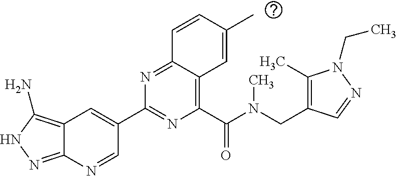 [SMILES: CCn1ncc(CN(C)C(=O)c2nc(-c3cnc4n[nH]c(N)c4c3)nc3ccc(C)cc23)c1C] | 475.2 | 476.1 | 0.216 | 0.2221 | 0.3913 | 0.3972 |
| 622 | 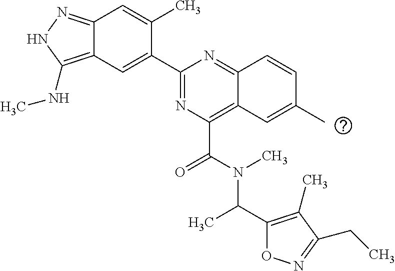 [SMILES: CCc1noc(C(C)N(C)C(=O)c2nc(-c3cc4c(NC)[nH]nc4cc3C)nc3ccc(C)cc23)c1C] | 517.2 | 518.1 | 0.1072 | 1.323 | 2.3 | 0.3826 |
| 623 | 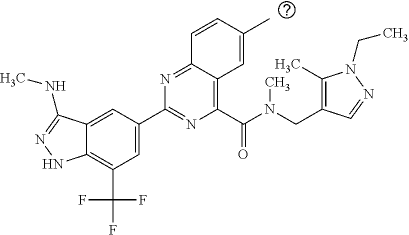 [SMILES: CCn1ncc(CN(C)C(=O)c2nc(-c3cc(C(F)(F)F)c4[nH]nc(NC)c4c3)nc3ccc(C)cc23)c1C] | 556.2 | 557 | 0.04386 | 0.2299 | 0.711 | 0.0283 |
| 624 | 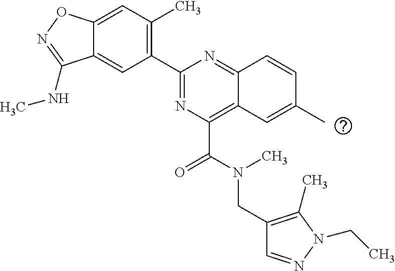 [SMILES: CCn1ncc(CN(C)C(=O)c2nc(-c3cc4c(NC)noc4cc3C)nc3ccc(C)cc23)c1C] | 503.2 | 504.1 | 0.01835 | 0.1126 | 0.2838 | 0.0092 |
| 625 | 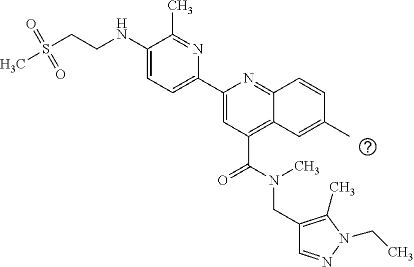 [SMILES: CCn1ncc(CN(C)C(=O)c2cc(-c3ccc(NCCS(C)(=O)=O)c(C)n3)nc3ccc(C)cc23)c1C] | 554.2 | 555.1 | 0.01921 | 0.114 | 0.163 | 0.0113 |
| 626 | 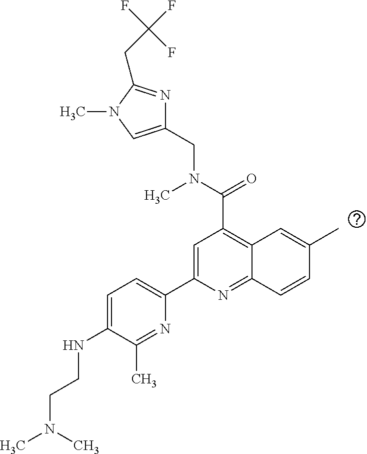 | 573.2 | 574.2 | 3.31 | 3.212 | 5.776 | 4.238 |
| 627 | 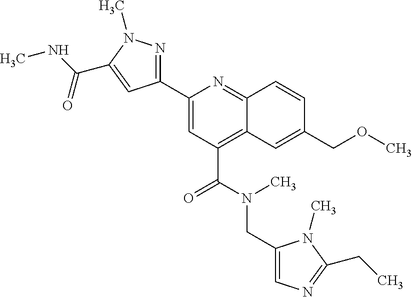 | 489.2 | 490.1 | 0.04956 | 0.2712 | 0.506 | 0.0156 |
| 628 | 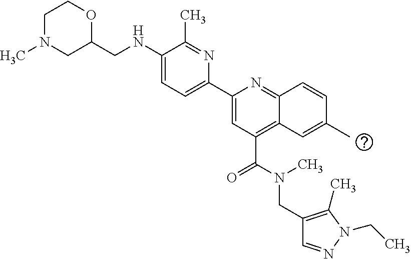 [SMILES: CCn1ncc(CN(C)C(=O)c2cc(-c3ccc(NCC4CN(C)CCO4)c(C)n3)nc3ccc(C)cc23)c1C] | 561.3 | 562.1 | 3.629 | 3.296 | 0.997 | 1.731 |
| 629 | 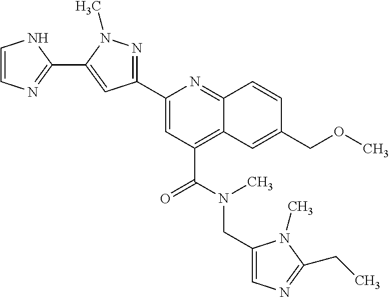 [SMILES: CCc1ncc(CN(C)C(=O)c2cc(-c3cc(-c4ncc[nH]4)n(C)n3)nc3ccc(COC)cc23)n1C] | 498.2 | 499.1 | 0.034 | 0.2561 | 0.292 | 0.0168 |
| 630 | 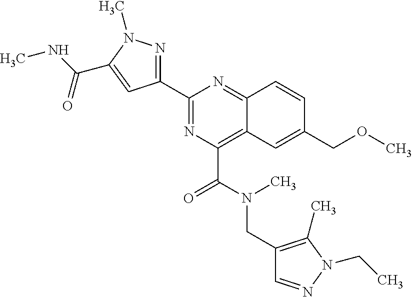 | 490.2 | 491.2 | 0.1488 | 0.341 | | 0.072 |

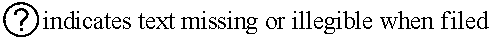

TABLE 3
Exemplary Compounds and Data

| | | | | CREBBP | | | EP300 |
|---|---|---|---|---|---|---|---|
| | | | Mass | Bioche- | CREBBP | CREBBP | Bioche- |
| Com- | | | De- | mistry | ICW | HTP | mistry |
| pound | | | tec- | IC50 | IC50 | IC50 | IC50 |
| Num- | | | ted | (micro- | (micro- | (micro- | (micro- |
| ber | STRUCTURE | MW | M + 1 | molar) | molar) | molar) | molar) |
| 631 | 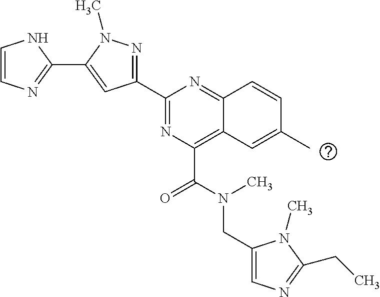 [SMILES: CCc1ncc(CN(C)C(=O)c2nc(-c3cc(-c4ncc[nH]4)n(C)n3)nc3ccc(C)cc23)n1C] | 489.2 | 490.1 | 0.0167 | 0.1246 | 0.1939 | 0.0088 |
| 632 | 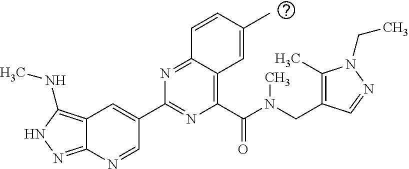 [SMILES: CCn1ncc(CN(C)C(=O)c2nc(-c3cnc4n[nH]c(NC)c4c3)nc3ccc(C)cc23)c1C] | 489.2 | 490.1 | 0.0032 | 0.0421 | 0.105 | 0.0046 |
| 633 | 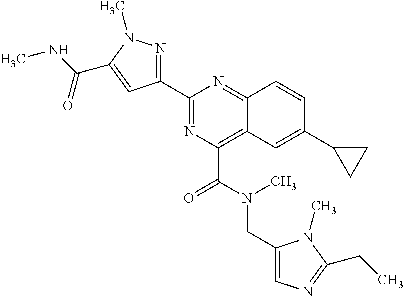 [SMILES: CCc1ncc(CN(C)C(=O)c2nc(-c3cc(C(=O)NC)n(C)n3)nc3ccc(C4CC4)cc23)n1C] | 486.2 | 487.1 | 0.0343 | 0.2266 | 0.3688 | 0.0146 |
| 634 | 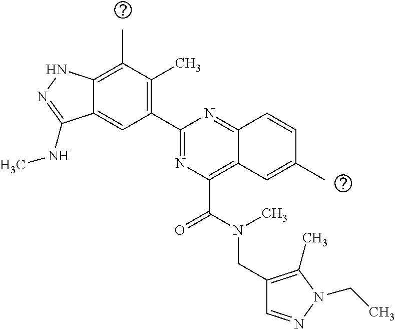 [SMILES: CCn1ncc(CN(C)C(=O)c2nc(-c3cc4c(NC)n[nH]c4c(C)c3C)nc3ccc(C)cc23)c1C] | 520.2 | 521.2 | 50 | 20 | 20 | 50 |
| 635 | 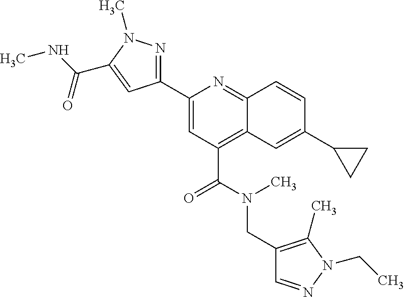 [SMILES: CCn1ncc(CN(C)C(=O)c2cc(-c3cc(C(=O)NC)n(C)n3)nc3ccc(C4CC4)cc23)c1C] | 485.3 | 486.1 | 0.0242 | 0.0526 | 0.143 | 0.0066 |
| 636 | 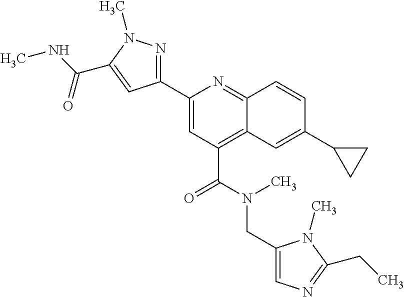 [SMILES: CCc1ncc(CN(C)C(=O)c2cc(-c3cc(C(=O)NC)n(C)n3)nc3ccc(C4CC4)cc23)n1C] | 485.3 | 486.1 | 0.0248 | 0.0654 | 0.1324 | 0.0056 |
| 637 | 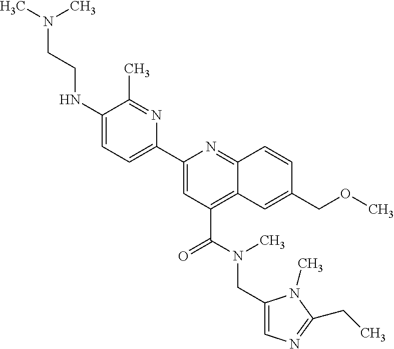 | 529.3 | 530.2 | 0.0987 | 0.4199 | 0.2949 | 0.0344 |
| 638 | 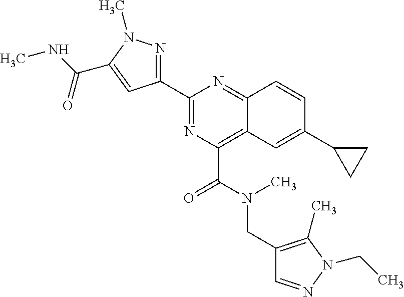 [SMILES: CCn1ncc(CN(C)C(=O)c2nc(-c3cc(C(=O)NC)n(C)n3)nc3ccc(C4CC4)cc23)c1C] | 486.2 | 487.1 | 0.1162 | 0.1241 | 0.357 | 0.0662 |
| 639 | 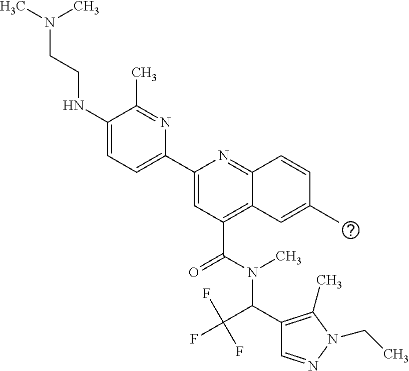 [SMILES: CCn1ncc(C(N(C)C(=O)c2cc(-c3ccc(NCCN(C)C)c(C)n3)nc3ccc(C)cc23)C(F)(F)F)c1C] | 587.2 | 588.1 | 50 | 20 | 2.171 | 50 |
| 640 | 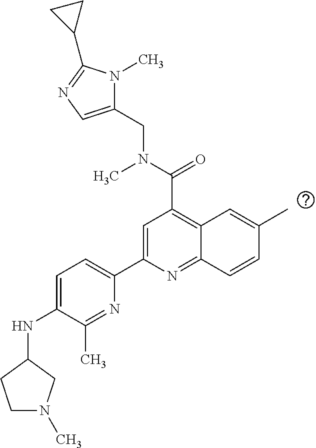 [SMILES: Cc1ccc2nc(-c3ccc(NC4CCN(C)C4)c(C)n3)cc(C(=O)N(C)Cc3cnc(C4CC4)n3C)c2c1] | 543.3 | 544.1 | 0.0246 | 0.1353 | 0.0487 | 0.009 |
| 641 | 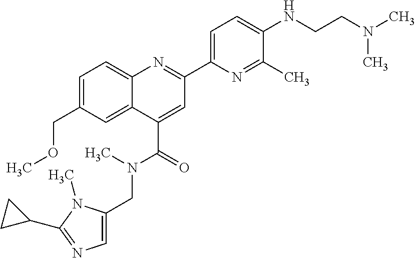 [SMILES: COCc1ccc2nc(-c3ccc(NCCN(C)C)c(C)n3)cc(C(=O)N(C)Cc3cnc(C4CC4)n3C)c2c1] | 541.3 | 542.2 | 0.0733 | 0.1997 | 0.111 | 0.0221 |
| 642 | 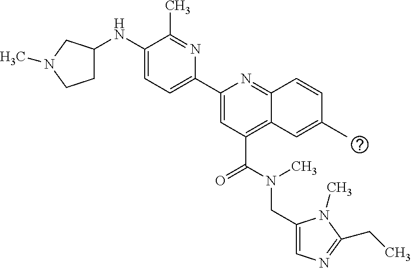 [SMILES: CCc1ncc(CN(C)C(=O)c2cc(-c3ccc(NC4CCN(C)C4)c(C)n3)nc3ccc(C)cc23)n1C] | 531.3 | 532.1 | 0.0281 | 0.0866 | 0.0334 | 0.0091 |
| 643 | 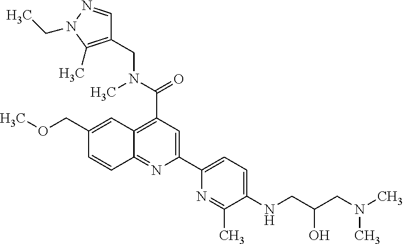 | 559.3 | 560.2 | | 0.327 | 0.233 | |
| 644 | 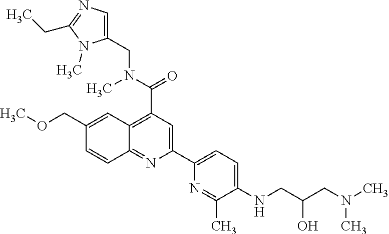 | 559.3 | 560.2 | | 0.7005 | 0.2692 | |
| 645 | 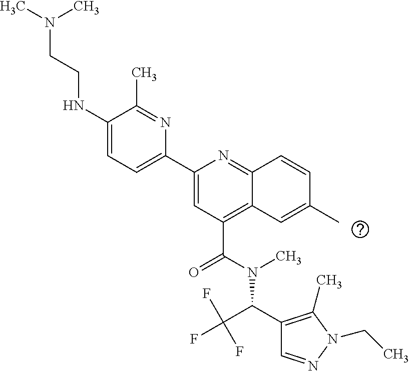 [SMILES: CCn1ncc([C@@H](N(C)C(=O)c2cc(-c3ccc(NCCN(C)C)c(C)n3)nc3ccc(C)cc23)C(F)(F)F)c1C] | 587.2 | 588.1 | | 19.95 | 2.43 | |
| 646 | 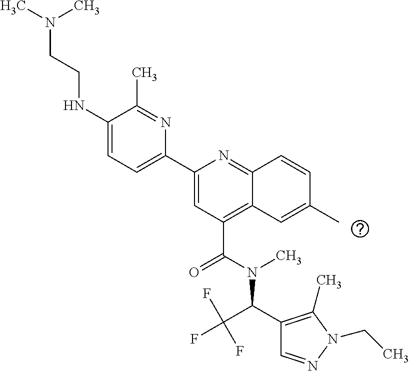 [SMILES: CCn1ncc([C@H](N(C)C(=O)c2cc(-c3ccc(NCCN(C)C)c(C)n3)nc3ccc(C)cc23)C(F)(F)F)c1C] | 587.2 | 588.1 | | 19.15 | 2.768 | |
| 647 | 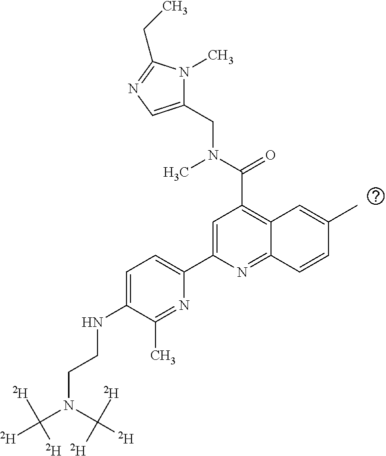 [SMILES: [2H]C([2H])([2H])N(CCNc1ccc(-c2cc(C(=O)N(C)Cc3cnc(CC)n3C)c3cc(C)ccc3n2)nc1C)C([2H])([2H])[2H]] | 525.3 | 526.2 | | 0.1569 | 0.0597 | |

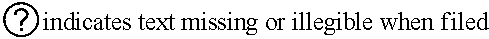

1. A compound represented by the following formula: 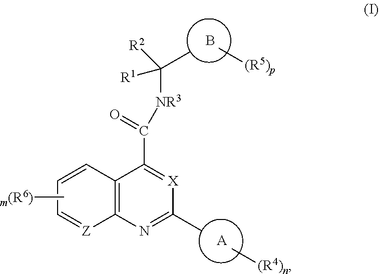
or a pharmaceutically acceptable salt thereof, wherein:
X is CH or N;
Z is N, CH, or CR⁶;
Ring A is a monocyclic or bicyclic aryl or a monocyclic or bicyclic heterocyclyl;
Ring B is a 5-membered N-containing heteroaryl;
R¹ and R² are each independently selected from H, C₁₋₆alkyl, halo, —CN, —C(O)R^{1a}, —C(O)₂R^{1a}, —C(O)N(R^{1a})₂, —N(R^{1a})₂, —N(R^{1a})C(O)R^{1a}, —N(R^{1a})C(O)₂R^{1a}, —N(R^{1a})C(O)N(R^{1a})₂, —N(R^{1a})S(O)₂R^{1a}, —OR^{1a}, —OC(O)R^{1a}, —OC(O)N(R^{1a})₂, —SR^{1a}, —S(O)R^{1a}, —S(O)₂R^{1a}, —S(O)N(R^{1a})₂, and —S(O)₂N(R^{1a})₂;
R^{1a} in each occurrence is independently selected from H, C₁₋₆alkyl, C₂₋₆alkenyl, C₂₋₆alkynyl, carbocyclyl, and heterocyclyl, or two R^{1a} together with the nitrogen atom from which they are attached form a 4 to 7-membered ring, wherein the 4 to 7-membered ring optionally contains 1 or 2 heteroatoms independently selected from N, O, and S;
R³ is H or C₁₋₆alkyl;
R⁴ in each occurrence is independently selected from C₁₋₆alkyl, C₂₋₆alkenyl, C₂₋₆alkynyl, carbocyclyl, heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OC(O)R^{4a}, —OC(O)N(R^{4a})₂, —SR^{4a}, —S(O)R^{4a}, —S(O)₂R^{4a}, —S(O)N(R^{4a})₂, —S(O)₂N(R^{4a})₂ and P(O)(R^{4a})₂;
R^{4a} in each occurrence is independently selected from H, C₁₋₆alkyl, C₂₋₆alkenyl, C₂₋₆alkynyl, carbocyclyl, heterocyclyl, and P(O)(R^{7a})₂, or two R^{4a} together with the nitrogen atom from which they are attached form a 4 to 7-membered ring, wherein the 4 to 7-membered ring optionally contains 1 or 2 heteroatoms independently selected from N, O and S;
R⁵ in each occurrence is independently C₁₋₆alkyl or carbocyclyl, or two R⁵ together with the atoms from which they are attached form a 4 to 7-membered ring, wherein the 4 to 7-membered ring optionally contains 1 or 2 heteroatoms independently selected from N, O and S;
R⁶ in each occurrence is independently selected from C₁₋₆alkyl, C₂₋₆alkenyl, C₂₋₆alkynyl, carbocyclyl, heterocyclyl, halo, —CN, —C(O)R^{6a}, —C(O)₂R^{6a}, —C(O)N(R^{6a})₂, —N(R^{6a})₂, —N(R^{6a})C(O)R^{6a}, —N(R^{6a})C(O)₂R^{6a}, —N(R^{6a})C(O)N(R^{6a})₂, —N(R^{6a})S(O)₂R^{6a}, —OC(O)R^{6a}, —OC(O)N(R^{6a})₂, —SR^{6a}, —S(O)R^{6a}, —S(O)₂R^{6a}, —S(O)N(R^{6a})₂, —S(O)₂N(R^{6a})₂, and —P(O)(R^{6a})₂;
R^{6a} in each occurrence is independently selected from H, C₁₋₆alkyl, C₂₋₆alkenyl, C₂₋₆alkynyl, carbocyclyl, and heterocyclyl; or two R^{6a} together with the nitrogen atom from which they are attached form a 4 to 7-membered ring, wherein the 4 to 7-membered ring optionally contains 1 or 2 heteroatoms independently selected from N, O, and S;
m is 0, 1, 2, or 3;
p is 0, 1, 2 or 3; and
n is 0, 1, 2, 3, 4, 5, or 6;
wherein each C₁₋₆alkyl, C₂₋₆alkenyl, C₂₋₆alkynyl, carbocyclyl, and heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, —SR⁷, —S(O)R⁷, —S(O)₂R⁷, —S(O)N(R⁷)₂, —S(O)₂N(R⁷)₂, and —P(O)(R⁷)₂, and
R⁷ in each occurrence is independently selected from H, C₁₋₆alkyl, C₂₋₆alkenyl, C₂₋₆alkynyl, carbocyclyl, and heterocyclyl, wherein each C₁₋₆alkyl, C₂₋₆alkenyl, C₂₋₆alkynyl, carbocyclyl, and heterocyclyl are optionally substituted with one or more substituents independently selected from R^{7a}, halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)R^{7a}, —OC(O)N(R^{7a})₂, —SR^{7a}, —S(O)R^{7a}, —S(O)₂R^{7a}, —S(O)N(R^{7a})₂, —S(O)₂N(R^{7a})₂, and —P(O)R^{7a}; and
R^{7a} in each occurrence is independently selected from H and C₁₋₄alkyl.

2. The compound of claim 1, wherein X is N and Z is N.

3. The compound of claim 1, wherein only one of X and Z is N.

4. The compound of claim 1, wherein the compound is represented by the following formula: 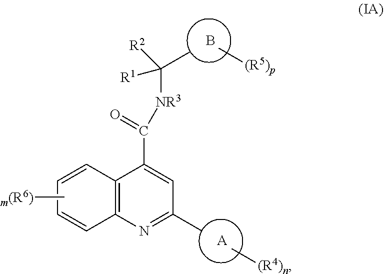
or a pharmaceutically acceptable salt thereof.

5. The compound of claim 1, wherein the compound is represented by the following formula: 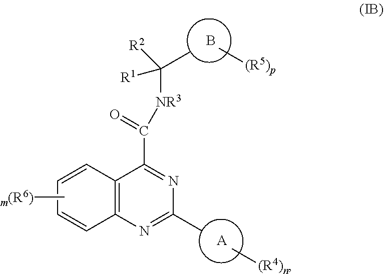
or a pharmaceutically acceptable salt thereof.

6. The compound of claim 1, wherein the compound is represented by the following formula: 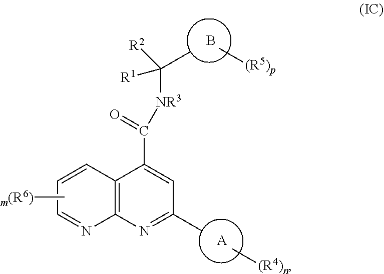
or a pharmaceutically acceptable salt thereof.

7. The compound of any one of claims 1-6, wherein Ring B is a N-containing heteroaryl including one nitrogen atom.

8. The compound of any one of claims 1-6, wherein Ring B is a N-containing heteroaryl including two nitrogen atoms.

9. The compound of any one of claims 1-6, wherein Ring B is pyrrole, pyrazole, imidazole, oxazole, isoxazole, thiazole or isothiazole.

10. The compound of any one of claims 1-6, wherein Ring B is pyrazole or imidazole.

11. The compound of any one of claims 1-6, wherein Ring B is pyrazole.

12. The compound of any one of claims 1-6, wherein Ring B is imidazole.

13. The compound of any one of claims 1-12, wherein R¹ and R² are each independently selected from H, C₁₋₆alkyl, and halo.

14. The compound of any one of claims 1-13, wherein R¹ is H and R² is C₁₋₆alkyl or halo.

15. The compound of any one of claims 1-13, wherein R¹ and R² are both H.

16. The compound of any one of claims 1-15, wherein R¹ and R² are both H, and R³ is methyl.

17. The compound of any one of claims 1-6 and 8-16, wherein the compound is represented by the following formula: 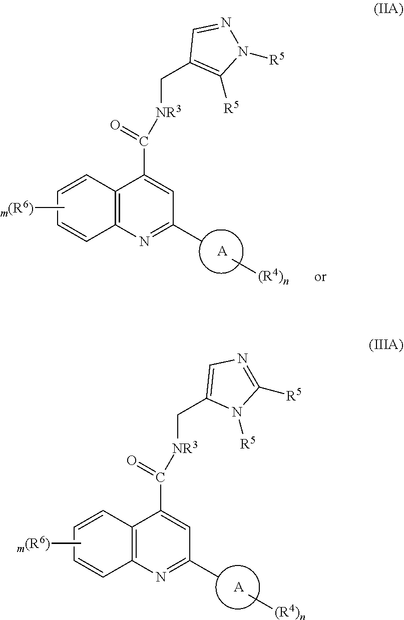
or a pharmaceutically acceptable salt thereof.

18. The compound of any one of claims 1-6 and 8-16, wherein the compound is represented by the following formula: 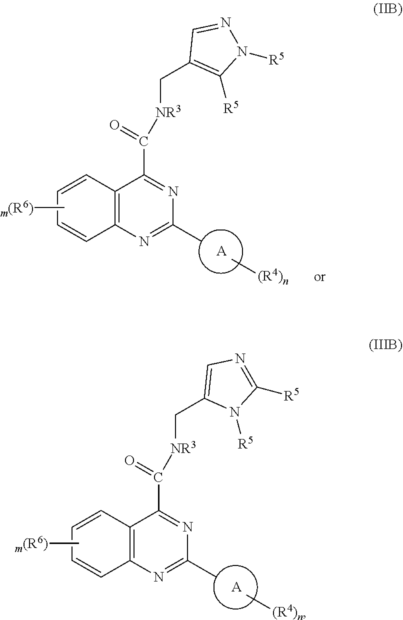
or a pharmaceutically acceptable salt thereof.

19. The compound of any one of claims 1-6 and 8-16, wherein the compound is represented by the following formula: 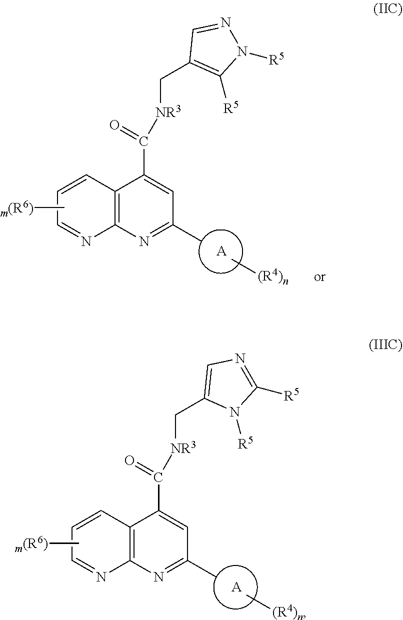
or a pharmaceutically acceptable salt thereof.

20. The compound of any one of claims 1-6 and 8-16, wherein the compound is represented by the following formula: 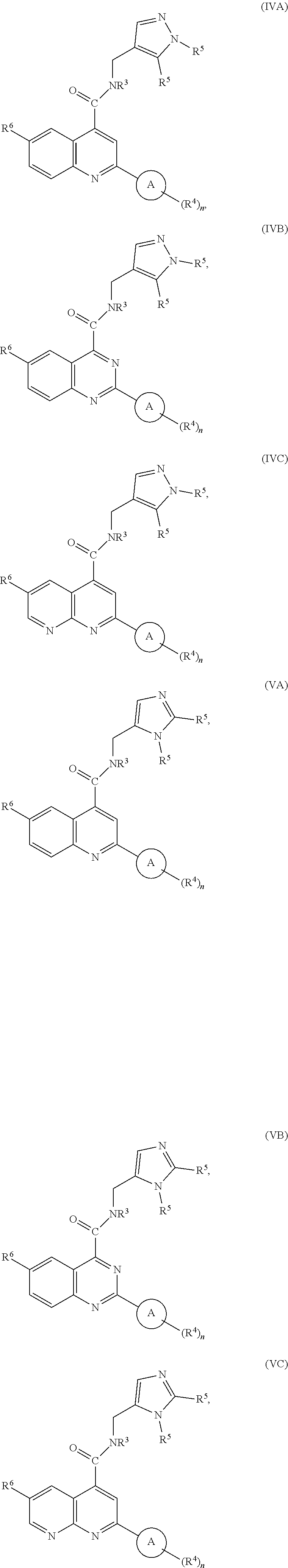
or a pharmaceutically acceptable salt thereof.

21. The compound of any one of claims 1-6 and 8-16, wherein the compound is represented by the following formula: 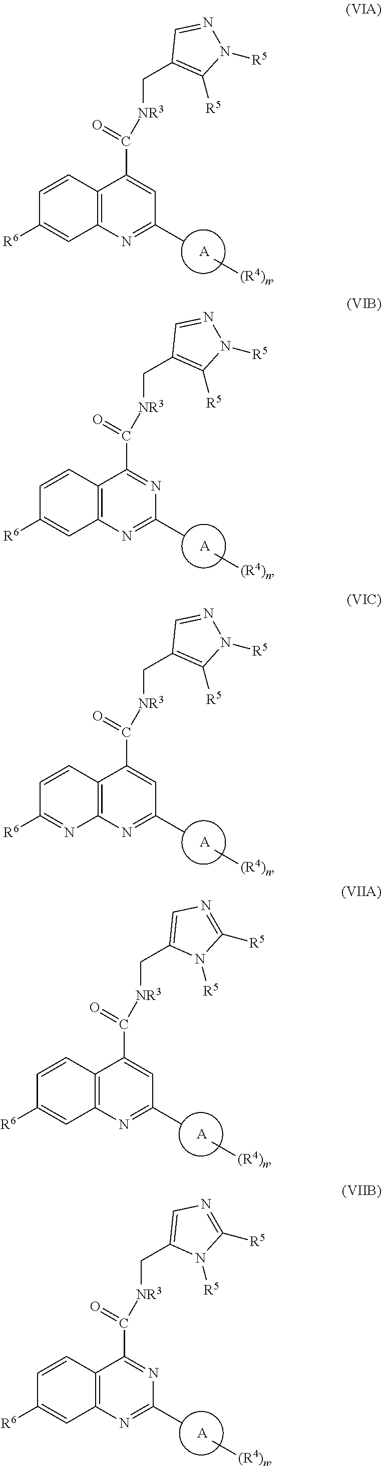 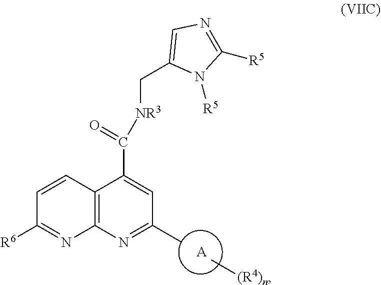
or a pharmaceutically acceptable salt thereof.

22. The compound of any one of claims 1-21, wherein:
R⁶ in each occurrence is independently selected from C₁₋₆alkyl, phenyl, 4 to 6-membered heterocyclyl, halo, —CN, —OR^{6a}, —N(R^{6a})₂, —S(O)₂R^{6a}, and —P(O)(R^{6a})₂; and
R^{6a} in each occurrence is independently selected from H and C₁₋₆alkyl;
wherein each of the C₁₋₆alkyl, phenyl and 5 to 6-membered heterocyclyl are optionally substituted with one or more substituents independently selected from halo, —N(R⁷)₂, —OR⁷ and phenyl optionally substituted with one or more substituents independently selected from —CN, halo, and —OR^{7a};
R⁷ is H or C₁₋₄alkyl; and
R^{7a} in each occurrence is independently selected from H and C₁₋₄alkyl.

23. The compound of claim 22, wherein:
R⁶ is Cl, Br, F, —CN, —OCH₃, —CH₃, —CH₂CH₃, —OCH₂CH₃, —NH₂, —NHCH₃, —N(CH₃)₂, —C₂H₄NHCH₃, —OCH₂CH(OH)CH₂NHCH₃, morpholine, or —CH₂OCH₃.

24. The compound of any one of claims 1-21, wherein R⁶ is —OR^{6a}.

25. The compound of claim 24, wherein R^{6a} is C₁₋₆alkyl.

26. The compound of any one of claims 1-21, wherein R⁶ is C₁₋₆alkyl substituted with —OR⁷, wherein R⁷ is H or C₁₋₆alkyl.

27. The compound of any one of claims 1-21, wherein R⁶ is halogen.

28. The compound of claim 27, wherein R⁶ is fluoro.

29. The compound of claim 27, wherein R⁶ is chloro.

30. The compound of any one of claims 1-29, wherein R³ is H or C₁₋₆alkyl optionally substituted with halo, —OR⁷, or —N(R⁷)₂; and R⁷ is H or C₁₋₃alkyl.

31. The compound of claim 30, wherein R³ is C₁₋₃alkyl optionally substituted with halo, —OH or C₁₋₃alkoxy.

32. The compound of any one of claims 1-31, wherein R³ is H, methyl, ethyl, —CH₂CH₂OH.

33. The compound of claim 32, wherein R³ is methyl or ethyl.

34. The compound of any one of claims 1-33, wherein R⁵ in each occurrence is independently selected from C₁₋₄alkyl and C₃₋₆cycloalkyl, wherein each of the C₁₋₄alkyl and C₃₋₆cycloalkyl are optionally substituted with one to three halogen.

35. The compound of claim 34, wherein R⁵ in each occurrence is independently selected from methyl, ethyl, propyl, isopropyl, cyclopropyl and —CH₂CF₃.

36. The compound of claim 34, wherein R⁵ in each occurrence is independently C₁₋₄alkyl.

37. The compound of any one of claims 1-16 and 22-36, wherein 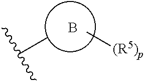
has the structure

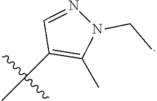 [SMILES: *c1cnn(CC)c1C]

38. The compound of any one of claims 1-16 and 22-36, wherein 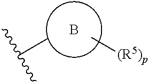
has the structure

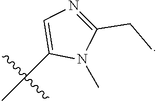 [SMILES: *c1cnc(CC)n1C]

39. The compound of any one of claims 1-16 and 22-36, wherein:
R¹ and R² are both H;
R³ is methyl; and 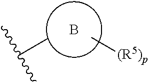
has the structure

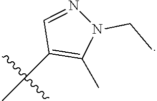 [SMILES: *c1cnn(CC)c1C]

40. The compound of any one of claims 1-16 and 22-36, wherein:
R¹ and R² are both H;
R³ is methyl; and 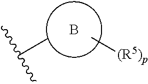
has the structure

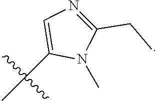 [SMILES: *c1cnc(CC)n1C]

41. The compound of any one of claims 1-40, wherein m is 0.

42. The compound of any one of claims 1-41, Ring A is phenyl, 5 or 6-membered heteroaryl, 9 or 10-membered bicyclic heteroaryl, 5 to 7-membered saturated monocyclic heterocyclyl, or 9- and 10-membered bicyclic non-aromatic heterocyclyl.

43. The compound of claim 42, wherein Ring A is phenyl or 5- or 6-membered heteroaryl.

44. The compound of claim 42, wherein Ring A is phenyl, pyridine, benzotriazole, benzoimidazole, thiazole, pyrrole, pyrazole, indole, imidazole, isoxazole, isothiazole, pyrrolidine, piperidine, piperazine, pyrimidine, triazole, 1H-indazole, 2H-indazole, 1,4-diazepane, 4,5,6,7-tetrahydro-1H-imidazo[4,5-c]pyridine, 4,5,6,7-tetrahydro-1H-pyrazolo[3,4-c]pyridine, 4,5,6,7-tetrahydro-2H-pyrazolo[3,4-c]pyridine, 4,5,6,7-tetrahydro-1H-imidazo[4,5-c]pyridine, 5,6,7,8-tetrahydro-[1,2,4]triazolo[1,5-a]pyrazine, or 5,6,7,8-tetrahydroimidazo[1,2-a]pyrazine.

45. The compound of any one of claims 1-41, wherein Ring A is: 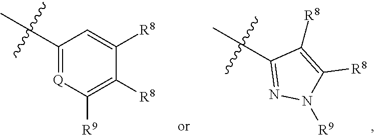
wherein R⁸ in each occurrence is independently selected from C₁₋₆alkyl, C₂₋₆alkenyl, C₂₋₆alkynyl, carbocyclyl, heterocyclyl, halo, —CN, —C(O)R^{8a}, —C(O)₂R^{8a}, —C(O)N(R^{8a})₂, —N(R^{8a})₂, —N(R^{8a})C(O)R^{8a}, —N(R^{8a})C(O)₂R^{8a}, —N(R^{8a})C(O)N(R^{8a})₂, —N(R^{8a})S(O)₂R^{8a}, —OR^{8a}, —OC(O)R^{8a}, —OC(O)N(R^{8a})₂, —SR^{8a}, —S(O)R^{8a}, —S(O)₂R^{8a}, —S(O)N(R^{8a})₂, and —S(O)₂N(R^{8a})₂; or two R⁸ together with the carbon atoms from which they are attached form a 4 to 7-membered ring, wherein the 4 to 7-membered ring optionally contains 1, 2 or 3 heteroatoms independently selected from N, O, and S;
R^{8a} is in each occurrence is independently selected from H, C₁₋₆alkyl, C₂₋₆alkenyl, C₂₋₆alkynyl, carbocyclyl, and heterocyclyl, or two R^{8a} together with the nitrogen atom from which they are attached form a 4 to 7-membered ring, wherein the 4 to 7-membered ring optionally contains 1 or 2 heteroatoms independently selected from N, O and S;
R⁹ is selected from C₁₋₆alkyl, C₂₋₆alkenyl, C₂₋₆alkynyl, carbocyclyl, heterocyclyl, halo, —CN, —C(O)R^{9a}, —C(O)₂R^{9a}, —C(O)N(R^{9a})₂, —N(R^{9a})₂, —N(R^{9a})C(O)R^{9a}, —N(R^{9a})C(O)₂R^{9a}, —N(R^{9a})C(O)N(R^{9a})₂, —N(R^{9a})S(O)₂R^{9a}, —OC(O)R^{9a}, —OC(O)N(R^{9a})₂, —SR^{9a}, —S(O)R^{9a}, —S(O)₂R^{9a}, —S(O)N(R^{9a})₂, —S(O)₂N(R^{9a})₂, and —P(O)(R^{9a})₂;
R^{9a} in each occurrence is independently selected from H, C₁₋₆alkyl, C₂₋₆alkenyl, C₂₋₆alkynyl, carbocyclyl, and heterocyclyl, or two R^{9a} together with the nitrogen atom from which they are attached form a 4 to 7-membered ring, wherein the 4 to 7-membered ring optionally contains 1 or 2 heteroatoms independently selected from N, O and S; and
Q is N, CH or CR⁸;
wherein each C₁₋₆alkyl, C₂₋₆alkenyl, C₂₋₆alkynyl, carbocyclyl, and heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, —SR⁷, —S(O)R⁷, —S(O)₂R⁷, —S(O)N(R⁷)₂, —S(O)₂N(R⁷)₂, and —P(O)(R⁷)₂.

46. The compound of claim 45, wherein R⁹ is methyl or halogen.

47. The compound of claim 45, wherein R⁹ is chloro.

48. The compound of any one of claims 1-47, wherein:
R⁴ in each occurrence is independently selected from C₁₋₆alkyl, C₃₋₆cycloalkyl, 5 to 6-membered heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OC(O)R^{4a}, —OC(O)N(R^{4a})₂, and —S(O)₂R^{4a};
R^{4a} in each occurrence is independently selected from H, C₁₋₆alkyl, C₃₋₆cycloalkyl, and 5 to 6-membered heterocyclyl;
wherein each C₁₋₆alkyl, C₃₋₆cycloalkyl, and 5 to 6-membered heterocyclyl above are optionally substituted with one or more substituents independently selected from R⁷, halo, —CN, —C(O)R⁷, —C(O)₂R⁷, —C(O)N(R⁷)₂, —N(R⁷)₂, —N(R⁷)C(O)R⁷, —N(R⁷)C(O)₂R⁷, —N(R⁷)C(O)N(R⁷)₂, —N(R⁷)S(O)₂R⁷, —OR⁷, —OC(O)R⁷, —OC(O)N(R⁷)₂, and —S(O)₂R⁷, and
R⁷ in each occurrence is independently selected from H, C₁₋₆alkyl, phenyl, C₃₋₆cycloalkyl, and 5 to 6-membered heterocyclyl, wherein each C₁₋₆alkyl, phenyl, C₃₋₆cycloalkyl, and 5 to 6-membered heterocyclyl are optionally substituted with one or more substituents independently selected from R^{7a}, halo, —CN, —C(O)R^{7a}, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, —OC(O)R^{7a}, —OC(O)N(R^{7a})₂ and —S(O)₂R^{7a}; and
R^{7a} in each occurrence is independently selected from H and C₁₋₄alkyl.

49. The compound of claim 48, wherein:
R⁴ in each occurrence is independently selected from H, Cl, F, Br, —CN, NH₂, —CH₃, —CH₂CH₃, —CF₃, —CH₂OH, —CH₂OCH₃, —CH₂NHCH₃, —CH₂N(CH₃)₂, —C₂H₄OCH₃, —C₂H₄NHCH₃, —C₃H₆OH, —CH₂-NH-tetrahydopyran, —C₃H₆NHCH₃, -cyclopropyl, pyrazole, azetidine, pyrrolidine, morpholine, —CH₂-pyrrolidine, —C₃H₆-pyrrolidine, —CH₂NH-tetrahydropyran, —CH₂-piperazine, —CH₂-morpholine, —CH₂-phenyl-OCH₃, —CH₂CH₂CN, —OCH₃, —OC₂H₄OH, —OC₃H₆OH, —OC₃H₆-piperidine, —OC₂H₄-pyrrolidine, —OC₃H₆-pyrrolidine, —OC₃H₆-tetrahydropyran, —OCH₂CH(OH)CH₂NHCH₃, —OC₂H₄OCH₃, —OC₂H₄NH₂, —OC₂H₄NHCH₃, —OC₃H₆NHCH₃, —OC₂H₄NHC(O)CH₃, —OC₂H₄N(CH₃)S(O)₂CH₃, —CH₂C(O)NH₂, —CH₂C(O)NHCH₃, —C(O)NHCH₃, —C(O)NHC₃H₆-pyrrolidine, —C(O)NHC₂H₄-pyrrolidine, —C(O)NH₂, —C(O)NHCH₃, —S(O)₂CH₃, —C(O)CH₃, —N(CH₃)₃, —NHC(O)CH₃, —NHCH₃, —NH-piperidine, —NHC₂H₄NHCH₃, —NHC₃H₆NHCH₃, —NHC(O)NHCH₃, —NHC(O)OC₄H₉, —NH(CO)CH₂NHCH₃, —NHC₂H₄N(CH₃)C(O)OC₄H₉, —C₂H₄NHCOOC₄H₉, —CH₂N(CH₃)C(O)OC₄H₉, —C₂H₄N(CH₃)C(O)OC₄H₉, —C₃H₆NHC(O)OC₄H₉, —C₃H₆N(CH₃)C(O)OC₄H₉, —OC₂H₄C(O)NHCH₃, —OC₂H₄NHC(O)OC₄H₉, —OC₂H₄N(CH₃)C(O)OC₄H₉, —OC₃H₆NHC(O)OC₄H₉, —OC₃H₆N(CH₃)C(O)OC₄H₉, —C(O)OC₄H₉, —C₃H₆-pyrrolidine, —CH₂CH₂CH(OH)CH₂-pyrrolidine, —NH-piperidine, —NH-(N-methyl)piperidine, —NH-tetrahydropyran, —OCH₂CH(OH)CH₂NHCH₃—OCH₂CH₂NHCH₃—CH₂CH₂CH(OH)CH₂NHCH₃, —C(O)NH-tetrahydropyridine, —C(O)NH-piperidine, 1-(4-methoxybenzyl), —C(O)NH—C₃H₆-pyrrolidine, —C(O)NH—C₂H₄-pyrrolidine, —O—Ph—CH₂N(CH₃)₂, pyrrolidine-C(O)OC₄H₉, —NH—C₂H₄-pyrrolidine, OCH₂CH(OH)CH₂-pyrrolidine, —OCH₂CH₂-pyrrolidine, —CO—NH—N-1-methylpiperidin-4-yl), —OCH₂CH(OH)CH₂-pyrrolidine and

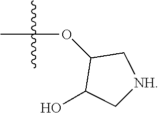 [SMILES: *OC1CNCC1O]

50. The compound of claim 1, wherein the compound is represented by the following formula: 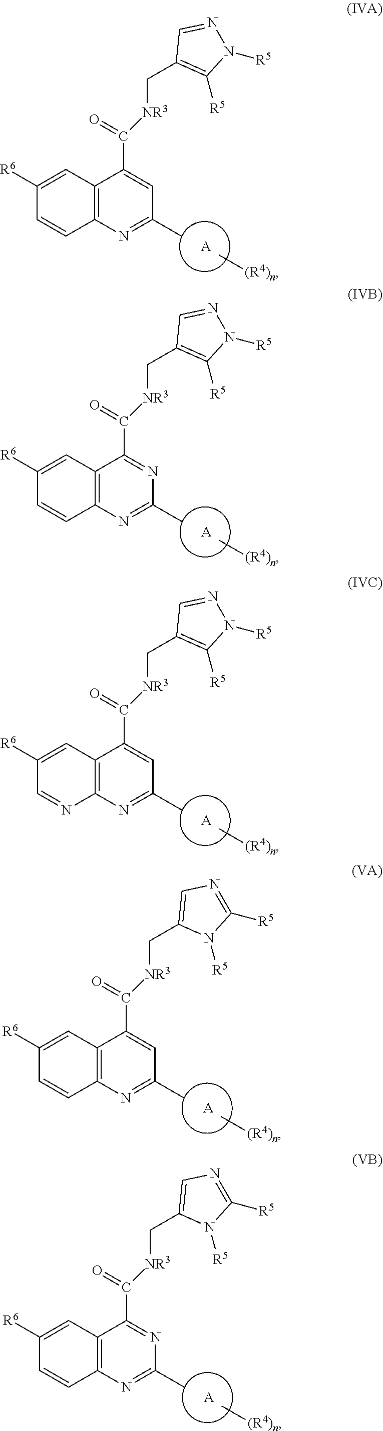 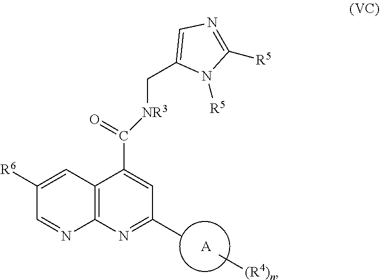
or a pharmaceutically acceptable salt thereof, wherein:
R³ is C₁₋₃alkyl optionally substituted with halo, —OH, or C₁₋₃alkoxy;
R⁵ in each occurrence is independently selected from C₁₋₄alkyl, and C₃₋₆cycloalkyl, wherein the C₁₋₄alkyl and C₃₋₆cycloalkyl are optionally substituted with one to three halogen;
R⁶ is halo, C₁₋₄alkyl, or 4 to 6-membered saturated heterocyclyl, wherein the C₁₋₄alkyl and 4 to 6-membered saturated heterocyclyl are optionally substituted with one or more substituents independently selected from halo, —OR⁷ and —N(R⁷)₂;
R⁷ is H or C₁₋₃alkyl;
Ring A is phenyl or 5 or 6-membered heteroaryl;
R⁴ in each occurrence is independently selected from C₁₋₆alkyl, C₃₋₆cycloalkyl, 5 to 6-membered heterocyclyl, halo, —CN, —C(O)R^{4a}, —C(O)₂R^{4a}, —C(O)N(R^{4a})₂, —N(R^{4a})₂, —N(R^{4a})C(O)R^{4a}, —N(R^{4a})C(O)₂R^{4a}, —N(R^{4a})C(O)N(R^{4a})₂, —N(R^{4a})S(O)₂R^{4a}, —OR^{4a}, —OC(O)R^{4a}, —OC(O)N(R^{4a})₂, and —S(O)₂R^{4a};
R^{4a} in each occurrence is independently selected from H, C₁₋₆alkyl, C₃₋₆cycloalkyl, and 5 to 6-membered heterocyclyl;
wherein each C₁₋₆alkyl, C₃₋₆cycloalkyl, and 5 to 6-membered heterocyclyl above are optionally substituted with one or more substituents independently selected from R⁷, halo, —CN, —C(O)N(R^{7a})₂, —N(R⁷)₂, —N(R⁷)C(O)R⁷, —N(R⁷)C(O)₂R⁷, —N(R⁷)S(O)₂R⁷, and —OR⁷, and
R⁷ in each occurrence is independently selected from H, C₁₋₆alkyl, phenyl, C₃₋₆cycloalkyl, and 5 to 6-membered heterocyclyl, wherein each C₁₋₆alkyl, phenyl, C₃₋₆cycloalkyl, a 5 to 6-membered heterocyclyl are optionally substituted with one or more substituents independently selected from R^{7a}, halo, —C(O)₂R^{7a}, —C(O)N(R^{7a})₂, —N(R^{7a})₂, —N(R^{7a})C(O)R^{7a}, —N(R^{7a})C(O)₂R^{7a}, —N(R^{7a})C(O)N(R^{7a})₂, —N(R^{7a})S(O)₂R^{7a}, and —OR^{7a};
R^{7a} in each occurrence is independently selected from H and C₁₋₄alkyl; and
n is 0, 1, or 2.

51. The compound of claim 50, wherein the compound is represented by the following formula: 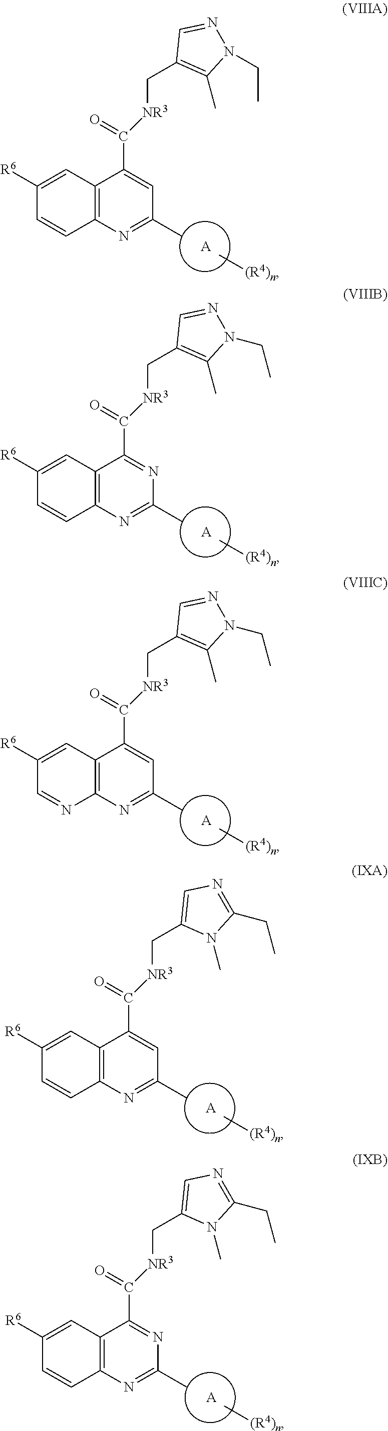 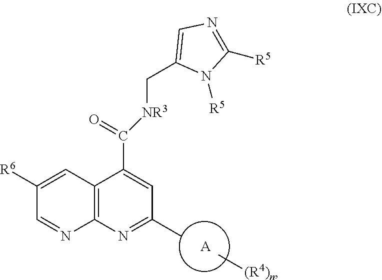
or a pharmaceutically acceptable salt thereof.

52. The compound of claim 50 or 51, wherein:
R³ is C₁₋₃alkyl;
R⁵ in each occurrence is independently C₁₋₄alkyl; and
R⁶ is halo.

53. The compound of claim 52, wherein R³ is methyl, R⁵ in each occurrence is independently methyl, ethyl or isopropyl; and R⁶ is chloro.

54. A compound, wherein the compound has a structure as shown in Table 1.

55. A compound, wherein the compound has a structure as shown in Table 2.

56. A compound, wherein the compound has a structure as shown in Table 3.

57. The compound of any one of claims 1-56, wherein the compound is a pharmaceutically acceptable salt.

58. A pharmaceutical composition comprising a compound of any one of claims 1-57 and a pharmaceutically acceptable carrier.

59. A method of treating cancer in a subject in need thereof comprising administering to the subject a therapeutically effective amount of a compound of any one of claims 1-57.
